SEQUENCE LISTING (2 sequences)

SEQ ID NO: 1 - protein, 11 aa, baculovirus infected T.ni cells
MKKGHHHHHHD

SEQ ID NO: 2 - protein, 9 aa, Artificial Sequence
  optimized PLK peptide substrate
SFNDTLDFD

(19) United States
(12) Patent Application Publication
(10) Pub. No.: US 2006/0074119 A1
(43) Pub. Date: Apr. 6, 2006

(54) Thiophene compounds

(76) Inventors: Clarence W Andrews III, Durham, NC (US); Mui Cheung, Durham, NC (US); Ronda G Davis-Ward, Durham, NC (US); David Harold Drewry, Durham, NC (US); Kyle Allen Emmitte, Durham, NC (US); Robert Dale Hubbard, Durham, NC (US); Kevin W Kuntz, Durham, NC (US); James Andrew Linn, Durham, NC (US); Robert Anthony Mook JR., Durham, NC (US); Gary Keith Smith, Durham, NC (US); James Marvin Veal, Apex, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/522,958

(22) PCT Filed: Aug. 4, 2003
(86) PCT No.: PCT/US03/24272

§ 371 Date: Jan. 31, 2005

Related U.S. Application Data
(60) Provisional application No. 60/402,008, filed on Aug. 8, 2002.

(51) Int. Cl. A61K 31/4184; C07D 409/14

(52) U.S. Cl. 514/394; 548/304.4; 514/234.2; 514/227.8; 514/254.06; 514/322; 544/60; 544/139; 544/368; 546/199

(57) ABSTRACT

The present invention provides compounds of formula (1): 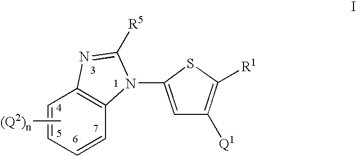 pharmaceutical compositions containing the same, processes for preparing the same and their use as pharmaceutical agents.

### BACKGROUND OF THE INVENTION

The present invention relates to novel compounds, pharmaceutical formulations comprising these compounds, and the use of these compounds in therapy. More particularly, the present invention relates to novel compounds and methods for treating conditions mediated by Polo-like Kinase, susceptible neoplasms, and other conditions.

Polo-like kinases (“PLK”) are evolutionarily conserved serine/threonine kinases that play critical roles in regulating processes in the cell cycle. PLK plays a role in the entry into and the exit from mitosis in diverse organisms from yeast to mammalian cells. PLK includes PLK1, PLK2, and PLK3.

Polo-like kinases are known to be essential for mitosis in yeast, Drosophila, and Xenopus. For example, mutants of the homologous PLK genes in these organisms result in disordered mitotic spindles, and in Drosophila mutations can be embryonic lethal. RNA interference experiments on Drosophila polo have shown that ablation of polo in S2 cells results in G2/M arrest and apoptosis. PLK1 is the human homolog of Drosophila polo. It is believed to be involved in the entry into mitosis through the activation of cdk1 by phosphorylating and activating the phosphatase cdc25C, which in turn removes inhibitory phosphates from cdk1. This sets up an activation loop for cdk1 that leads to mitotic entry. PLK1 also phosphorylates cyclin B1, the cyclin partner of cdk1, resulting in nuclear localization. During mitosis, PLK1 has been shown to play roles in centrosome maturation and microtubule dynamics involved in formation of the mitotic spindle. PLK1 is also involved in the exit of cells from mitosis by phosphorylating and activating subunits of the anaphase-promoting complex (cdc16 and cdc27). PLK1 also phosphorylates cohesin proteins that hold sister chromatids together, exposing separase cleavage sites, and allowing separation of sister chromatids during anaphase. PLK1 may also play a role in cytokinesis through phosphorylation of the kinesin-like motor protein MKLP1. Inhibition of PLK1 thus has the potential to interfere with several stages of mitosis. Expression and activity of PLK protein increases during the cell cycle, reaching its peak during mitosis when it is also maximally phosphorylated. PLK1 mRNA is highly expressed in cells with a high mitotic index. PLK2 (serum-inducible kinase, SNK) and PLK3 (Proliferation-related kinase PRK Fibroblast Growth Factor-inducible kinase, FNK) were originally identified as immediate-early genes. PLK2 is not very well characterized, but PLK3 appears to be involved in regulation of cell cycle progression through M phase but functions differently from PLK1. Recent published work suggests that PLK3 plays an important role in the regulation of microtubule dynamics and function of the centrosome during mitosis.

Overexpression of PLK1 appears to be strongly associated with neoplastic cells (including cancers). A published study has shown high levels of PLK1 RNA expression in >80% of lung and breast tumors, with little to no expression in adjacent normal tissue. Several studies have shown correlations between PLK expression, histological grade, and prognosis in several types of cancer. Significant correlations were found between percentages of PLK-positive cells and histological grade of ovarian and endometrial cancer (P<0.001). These studies noted that PLK is strongly expressed in invading endometrial carcinoma cells and that this could reflect the degree of malignancy and proliferation in endometrial carcinoma. Using RT-PCR analysis, PLK overexpression was detected in 97% of esophageal carcinomas and 73% of gastric carcinomas as compared to the corresponding normal tissues. Further, patients with high levels of PLK overexpression in esophageal carcinoma represented a significantly poorer prognosis group than those with low levels of PLK overexpression. In head and neck cancers, elevated mRNA expression of PLK1 was observed in most tumors; a Kaplan-Meier analysis showed that those patients with moderate levels of PLK1 expression survived longer than those with high levels of PLK1 expression. Analysis of patients with non-small cell lung carcinoma showed similar outcomes related to PLK1 expression.

Disruption of mitosis with anti-microtubule drugs has been a successful approach in cancer chemotherapy. The taxanes and vinca alkaloids have been effectively used in the clinic, but they have undesirable side effects. In addition, many tumors appear to have weakened G2/M cell cycle checkpoints; in response to mitotic disruption these tumors attempt to bypass mitosis, leading to mitotic catastrophe and cell death. Several studies suggest that the disruption of mitosis by targeting PLK may be a feasible approach to selective tumor cell destruction. There remains a need in the art for new approaches to the treatment of neoplasms.

### BRIEF SUMMARY OF THE INVENTION

According to a first aspect of the invention there is provided a compound of formula (I): 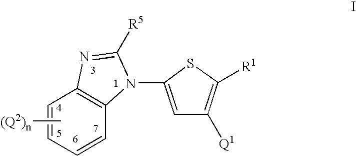
wherein:
R¹ is selected from the group consisting of H, alkyl, alkenyl, alkynyl, —C(O)R⁷, —CO₂R⁷, —C(O)NR⁷R⁸, —C(O)N(R⁷)OR⁸, —C(O)N(R⁷)—R²—OR⁸, —C(O)N(R⁷)-Ph, —C(O)N(R⁷)—R²-Ph, —C(O)N(R⁷)C(O)R⁸, —C(O)N(R⁷)CO₂R⁸, —C(O)N(R⁷)C(O)NR⁷R⁸, —C(O)N(R⁷)S(O)₂R⁸, —R²—OR⁷, —R²—O—C(O)R⁷, —C(S)R⁷, —C(S)NR⁷R⁸, —C(S)N(R⁷)-Ph, —C(S)N(R⁷)—R²-Ph, —R²—SR⁷, —C(═NR⁷)NR⁷R⁸, —C(═NR⁷)N(R⁸)-Ph, —C(═NR⁷)N(R⁸)—R²-Ph, —R²—NR⁷R⁸, —CN, —OR⁷, —S(O)_{f}R⁷⁷—S(O)₂NR⁷R⁸, —S(O)₂N(R⁷)-Ph, —S(O)₂N(R⁷)—R²-Ph, —NR⁷R⁸, —N(R⁷)-Ph, —N(R⁷)—R²-Ph, —N(R⁷)—SO₂R⁸ and Het;
Ph is phenyl optionally substituted from 1 to 3 times with a substituent selected from the group consisting of halo, alkyl, —OH, —R²—OH, —O-alkyl, —R²—O-alkyl, —NH₂, —N(H)alkyl, —N(alkyl)₂, —CN and —N₃;
Het is a 5-7 membered heterocycle having 1, 2, 3 or 4 heteroatoms selected from N, O and S, or a 5-6 membered heteroaryl having 1, 2, 3 or 4 heteroatoms selected from N, O and S, each optionally substituted from 1 to 2 times with a substituent selected from the group consisting of halo, alkyl, oxo, —OH, —R²—OH, —O-alkyl, —R²—O-alkyl, —NH₂, —N(H)alkyl, —N(alkyl)₂, —CN and —N₃;
Q¹ is a group of formula:
   —(R²)ₐ—(Y¹)_{b}—(R²)_{c}—R³
a, b and c are the same or different and are each independently 0 or 1 and at least one of a or b is 1;
n is 0, 1, 2, 3 or 4;
Q² is a group of formula:
   —(R²)ₐₐ—(Y²)_{bb}—(R²)_{cc}—R⁴
   or two adjacent Q² groups are selected from the group consisting of alkyl, alkenyl, —OR⁷, —S(O)_{f}R⁷ and —NR⁷R⁸ and together with the carbon atoms to which they are bound, they form a C₅₋₆cycloalkyl, C₅₋₆cycloalkenyl, phenyl, 5-7 membered heterocycle having 1 or 2 heteroatoms selected from N, O and S, or 5-6 membered heteroaryl having 1 or 2 heteroatoms selected from N, O and S;
aa, bb and cc are the same or different and are each independently 0 or 1;
each Y¹ and Y² is the same or different and is independently selected from the group consisting of —O—, —S(O)_{f}—, —N(R⁷)—, —C(O)—, —OC(O)—, —CO₂—, —C(O)N(R⁷)—, —C(O)N(R⁷)S(O)₂—, —OC(O)N(R⁷)—, —OS(O)₂—, —S(O)₂N(R⁷)—, —S(O)₂N(R⁷)C(O)—, —N(R⁷)S(O)₂—, —N(R⁷)C(O)—, —N(R⁷)CO₂— and —N(R⁷)C(O)N(R⁷)—;
each R² is the same or different and is independently selected from the group consisting of alkylene, alkenylene and alkynylene;
each R³ and R⁴ is the same or different and is each independently selected from the group consisting of H, halo, alkyl, alkenyl, alkynyl, —C(O)R⁷, —C(O)NR⁷R⁸, —CO₂R⁷, —C(S)R⁷, —C(S)NR⁷R⁸, —C(═NR⁷) R⁸, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN, —N₃ and a group of formula (ii): 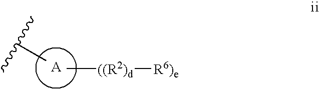
   wherein:
   Ring A is selected from the group consisting of C₅₋₁₀cycloalkyl, C₅₋₁₀cycyloalkenyl, aryl, 5-10 membered heterocycle having 1, 2 or 3 heteroatoms selected from N, O and S and 5-10 membered heteroaryl having 1, 2 or 3 heteroatoms selected from N, O and S
   each d is 0 or 1;
   e is 0, 1, 2, 3 or 4;
   each R⁶ is the same or different and is independently selected from the group consisting of H, halo, alkyl, alkenyl, alkynyl, cycloalkyl, cycloalkenyl, Ph, Het, —CH(OH)—R²—OH, —C(O)R⁷, —CO₂R⁷, —CO₂—R²-Ph, —CO₂—R²-Het, —C(O)NR⁷R⁸, —C(O)N(R⁷)C(O)R⁷, —C(O)N(R⁷)CO₂R⁷, —C(O)N(R⁷)C(O)NR⁷R⁸, —C(O)N(R⁷)S(O)₂R⁷, —C(S)R⁷, C(S)NR⁷R⁸, —C(═NR⁷)R⁸, —C(═NR⁷)NR⁷R⁸, —CR⁷═N—OR⁸, ═O, —OR⁷, —OC(O)R⁷, —OC(O)Ph, —OC(O)Het, —OC(O)NR⁷R⁸, —O—R²—S(O)₂R⁷, —S(O)_{f}R⁷, —S(O)₂NR⁷R⁸, —S(O)₂Ph, —S(O)₂Het, —NR⁷R⁸, —N(R⁷)C(O)R⁸, —N(R⁷)CO₂R⁸, —N(R⁷)—R²—CO₂R⁸, —N(R⁷)C(O)NR⁷R⁸, —N(R⁷)—R²—C(O)NR⁷R⁸, —N(R⁷)C(O)Ph, —N(R⁷)C(O)Het, —N(R⁷)Ph, —N(R⁷)Het, —N(R⁷)C(O)NR⁷—R²—NR⁷R⁸, —N(R⁷)C(O)N(R⁷)Ph, —N(R⁷)C(O)N(R⁷)Het, —N(R⁷)C(O)N(R⁷)—R²-Het, —N(R⁷)S(O)₂R⁸, —N(R⁷)—R²—S(O)₂R⁸, —NO₂, —CN and —N₃;
wherein when Q¹ is defined where b is 1 and c is 0, R³ is not halo, —C(O)R⁷, —C(O)NR⁷R⁸, —CO₂R⁷, —C(S)R⁷, —C(S)NR⁷R⁸, —C(═NR⁷)R⁸, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN or —N₃;
wherein when Q² is defined where bb is 1 and cc is 0, R⁴ is not halo, —C(O)R⁷, —C(O)NR⁷R⁸, —CO₂R⁷, —C(S)R⁷, —C(S)NR⁷R⁸, —C(═NR⁷)₂R⁸, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN or —N₃;
R⁵ is selected from the group consisting of H, halo, alkyl, cycloalkyl, OR⁷, —S(O)_{f}R⁷, —NR⁷R⁸, —NHC(O)R⁷, —NHC(O)NR⁷R⁸ and —NHS(O)₂R⁷;
f is 0, 1 or 2; and
each R⁷ and each R⁸ are the same or different and are each independently selected from the group consisting of H, alkyl, alkenyl, alkynyl, cycloalkyl and cycloalkenyl;
wherein when R₁ is —CO₂CH₃ and n is 0, Q¹ is not —OH;
   or a pharmaceutically acceptable salt, solvate or physiologically functional derivative thereof.

In another aspect of the invention there is provided a pharmaceutical composition comprising a compound of formula (I). In one embodiment, the pharmaceutical composition further comprises a pharmaceutically acceptable carrier, diluent or excipient.

In a third aspect of the invention, there is provided a method for treating a condition mediated by PLK in an animal. The method comprises administering to the animal a therapeutically effective amount of a compound of formula (I) or a pharmaceutically acceptable salt, solvate or physiologically functional derivative thereof.

In a fourth aspect of the invention, there is provided a method for treating a susceptible neoplasm in an animal. The method comprises administering to the animal a therapeutically effective amount of a compound of formula (I) or a pharmaceutically acceptable salt, solvate or physiologically functional derivative thereof. The susceptible neoplasm may be selected from the group consisting of breast cancer, colon cancer, lung cancer, prostate cancer, lymphoma, leukemia, endometrial cancer, melanoma, pancreatic cancer, ovarian cancer, squamous carcinoma, carcinoma of the head and neck, and esophageal carcinoma.

In a fifth aspect of the invention, there is provided a method for treating a condition characterized by inappropriate cellular proliferation. The method comprises contacting the cell with a therapeutically effective amount of a compound of formula (I) or a pharmaceutically acceptable salt, solvate or physiologically functional derivative thereof.

In a sixth aspect, the present invention provides a method for inhibiting proliferation of a cell. The method comprises contacting the cell with an amount of a compound of formula (I) or a pharmaceutically acceptable salt, solvate or physiologically functional derivative thereof sufficient to inhibit proliferation of the cell.

In another aspect, the present invention provides a method for inhibiting mitosis in a cell. The method comprises administering to the cell an amount of a compound of formula (I) or a pharmaceutically acceptable salt, solvate or physiologically functional derivative thereof sufficient to inhibit mitosis in the cell.

In another aspect, there is provided a process for preparing a compound of formula (I) comprising reacting a compound of formula (III): 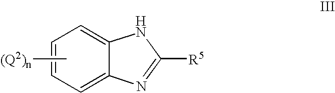
with a compound of formula (IV): 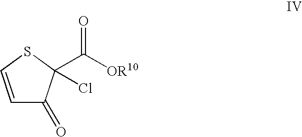
wherein R¹⁰ is selected from the group consisting of alkyl, alkenyl, alkynyl, cycloalkyl, cycloalkenyl and suitable carboxylic acid protecting groups.

In another aspect, the present invention provides a radiolabeled compound of formula (I) or a pharmaceutically acceptable salt, solvate or physiologically functional derivative thereof. In one embodiment, the present invention provides a tritiated compound of formula (I) or a pharmaceutically acceptable salt, solvate or physiologically functional derivative thereof. In another aspect, the present invention provides a biotinylated compound of formula (I) or a pharmaceutically acceptable salt, solvate or physiologically functional derivative thereof.

In another aspect, the present invention provides a compound of formula (I) or a pharmaceutically acceptable salt, solvate or physiologically functional derivative thereof for use in therapy.

In yet another aspect, the present invention provides a compound of formula (I) or a pharmaceutically acceptable salt, solvate or physiologically functional derivative thereof for use in the treatment of a condition mediated by PLK in an animal.

In yet another aspect, the present invention provides a compound of formula (I) or a pharmaceutically acceptable salt, solvate or physiologically functional derivative thereof for use in the treatment of a susceptible neoplasm in an animal.

In another aspect, the present invention provides a compound of formula (I) or a pharmaceutically acceptable salt, solvate or physiologically functional derivative thereof for use in the treatment of a condition characterized by inappropriate cellular proliferation.

In yet another aspect, the present invention provides a compound of formula (I) or a pharmaceutically acceptable salt, solvate or physiologically functional derivative thereof for use in inhibiting proliferation of a cell.

In yet another aspect, the present invention provides a compound of formula (I) or a pharmaceutically acceptable salt, solvate or physiologically functional derivative thereof for use in inhibiting mitosis in a cell.

In yet another aspect, the present invention provides the use of a compound of formula (I) or a pharmaceutically acceptable salt, solvate or physiologically functional derivative thereof for the preparation of a medicament for the treatment of condition mediated by PLK in an animal.

In yet another aspect, the present invention provides the use of a compound of formula (I) or a pharmaceutically acceptable salt, solvate or physiologically functional derivative thereof for the preparation of a medicament for the treatment of a susceptible neoplasm in an animal.

In yet another aspect, the present invention provides the use of a compound of formula (I) or a pharmaceutically acceptable salt, solvate or physiologically functional derivative thereof for the preparation of a medicament for the treatment of a condition characterized by inappropriate cellular proliferation in an animal.

In yet another aspect, the present invention provides the use of a compound of formula (I) or a pharmaceutically acceptable salt, solvate or physiologically functional derivative thereof for the preparation of a medicament for inhibiting proliferation of a cell.

In yet another aspect, the present invention provides the use of a compound of formula (I) or a pharmaceutically acceptable salt, solvate or physiologically functional derivative thereof for the preparation of a medicament for inhibiting mitosis in a cell. In yet another aspect, the present invention provides a pharmaceutical composition comprising a compound of formula (I) for use in the treatment of a susceptible neoplasm in an animal.

### DETAILED DESCRIPTION OF THE INVENTION

As used herein, “a compound of the invention” or “a compound of formula (I)” means a compound of formula (I) or a pharmaceutically acceptable salt, solvate, or physiologically functional derivative thereof. Similarly, with respect to isolatable intermediates such as for example, compounds of formula (III) and (VIII) the phrase “a compound of formula (number)” means a compound having that formula and pharmaceutically acceptable salts, solvates and physiologically functional derivatives thereof.

As used herein, the terms “alkyl” (and “alkylene”) refer to straight or branched hydrocarbon chains containing from 1 to 8 carbon atoms. Examples of “alkyl” as used herein include, but are not limited to, methyl, ethyl, n-propyl, n-butyl, n-pentyl, isobutyl, isopropyl, and tert-butyl. Examples of “alkylene” as used herein include, but are not limited to, methylene, ethylene, propylene, butylene, and isobutylene. “Alkyl” also includes substituted alkyl. The alkyl groups may be optionally substituted one or more times with a halogen. Thus, the term “alkyl” includes trifluoromethyl and trifluoroethyl, among other halogenated alkyls.

As used herein, the term “alkenyl” refers to straight or branched hydrocarbon chains containing from 2 to 8 carbon atoms (unless a different number of atoms is specified) and at least one and up to three carbon-carbon double bonds. Examples of “alkenyl” as used herein include, but are not limited to ethenyl and propenyl. “Alkenyl” also includes substituted alkenyl. The alkenyl groups may optionally be substituted one or more times with a halogen.

As used herein, the term “alkynyl” refers to straight or branched hydrocarbon chains containing from 2 to 8 carbon atoms (unless a different number of atoms is specified) and at least one and up to three carbon-carbon triple bonds. Examples of “alkynyl” as used herein include, but are not limited to ethynyl and propynyl. “Alkynyl” also includes substituted alkynyl. The alkynyl groups may optionally be substituted one or more times with a halogen.

As used herein, the term “cycloalkyl” refers to a non-aromatic monocyclic carbocyclic ring having from 3 to 8 carbon atoms (unless a different number of atoms is specified) and no carbon-carbon double bonds. “Cycloalkyl” includes by way of example cyclopropyl, cyclobutyl, cyclopentyl, cyclohexyl, cycloheptyl and cyclooctyl. “Cycloalkyl” also includes substituted cycloalkyl. The cycloalkyl may optionally be substituted on any available carbon with one or more substituents selected from the group consisting of halo, C₁₋₃alkyl (including haloalkyl, e.g., perfluoroalkyl), —OH, —O—C₁₋₃alkyl, —NH₂, —NH(C₁₋₃alkyl)-N(C₁₋₃alkyl)₂, —CN and —N₃. Preferred cycloalkyl groups include C₃₋₆cycloalkyl and substituted C₃₋₆cycloalkyl.

As used herein, the term “cycloalkenyl” refers to a non-aromatic monocyclic carbocyclic ring having from 3 to 8 carbon atoms (unless a different number of atoms is specified) and up to 3 carbon-carbon double bonds. “Cycloalkenyl” includes by way of example cyclobutenyl, cyclopentenyl and cyclohexenyl. “Cycloalkenyl” also includes substituted cycloalkenyl. The cycloalkenyl may optionally be substituted on any available carbon with one or more substituents selected from the group consisting of halo, C₁₋₃alkyl (including haloalkyl, e.g., perfluoroalkyl), —OH, —O—C₁₋₃alkyl, —NH₂, —NH(C₁₋₃alkyl)-N(C₁₋₃alkyl)₂, —CN and —N₃.

The term “halo” or “halogen” refers to fluorine, chlorine, bromine and iodine.

The term “oxo” as used herein refers to the group ═O attached directly to a carbon atom of a hydrocarbon ring (i.e., cycloalkenyl, aryl, heterocycle or heteroaryl ring) as well as —N-oxides, sulfones and sulfoxides wherein the N or S are atoms of a heterocyclic or heteroaryl ring.

The term “aryl” refers to monocyclic carbocyclic groups and fused bicyclic carbocyclic groups having from 6 to 13 carbon atoms (unless a different number of atoms is specified) and having at least one aromatic ring. Examples of particular aryl groups include but are not limited to phenyl and naphthyl. One particular aryl group according to the invention is phenyl.

The terms “heterocycle” and “heterocyclic” refer to monocyclic saturated or unsaturated non-aromatic groups and fused bicyclic saturated or unsaturated non-aromatic groups, having the specified number of members and containing 1, 2, 3 or 4 heteroatoms selected from N, O and S (unless a different number of heteroatoms is specified). Examples of particular heterocyclic groups include but are not limited to tetrahydrofuran, dihydropyran, tetrahydropyran, pyran, tetrahydropyran, thietane, 1,4-dioxane, 1,3-dioxane, 1,3-dioxalane, piperidine, piperazine, tetrahydropyrimidine, pyrrolidine, morpholine, thiomorpholine, thiazolidine, oxazolidine, tetrahydrothiopyran, tetrahydrothiophene, and the like.

The term “heteroaryl” refers to aromatic monocyclic groups and fused bicyclic groups wherein at least one ring is aromatic, having the specified number of members and containing 1, 2, 3, or 4 heteroatoms selected from N, O and S (unless a different number of heteroatoms is specified). Examples of particular heteroaryl groups include but are not limited to furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, and indazole.

The term “members” (and variants thereof e.g., “membered”) in the context of heterocyclic and heteroaryl groups refers to the total atoms, carbon and heteroatoms N, O and/or S, which form the ring. Thus, an example of a 6-membered heterocyclic ring is piperidine and an example of a 6-membered heteroaryl ring is pyridine.

As used herein, the term “optionally” means that the subsequently described event(s) may or may not occur, and includes both event(s) that occur and events that do not occur.

The present invention provides compounds of formula (I): 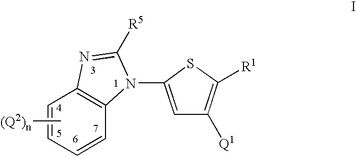
wherein:
R¹ is selected from the group consisting of H, alkyl, alkenyl, alkynyl, —C(O)R⁷, —CO₂R⁷, —C(O)NR⁷R⁸, —C(O)N(R⁷)OR₈, —C(O)N(R⁷)—R²—OR⁸, —C(O)N(R⁷)-Ph, —C(O)N(R⁷)—R²-Ph, —C(O)N(R⁷)C(O)R⁸, —C(O)N(R⁷)CO₂R⁸, —C(O)N(R⁷)C(O)NR⁷R⁸, —C(O)N(R⁷)S(O)₂R⁸, —R²—OR⁷, —R²—O—C(O)R⁷, —C(S)R⁷, —C(S)NR⁷R⁸, —C(S)N(R⁷)-Ph, —C(S)N(R⁷)—R²-Ph, —R²—SR⁷, —C(═NR⁷)NR⁷R⁸, —C(═NR⁷)N(R⁸)-Ph, —C(═NR⁷)N(R⁸)—R²-Ph, —R²—NR⁷R⁸, —CN, —OR⁷, —S(O)_{f}R⁷, —S(O)₂NR⁷R⁸, —S(O)₂N(R⁷)-Ph, —S(O)₂N(R⁷)—R²-Ph, —NR⁷R⁸, —N(R⁷)-Ph, —N(R⁷)—R²-Ph, —N(R⁷)—SO₂R⁸ and Het;
Ph is phenyl optionally substituted from 1 to 3 times with a substituent selected from the group consisting of halo, alkyl, —OH, —R²—OH, —O-alkyl, —R²—O-alkyl, —NH₂, —N(H)alkyl, —N(alkyl)₂, —CN and —N₃;
Het is a 5-7 membered heterocycle having 1, 2, 3 or 4 heteroatoms selected from N, O and S, or a 5-6 membered heteroaryl having 1, 2, 3 or 4 heteroatoms selected from N, O and S, each optionally substituted from 1 to 2 times with a substituent selected from the group consisting of halo, alkyl, oxo, —OH, —R²—OH, —O-alkyl, —R²—O-alkyl, —NH₂, —N(H)alkyl, —N(alkyl)₂, —CN and —N₃;
Q¹ is a group of formula:
   —(R²)ₐ—(Y¹)_{b}—(R²)_{c}—R³
a, b and c are the same or different and are each independently 0 or 1 and at least one of a or b is 1;
n is 0, 12, 3 or 4;
Q² is a group of formula:
   —(R²)ₐₐ—(Y²)_{bb}—(R²)_{cc}—R⁴
   or two adjacent Q² groups are selected from the group consisting of alkyl, alkenyl, —OR⁷, —S(O)_{f}R⁷ and —NR⁷R⁸ and together with the carbon atoms to which they are bound, they form a C₅₋₆cycloalkyl, C₅₋₆cycloalkenyl, phenyl, 5-7 membered heterocycle having 1 or 2 heteroatoms selected from N, O and S, or 5-6 membered heteroaryl having 1 or 2 heteroatoms selected from N, O and S;
aa, bb and cc are the same or different and are each independently 0 or 1;
each Y¹ and Y² is the same or different and is independently selected from the group consisting of —O—, —S(O)_{f}—, —N(R⁷)—, —C(O)—, —OC(O)—, —CO₂—, —C(O)N(R⁷)—, —C(O)N(R⁷)S(O)₂—, —OC(O)N(R⁷)—, —OS(O)₂—, —S(O)₂N(R⁷)—, —S(O)₂N(R⁷)C(O)—, —N(R⁷)S(O)₂—, —N(R⁷)C(O)—, —N(R⁷)CO₂— and —N(R⁷)C(O)N(R⁷)—;
each R² is the same or different and is independently selected from the group consisting of alkylene, alkenylene and alkynylene;
each R³ and R⁴ is the same or different and is each independently selected from the group consisting of H, halo, alkyl, alkenyl, alkynyl, —C(O)R⁷, —C(O)NR⁷R⁸, —CO₂R⁷, —C(S)R⁷, —C(S)NR⁷R⁸, —C(═NR⁷)R⁸, —C(═NR⁷)NR⁷R⁸, —CR⁷═N—OR⁷, —OR⁷—S(O)_{f}R⁷, —S(O)₂NR⁷R⁸, —NR⁷R⁸, —N(R⁷C(O)R⁸, —N(R⁷)S(O)₂R⁸, —NO₂, —CN, —N₃ and a group of formula (ii): 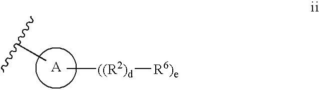
   wherein:
Ring A is selected from the group consisting of C₅₋₁₀cycloalkyl, C₅₋₁₀cycyloalkenyl, aryl, 5-10 membered heterocycle having 1, 2 or 3 heteroatoms selected from N, O and S and 5-10 membered heteroaryl having 1, 2 or 3 heteroatoms selected from N, O and S
each d is 0 or 1;
e is 0, 1, 2, 3 or 4;
each R⁶ is the same or different and is independently selected from the group consisting of H, halo, alkyl, alkenyl, alkynyl, cycloalkyl, cycloalkenyl, Ph, Het, —CH(OH)—R²—OH, —C(O)R⁷, —CO₂R⁷⁷—CO₂—R²-Ph, —CO₂—R²-Het, —C(O)NR⁷R⁸, —C(O)N(R⁷)C(O)R⁷, —C(O)N(R⁷CO₂R⁷, —C(O)N(R⁷)C(O)NR⁷R⁸, —C(O)N(R⁷)S(O)₂R⁷, —C(S)R⁷, —C(S)NR⁷R⁸, —C(═NR⁷) R⁸, —C(═NR⁷)NR⁷R⁸, —CR⁷═N—OR⁸, ═O, —OR⁷, —OC(O)R⁷, —OC(O)Ph, —OC(O)Het, —OC(O)NR⁷R⁸, —O—R²—S(O)₂R⁷, —S(O)_{f}R⁷, —S(O)₂NR⁷R⁸, —S(O)₂Ph, —S(O)₂Het, —NR⁷R⁸, —N(R⁷)C(O)R⁸, —N(R⁷)CO₂R⁸, —N(R⁷)—R²—CO₂R⁸, —N(R⁷)C(O)NR⁷R⁸, —N(R⁷)—R²—C(O)NR⁷R⁸, —N(R⁷)C(O)Ph, —N(R⁷)C(O)Het, —N(R⁷)Ph, —N(R⁷)Het, —N(R⁷)C(O)NR⁷—R²—NR⁷R⁸, —N(R⁷)C(O)N(R⁷)Ph, —N(R⁷)C(O)N(R⁷)Het, —N(R⁷)C(O)N(R⁷)—R²-Het, —N(R⁷)S(O)₂R⁸, —N(R⁷)—R²—S(O)₂R⁸, —NO₂, —CN and —N₃;
R⁵ is selected from the group consisting of H, halo, alkyl, cycloalkyl, OR⁷, —S(O)_{f}R⁷, —NR⁷R⁸, —NHC(O)R⁷, —NHC(O)NR⁷R⁸ and —NHS(O)₂R⁷;
f is 0, 1 or 2; and
each R⁷ and each R⁸ are the same or different and are each independently selected from the group consisting of H, alkyl, alkenyl, alkynyl, cycloalkyl and cycloalkenyl;
   and pharmaceutically acceptable salts, solvates and physiologically functional derivatives thereof.

In one embodiment, the compounds of formula (I) are defined wherein R¹ is selected from the group consisting of alkyl, alkenyl, alkynyl, —C(O)R⁷, —CO₂R⁷, —C(O)NR⁷R⁸, —C(O)N(R⁷)—R²—OR⁸, —R²—OR⁷, —C(S)NR⁷Re, —C(═NR⁷)NR⁷R⁸, —CN, —S(O)_{f}R⁷, —S(O)₂NR⁷Re, and Het, or any subset thereof. In one embodiment, the compounds of formula (I) are defined wherein R¹ is selected from the group consisting of —C(O)R⁷, —CO₂R⁷, —C(S)NR⁷R⁸, Het, and —C(O)NR⁷R⁸, or any subset thereof. In one embodiment, the compounds of formula (I) are defined wherein R¹ is selected from the group consisting of —C(O)R⁷, —CO₂R⁷ and —C(O)NR⁷R⁸, or any subset thereof. In one particular embodiment, R¹ is selected from the group consisting of —CO₂R⁷ and —C(O)NR⁷Re, or any subset thereof. In one embodiment, R¹ is —CO₂R⁷. In one embodiment, R¹ is —C(O)NR⁷R⁸.

Specific examples of groups defining R¹ include but are not limited to —COH, —COCH₃, —COOH, —COOCH₃, —C(O)NH₂, —CONH(alkyl), —CON(alkyl)(alkyl), —CONH(Et-OH), —CONH(benzyl), —CONH(phenyl), —S(O)₂NH₂ and —S(O)₂N(H)CH₃, —CH₂OH, —C(S)NH₂, —CN, and -tetrazole, or any subset thereof. In one particular embodiment, R¹ is selected from the group consisting of —CO₂H and —C(O)NH₂.

Q¹ is defined as a group of formula:
—(R²)ₐ—(Y¹)_{b}—(R²)_{c}—R³.

In the foregoing formula, a, b and c are the same or different and are each independently 0 or 1.

In one embodiment, Q¹ is defined wherein a is 0. In the embodiment wheren a is 1 and thus the (R²)ₐ group is present, R² is typically alkylene or alkenylene, more particularly alkylene. In one particular embodiment, Q¹ is defined where a is 1 and (R²)ₐ is C₁₋₃alkylene.

In one embodiment, Q¹ in the compounds of formula (I) is defined where b is 1; thus Y¹ is present. In one such embodiment, Y¹ is selected from —O—, —S(O)_{f}—, —N(R⁷)—, —C(O)—, —OC(O)—, —CO₂—, —C(O)N(R⁷)—, —C(O)N(R⁷)S(O)₂—, —OC(O)N(R⁷)—, —OS(O)₂—, —S(O)₂N(R⁷)—, —S(O)₂N(R⁷)C(O)—, —N(R⁷)S(O)₂—, —N(R⁷)C(O)—, —N(R⁷)CO₂— and —N(R⁷)C(O)N(R⁷)—. In one particular embodiment, Y¹ is selected from —O—, —N(R⁷)—, —C(O)—, —OC(O)—, —C(O)N(R⁷)—, —OS(O)₂—, —S(O)₂N(R⁷)—, —N(R⁷)S(O)₂—, and —N(R⁷)C(O)—, or any subset thereof. In another particular embodiment, Y¹ is selected from —O—, —N(R⁷)—, —C(O)—, —OS(O)₂—, —N(R⁷)S(O)₂—, and —N(R⁷)C(O)—, or any subset therof. In one particular embodiment, b is 1 and Y¹ is —O—, —N(R⁷)—, —C(O)— or —OS(O)₂—, or any subset thereof. In one particular embodiment, b is 1 and Y¹ is —O—. In another paticular embodiment, b is 1 and Y¹ is —N(R⁷)— and R⁷ is H or alkyl, more particularly H. In another particular embodiment, b is 1 and Y¹ is —C(O)—. In another particular embodiment, b is 1 and Y¹ is —OS(O)₂—.

The variable c in the formula Q¹ can be 0 or 1. In one embodiment, c is 1. In one such embodiment (R²)C is alkylene or alkenylene, more particularly alkylene. In one particular embodiment, Q¹ is defined where c is 1 and (R²)_{c} is C₁₋₃alkylene.

In one embodiment of the invention, the compounds of formula (I) are defined to include a substitution at the position indicated by Q¹; thus, when a, b and c are all 0, then R³ is not H. In one particular embodiment the compounds of the present invention are defined wherein, at least one of a or b is 1. In one particular embodiment, Q¹ is defined wherein both b and c are 1. In one particular embodiment, Q¹ is defined wherein a is 0 and both b and c are 1.

Consistent with the definition of b, Y¹ and c, the group R³ may be selected from the group consisting of H, halo, alkyl, alkenyl, alkynyl, —C(O)R⁷, —C(O)NR⁷R⁸, —CO₂R⁷, —C(S)R⁷, —C(S)NR⁷R⁸, —C(═NR⁷)R⁸, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN, —N₃ and a group of formula (ii):

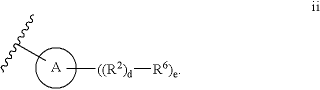

In one embodiment, R³ in the definition of Q¹ is selected from the group consisting of H, alkyl, alkenyl, alkynyl, and a group of formula (ii), or any subset thereof. In one particular embodiment, R³ is selected from the group consisting of H, alkyl, alkenyl and alkynyl, or any subset thereof. In one embodiment, when R³ is alkyl, R³ is C₂₋₆alkyl.

In one particular embodiment, R³ is a group of formula (ii). 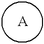
in formula (ii) is referred to herein as “Ring A.” Ring A is selected from C₅₋₁₀cycloalkyl, C₅₋₁₀cycloalkenyl, aryl, 5-10 membered heterocycle having 1, 2 or 3 heteroatoms selected from N, O and S and 5-10 membered heteroaryl having 1, 2 or 3 heteroatoms selected from N, O and S. In Q¹, Ring A may be bonded to R², Y¹ (when c is 0) or the thiophene ring (when a, b and c are 0) through any suitable carbon or heteroatom. In one embodiment, Q¹ is defined wherein R³ is a group of formula (ii) and Ring A is selected from C₅₋₁₀cycloalkyl, C₅₋₁₀cycloalkenyl, aryl, 5-10 membered heterocycle having 1, 2 or 3 heteroatoms selected from N, O and S and 5-10 membered heteroaryl having 1, 2 or 3 heteroatoms selected from N, O and S. In one embodiment, Q¹ is defined wherein R³ is a group of formula (ii) and Ring A is selected from aryl, 5-10 membered heterocycle having 1, 2 or 3 heteroatoms selected from N, O and S and 5-10 membered heteroaryl having 1, 2 or 3 heteroatoms selected from N, O and S. In one particular embodiment, Q¹ is defined wherein R³ is a group of formula (ii) and Ring A is selected from aryl and 5-10 membered heteroaryl having 1, 2 or 3 heteroatoms selected from N, O and S.

In one embodiment, Q¹ is defined wherein R³ is a group of formula (ii) and Ring A is selected from the group consisting of cycloalkyl, tetrahydropyran, tetrahydrofuran, morpholine, piperidine, phenyl, naphthyl, thiophene, furan, pyrrole, pyrrolidine, pyrrolidinone, imidazole, benzofuran, benzimidazole, pyridyl, 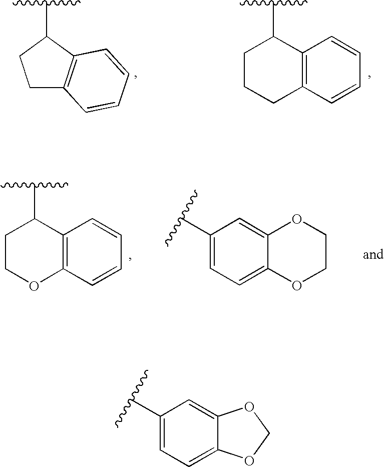
or any subset thereof. In one particular embodiment, Ring A is phenyl. In one particular embodiment Ring A is pyridyl.

Particular, more specific, examples of groups defining Q¹ in the compounds of formula (I) are selected from the group consisting of: 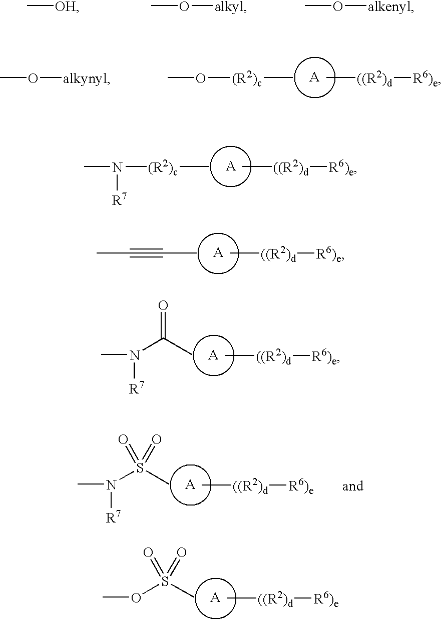
or any subset thereof.

One particular group defining Q¹ is

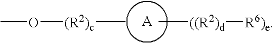

In one particular embodiment, Q¹ is

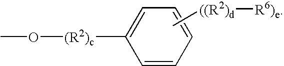

In one particular embodiment, Q¹ is

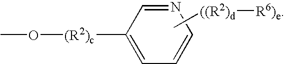

In one particular embodiment, Q¹ is

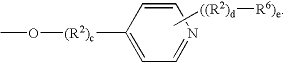

In one embodiment the compounds of formula (I) are defined wherein R³ is a group of formula (ii) and d is 0 or 1. In a particular embodiment, wherein R³ is a group of formula (ii) and d is 1, R² is C₁₋₃alkylene. In one embodiment, d is 0.

In one embodiment, wherein the compounds of formula (I) are defined wherein R³ is a group of formula (ii), e is 0, 1, 2 or 3. In one particular embodiment, e is 0 or 1. In one embodiment, e is 1. In one embodiment, e is 2.

In one embodiment, wherein the compounds of formula (I) are defined wherein R³ is a group of formula (ii), each R⁶ is the same or different and is independently selected from the group consisting of H, halo, alkyl, alkenyl, alkynyl, cycloalkyl, Ph, Het, —CH(OH)—R²—OH, —C(O)R⁷, —C(O)NR⁷R⁶, ═O, —OR⁷, —S(O)_{f}R⁷⁷—S(O)₂NR⁷R⁸, —SO₂Ph, —NR⁷R⁸, —N(R⁷C(O)CR⁸, —N(R⁷)CO₂R⁸, —N(R⁷)S(O)₂R⁸, —NO₂, —CN and —N₃, or any subset thereof. In one particular embodiment, R³ is a group of formula (ii) and each R⁶ is the same or different and is independently selected from the group consisting of H, halo, alkyl, alkenyl, alkynyl, cycloalkyl, —OR⁷, —S(O)_{f}R⁷, —S(O)₂NR⁷R⁸, —NR⁷R⁸, —N(R⁷)S(O)₂R⁸, —NO₂ and —CN or any subset thereof. In one particular embodiment, R³ is a group of formula (ii) and each R⁶ is the same or different and is independently selected from the group consisting of H, halo, alkyl, —OR⁷, —S(O)_{f}R⁷, —S(O)₂NR⁷R⁸ and —NO₂, or any subset thereof.

More specifically, in one embodiment wherein R³ is a group of formula (ii), each R⁶ is the same or different and is independently selected from the group consisting of H, F, Cl, Br, I, methyl, trifluoromethyl, ethyl, propyl, isopropyl, cyclopropyl, iso-butyl, t-butyl, ethenyl, propenyl, acetylene, O-methyl, O-difluoromethyl, O-trifluoromethyl, O-ethyl, O-propyl, O-isopropyl, O-cyclopropyl, —SO₂-methyl, —SO₂NH₂, —NH₂, —NH(alkyl), —N(alkyl)alkyl, —NH(cyclopropyl), —NHSO₂-methyl, —NO₂, and —CN, or any subset thereof.

In one embodiment, Q¹ is defined such that when b is 1 and c is 0, R³ is not halo, —C(O)R⁷, —C(O)NR⁷R⁸, —CO₂R⁷, —C(S)R⁷, —C(S)NR⁷R⁸, —C(═NR⁷)R⁸, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN or —N₃.

In one embodiment, wherein when R¹ is —CO₂CH₃ and n is 0, Q¹ is not —OH. In one embodiment, Q¹ is not —OH.

In one embodiment, n is 0, 1 or 2, or any subset thereof. In one particular embodiment, n is 0, and thus the benzimidazole ring is unsubstituted at positions C-4, C-5, C-6 and C-7. In one embodiment, n is 2 and Q² is at C-5 and C-6. In another particular embodiment, n is 1. In one particular embodiment n is 2.

Q² is a group of formula —(R²)ₐₐ—(Y²)_{bb}—(R²)_{cc}—R⁴. Q² may be located at any of C-4, C-5, C-6 and/or C-7 of the benzimidazole ring. In one embodiment, n is 1 and Q² is at C-5.

In one embodiment, n is 1 and Q² is at C-6.

In the foregoing formula, aa, bb and cc are the same or different and are each independently 0 or 1.

In one embodiment, aa is 0; thus the group (R²)ₐₐ is not present. In the embodiment wherein aa is 1, (R²)ₐₐ is typically alkylene or alkenylene, more particularly alkylene. In one particular embodiment, Q² is defined where aa is 1 and (R²)ₐₐ is C₁₋₃alkylene.

In one embodiment, the compounds of formula (I) are defined wherein bb is 0. In another embodiment, Q² in the compounds of formula (I) is defined where bb is 1; thus Y² is present. In one such embodiment, Y² is selected from —O—, —S(O)_{f}—, —N(R⁷)—, —C(O)—, —OC(O)—, —CO₂—, —C(O)N(R⁷)—, —C(O)N(R⁷)S(O)₂—, —OC(O)N(R⁷)—, —OS(O)₂—, —S(O)₂N(R⁷)—, —S(O)₂N(R⁷)C(O)—, —N(R⁷)S(O)₂—, —N(R⁷)C(O)—, —N(R⁷)CO₂— and —N(R⁷)C(O)N(R⁷)—. In one particular embodiment, bb is 1 and Y² is selected from —O—, —S(O)_{f}—, —N(R⁷)—, —C(O)—, —OC(O)—, —CO₂—, —C(O)N(R⁷)—, —OS(O)₂—, —N(R⁷)S(O)₂—, —N(R⁷)C(O)—, —N(R⁷)CO₂— and —N(R⁷)C(O)N(R⁷)—, or any subset thereof. In another particular embodiment, bb is 1 and Y² is selected from —O—, —S(O)_{f}—, —N(R⁷)—, —CO₂—, —C(O)N(R⁷)—, —N(R⁷)S(O)₂—, and —N(R⁷C(O)—, —N(R⁷)CO₂—, —N(R⁷)C(O)N(R⁷)—, or any subset thereof. In one particular embodiment, Q² is defined wherein bb is 1 and Y² is selected from —O—, —S(O)_{f}—, —N(R⁷)—, —CO₂— and —C(O)N(R⁷)—, or any subset thereof. In one particular embodiment, Q² is defined wherein bb is 1 and Y² is —O—. In one particular embodiment, Q² is defined wherein bb is 1 and Y¹ is —S(O)_{f}—, wherein f is 2. In another particular embodiment, bb is 1 and Y¹ is —N(R⁷)— and R⁷ is H or alkyl, more particularly H. In another particular embodiment, bb is 1 and Y¹ is —CO₂—. In another particular embodiment, bb is 1 and Y² is —C(O)N(R⁷)—.

The variable cc in the formula Q² can be 0 or 1. In one embodiment, cc is 1. In one such embodiment (R²)_{c} is alkylene or alkenylene, more particularly alkylene. In one particular embodiment, Q² is defined where cc is 1 and (R²)_{c} is C₁₋₃alkylene.

Consistent with the definition of bb, Y² and cc, the group R⁴ may be selected from the group consisting of H, halo, alkyl, alkenyl, alkynyl, —C(O)R⁷, —C(O)NR⁷R⁸, —CO₂R⁷, —C(S)R⁷, —C(S)NR⁷R⁸, —C(═NR⁷R⁸, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN, —N₃ and a group of formula (ii):

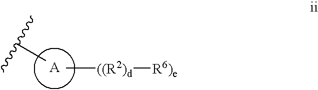

In one embodiment, R⁴ in the definition of Q² is selected from the group consisting H, halo, alkyl, alkenyl, alkynyl, —C(O)NR⁷R⁸, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN, —N₃ and a group of formula (ii), or any subset thereof. In one particular embodiment, R⁴ is selected from the group consisting of H, halo, alkyl, —OR⁷, —S(O)_{f}R⁷, —S(O)₂NR⁷R⁸, —NR⁷R⁸, and a group of formula (ii), or any subset thereof. In one embodiment, R⁴ is selected from H, halo, alkyl, —OR⁷—NR⁷R⁸, and a group of formula (ii), or any subset thereof.

In one particular embodiment, R⁴ is a group of formula (ii). In the embodiment, wherein R⁴ is a group of formula (ii), Ring A is selected from C₅₋₁₀cycloalkyl, C₅₋₁₀cycloalkenyl, aryl, 5-10 membered heterocycle having 1, 2 or 3 heteroatoms selected from N, O and S and 5-10 membered heteroaryl having 1, 2 or 3 heteroatoms selected from N, O and S. In one embodiment, wherein R⁴ is a group of formula (ii), Ring A is selected from C₅₋₆cycloalkyl, C₅₋₆cycloalkenyl, aryl, 5-10 membered heterocycle having 1, 2 or 3 heteroatoms selected from N, O and S and 5-10 membered heteroaryl having 1, 2 or 3 heteroatoms selected from N, O and S. In Q², Ring A may be bonded to the R², Y² (when cc is 0) or the benzimidazole (when aa, bb and cc are 0) through any suitable carbon or heteroatom. In one embodiment, Q² is defined wherein R⁴ is a group of formula (ii) and Ring A is selected from aryl, 5-10 membered heterocycle having 1, 2 or 3 heteroatoms selected from N, O and S and 5-10 membered heteroaryl having 1, 2 or 3 heteroatoms selected from N, O and S. In one particular embodiment, Q² is defined wherein R⁴ is a group of formula (ii) and Ring A is selected from aryl and 5-10 membered heterocycle having 1, 2 or 3 heteroatoms selected from N, O and S.

In one embodiment, Q² is defined wherein R⁴ is a group of formula (ii) and Ring A is selected from the group consisting of cycloalkyl, oxetane, oxazole, thiazole, morpholine, piperidine, piperazine, phenyl, naphthyl, thiophene, furan, pyrrolidine, pyrrolidinone, imidazole, triazole, imidazolidinone, benzofuran, benzodioxolane, benzimidazole and pyridyl, or any subset thereof. In one particular embodiment, Ring A is selected from morpholine, piperidine, piperazine, phenyl, pyrrolidinone, imidazolidinone and pyrrolidine, or any subset thereof.

More specifically, in one embodiment, each R⁴ is the same or different and is independently selected from the group consisting of H, F, Cl, Br, I, methyl, trifluoromethyl, ethyl, propyl, isopropyl, cyclopropyl, iso-butyl, t-butyl, ethenyl, propenyl, acetylene, O-methyl, O-trifluoromethyl, O-ethyl, O-propyl, O-isopropyl, O-cyclopropyl, —SO₂-methyl, —SO₂NH₂, —NH₂, —N H(alkyl), —N(alkyl)alkyl, —NH(cyclopropyl), —NHC(O)-methyl, —NHC(O)NH₂, —NHSO₂-methyl, morpholino and piperizinyl, or any subset thereof.

Particular, more specific, examples of groups defining Q² in the compounds of formula (I) are selected from the group consisting of:

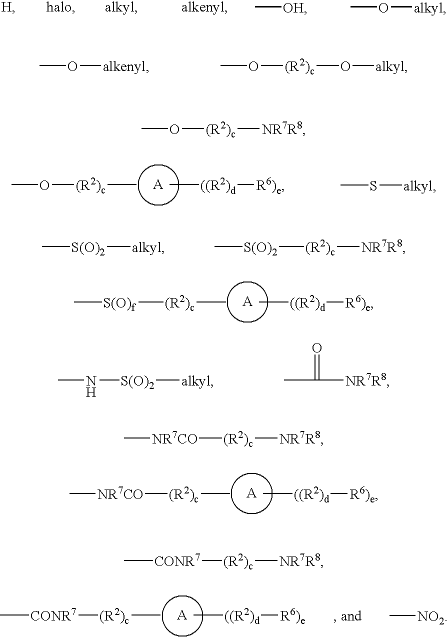

In one embodiment, Q² is —O-alkyl. In one particular embodiment, Q² is halo.

In one embodiment the compounds of formula (I) are defined wherein R⁴ is a group of formula (ii) and d is 0 or 1. In a particular embodiment, wherein R⁴ is a group of formula (ii) and d is 1, R² is C₁₋₃alkylene. In one embodiment, d is O.

In one embodiment, wherein the compounds of formula (I) are defined wherein R⁴ is a group of formula (ii), e is 0, 1, 2 or 3. In one particular embodiment, e is 0 or 1. In one embodiment, e is 0. In one embodiment, e is 1. In one embodiment, e is 2.

In one embodiment, wherein the compounds of formula (I) are defined wherein R⁴ is a group of formula (ii), each R⁶ is the same or different and is independently selected from the group consisting of H, halo, alkyl, alkenyl, alkynyl, Het, —C(O)R⁷, —CO₂R⁷, —C(O)NR⁷R⁸, ═O, —OR⁷, —S(O)_{f}R⁷, —S(O)₂NR⁷R⁸, —NR⁷R⁸ and —N(R⁷)S(O)₂R⁸, or any subset thereof. In one particular embodiment, each R⁶ is the same or different and is independently selected from the group consisting of H, halo, alkyl, ═O, —OR⁷, —S(O)_{f}R⁷, —S(O)₂NR⁷R⁸ and —NR⁷R⁸, or any subset thereof.

More specifically, in one embodiment, each R⁶ is the same or different and is independently selected from the group consisting of H, methyl, ethyl, propyl, isopropyl, iso-butyl, t-butyl, ethenyl, propenyl, cyclopropyl, pyrimidyl, —C(O)-alkyl, —CO₂-alkyl, —C(O)NH₂, acetylene, oxo, O-methyl, O-ethyl, O-propyl, O-isopropyl, O-cyclopropyl, —SO₂-methyl, —SO₂NH₂, —NH₂, —NH(alkyl), —N(alkyl)alkyl, —NH(cyclopropyl) and —NHSO₂-methyl, or any subset thereof.

In another embodiment of the present invention, two adjacent Q² groups are selected from the group consisting of alkyl, alkenyl, —OR⁷, —S(O)_{f}R⁷ and —NR⁷R⁸ and together with the carbon atoms to which they are bound, they form a C₅₋₆cycloalkyl, C₅₋₆cycloalkenyl, phenyl, 5-7 membered heterocycle having 1 or 2 heteroatoms selected from N, O and S, or 5-6 membered heteroaryl having 1 or 2 heteroatoms selected from N, O and S. By “two adjacent Q² groups” is meant that two Q² groups are bonded to adjacent carbon atoms (e.g., C-4 and C-5). For example, in one embodiment two adjacent Q² groups are —OR⁷ and together with the atoms to which they are bonded, they form a heterocyclic group such as:

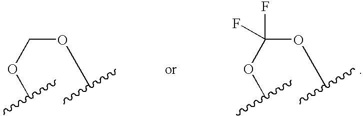

In another embodiment, two adjacent Q² groups are alkyl and together with the atoms to which they are bonded, they form a cycloalkyl group such as:

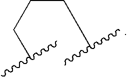

In another embodiment two adjacent Q² groups are defined as —OR⁷ and —NR⁷R⁸ respectively and together with the atoms to which they are bonded, they form a heterocyclic group such as:

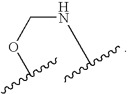

From these examples, additional embodiments can be readily ascertained by those skilled in the art. Preferably the compounds of formula (I) are defined wherein when n is 2, two adjacent Q² groups together with the atoms to which they are bonded do not form a C₅₋₆cycloalkyl, C₅₋₆cycloalkenyl, phenyl, 5-7 membered heterocycle having 1 or 2 heteroatoms selected from N, O and S, or 5-6 membered heteroaryl having 1 or 2 heteroatoms selected from N, O and S.

In one embodiment, Q² is defined such that when bb is 1 and cc is 0, R⁴ is not halo, —C(O)R⁷, —C(O)NR⁷R⁸, —CO₂R⁷, —C(S)R⁷, —C(S)NR⁷R⁸, —C(═NR⁷)R⁸, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN or —N₃;

In one embodiment, R⁵ is selected from the group consisting of H, halo, alkyl, —NR⁷R⁸ and —S(O)_{f}R⁷, or any subset thereof. In another embodiment, R⁵ is selected from the group consisting of H, halo, alkyl and —NR⁷R⁸, or any subset thereof. In one particular embodiment, R⁵ is H. In one particular embodiment, R⁵ is —NH₂.

More specifically, in one embodiment, R⁵ is selected from the group consisting of H, F, Cl, Br, 1, methyl, trifluoromethyl, ethyl, propyl, isopropyl, —S-methyl, —SO₂-methyl and —NH₂, or any subset thereof.

The compounds of the present invention also include, compounds of formula (Ia): 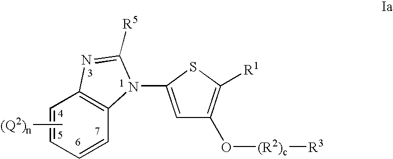
wherein all variables are as defined above, and pharmaceutically acceptable salts, solvates and physiologically functional derivatives thereof.

The present invention also provides compounds of formula (Ib): 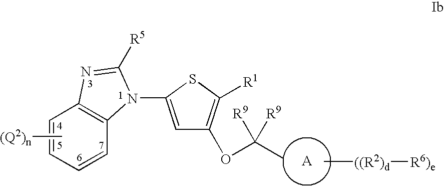
wherein each R⁹ is the same or different and is selected from H, halo and alkyl; and all other variables are as defined above, and pharmaceutically acceptable salts, solvates and physiologically functional derivatives thereof.

It is to be understood that the present invention includes all combinations and subsets of the particular groups defined hereinabove.

Specific compounds of formula (I) include but are not limited to those compounds described in the Example section that follows. Some particular compounds of formula (I) include but are not limited to:
5-(5,6-Dimethoxy-1H-benzimidazol-1-yl)-3-{[2-(trifluoromethyl)-benzyl]oxy}thiophene-2-carboxamide;
5-(5-(Methyloxy)-6-{[2-(4-methyl-1-piperazinyl)ethyl]oxy}-1H-benzimidazol-1-yl)-3-({[2-(trifluoromethyl)phenyl]methyl}oxy)-2-thiophenecarboxamide;
3-[1-(2-Chlorophenyl)ethoxy]-5-(5,6-dimethoxy-1H-benzimidazol-1-yl)thiophene-2-carboxamide;
5-(5,6-Dimethoxy-1H-benzimidazol-1-yl)-3-[1-(2-methylphenyl)ethoxy]thiophene-2-carboxamide;
5-(5-Amino-1H-benzimidazol-1-yl)-3-[1-(2-chlorophenyl)ethoxy]thiophene-2-carboxamide;
5-{6-[(4-Piperidinylmethyl)oxy]-1H-benzimidazol-1-yl}-3-({[2-(trifluoromethyl)phenyl]-methyl}oxy)-2-thiophenecarboxamide;
5-(6-(Methyloxy)-5-{[3-(2-oxo-1-pyrrolidinyl)propyl]oxy}-1H-benzimidazol-1-yl)-3-({[2-(trifluoromethyl)phenyl]methyl}oxy)-2-thiophenecarboxamide;
5-[6-{[3-(Dimethylamino)propyl]oxy}-5-(methyloxy)-1H-benzimidazol-1-yl]-3-({[2-(trifluoromethyl)phenyl]methyl}oxy)-2-thiophenecarboxamide;
5-(5-(Methyloxy)-6-{[2-(4-morpholinyl)ethyl]oxy}-1H-benzimidazol-1-yl)-3-({[2-(trifluoromethyl)phenyl]methyl}oxy)-2-thiophenecarboxamide;
5-[6-(2-Morpholin-4-ylethoxy)-1H-benzimidazol-1-yl]-3-{[2-(trifluoromethyl)benzyl]oxy}thiophene-2-carboxamide;
5-[6-(2-Pyrrolidin-1-ylethoxy)-1H-benzimidazol-1-yl]-3-{[2-(trifluoromethyl)benzyl]oxy}thiophene-2-carboxamide;
5-[5-Fluoro-6-(2-morpholin-4-ylethoxy)-1H-benzimidazol-1-yl]-3-{[2-(trifluoromethyl)benzyl]oxy}thiophene-2-carboxamide;
5-[6-(Methylsulfonyl)-1H-benzimidazol-1-yl]-3-{[2-(trifluoromethyl)benzyl]oxy}-thiophene-2-carboxamide;
3-[(3-Bromopyridin-4-yl)methoxy]-5-(5,6-dimethoxy-1H-benzimidazol-1-yl)thiophene-2-carboxamide;
5-(5,6-Dimethoxy-1H-benzimidazol-1-yl)-3-{[2-(trifluoromethoxy)benzyl]oxy}thiophene-2-carboxamide;
3-{[2-(Difluoromethoxy)benzyl]oxy}-5-(5,6-dimethoxy-1H-benzimidazol-1-yl)thiophene-2-carboxamide;
3-[(2-Chloropyridin-3-yl)methoxy]-5-(5,6-dimethoxy-1H-benzimidazol-1-yl)thiophene-2-carboxamide;
5-(5,6-Dimethoxy-1H-benzimidazol-1-yl)-3-[(2-fluoropyridin-3-yl)methoxy]thiophene-2-carboxamide;
3-[(2-Aminopyridin-4-yl)methoxy]-5-(5,6-dimethoxy-1H-benzimidazol-1-yl)thiophene-2-carboxamide;
3-[(6-Chloro-1,3-benzodioxol-5-yl)methoxy]-5-(5,6-dimethoxy-1H-benzimidazol-1-yl)thiophene-2-carboxamide;
5-(5,6-Dimethoxy-1H-benzimidazol-1-yl)-3-[(2-nitrobenzyl)oxy]thiophene-2-carboxamide;
3-[(3-Aminobenzyl)oxy]-5-(5,6-dimethoxy-1H-benzimidazol-1-yl)thiophene-2-carboxamide;
5-(6-Bromo-1H-benzimidazol-1-yl)-3-{[2-(trifluoromethyl)benzyl]-oxy}thiophene-2-carboxamide;
3-[(2,6-Dichlorobenzyl)oxy]-5-(5,6-dimethoxy-1H-benzimidazol-1-yl)thiophene-2-carboxamide;
3-[(2-Bromobenzyl)oxy]-5-(5,6-dimethoxy-1H-benzimidazol-1-yl)thiophene-2-carboxamide;
5-(5,6-Dimethoxy-1H-benzimidazol-1-yl)-3-[(2-formylbenzyl)oxy]thiophene-2-carboxamide;
5-(1H-Benzimidazol-1-yl)-3-{[2-(trifluoromethyl)benzyl]oxy}thiophene-2-carboxamide;
5-(1H-Benzimidazol-1-yl)-3-[(2-nitrobenzyl)oxy]thiophene-2-carboxamide;
5-(6-Methoxy-1H-benzimidazol-1-yl)-3-{[2-(trifluoromethyl)benzyl]oxy}thiophene-2-carboxamide;
2-(Aminocarbonyl)-5-(5,6-dimethoxy-1H-benzimidazol-1-yl)thien-3-yl 2-methylbenzenesulfonate
   and pharmaceutically acceptable salts, solvates and physiologically functional derivatives thereof.

It will be appreciated by those skilled in the art that the compounds of the present invention may also be utilized in the form of a pharmaceutically acceptable salt or solvate or physiologically functional derivative thereof. The pharmaceutically acceptable salts of the compounds of formula (I) include conventional salts formed from pharmaceutically acceptable inorganic or organic acids or bases as well as quaternary ammonium salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic (mesylate), naphthalene-2-sulfonic, benzenesulfonic hydroxynaphthoic, hydroiodic, malic, steroic, tannic and the like.

Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts.

The term “solvate” as used herein refers to a complex of variable stoichiometry formed by a solute (a compound of formula (I)) and a solvent Solvents, by way of example, include water, methanol, ethanol, or acetic acid.

The term “physiologically functional derivative” as used herein refers to any pharmaceutically acceptable derivative of a compound of the present invention, for example, an ester or an amide of a compound of formula (I), which upon administration to an animal, particularly a mammal, such as a human, is capable of providing (directly or indirectly) a compound of the present invention or an active metabolite thereof. See, for example, Burger's Medicinal Chemistry And Drug Discovery, 5th Edition, Vol 1: Principles And Practice.

Processes for preparing pharmaceutically acceptable salts, solvates and physiologically functional derivatives of the compounds of formula (I) are conventional in the art. See, e.g., Burger's Medicinal Chemistry And Drug Discovery 5th Edition, Vol 1: Principles And Practice.

As will be apparent to those skilled in the art, in the processes described below for the preparation of compounds of formula (I), certain intermediates, may be in the form of pharmaceutically acceptable salts, solvates or physiologically functional derivatives of the compound. Those terms as applied to any intermediate employed in the process of preparing compounds of formula (I) have the same meanings as noted above with respect to compounds of formula (I). Processes for preparing pharmaceutically acceptable salts, solvates and physiologically functional derivatives of such intermediates are known in the art and are analogous to the process for preparing pharmaceutically acceptable salts, solvates and physiologically functional derivatives of the compounds of formula (I).

Certain compounds of formula (I) may exist in stereoisomeric forms (e.g. they may contain one or more asymmetric carbon atoms or may exhibit cis-trans isomerism). The individual stereoisomers (enantiomers and diastereomers) and mixtures of these are included within the scope of the present invention. The present invention also covers the individual isomers of the compounds represented by formula (I) as mixtures with isomers thereof in which one or more chiral centres are inverted. Certain compounds of formula (I) may be prepared as a mixture of regioisomers. The present invention covers both the mixture of regioisomers as well as the individual compounds. Likewise, it is understood that compounds of formula (I) may exist in tautomeric forms other than that shown in the formula and these are also included within the scope of the present invention. In one particular embodiment of the present invention, the chiral compounds are present in the R conformation (i.e., the R-isomer of the compound).

The compounds of the present invention are typically inhibitors of PLK. By PLK inhibitor is meant a compound which exhibits pIC₅₀ greater than 4 in the PLK Inhibition assay described below in the examples or an IC₅₀ less than 100 μM in the Methylene Blue Growth Inhibition assay described below in the examples; more particularly a PLK inhibitor is a compound which exhibits a pIC₅₀ greater than 5 or an IC₅₀ less than 10 μM using the methods described in the examples below.

The present invention further provides compounds of formula (I) for use in medical therapy in an animal, e.g. a mammal such as a human. In particular, the present invention provides compounds of formula (I) for use in the treatment of a condition mediated by PLK. The present invention also provides compounds of formula (I) for use in the treatment of a susceptible neoplasm. The present invention provides compounds of formula (I) for use in treating a condition characterized by inappropriate cellular proliferation. The present invention also provides compounds of formula (I) for use in inhibiting proliferation of a cell. The present invention also provides compounds of formula (I) for use in inhibiting mitosis in a cell.

The present invention provides methods for the treatment of several conditions or diseases, all of which comprise the step of administering a therapeutically effective amount of a compound of formula (I). As used herein, the term “treatment” refers to alleviating the specified condition, eliminating or reducing the symptoms of the condition, slowing or eliminating the progression of the condition and preventing or delaying the reoccurrance of the condition in a previously afflicted subject.

As used herein, the term “therapeutically effective amount” means an amount of a compound of formula (I) which is sufficient, in the subject to which it is administered, to elicit the biological or medical response of a cell culture, tissue, system, animal (including human) that is being sought, for instance, by a researcher or clinician. For example, a therapeutically effective amount of a compound of formula (I) for the treatment of a condition mediated by PLK is an amount sufficient to treat the PLK mediated condition in the subject. Similarly, a therapeutically effective amount of a compound of formula (I) for the treatment of a susceptible neoplasm is an amount sufficient to treat the susceptible neoplasm in the subject. In one embodiment of the present invention, the therapeutically effective amount of a compound of formula (I) is an amount sufficient to inhibit cell mitosis. In one embodiment of the present invention, a therapeutically effective amount of a compound of formula (I) is an amount sufficient to regulate, modulate, bind or inhibit PLK.

The precise therapeutically effective amount of the compounds of formula (I) will depend on a number of factors including, but not limited to, the age and weight of the subject being treated, the precise disorder requiring treatment and its severity, the nature of the formulation, and the route of administration, and will ultimately be at the discretion of the attendant physcian or veternarian. Typically, the compound of formula (I) will be given for treatment in the range of 0.1 to 200 mg/kg body weight of recipient (animal) per day and more usually in the range of 1 to 100 mg/kg body weight per day. Acceptable daily dosages, may be from about 0.1 to about 2000 mg/day, and preferably from about 0.1 to about 100 mg/day.

As one aspect, the present invention provides methods of regulating, modulating, binding, or inhibiting PLK for the treatment of conditions mediated by PLK. “Regulating, modulating, binding or inhibiting PLK” refers to regulating, modulating, binding or inhibiting PLK activity, as well as regulating, modulating, binding or inhibiting overexpression of PLK. Such conditions include certain neoplasms (including cancers and tumors) which have been associated with PLK and conditions characterized by inappropriate cellular proliferation.

The present invention provides a method for treating a condition mediated by PLK in an animal such as a mammal (e.g., a human), which method comprises administering to the animal a therapeutically effective amount of the compound of formula (I). Conditions which are mediated by PLK are known in the art and include but are not limited to neoplasms and conditions characterized by inappropriate cellular proliferation.

The present invention also provides a method for treating a susceptible neoplasm (cancer or tumor) in an animal such as a mammal (e.g., a human), which method comprises administering to the animal a therapeutically effective amount of the compound of formula (I). “Susceptible neoplasm” as used herein refers to neoplasms which are susceptible to treatment with a PLK inhibitor. Neoplasms which have been associated with PLK and are therefor susceptible to treatment with a PLK inhibitor are known in the art, and include both primary and metastatic tumors and cancers. For example, susceptible neoplasms within the scope of the present invention include but are not limited to breast cancer, colon cancer, lung cancer (including small cell lung cancer and non-small cell lung cancer), prostate cancer, lymphoma, leukemia, endometrial cancer, melanoma, ovarian cancer, pancreatic cancer, squamous carcinoma, carcinoma of the head and neck, and esophageal carcinoma. The compounds of formula (I) can be used alone in the treatment of such susceptible neoplasms or can be used to provide additive or synergistic effects with certain existing chemotherapies, and/or be used to restore effectiveness of certain existing chemotherapies and radiation.

The present invention also provides a method for treating a condition characterized by inappropriate cellular proliferation. By “inapproriate cellular proliferation” is meant cellular proliferation resulting from inappropriate cell growth, cellular proliferation resulting from excessive cell division, cellular proliferation resulting from cell division at an accelerated rate, cellular proliferation resulting from inappropriate cell survival, and/or cellular proliferation in a normal cell occurring at a normal rate, which is neverthless undesired. Conditions characterized by inappropriate cellular proliferation include but are not limited to neoplasms, blood vessel proliferative disorders, fibrotic disorders, mesangial cell proliferative disorders and metabolic diseases. Blood vessel proliferative disorders include arthritis and restenosis. Fibrotic disorders include hepatic cirrhosis and atherosclerosis. Mesangial cell proliferative disorders include glomerulonephritis, malignant nephrosclerosis, thrombotic microangiopathy syndromes, organ transplant rejection and glomerulopathies. Metabolic disorders include psoriasis, chronic wound healing, inflammation and neurodegenerative diseases. Osteoarthritis and other osteoclast proliferation dependent diseases of excess bone resorbtion are examples of conditions characterized by inapproprate cellular proliferation in which the cellular proliferation occurs in normal cells at a normal rate, but is nevertheless undesired.

The present invention also provides a method for inhibiting proliferation of a cell, which method comprises contacting the cell with an amount of a compound of formula (I) sufficient to inhibit proliferation of the cell. In one particular embodiment, the cell is a neoplastic cell. In one particular embodiment, the cell is an inappropriately proliferative cell. The term “inappropriately proliferative cell” as used herein refers to cells that grow inappropriately (abnormally), cells that divide excessively or at an accelerated rate, cells that inappropriately (abnormally) survive and/or normal cells that proliferate at a normal rate but for which proliferation is undesired. Neoplastic cells (including cancer cells) are an example of inappropriately proliferative cells but are not the only inappropriately proliferative cells.

PLK is essential for cellular mitosis and accordingly, the compounds of formula (I) are effective for inhibiting mitosis. “Inhibiting mitosis” refers to inhibiting the entry into the M phase of the cell cycle, inhibiting the normal progression of the M phase of the cell cycle once M phase has been entered and inhibiting the normal exit from the M phase of the cell cycle. Thus, the compounds of the present invention may inhibit mitosis by inhibiting the cell's entry into mitosis, by inhibiting the cell's progression through mitosis or by inhibiting the cell's exit from mitosis. As one aspect, the present invention provides a method for inhibiting mitosis in a cell, which method comprises administering to the cell an amount of a compound of formula (I) sufficient to inhibit mitosis. In one particular embodiment, the cell is a neoplastic cell. In one particular embodiment, the cell is an inappropriately proliferative cell.

The present invention also provides the use of a compound of formula (I) for the preparation of a medicament for the treatment of condition mediated by PLK in an animal, such as a mammal (e.g., a human). The present invention further provides the use of a compound of formula (I) for the preparation of a medicament for the treatment of a susceptible neoplasm in an animal. The present invention further provides the use of a compound of formula (I) for the preparation of a medicament for the treatment of a condition characterized by inappropriate cellular proliferation. The present invention further provides the use of a compound of formula (I) for the preparation of a medicament for inhibiting proliferation of a cell. The present invention further provides the use of a compound of formula (I) for the preparation of a medicament for inhibiting mitosis in a cell.

While it is possible that, for use in therapy, a therapeutically effective amount of a compound of formula (I) may be administered as the raw chemical, it is typically presented as the active ingredient of a pharmaceutical composition or formulation.

Accordingly, the invention further provides a pharmaceutical composition comprising a compound of the formula (I). The pharmaceutical composition may further comprise one or more pharmaceutically acceptable carriers, diluents, and/or excipients. The carrier(s), diluent(s) and/or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I) with one or more pharmaceutically acceptable carriers, diluents and/or excipients.

Pharmaceutical formulations may be presented in unit dose form containing a predetermined amount of active ingredient per unit dose. Such a unit may contain a therapeutically effective dose of the compound of formula (I) or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of active ingredient Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I) can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula (I) may also be delivered by the use of monoclonal antibodies as individual carriers to which the compound molecules are coupled. The compounds may also be coupled with soluble polymers as targetable drug carriers. Such polymers can include peptides, polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6):318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

In the above-described methods of treatment and uses, a compound of formula (I) may be employed alone, in combination with one or more other compounds of formula (I) or in combination with other therapeutic agents. In particular, in methods of treating conditions mediated by PLK and methods of treating susceptible neoplasms, combination with other chemotherapeutic, hormonal and/or antibody agents is envisaged as well as combination with surgical therapy and radiotherapy. The term “chemotherapeutic” as used herein refers to any chemical agent having a therapeutic effect on the subject to which it is administered. “Chemotherapeutic” agents include but are not limited to anti-neoplastic agents, analgesics and anti-emetics. As used herein, “anti-neoplastic agents” include both cytostatic and cytotoxic agents. Combination therapies according to the present invention thus comprise the administration of at least one compound of formula (I) and the use of at least one other cancer treatment method. In one embodiment, combination therapies according to the present invention comprise the administration of at least one compound of formula (I) and at least one other chemotherapeutic agent. In one particular embodiment, the present invention comprises the administration of at least one compound of formula (I) and at least one anti-neoplastic agent. As an additional aspect, the present invention provides the methods of treatment and uses as described above, which comprise administering a compound of formula (I) together with at least one chemotherapeutic agent. In one particular embodiment, the chemotherapeutic agent is an anti-neoplastic agent. In another embodiment, the present invention provides a pharmaceutical composition as described above further comprising at least one other chemotherapeutic agent, more particularly, the chemotherapeutic agent is an anti-neoplastic agent.

Typically, any chemotherapeutic agent that has activity versus a susceptible neoplasm being treated may be utilized in combination with the compounds of formula (I), provided that the particular agent is clinically compatible with therapy employing a compound of formula (I). Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphor-ines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids. Examples of diterpenoids include, but are not limited to, paclitaxel and its analog docetaxel. Examples of vinca alkaloids include, but are not limited to, vinblastine, vincristine, and vinorelbine.

Platinum coordination complexes are non-phase specific anti-neoplastic agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Alkylating agents are non-phase anti-neoplastic specific agents and strong electrophiles. Typically, alkylating agents form covalent linkages, by alkylation, to DNA through nucleophilic moieties of the DNA molecule such as phosphate, amino, and hydroxyl groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Antibiotic chemotherapeutic agents are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Topoisomerase II inhibitors include, but are not limited to, epipodophyllotoxins. Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide. Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine and thioguanine.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin.

Hormones and hormonal analogues are useful compounds for treating cancers in which there is a relationship between the hormone(s) and growth and/or lack of growth of the cancer. Examples of hormones and hormonal analogues believed to be useful in the treatment of neoplasms include, but are not limited to, adrenocorti-costeroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene and iodoxyfene useful in the treatment of hormone dependent breast carcinoma; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors are those inhibitors which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are sometimes termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr, ErbB2 and ErbB4,), platelet derived growth factor receptor (PDGFr), vascular endothelial growth factor receptor (VEGFR), tyrosine kinase with immunoglobulin-like and epidermal growth factor homology domains (TIE-2), insulin growth factor-I receptor (IGF-I), macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth factor receptors are under development and include ligand antagonists, antibodies, tyrosine kinase inhibitors, anti-sense oligonucleotides and aptamers. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth Factor Receptors as Targets”, New Molecular Targets for Cancer Chemotherapy, Ed. Workman, Paul and Kerr, David, CRC Press 1994, London.

Tyrosine kinases, which are not growth factor receptor kinases are termed non-receptor tyrosine kinases. Non-receptor tyrosine kinases useful in the present invention, which are targets or potential targets of anti-neoplastic drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual Review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (Rafk), Mitogen or Extracellular Regulated Kinase (MEKs), and Extracellular Regulated Kinases (ERKs); and Protein kinase C family member blockers including blockers of subtypes of PKCs (alpha, beta, gamma, epsilon, mu, lambda, iota, zeta), IkB kinase family (IKKa, IKKb), PKB family kinases, Akt kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; and Martinez-Iacaci, L., et al, Int. J. Cancer (2000), 88(1), 44-52.

Inhibitors of Phosphotidyl Inositol-3 Kinase family members including blockers of PI3-kinase, ATM, DNA-PK, and Ku are also useful in combination with the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer Res, (2000) 60(6), 1541-1545.

Also useful in combination with the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC Press 1994, London.

Another group of signal transduction pathway inhibitors useful in combination with the present invention are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block Ras activation in cells containing wild type mutant Ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9(2)99-102; and BioChim. Biophys. Acta, (1989) 1423(3):19-30.

As mentioned above, antibodies to receptor kinase ligand binding may also serve as signal transduction inhibitors. This group of signal transduction pathway inhibitors includes the use of humanized antibodies to the extracellular ligand binding domain of receptor tyrosine kinases. For example, Imclone C225 EGFR specific antibody (see Green, M. C. et al, Monoclonal Antibody Therapy for Solid Tumors, Cancer Treat. Rev., (2000), 26(4), 269-286); Herceptin® ErbB2 antibody (see Tyrosine Kinase Signaling in Breast Cancer:ErbB Family Receptor Tyrosine Kinases, Breast Cancer Res., 2000, 2(3), 176-183); and 2CB VEGFR2 specific antibody (see Brekken, R. A. et al, Selective Inhibition of VEGFR2 Activity by a Monoclonal Anti-VEGF Antibody Blocks Tumor Growth in Mice, Cancer Res. (2000) 60, 5117-5124).

Receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Other inhibitors may be used in combination with the compounds of the present invention. For example, anti-VEGF antibodies, which do not recognize VEGFR (the receptor tyrosine kinase), but bind to the ligand; small molecule inhibitors of integrin (alphaᵥ beta₃) that will inhibit angiogenesis; endostatin and angiostatin (non-RTK) may also prove useful in combination with PLK inhibitors.

Agents used in immunotherapeutic regimens may also be useful in combination with the compounds of formula (I).

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al., J. Clin. Oncol. 18:1812-1823 (2000); and Kitada S et al., Antisense Res. Dev. 4:71-79 (1994).

Cell cycle signaling inhibitors inhibit molecules involved in the control of the cell cycle. Cyclin dependent kinases (CDKs) and their interaction cyclins control progression through the eukaryotic cell cycle. The coordinated activation and inactivation of different cyclin/CDK complexes is necessary for normal progression through the cell cycle. Several inhibitors of cell cycle signaling are under development. For instance, examples of cyclin dependent kinases, including CDK2, CDK4, and CDK6 and inhibitors for the same are described in, for instance, Rosania, et al., Exp. Opin. Ther. Patents 10(2):215-230 (2000).

In one embodiment, the methods of the present invention comprise administering to the animal a compound of formula (I) in combination with a signal transduction pathway inhibitor, particularly gefitinib (IRESSA®).

The methods and uses employing these combinations may comprise the administration of the compound of formula (I) and the other chemotherapeutic/anti-neoplastic agent either sequentially in any order or simultaneously in separate or combined pharmaceutical compositions. When combined in the same formulation it will be appreciated that the two compounds must be stable and compatible with each other and the other components of the formulation and may be formulated for administration. When formulated separately they may be provided in any convenient formulation, in such a manner as are known for such compounds in the art.

When a compound of formula (I) is used in combination with a chemotherapeutic agent, the dose of each compound may differ from that when the compound is used alone. Appropriate doses will be readily appreciated by those skilled in the art The appropriate dose of the compound(s) of formula (I) and the other therapeutically active agent(s) and the relative timings of administration will be selected in order to achieve the desired combined therapeutic effect, and are within the expertise and discretion of the attendent clinician.

Compounds of formula (I) may be conveniently prepared by the methods outlined in Scheme 1 below. 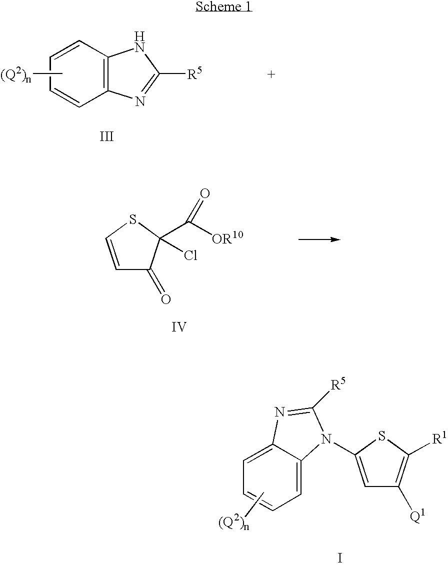
wherein:
R¹ is selected from the group consisting of H, alkyl, alkenyl, alkynyl, —C(O)R⁷, —CO₂R⁷, —C(O)NR⁷R⁸, —C(O)N(R⁷)OR⁸, —C(O)N(R⁷)—R²—OR⁸, —C(O)N(R⁷)-Ph, —C(O)N(R⁷)—R²-Ph, —C(O)N(R⁷)C(O)R⁸, —C(O)N(R⁷)CO₂R⁸, —C(O)N(R⁷)C(O)NR⁷R⁸, —C(O)N(R⁷)S(O)₂R⁸, —R²—OR⁷, —R²—O—C(O)R⁷, —C(S)R⁷, —C(S)NR⁷R⁸, —C(S)N(R⁷)-Ph, —C(S)N(R⁷)—R²-Ph, —R²—SR⁷, —C(═NR⁷)NR⁷R⁸, —C(═NR⁷)N(R⁸)-Ph, —C(═NR⁷)N(R⁸)—R²-Ph, —R²—NR⁷R⁸, —CN, —OR⁷, —S(O)_{f}R⁷, —S(O)₂NR⁷R⁶, —S(O)₂N(R⁷)-Ph, —S(O)₂N(R⁷)—R²-Ph, —NR⁷R⁸, —N(R⁷)-Ph, —N(R⁷)—R²-Ph, —N(R⁷)—SO₂R⁸ and Het;
Ph is phenyl optionally substituted from 1 to 3 times with a substituent selected from the group consisting of halo, alkyl, —OH, —R²—OH, —O-alkyl, —R²—O-alkyl, —NH₂, —N(H)alkyl, —N(alkyl)₂, —CN and —N₃;
Het is a 5-7 membered heterocycle having 1, 2, 3 or 4 heteroatoms selected from N, O and S, or a 5-6 membered heteroaryl having 1, 2, 3 or 4 heteroatoms selected from N, O and S, each optionally substituted from 1 to 2 times with a substituent selected from the group consisting of halo, alkyl, oxo, —OH, —R²—OH, —O-alkyl, —R²—O-alkyl, —NH₂, —N(H)alkyl, —N(alkyl)₂, —CN and —N₃;
Q¹ is a group of formula:
   —(R²)ₐ—(Y¹)_{b}—(R²)_{c}—R³
a, b and c are the same or different and are each independently 0 or 1 and at least one of a or b is 1;
n is 0, 1, 2, 3 or 4;
Q² is a group of formula:
   —(R²)ₐₐ—(Y²)_{bb}—(R²)_{cc}—R⁴
   or two adjacent Q² groups are selected from the group consisting of alkyl, alkenyl, —OR⁷, —S(O)_{f}R⁷ and —NR⁷R⁸ and together with the carbon atoms to which they are bound, they form a C₅₋₆cycloalkyl, C₅₋₆cycloalkenyl, phenyl, 5-7 membered heterocycle having 1 or 2 heteroatoms selected from N, O and S, or 5-6 membered heteroaryl having 1 or 2 heteroatoms selected from N, O and S;
aa, bb and cc are the same or different and are each independently 0 or 1;
each Y¹ and Y² is the same or different and is independently selected from the group consisting of —O—, —S(O)_{f}—, —N(R⁷)—, —C(O)—, —OC(O)—, —CO₂—, —C(O)N(R⁷)—, —C(O)N(R⁷)S(O)₂—, —OC(O)N(R⁷)—, —OS(O)₂—, —S(O)₂N(R⁷)—, —S(O)₂N(R⁷)C(O)—, —N(R⁷)S(O)₂—, —N(R⁷)C(O)—, —N(R⁷)CO₂— and —N(R⁷)C(O)N(R⁷)—;
each R² is the same or different and is independently selected from the group consisting of alkylene, alkenylene and alkynylene;
each R³ and R⁴ is the same or different and is each independently selected from the group consisting of H, halo, alkyl, alkenyl, alkynyl, —C(O)R⁷, —C(O)NR⁷R⁸, —CO₂R⁷, —C(S)R⁷, —C(S)NR⁷R⁸, —C(═NR⁷)R⁸, —C(═NR⁷)NR⁷R⁸, —CR⁷═N—OR⁷, —OR⁷—S(O)_{f}R⁷, —S(O)₂NR⁷R⁸, —NR⁷R⁸, —N(R⁷)C(O)R⁸, —N(R⁷)S(O)₂R⁸, —NO₂, —CN, —N₃ and a group of formula (ii): 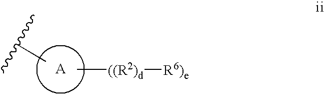
   wherein:
Ring A is selected from the group consisting of C₅₋₁₀cycloalkyl, C₅₋₁₀cycloalkenyl, aryl, 5-10 membered heterocycle having 1, 2 or 3 heteroatoms selected from N, O and S and 5-10 membered heteroaryl having 1, 2 or 3 heteroatoms selected from N, O and S
each d is 0 or 1;
e is 0, 1, 2, 3 or 4;
each R⁶ is the same or different and is independently selected from the group consisting of H, halo, alkyl, alkenyl, alkynyl, cycloalkyl, cycloalkenyl, Ph, Het, —CH(OH)—R²—OH, —C(O)R⁷, —CO₂R⁷⁷—CO₂—R²-Ph, —CO₂—R²-Het, —C(O)NR⁷R⁸, —C(O)N(R⁷)C(O)R⁷, —C(O)N(R⁷)CO₂R⁷, —C(O)N(R⁷)C(O)NR⁷R⁸, —C(O)N(R⁷)S(O)₂R⁷, —C(S)R⁷, —C(S)NR⁷R⁸, —C(═NR⁷)R⁸, —C(═NR⁷)NR⁷R⁸, —CR⁷═N—OR⁸, ═O, —OR⁷, —OC(O)R⁷, —OC(O)Ph, —OC(O)Het, —OC(O)NR⁷R⁸, —O—R²—S(O)₂R⁷, —S(O)_{f}R⁷, —S(O)₂NR⁷R⁸, —S(O)₂Ph, —S(O)₂Het, —NR⁷R⁸, —N(R⁷)C(O)R⁸, —N(R⁷)CO₂R⁸, —N(R⁷)—R²—CO₂R⁸, —N(R⁷)C(O)NR⁷R⁸, —N(R⁷)—R²—C(O)NR⁷R⁸, —N(R⁷)C(O)Ph, —N(R⁷)C(O)Het, —N(R⁷)Ph, —N(R⁷)Het, —N(R⁷)C(O)NR⁷—R²—NR⁷R⁸, —N(R⁷)C(O)N(R⁷)Ph, —N(R⁷)C(O)N(R⁷)Het, —N(R⁷)C(O)N(R⁷)—R²-Het, —N(R⁷)S(O)₂R⁸, —N(R⁷)—R²—S(O)₂R⁸, —NO₂, —CN and —N₃;
wherein when Q¹ is defined where b is 1 and c is 0, R³ is not halo, —C(O)R⁷, —C(O)NR⁷R⁸, —CO₂R⁷, —C(S)R⁷, —C(S)NR⁷R⁸, —C(═NR⁷)R⁸, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN or —N₃;
wherein when Q² is defined where bb is 1 and cc is 0, R⁴ is not halo, —C(O)R⁷, —C(O)NR⁷R⁸—CO₂R⁷⁷—C(S)R⁷, —C(S)NR⁷R⁸, —C(═NR⁷)R⁸, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN or —N₃;
R⁵ is selected from the group consisting of H, halo, alkyl, cycloalkyl, OR⁷, —S(O)_{f}R⁷, —NR⁷R⁸, —NHC(O)R⁷, —NHC(O)NR⁷R⁸ and —NHS(O)₂R⁷;
f is 0, 1 or 2; and
each R⁷ and each R⁸ are the same or different and are each independently selected from the group consisting of H, alkyl, alkenyl, alkynyl, cycloalkyl and cycloalkenyl; and
R¹⁰ is selected from the group consisting of alkyl, alkenyl, alkynyl, cycloalkyl, cycloalkenyl and suitable carboxylic acid protecting groups.

Generally, the process for preparing the compounds of formula (I) (all formulas and all variables having been defined above in connection with Scheme 1) comprises the steps of:

a) reacting a compound of formula (III) with a compound of formula (IV) to prepare a compound of formula (I);

b) optionally converting the compound of formula (I) to a pharmaceutically acceptable salt, solvate or physiologically functional derivative thereof; and

c) optionally converting the compound of formula (I) or a pharmaceutically acceptable salt, solvate or physiologically functional derivative thereof to a different compound of formula (I) or a pharmaceutically acceptable salt, solvate or physiologically functional derivative thereof.

More specifically, compounds of formula (I) can be prepared by reacting a compound of formula (IV) with a compound of formula (III) to prepare a compound of formula (I-A). 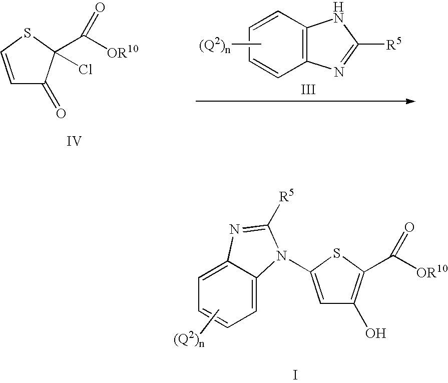
wherein all variables are as defined in connection with Scheme 1.

A compound of formula (I-A) may be converted into a pharmaceutically acceptable salt, solvate or physiologically functional derivative thereof or may be converted to a different compound of formula (I) or a pharmaceutically acceptable salt, solvate or physiologically functional derivative thereof using techniques described hereinbelow and those conventional in the art.

The reaction of a compound of formula (III) with a compound of formula (IV) is typically carried out in an inert solvent at room temperature. Typically two molar equivalents of a compound of formula (III) are combined with one molar equivalent of a compound of formula (IV). Examples of suitable inert solvents for this reaction include but are not limited to, chloroform, dichloromethane, tetrahydrofuran, dioxane, and toluene.

A compound of formula (IV) can be prepared by reacting a compound of formula MV) with sulfuryl chloride. 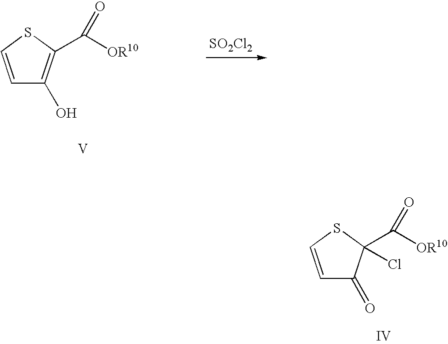
wherein all variables are as defined in connection with Scheme 1.

Compounds of formula (V) are commercially available or can be prepared using conventional knowledge in the art Typically, reaction of a compound of formula (V) with sulfuryl chloride at room temperature provides a compound of formula (IV). Excess sulfuryl chloride may be used if desired. Examples of suitable solvents include but are not limited to chloroform, dichloromethane, and toluene. See, Corral, C.; Lissavetzky, J. Synthesis 847-850 (1984).

A compound of formula (III) can be prepared by several methods. According to one method, a compound of formula (III) is prepared according to Scheme 2 below. 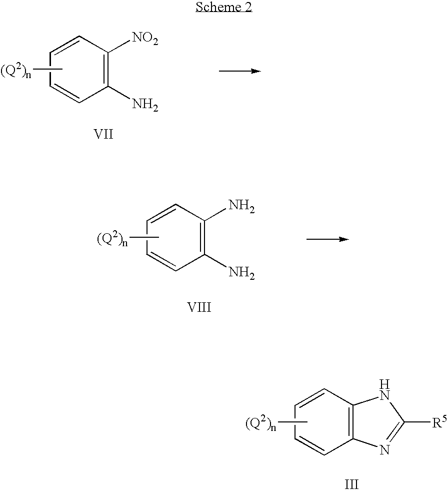
wherein all variables are as defined in connection with Scheme 1.

Generally, this process for preparing a compound of formula (III) (all formulas and all variables having been defined above in connection with Scheme 1) comprises the steps of:

a) reducing the compound of formula (VII) to prepare a compound of formula (VIII); and

b) reacting the compound of formula (VIII) with a ring forming reagent to prepare a compound of formula (III).

The order of the foregoing steps is not critical to the practice of the invention and the process may be practiced by performing the steps in any suitable order based on the knowledge of those skilled in the art.

More specifically, a compound of formula (III) can be prepared by reacting a compound of formula (VIII) with a ring forming reagent. There are several ring forming reagents which may be employed in this process step. In one embodiment, the compound formula (III-A) (i.e., a compound of formula (III) wherein R⁵ is H or alkyl) is prepared by reacting a compound of formula (VIII) with a ring forming reagent of formula (IX). 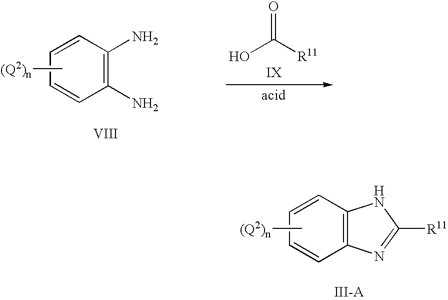
wherein R¹¹ is H or alkyl and all other variables are as defined in connection with Scheme 1.

This reaction may be carried out using conventional techniques. See, White, A., et al., J. Med. Chem. 43:4084-4097 (2000); Jiang, J.-L, et al., Synthetic Comm. 28:4137-4142 (1998); Tanaka, A., et al., Chem. Pharm. Bull. 42:560-569 (1994); Tian, W., et al., Synthesis 12:1283-1286 (1992); Buckle, D. R., et al., J. Med. Chem. 30:2216-2221 (1987); and Raban, M., et al., J. Org. Chem. 50:2205-2210 (1985). This reaction may be carried out neat or in a suitable solvent. The reaction may optionally be heated to a temperature of from about 50 to about 230 DC. The reaction is typically carried out with an excess of the compound of formula (IX). An additional acid may be used.

Examples of suitable acids include but are not limited to, hydrochloric acid, hydrobromic acid, perchloric acid, sulfuric acid, p-toluenesulfonic acid, methanesulfonic acid, and trifluoromethanesulfonic acid. Examples of suitable solvents for this reaction include but are not limited to water, methanol, ethanol, isopropanol, tetrahydrofuran, dichloromethane, toluene, N,N-dimethylformamide, dimethylsulfoxide, and acetonitrile. The compounds of formula (IX) are commercially available.

A compound of formula (VIII) may be prepared by reducing a compound of formula (VII). 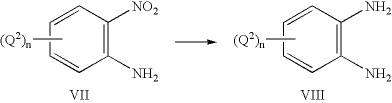
wherein all variables are as defined in connection with Scheme 1.

The reduction can be carried out using conventional techniques and reducing agents. See, Rangarajan, M., et al., Bioorg. Med. Chem. 8:2591-2600 (2000); White, A. W., et al., J. Med. Chem. 43: 4084-4097 (2000); Silvestri, R., et al., Bioorg. Med. Chem. 8:2305-2309 (2000); Nagaraja, D., et al., Tetrahedron Lett 40:7855-7856 (1999); Jung, F., et al., J. Med. Chem. 34:1110-1116 (1991); Srivastava, R. P., et al., Pharmazie 45:34-37 (1990); Hankovszky, H. O., et al., Can. J. Chem. 67:1392-1400 (1989); Ladd, D. L., et al., J. Org. Chem. 53:417-420 (1988); Mertens, A., et al., J. Med. Chem. 30:1279-1287 (1987); and Sharma, K. S., et al., Synthesis 4:316-318 (1981). Examples of suitable reducing agents for this reaction include but are not limited to, palladium with hydrogen, palladium with ammonium formate, platinum oxide with hydrogen, nickel with hydrogen, tin(II) chloride, iron with acetic acid, aluminum with ammonium chloride, borane, sodium dithionite, and hydrazine. The reaction may optionally be heated to between about 50 and about 120° C. Suitable solvents for this reaction vary and include but are not limited to, water, methanol, ethanol, ethyl acetate, tetrahydrofuran, and dioxane.

A compound of formula (VII) may be prepared by several methods. In one embodiment, the compound of formula (VII) is prepared by reacting a compound of formula (VI) with ammonia. 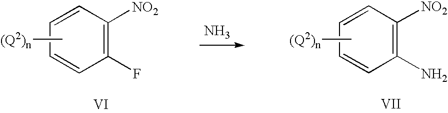
wherein all variables are as defined in connection with Scheme 1.

This reaction may be carried out using conventional techniques. See, Silvestri, R., et al., Bioorg. Med. Chem. 8:2305-2309 (2000); Hankovszky, H. O., et al., Can. J. Chem. 67:1392-1400 (1989); Nasielski-Hinkens, R.; et al., Heterocycles 26:2433-2442 (1987); Chu, K. Y., et al., J. Chem. Soc., Perkin Trans. 1 10:1194-1198 (1978). This reaction is typically carried out with an excess of ammonia and may be optionally heated to a temperature of from about 50 to about 100° C. Examples of suitable solvents for this reaction include but are not limited to, water, methanol, ethanol, isopropanol, tetrahydrofuran, dioxane, and 1,2-dimethoxyethane.

The compounds of formula (VI) are commercially available or may be prepared using conventional techniques and reagents.

In another embodiment, the compound of formula (VII) can be prepared by reacting a protected compound of formula (X) under nitration conditions to prepare a protected compound of formula (VII) (i.e., VII-A) and then removing the protecting group from the compound of formula (VII-A). 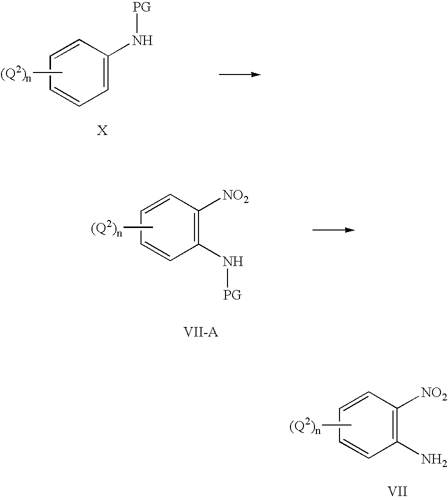
wherein PG is a protecting group and all other variables are as defined in connection with Scheme 1.

The protection of anilines is a common transformation well known to one skilled in the art. See, Kocienski, P. J. Protecting Groups, Georg Thieme Verlag, Stuttgart, 1994; and Greene, T. W., Wuts, P. G. M. Protecting Groups in Organic Synthesis (2^{nd} Edition), J. Wiley and Sons, 1991. Suitable protecting groups for this application include but are not limited to acetyl, trifluoroacetyl, benzyloxycarbonyl, allyloxycarbonyl, 2-(rimethylsilyl)ethoxycarbonyl, phenylsulfonyl, and p-toluenesulfonyl. Reagents and conditions vary according to the nature of the particular protecting group. Some typical reagents include but are not limited to acetic anhydride, trifluoroacetic anhydride, benzyl chloroformate, allyl chloroformate, 4-nitrophenyl 2-(trimethylsilyl)ethyl carbonate, phenylsulfonyl chloride, and p-toluensulfonyl chloride. In certain cases the addition of some base is required. Examples of suitable bases include but are not limited to potassium carbonate, sodium carbonate, trialkylamines, pyridine, and potassium t-butoxide. Suitable solvents for these conversions include but are not limited to dichloromethane, chloroform, tetrahydrofuran, acetic acid, methanol, ethanol, water, toluene, and diethyl ether.

The nitration of anilines is also well documented in the literature and the foregoing reaction may be carried out using these conventional techniques. See, Wissner, A., et. al., J. Med. Chem. 46: 49-63 (2003); Duggan, S. A., et. al., J. Org. Chem. 66: 4419-4426 (2001); Clews, J., et. al., Tetrahedron 56: 8735-8746 (2000); and Kagechika, H., J. Med. Chem. 31: 2182-2192 (1988). The nitration may be carried out with a variety of nitrating reagents including but not limited to 70% aqueous nitric acid, red fuming nitric acid, ammonium nitrate with trifluoroacetic anhydride, and potassium nitrate with trifluoromethanesulfonic acid. The reaction is typically conducted at room temperature, but may be optionally heated to a temperature of from about 40 to about 100° C. in certain cases. Suitable solvents include but are not limited to acetic acid, sulfuric acid, acetic anhydride, dichloromethane, and chloroform.

The nitration results in a compound of formula (VII-A), (i.e., a protected compound of formula (VII)). The cleavage of the aniline protecting group, to result in a compound of formula (VII) can be accomplished through many different conventional methods. See, Kocienski, P. J. Protecting Groups, Georg Thieme Verlag, Stuttgart, 1994; and Greene, T. W., Wuts, P. G. M. Protecting Groups in Organic Synthesis (2^{nd} Edition), J. Wiley and Sons, 1991.

The compounds of formula (X) may be prepared by installing a protecting group on the corresponding aniline. Such Anilines are commercially available or may be prepared using conventional techniques.

A compound of formula (III-A) may optionally be converted to a compound of formula (III-B). This conversion may be effected by halogenating the compound of formula (III-A) to prepare a compound of formula (III-B). 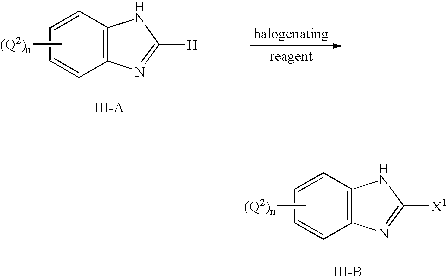
wherein X¹ is halo (particularly Cl, Br or I) and all other variables are as defined in connection with Scheme 1.

This type of transformation is well established in the literature. See, Taylor, E. C., et al., J. Org. Chem. 56:6937-6939 (1991); Mistry, A. G., et al., Tetrahedron Lett. 27:1051-1054 (1986); and Apen, P. G., et al., Heterocycles 29:1325-1329 (1989). Suitable halogenating agents include but are not limited to, N-chlorosuccinimide, N-bromosuccinimide, N-iodosuccinimide, chlorine, bromine, and iodine. Examples of suitable solvents include but are not limited to, dichloromethane, chloroform, diethyl ether, tetrahydrofuran, and acetone.

A compound of formula (III-B) may also be prepared directly from a compound of formula (VIII). The process comprises the steps of i) reacting a compound of formula (VIII) with a phosgene or phosgene equivalent compound to prepare a compound of formula (XII) and ii) reacting the compound of formula (XII) with phosphorous oxy halide to prepare a compound of formula (III-B). 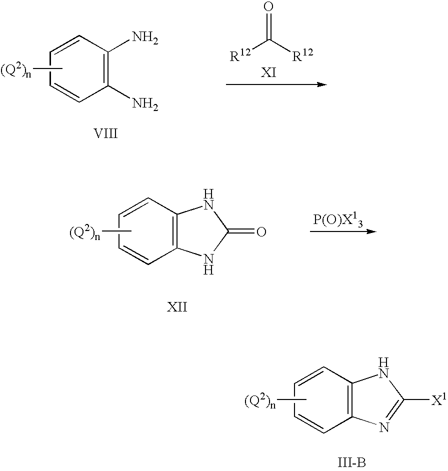
wherein:
each R¹² is the same or different and is independently selected from the group consisting of Cl, methoxy, ethoxy, trichloromethoxy, amino and N-imidazolyl;
X¹ is halo (particularly Cl, Br or I; more particularly Cl or Br); and
all other variables are as defined in connection with Scheme 1.

The phosgene or phosgene equivalent compound is the ring forming reagent and is typically a compound of formula (XI) as shown above. Phosgene and phosgene equivalent compounds of formula (XI) are commercially available. Examples of suitable compounds of formula (XI) include but are not limited to phosgene, dimethyl carbonate, diethyl carbonate, 1,1′-carbonyldiimidazole, urea, and triphosgene. The reaction of a compound of formula (VIII) with the phosgene or phosgene equivalent compound can be carried out using conventional techniques. See, Silvestri, R., et al., Bioorg. Med. Chem. 8:2305-2309 (2000); Wright, J. L., et al., J. Med. Chem. 43:3408-3419 (2000); Penieres, G. C., et al., Synthetic Comm. 30:2191-2195 (2000); and Von der Saal, W., et al., J. Med. Chem. 32:1481-1491 (1989). The reaction is typically run in an inert solvent or neat. The reaction may be optionally heated to a temperature of from about 50 to about 250° C. The optional addition of a suitable base to the reaction may be desirable. Examples of such bases include but are not limited to, trialkylamines, pyridine, 2,6-lutidine, potassium carbonate, sodium carbonate, and sodium bicarbonate. Examples of suitable solvents for this reaction include but are not limited to dichloromethane, chloroform, N,N-dimethylformamide, tetrahydrofuran, toluene, and acetone.

The reaction of the compound of formula (XII) with the phosphorous oxy halide to prepare a compound of formula (III-B) can be carried out using conventional techniques. See, Blythin, D. J., et al., J. Med. Chem. 29:1099-1113 (1986); and Crank, G., Aust. J. Chem. 35:775-784 (1982). Examples of suitable reagents include but are not limited to phosphorous oxychloride and phosphorous oxybromide. Suitable solvents include but are not limited to, dichloromethane, chloroform, dichloroethane, and toluene. Optional heat ranging from about 50 to about 150° C. may be used.

A compound of formula (III-B), prepared by any method, may optionally be converted to a compound of formula (III-C) by reacting with an amine of formula HNR⁷R⁸. 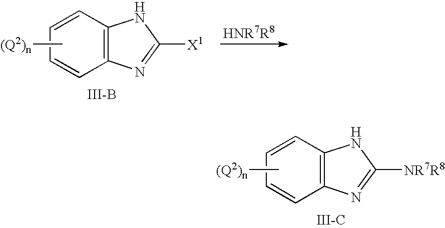
wherein all variables are as defined above.

The reaction of a halo-substituted benzimidazole of formula (III-B) with an amine to prepare a compound of formula (III-C) can be carried out using conventional techniques. See, Alcalde, E., et al., J. Org. Chem. 56:4233-4238 (1991); Katsushima, T., et al., J. Med. Chem. 33:1906-1910 (1990); Young, R. C., et al., J. Med. Chem. 33:2073-2080 (1990); Iemura, R., et al., J. Med. Chem. 29:1178-1183 (1986); and Benassi, R., et al., J. Chem. Soc, Perkin Trans. 210:1513-1521 (1985). An acid catalyst may be employed if desired. Examples of suitable acid catalysts include but are not limited to, hydrochloric acid and p-toluenesulfonic acid. The reaction can optionally be heated to a temperature of from about 50 to about 220° C. Suitable solvents for this reaction include but are not limited to, water, ethanol, isopropanol, 1-methyl-2-pyrrolidinone, N,N-dimethylformamide, dimethylsulfoxide, toluene, xylenes and tetrahydrofuran.

In another embodiment, a compound of formula (III-D) (i.e., a compound of formula ((III) wherein R⁵ is H or alkyl) is prepared according to the process outlined in Scheme 3 below. 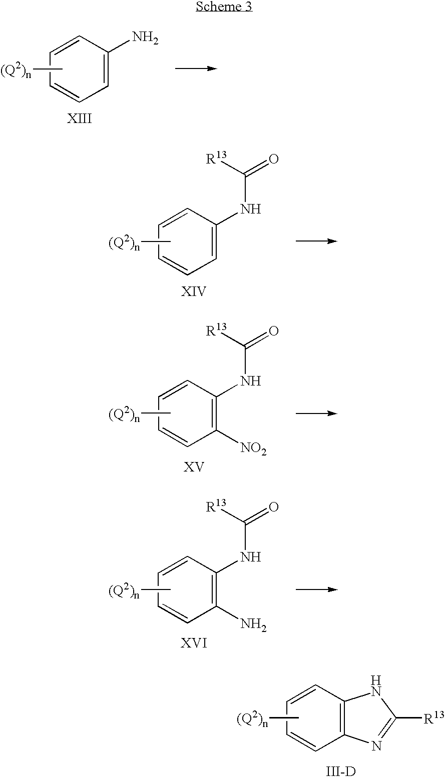
wherein R¹³ is H or alkyl and all other variables are as defined in connection with Scheme 1.

Generally, this process for preparing the a compound of formula (III-D) (all formulas and all variables having been defined above in connection with Scheme 1) comprises the steps of:

a) reacting a compound of formula (XIII) with a suitable acylating agent to prepare a compound of the formula (XIV);

b) reacting a compound of formula (XIV) under nitration conditions to prepare a compound of the formula (XV);

c) reducing a compound of formula (XV) to prepare a compound of formula (XVI); and

d) cyclizing a compound of formula (XVI) to prepare a compound of formula (III-D).

The order of the foregoing steps is not critical to the practice of the invention and the process may be practiced by performing the steps in any suitable order based on the knowledge of those skilled in the art.

More specifically, a compound of formula (III-D) can be prepared by cyclizing a compound of formula (XVI). 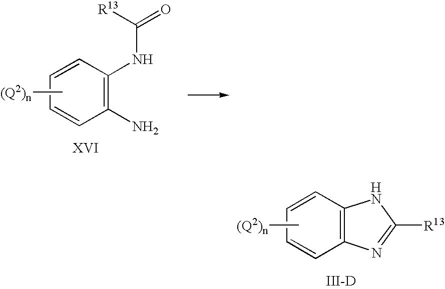
wherein all variables are as defined in connection with Schemes 1-3.

This type of cyclization reaction is well documented in the literature. See, Brana, M. F., et al., J. Med. Chem. 45: 5813-5816 (2002); Fonseca, T., et. al., Tetrahedron 57: 1793-1799 (2001); White, A. W., et. al., J. Med. Chem. 43: 4084-4097 (2000); and Tamura, S. Y., et. al., Biorg. Med. Chem. Lett 7: 1359-1364 (1997). This reaction may be carried out neat or in a suitable solvent. The reaction may optionally be heated to a temperature of from about 50 to about 200° C. Typically an excess of a suitable acid is used. Examples of suitable acids include but are not limited to acetic acid, trifluoroacetic acid, hydrochloric acid, hydrobromic acid, sulfuric acid, methanesulfonic acid, p-toluenesulfonic acid, and pyridinium p-toluenesulfonate. A dehydrating reagent may optionally be used as well. Examples of suitable dehydrating reagents include but are not limited to magnesium sulfate, sodium sulfate, phosphorous pentoxide, and molecular sieves. Examples of suitable solvents include but are not limited to dichloromethane, chloroform, toluene, xylenes, methanol, ethanol, and water.

A compound of formula (XVI) may be prepared by reducing a compound of formula (XV). 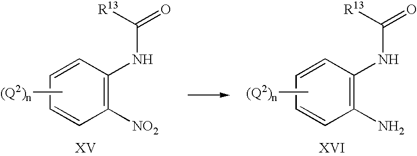
wherein all variables are as defined in connection with Schemes 1-3.

The reduction can be carried out using conventional techniques and reducing agents. See, Rangarajan, M., et al., Bioorg. Med. Chem. 8:2591-2600 (2000); White, A. W., et al., J. Med. Chem. 43: 4084-4097 (2000); Silvestri, R., et al., Bioorg. Med. Chem. 8:2305-2309 (2000); Nagaraja, D., et al., Tetrahedron Lett. 40:7855-7856 (1999); Jung, F., et al., J. Med. Chem. 34:1110-1116 (1991); Srivastava, R. P., et al., Pharmazie 45:34-37 (1990); Hankovszky, H. O., et al., Can. J. Chem. 67:1392-1400 (1989); Ladd, D. L., et al., J. Org. Chem. 53:417-420 (1988); Mertens, A., et al., J. Med. Chem. 30:1279-1287 (1987); and Sharma, K. S., et al., Synthesis 4:316-318 (1981). Examples of suitable reducing agents for this reaction include but are not limited to, palladium with hydrogen, palladium with ammonium formate, platinum oxide with hydrogen, nickel with hydrogen, tin(II) chloride, iron with acetic acid, aluminum with ammonium chloride, borane, sodium dithionite, and hydrazine. The reaction may optionally be heated to between about 50 and about 120° C. Suitable solvents for this reaction vary and include but are not limited to, water, methanol, ethanol, ethyl acetate, tetrahydrofuran, and dioxane.

A compound of formula (X) may be prepared by reacting a compound of formula (XIV) under nitration conditions. 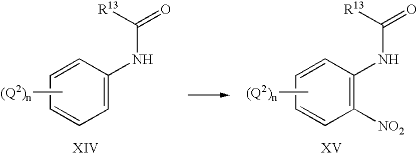
wherein all variables are as defined in connection with Schemes 1-3. The reaction of the compound of formula (XIV) under nitration conditions may be carried out in the same manner as described above for the nitration of a compound of formula (X).

A compound of formula (XIV) may be prepared by acylating a compound of formula (XIII). 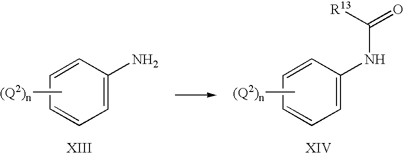
wherein all variables are as defined in connection with Schemes 1-3.

Acylation of anilines is a common transformation well known to one skilled in the art and such conventional acylation techniques may be employed for carrying out the foregoing reaction. See, Larock, R. C. Comprehensive Organic Transformations, VCH Publishers, Inc., New York, pp. 972-976, 979, 981 (1989). The acylation reaction is typically carried out using an acylating agent such as an acid halide, acid anhydride, or carboxylic acid, in the presence of a coupling reagent(s). Examples of suitable coupling reagents include but are not limited to N,N′-dicyclohexylcarbodiimide, 1-(3-dimethylaminopropyl)-3-ethylcarbodiimide hydrochloride, O-(7-azabenzotriazol-1-yl)-N,N,N,N′-tetramethyluronium hexafluorophosphate, and N,N′-carbonyldiimidazole. Suitable solvents include but are not limited to N,N-dimethylformamide, tetrahydrofuran, dioxane, toluene, benzene, 1,2-dimethoxyethane, and 1-methyl-2-pyrrolidinone. Anilines of formula (XIII) are commercially available or readily prepared from commercially available material using conventional techniques.

As will be apparent to those skilled in the art, a compound of formula (I) may be converted to another compound of formula (I) using techniques well known in the art For example, a compound of formula (I-A) may optionally be converted to a compound of formula (I-B) or (I-C) according to the process outlined in Scheme 4. 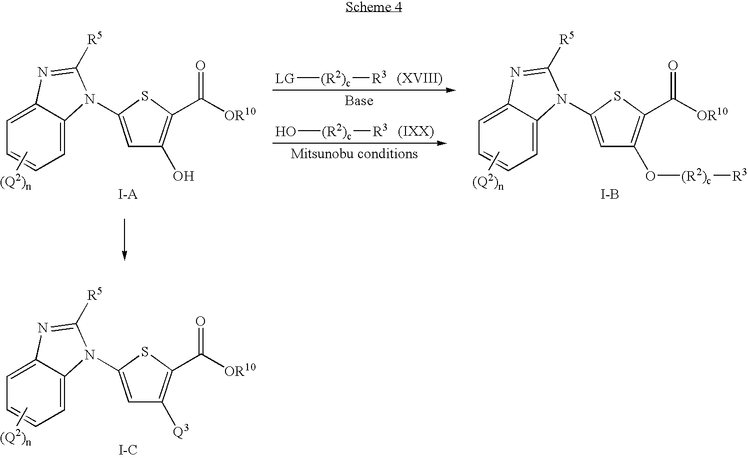
wherein
Q³ is a group of formula:
   —(R²)ₐ—(Y³)ⱼ—(R²)_{c}—R³
j is 0 or 1;
Y³ is selected from the group consisting of —S(O)_{f}—, —N(R⁷)—, —C(O)—, —OC(O)—, —CO₂, —C(O)N(R⁷)—, —C(O)N(R⁷)S(O)₂—, —OC(O)N(R⁷)—, —OS(O)₂—, —S(O)₂N(R⁷)—, —S(O)₂N(R⁷)C(O)—, —N(R⁷)S(O)₂—, —N(R⁷)C(O)—, —N(R⁷)CO₂— and —N(R⁷)C(O)N(R⁷)—;
LG is a suitable leaving group; and
all other variables are as defined in connection with Scheme 1 above.

In general the process for preparing a compound of formula (I-B) comprises the steps of:

a) reacting the compound of formula (I-A) with a base and a compound of formula (XVIII) to prepare a compound of the formula (I-B); or

b) reacting the compound of formula (I-A) with a compound of formula (IXX) under Mitsunobu conditions to prepare a compound of formula (I-B).

More specifically, a compound of formula (I-B) can be prepared by reacting a compound of formula (I-A) with a compound of formula (XVIII). The compounds of formula (XVIII) are commercially available or can be prepared using conventional knowledge in the art. The reaction may be carried out in an inert solvent, conveniently at room temperature, in the presence of a suitable base. The compound of formula (I-A) and the compound of formula (XVIII) may be present in equimolar amounts; however, a slight excess of the compound of formula (XVIII) may be employed if desired. Examples of suitable bases for this reaction include but are not limited to, potassium carbonate, sodium carbonate, cesium carbonate, sodium hydride, and potassium hydride. Examples of suitable inert solvents for this reaction include but are not limited to, N,N-dimethylformamide, tetrahydrofuran, dioxane, and 1,2-dimethoxyethane.

In another embodiment, a compound of formula (I-B) can be prepared by reacting a compound of formula (I-A) with a compound of formula (IXX). The compounds of formula (IXX) are commercially available or can be prepared using conventional knowledge in the art. The reaction is carried out in an inert solvent under standard Mitsunobu conditions. See, Hughes, D. L., Org. React 42:335-656 (1992); and Mitsunobu, O., Synthesis 1-28 (1981). Typically the compound of formula (I-A), the compound of formula (IXX), a triarylphosphine, and a dialkyl azodicarboxylate are reacted together at room temperature. Examples of suitable triarylphosphines include but are not limited to, triphenylphosphine, tri-p-tolylphosphine, and trimesitylphosphine. Examples of suitable dialkyl azodicarboxylates include but are not limited to, diethyl azodicarboxylate, diisopropyl azodicarboxylate, and di-tert-butyl azodicarboxylate. Examples of suitable inert solvents for this reaction include but are not limited to, tetrahydrofuran, dioxane, 1,2-dimethoxyethane, dichloromethane, and toluene.

A compound of formula (I-A) may also be converted to a compound of formula (I-C) according to the following Scheme 5. 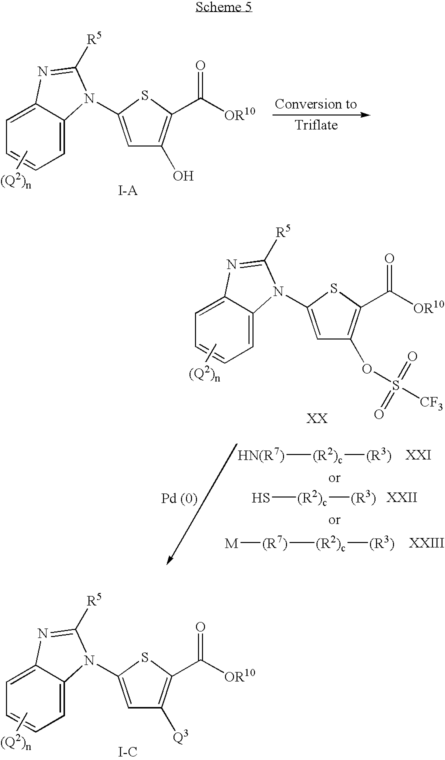
wherein M is —B(OH)₂, —B(OR¹⁴)₂, —Sn(R¹⁴)₂, Zn-halo, Zn—R¹⁴, Mg-halo, Cu-halo, Cu—R¹⁴ where R¹⁴ is alkyl or cycloalkyl, and all other variables are as defined in connection with Schemes 1-4 above.

Generally, the process for preparing a compound of formula (I-C) comprises the steps of:

a) reacting a compound of formula (I-A) with a suitable triflating reagent to prepare a compound of formula (XX); and

b) coupling the compound of formula (XX) with a compound selected from the group consisting of a compound of formula (XXI), (XXII), and (XXIII) using a palladium (0) catalyst to prepare a compound of the formula (I-C).

More specifically, a compound of formula (I-C) can be prepared by reacting a compound of formula (XX) with a compound selected from the group consisting of a compound of formula (XXI), (XXII), and (XXIII) using a palladium (0) catalyst. This reaction may be carried out in an inert solvent, in the presence of palladium (0). The reaction may optionally be heated to a temperature of from about 50 to about 150° C. Typically, the reaction is carried out by reacting an equimolar amount of a compound of formula (XX) with an equimolar amount of the compound selected from the group consisting of compounds of formula (XXI), (XXII) and (XXIII). The palladium (0) catalyst is typically present in 1-10 mole percent compared to the compound of formula (XX). Examples of suitable palladium catalysts include but are not limited to, tetrakis(triphenylphosphine)palladium (0) and tris(dibenzylideneacetone)dipalladium (0). It is also possible to generate the palladium (0) catalyst in situ using palladium (II) sources. Examples of suitable palladium (II) sources include but are not limited to, palladium (II) acetate, palladium (II) chloride, palladium (II) trifluoroacetate, dichlorobis(triphenyl-phosphine)palladium (II), and bis(diphenylphosphinoferrocene)-palladium (II) dichloride. Suitable solvents for this reaction include but are not limited to N,N-dimethylformamide, tetrahydrofuran, dioxane, toluene, benzene, 1,2-dimethoxyethane, and 1-methyl-2-pyrrolidinone. Bases and phosphines may be included as additives in the reaction if desired. Examples of suitable bases include but are not limited to cesium carbonate, sodium carbonate, and trialkylamines. Examples of suitable phosphine additives include but are not limited to triphenylphosphine, tributylphosphine, diphenylphosphinoethane, and 2,2′-bis(diphenylphosphino)-1,1′-binaphthyl. Compounds of the formula (XXI), (XXII) and (XXIII) may be obtained from commercial sources or prepared either as discreet compounds or generated in situ using conventional knowledge in the art See, Luker, T. J., et al., Tetrahedron Lett 41:7731-7735 (2000); Yin, J., et al., Org. Lett. 2:1101-1104 (2000); Wolfe, J. P., et al., Can. J. Chem. 78:957-962 (2000); Littke, A. F., et al., J. Am. Chem. Soc. 122:4020-4028 (2000); Hundertmark, T., et al., Org. Lett 2:1729-1731 (2000); Buchwald, S. L., Acc. Chem. Res. 31:805-818 (1998); Suzuki, A., J. Organomet. Chem. 576:147-168 (1999); Negishi, E., J., Organomet Chem. 576:179-194 (1999); Stanforth, S. P., Tetrahedron 54:263-303 (1998); Littke, A. F., Angew. Chem., Int Ed. 37:3387-3388 (1999); and Thorand, S., et al., J. Org. Chem. 63:8551-8553 (1998).

A compound of formula (XX) can be prepared from a compound of formula (I-A) using a suitable triflating reagent. This reaction is typically carried out in an inert solvent using a base and a reagent designed for conversion of alcohols into triflates (i.e., a triflating reagent). Examples of suitable bases include but are not limited to sodium carbonate, trialkylamines, pyridine, sodium hydride, and lithium bis(trimethylsilyl) amide. The reaction is preferably run at a temperature of from about 0 to about 25° C. Suitable triflating reagents for this reaction include but are not limited to, trifluoromethanesulfonic anhydride, trifluoromethanesulfonyl chloride, and N-phenyltrifluoromethanesulfonimide. Suitable inert solvents for this reaction include but are not limited to tetrahydrofuran, dichloromethane, toluene, chloroform, diethyl ether, and dioxane.

As a further example of methods for converting a compound of formula (I) to another compound of formula (I), a compound of formula (I-A), (I-B), or (I-C) (collectively referred to as a compound of formula “(I-D)” may be converted to a different compound of formula (I) 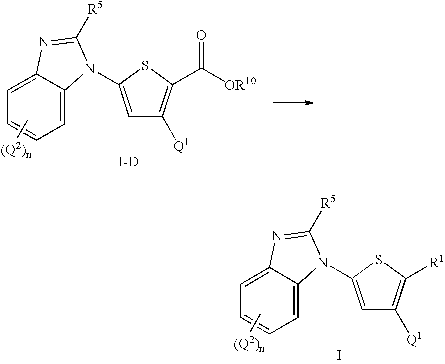
wherein:
R¹ is other than —CO₂R¹⁰;
and all other variables are as defined in connection with Schemes 1-5.

Several methods, using conventional techniques can be employed to convert a compound of formula (I-D) to a different compound of formula (I), depending upon the particular compound of formula (I) that is desired. For example, according to one method, a compound of formula (I-D) can be converted to a compound of formula (I-E) by removal of the carboxylic acid protecting group. 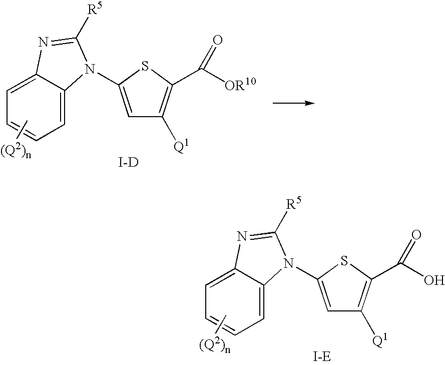
wherein all variables are as defined in connection with Schemes 1-5.

There are several options for carrying out this conversion. Examples of suitable conditions include but are not limited to, basic hydrolysis where R¹ is —CO₂Me, deprotection with protic acid where R¹ is —CO₂t-Bu, deprotection under palladium (0) catalysis where R¹ is CO₂CH₂CH═CH₂, deprotection with tetrabutylammonium fluoride where R¹ is CO₂CH₂CH₂Si(CH₃)₃, and hydrogenolysis where R¹ is CO₂CH₂Ph. Other suitable conditions for compounds with various R¹⁰ definitions will be apparent to those skilled in the art. The choice of protecting group and deprotection conditions will be apparent to one skilled in the art and, detailed information on this subject is available in the literature. See, Kocienski, P. J. Protecting Groups, Georg Thieme Verlag, Stuttgart, 1994; and Greene, T. W., Wuts, P. G. M. Protecting Groups in Organic Synthesis (2^{nd} Edition), J. Wiley and Sons, 1991.

A compound of formula (I-E) may be further converted to a compound of formula (I-F) by heating. 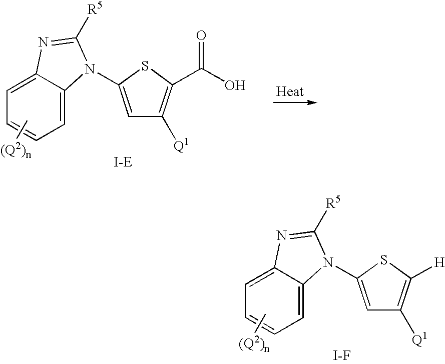
wherein all variables are as defined above in connection with Schemes 1-5.

This reaction may be performed in an inert solvent. Typically, the reaction is heated to a temperature of from about 80 to about 120° C. Examples of suitable solvents for this reaction include but are not limited to acetic acid, propionic acid, N,N-dimethylformamide, dimethylsulfoxide, ethanol, dioxane and toluene.

A compound of formula (I-E) may be further converted to a compound of formula (I-G) using conventional amide bond coupling reactions with an amine of formula HNR⁷R⁸. 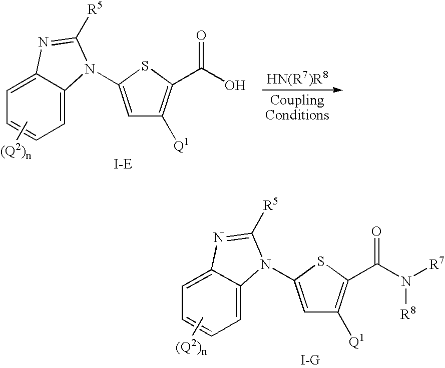
wherein all variables are as defined in connection with Schemes 1-5.

This reaction can be carried out in an inert solvent using a variety of commercially available coupling reagents. Suitable coupling reagents include but are not limited to N,N-dicyclohexylcarbodiimide, 1-(3-dimethylaminopropyl)-3-ethylcarbodiimide hydrochloride, 1,1′-carbonyldiimidazole, and benzotriazol-1-yloxytris(dimethyl-amino)phosphonium hexafluorophosphate. Other suitable coupling reagents will be readily apparent to those skilled in the art The carboxylic acid optionally may be converted into the corresponding acid chloride and subsequently treated with the amine of formula HNR⁷R⁸. Suitable reagents for the reaction of such acid chlorides include but are not limited to oxalyl chloride, thionyl chloride, and 1-chloro-N,N,2-trimethyl-1-propenylamine. Base may be optionally added to the coupling reaction. The reaction may optionally require heating to a temperature of from about 40 to about 100° C. Suitable bases include but are not limited to trialkylamines, pyridine, and 4-(dimethylamino)pyridine. Examples of suitable solvents for this reaction include but are not limited to dichloromethane, chloroform, benzene, toluene, N,N-dimethylformamide and dichloroethane.

In an alternative embodiment, a compound of formula (I-G′) is prepared directly from a compound of formula (I-D). 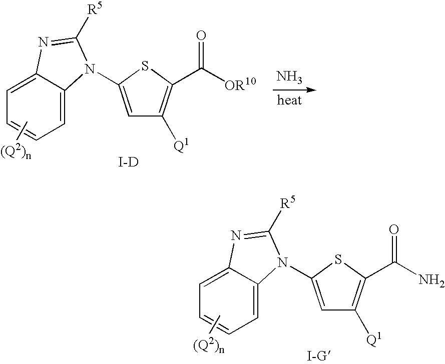
wherein all variables are as defined in connection with Schemes 1-5.

This reaction is typically performed in a sealed vessel with an excess of ammonia. The reaction is typically heated to a temperature of from about 50 to about 120° C. Suitable solvents for this reaction include but are not limited to methanol, ethanol, isopropanol, tetrahydrofuran, and dioxane.

Dehydration of the compound of formula (I-G′) may be used to prepare a compound of formula (I-H). 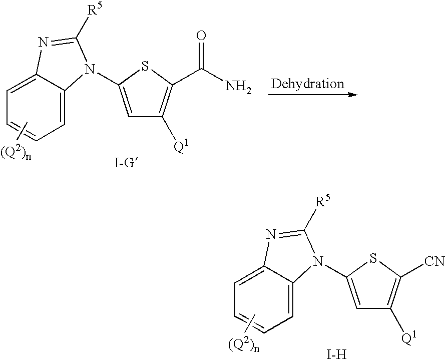
wherein all variables are as defined in connection with Schemes 1-5.

The dehydration reaction can be carried out using a variety of reagents. Suitable dehydration reagents include but are not limited to thionyl chloride, trifluoroacetic anhydride, phosphorous oxychloride, phosphorous pentoxide, and N,N-dicyclohexylcarbodiimide. The reaction may be optionally heated to from about 50 to about 150° C. Suitable solvents for this reaction include but are not limited to dichloromethane, chloroform, benzene, toluene, N,N-dimethylformamide, and dichloroethane.

A compound of formula (I-J) may be prepared through a two step conversion process, comprising a) converting a compound of formula (I-E) to a compound of formula (I-I) by coupling with N,O-dimethylhydroxylamine, and b) reacting the compound of formula (I-I) with a nucleophile of formula M¹-R⁷. 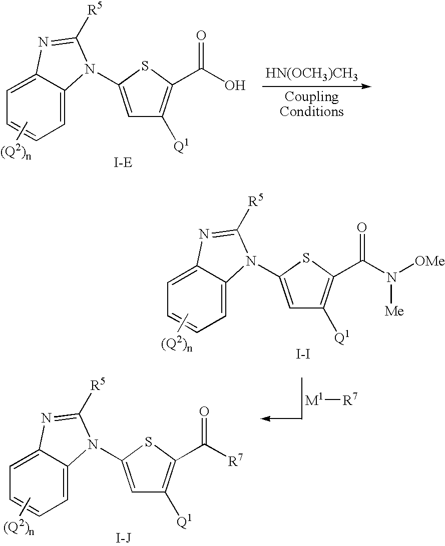
wherein
M¹ is Li, Mg-halo, Cu-halo or Ce-halo; and
all variables are as defined in connection with Schemes 1-5.

The coupling reaction with N,O-dimethylhydroxylamine may be carried out in the same manner as described above for the conversion of a compound of formula (I-E) to a compound of formula (I-G). The addition of the nucleophile to the Weinreb amide (I-I) is typically carried out at a temperature ranging from about −30 to about 5° C. Suitable solvents for this reaction include but are not limited to, tetrahydrofuran, dioxane, diethyl ether, toluene, 1,2-dimethoxyethane, and hexanes. See, Weinreb, S. M., et al., Tetrahedron Lett 22:3815-3818 (1981). Nucleophiles of formula M¹-R⁷ are commercially available or can be prepared using conventional knowledge in the art.

A compound of formula (I-K) may be prepared from a compound of formula (I-D) through a hydride reduction. 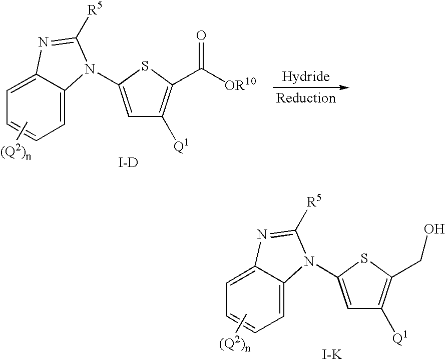
wherein all variables are as defined in connection with Schemes 1-5.

This reaction may be carried out in an inert solvent at a temperature ranging from about −78 to about 25° C. Suitable reducing agents include but are not limited to diisobutylaluminum hydride, lithium aluminum hydride, and lithium borohydride. Suitable solvents vary considerably depending on the chosen reducing agent. Appropriate selection of a solvent for this reaction will be apparent to those skilled in the art based upon the choice of reducing agent. Examples of suitable solvents include but are not limited to tetrahydrofuran, diethyl ether, 1,2-dimethoxyethane, dioxane, dichloromethane, toluene, and hexanes.

A compound of formula (I-K) may be oxidized to prepare a compound of formula (I-L). 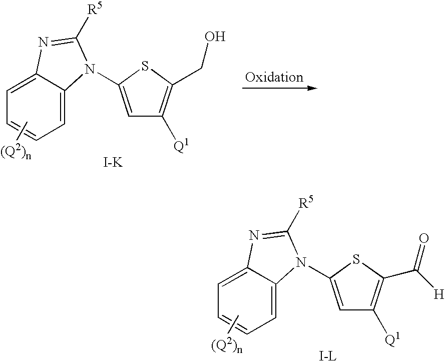
wherein all variables are as defined in connection with Schemes 1-5.

This reaction can be carried out using a wide variety of conventional oxidizing agents.

Suitable oxidizing agents include but are not limited to, manganese dioxide, dimethyl sulfoxide/oxalyl chloride/triethylamine, pyridinium chlorochromate, pyridinium dichromate, and tetrapropylammonium perruthenate/4-methylmorpholine N-oxide. Examples of suitable solvents for the oxidation reaction include but are not limited to, dichloromethane, chloroform, diethyl ether, toluene, and tetrahydrofuran.

A compound of formula (I-L) may be further converted to a compound of formula (I-M) by reacting with a nucleophile of formula M¹-R⁷. 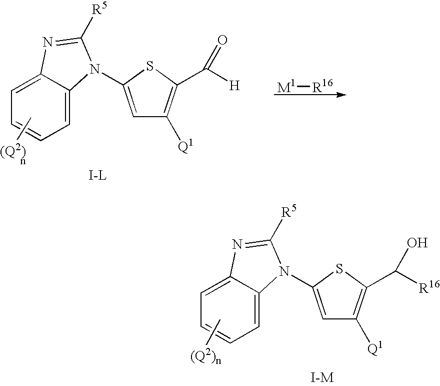
wherein M¹ is Li, Mg-halo, Cu-halo or Ce-halo,
R¹⁶ is H, alkyl, alkenyl or alkynyl; and
all other variables are as defined in connection with Schemes 1-5.

The addition of the nucleophile M¹-R¹⁶ to the aldehyde of formula (I-L) is typically carried out at a temperature ranging from about −78 to about 5° C. Suitable solvents for this reaction include but are not limited to, tetrahydrofuran, dioxane, diethyl ether, toluene, 1,2-dimethoxyethane, and hexanes.

As an alternative to the previously described method, a compound of the formula (I-J) may also be prepared by conversion from a compound of formula (I-M). More specifically, a compound of formula (I-J) may be prepared by oxidation of a compound of formula (I-M). 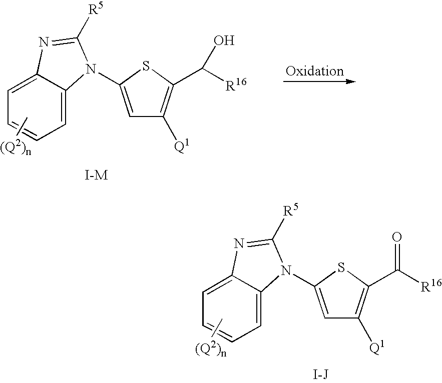
wherein R¹⁶ is H, alkyl, alkenyl or alkynyl; and
all other variables are as defined in connection with Schemes 1-5.

This reaction can be carried out using a wide variety of conventional oxidizing agents. Examples of suitable oxidizing agents include but are not limited to, manganese dioxide, dimethyl sulfoxide/oxalyl chloride/triethyl amine, pyridinium chlorochromate, pyridinium dichromate, and tetrapropylammonium perruthenate/4-methylmorpholine N-oxide. Suitable solvents for this reaction include but are not limited to, dichloromethane, chloroform, diethyl ether, toluene and tetrahydrofuran.

Further, a compound of formula (I-J) may be converted to a compound of formula (I-M′) by reacting with a nucleophile of formula M¹-R¹⁶. 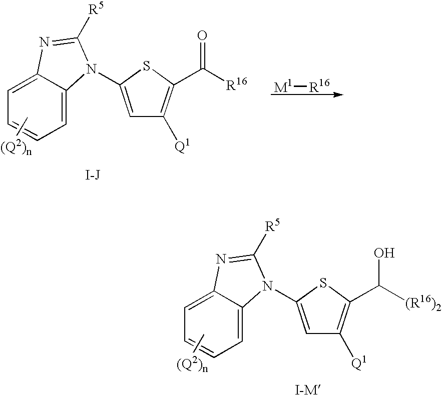
wherein M¹ is Li, Mg-halo, Cu-halo or Ce-halo;
R¹⁶ is H, alkyl, alkenyl or alkynyl; and
all other variables are as defined above in connection with Schemes 1-5.

Nucleophiles of formula M¹-R¹⁶ are commercially available or can be prepared using conventional knowledge in the art.

The addition of the nucleophile to the aldehyde of formula (I-J) is typically carried out at a temperature ranging from about −78 to about 5° C. Suitable solvents for this reaction include but are not limited to, tetrahydrofuran, dioxane, diethyl ether, toluene, 1,2-dimethoxyethane, and hexanes.

A compound of formula (I-M) may be further converted to a compound of formula (I-N) by halogenating the compound of formula (I-M). 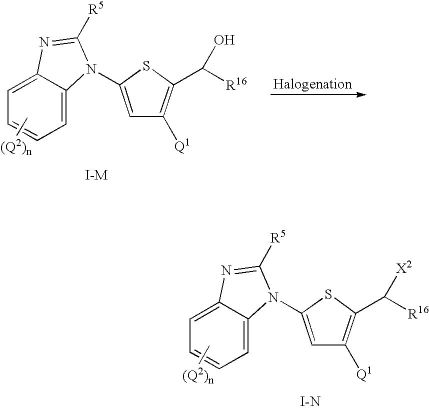
wherein X² is halo;
R¹⁶ is H, alkyl, alkenyl or alkynyl; and
all other variables are as defined in connection with Schemes 1-5.

This reaction may be carried out using any conventional halogenating reagent. Examples of suitable halogenating reagents include but are not limited to triphenylphosphine/iodine/imidazole, triphenylphosphine/carbon tetrabromide, phosphorous pentachloride, thionyl chloride, phosphorous tribromide, hydrofluoric acid/potassium fluoride, and dimethyl sulfide/N-bromosuccinimide. Suitable solvents for this reaction include but are not limited to tetrahydrofuran, dioxane, diethyl ether, dichloromethane, chloroform, acetonitrile, toluene, 1,2-dimethoxyethane, and hexanes.

A compound of formula (I-N) may be further converted to a compound of formula (I-O) using a reduction. 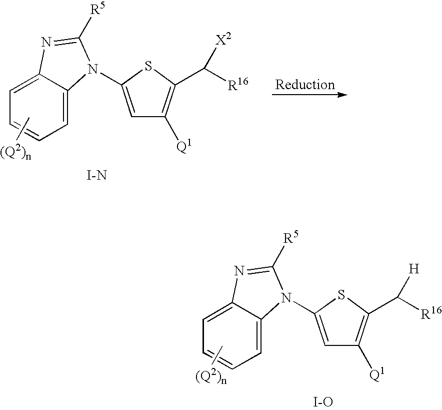
wherein X² is halo;
R¹⁶ is H, alkyl, alkenyl or alkynyl; and
all other variables are as defined above in connection with Scheme 2.

This reaction may be carried out in an inert solvent using a variety of conditions. Examples of suitable reducing agents for this reaction include but are not limited to, lithium/ammonia, zinc/acetic acid, lithium triethylborohydride, tributyltin hydride, lithium aluminum hydride, and samarium (II) iodide. Suitable solvents for this reaction vary considerably depending upon the chosen reducing agent. Examples of suitable solvents include but are not limited to, tetrahydrofuran, diethyl ether, 1,2-dimethoxyethane, dioxane, toluene, and hexanes.

A compound of formula (I-L) may be further converted to a compound of formula (I-P) by reacting with a compound of the formula (XXV). 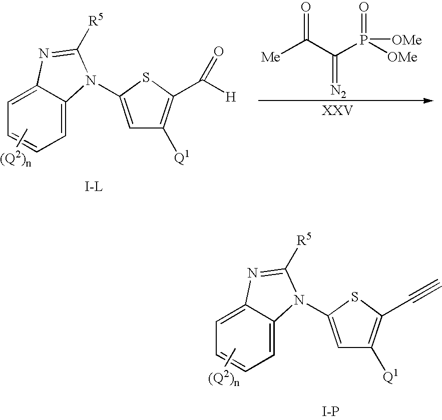
wherein all variables are as defined above in connection with Schemes 1-5.

This reaction is carried out in an inert solvent, conveniently at room temperature. The synthesis and use of the compound of formula (XXV) is analogous to that described in Mueller, S., et al., Synlett 6:521-522(1996). Typically, the reaction is carried out using methanol as the solvent and a base such as potassium carbonate.

In another embodiment, a compound of formula (I-Q) may be converted to a compound of formula (I-R), which may in turn be converted to a compound of formula (I-S), or a compound of formula (I-Q) may be converted directed to a compound of formula (I-S). 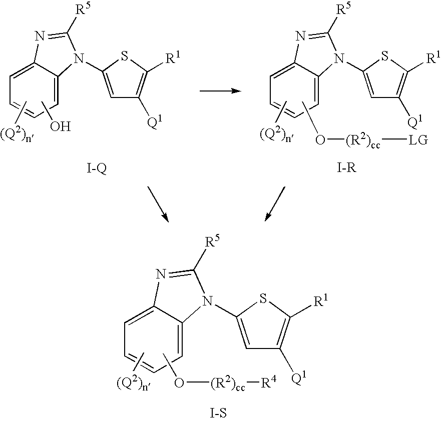
wherein
n′ is 0, 1, 2 or 3;
each LG is the same or different suitable leaving group; and
all other variables are as defined above in connection with Schemes 1-5.

Compounds of formula (I-Q) may be prepared according to any of the methods described herein above. The compound of formula (I-Q) may then be converted to a compound of formula (I-R) or a compound of formula (I-S).

The compound of formula (I-R) may be prepared by either of two methods. According to one method, a compound of formula (I-R) is prepared by reacting a compound of formula (I-Q) with a compound of formula: LG-(R²)_{cc}-LG (XXVII), wherein all variables are as defined above. Specific examples of suitable leaving groups include but are not limited to —Cl, —Br, —I, —OSO₂CH₃ and —OSO₂-Phenyl. Suitable compounds of formula (XXVII) are commercially available or may be prepared using conventional techniques. The reaction may be carried out in an inert solvent, conveniently at room temperature, in the presence of a suitable base. Examples of suitable bases for this reaction include but are not limited to, potassium carbonate, sodium carbonate, cesium carbonate, sodium hydride, and potassium hydride. Examples of suitable inert solvents for this reaction include but are not limited to, N,N-dimethylformamide, tetrahydrofuran, dioxane, and 1,2-dimethoxyethane.

According to a second method, a compound of formula (I-R) is prepared by reacting a compound of formula (I-Q) with a compound of formula: HO—(R²)_{cc}-LG (XXVIII), wherein all variables are as defined above. Specific examples of suitable leaving groups include those described above. Compounds of formula (XXVIII) are commercially available or can be prepared using conventional techniques. The reaction is carried out in an inert solvent under standard Mitsunobu conditions. See, Hughes, D. L., Org. React 42:335-656 (1992); and Mitsunobu, O., Synthesis 1-28 (1981). Typically the compound of formula (I-Q) and the compound of formula (XXVIII) are reacted together with a triarylphosphine, and a dialkyl azodicarboxylate at room temperature. Examples of suitable triarylphosphines include but are not limited to, triphenylphosphine, tri-p-tolylphosphine, and trimesitylphosphine. Examples of suitable dialkyl azodicarboxylates include but are not limited to, diethyl azodicarboxylate, diisopropyl azodicarboxylate, and di-tert-butyl azodicarboxylate. Examples of suitable inert solvents for this reaction include but are not limited to, tetrahydrofuran, dioxane, 1,2-dimethoxyethane, dichloromethane, and toluene.

The compound of formula (I-R) may be converted to a compound of formula (I-S) by reaction with a suitable nucleophile for installing the group R⁴. Examples of suitable nucleophiles include but are not limited to ammonia, primary and secondary amines, metal alkoxides, metal thioalkoxides, potassium cyanide, sodium azide, organolithium reagents, organocuprates, and Grignard reagents. The specific conditions for these displacements vary, but the use of these types of nucleophiles for the installation of a group as defined by R⁴ are conventional in the art. Displacement of the leaving group with such a nucleophile would either install the R⁴ functionality or provide an intermediate from which the R⁴ functional group could be readily installed according to conventional methods by one skilled in the art.

Alternatively, a compound of formula (I-S) may be prepared directly from a compound of formula (I-Q) using procedures analogous to those described above for the conversion of a compound of formula (I-Q) to a compound of formula (I-R). More specifically, a compound of formula (I-S) may be prepared by reacting a compound of formula (I-Q) with a compound of formula: LG-(R²)_{cc}—R⁴ (XXIX), using conditions analogous to those described above for the reaction of a compound of formula (I-Q) with a compound of formula (XXVII). Compounds of formula (XXIX) are commercially available or can be prepared using conventional techniques.

In another embodiment, a compound of formula (I-Q) is converted to a compound of formula (I-S) by reacting with a compound of formula: HO—(R²)_{cc}—R⁴ (XXX) under the conditions described above for the reaction of a compound of formula (I-Q) with a compound of formula (XXVIII). Compounds of formula (XXX) are commercially available or can be prepared using conventional techniques.

As a further example, a compound of formula (I-T) may be converted to a compound of formula (I-U), which may optionally be further converted to a compound of formula (I-V). 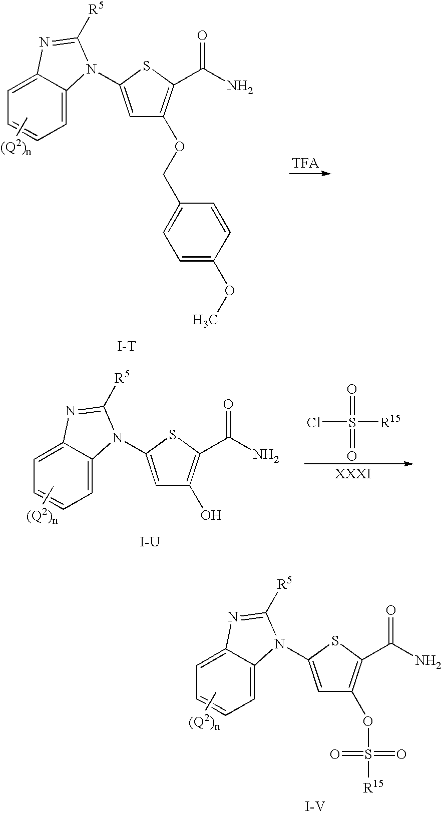
wherein:
R¹⁵ is alkyl or phenyl; and
all other variables are as defined in connection with Schemes 1-5 above.

A compound of formula (I-T) may be converted to a compound of formula (I-U) by reacting with a suitable acid, such as trifluoroacetic acid (TFA). This reaction may be carried out neat or in an inert solvent at ambient temperature. Suitable solvents for this reaction include but are not limited to, dichloromethane and chloroform.

The compound of formula (I-U) may be further converted to a compound of formula (I-V) by reacting with sulfonyl chlorides of formula (XXXI). The reaction may be carried out in an inert solvent at ambient temperature using a variety of bases. Examples of suitable bases include but are not limited to, triethylamine, N,N-diisopropylethylamine, and pyridine. Suitable solvents for this reaction include but are not limited to, dichloromethane, chloroform, tetrahydrofuran, 1,2-dimethoxyethane, dioxane, and N,N-dimethylformamide.

In another embodiment, a compound of formula (I-W) may be converted to a compound of formula (I-X). A compound of formula (I-X) may be further converted to a compound of formula (I-Y). 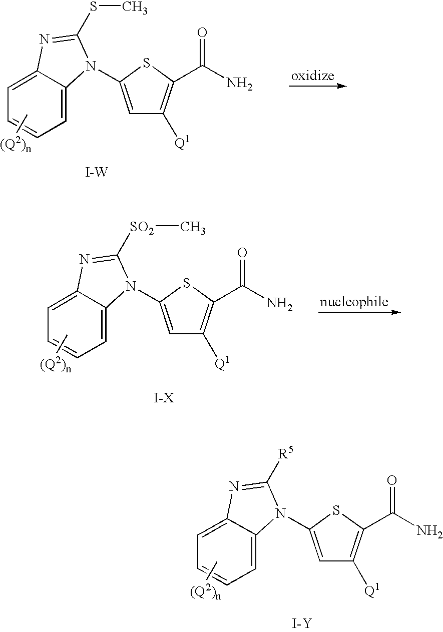
wherein R¹ is selected from the group consisting of —OR⁷ and —NR⁷R⁸; and
all other variables are as defined in connection with Schemes 1-5 above.

A compound of formula (I-W) may be oxidized to a compound of formula (I-X) using a conventional oxidizing agent, such as for example, 3-chloroperoxybenzoic acid. Reaction of the compound of formula (I-X) with a suitable nucleophile of formula R⁵² will convert a compound of formula (I-X) to a compound of formula (I-Y). Specific examples of suitable nucleophiles for this reaction include but are not limited to sodium hydroxide, sodium acetate, ammonia, and mono and di-substituted amines. The reaction with the nucleophile is typically carried out using equimolar or a slight excess of the nucleophile in an inert solvent, such as THF, at ambient or elevated temperatures. In another embodiment, a compound of formula (I-X) may be converted to a compound of formula (I-Y) in a sealed tube at elevated temperatures between 80° C. and 120° C., using excess ammonia in an appropriate solvent such as methanol, ethanol, isopropanol, tetrahydrofuran and dioxane.

Similarly, a compound of formula (I-AA) may also be converted to a compound of formula (I-BB) by oxidation, and the compound of formula (I-BB) may be converted to a compound of formula (I-CC) by reaction with ammonia. 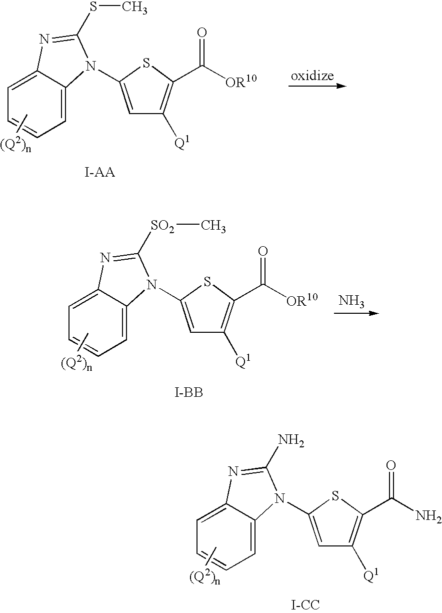
wherein all variables are as defined in connection Schemes 1-5 above.

The step of converting a compound of formula (I-AA) to a compound of formula (I-BB) may be carried out by reacting a compound of formula (I-AA) with a suitable oxidizing agent, such as for example 3-chloroperoxybenzoic acid. The compound of formula (I-BB) may be converted to a compound of formula (I-CC) by reaction with excess ammonia in a sealed tube at elevated temperature between about 80 and about 120° C. in a suitable solvent. Suitable solvents for this reaction include but are not limited to methanol, ethanol, isopropanol, tetrahydrofuran and dioxane.

A further example of a process for converting a compound of formula (I) to a different compound of formula (I) includes the reaction of a compound of formula (I-DD) with a thionating reagent to prepare a compound of formula (I-EE). 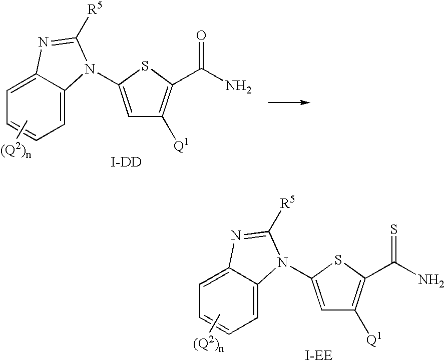
wherein all variables are as defined in connection with Schemes 1-5 above.

The reaction may be carried out in an inert solvent and optionally heated to a temperature of from about 65 to above about 100° C. Examples of suitable thionating reagents include but are not limited to phosphorus pentasulfide, 2,4-bis(4-methoxyphenyl)-1,3-dithia-2,4-diphosphetane-2,4-disulfide and the like. Suitable solvents include but are not limited to xylene, dioxane and toluene.

Further, a compound of formula (I-FF) may be converted to a compound of formula (I-GG) by reaction with an azide source in an inert solvent. 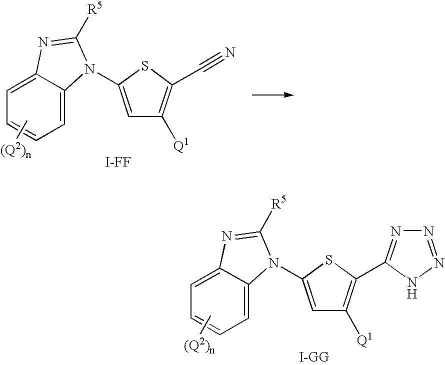
wherein all variables are as defined in connection with Schemes 1-5 above.

Examples of suitable azide sources include but are not limited to hydrazoic aicd, sodium azide with ammonium chloride, sodium azide with aluminum chloride, and sodium azide with zinc(II) bromide. By way of example some preferred solvents include but are not limited to dimehtylformamide, dimethylsulfoxide, N-methylpyrrolidinone, toluene and the like. The reaction may be optionally heated to a temperature of from about 23 to about 150° C.

In another embodiment, a compound of formula (I-HH) may be converted to a compound of formula (I-II) using a coupling protocol. 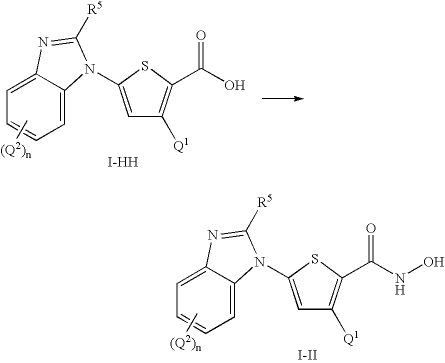
wherein all variables are as defined in any of Schemes 1-5.

The conversion reaction can be carried out by reacting a compound of formula (I-HH) with a suitable coupling reagent in an inert solvent, followed by the addition of a hydroxylamine source, and optionally a base. Suitable coupling reagents include but are not limited to 1,1-carbonyldiimidazole, oxalyl chloride, dicyclohexylcarbodiimide and 1-(N,N-diphenylcarbamoyl)pyridinium chloride. Preferrably the hydroxylamine is hydroxylamine hydrochloride. Suitable bases include but are not limited to triethylamine, sodium methoxide and diisoproylethylamine. The reaction may be optionally heated to a temperature of from about 0° C. to about 80° C. Examples of suitable solvents for this reaction include but are not limited to dimethylformamide, dichloromethane and tetrahydrofuran.

In yet another example of a conversion using a coupling protocol a compound of formula (I-KK) is prepared from a compound of formula (I-JJ) as follows. 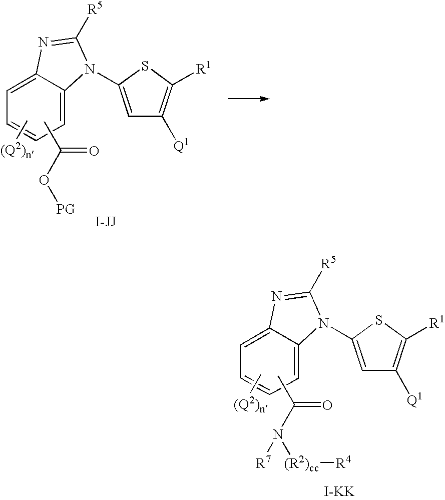
wherein n′ is 0, 1, 2 or 3;
PG is a protecting group and
all other variables are as defined in any of Schemes 1-5 above.

The protecting group is typically carboxylic acid protecting group which when removed yields the acid. The cleavage of the carboxylic acid protecting group can be accomplished through many different methods conventional in the art See, Kocienski, P. J. Protecting Groups, Georg Thieme Verlag, Stuttgart, 1994; and Greene, T. W., Wuts, P. G. M. Protecting Groups in Organic Synthesis (2^{nd} Edition), J. Wiley and Sons, 1991.

Following the removal of the protecting group, the resulting carboxylic acid is reacted using a coupling protocol to yield the compound of formula (I-KK). The reaction can be carried out by reacting the deprotected compound of formula (I-JJ) with a suitable coupling reagent in an inert solvent, followed by the addition of a primary or secondary amine, and optionally a base. Suitable coupling reagents include but are not limited to 1,1-carbonyldiimidazole, oxalyl chloride, dicyclohexylcarbodiimide and O-(7-azabenzotriazol-1-yl)-1,1,3,3-tetramethyluronium hexafluorophosphate. Suitable bases include but are not limited to triethylamine, diisoproylethylamine and the like. The reaction may be optionally heated to a temperature of from about 0° C. to about 80° C. Examples of suitable solvents include but are not limited to dimethylformamide, dichloromethane and tetrahydrofuran.

In yet another example of a conversion using a coupling protocol a compound of formula (I-MM) is prepared from a compound of formula (I-LL) as follows. 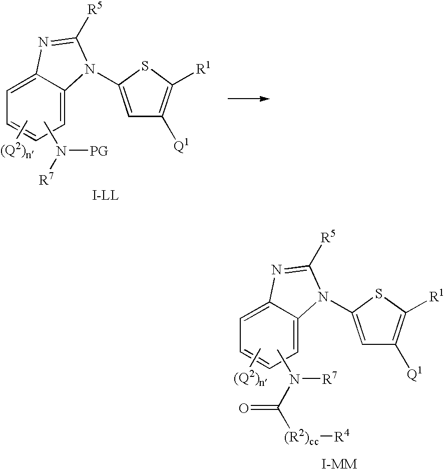
wherein n′ is 0, 1, 2 or 3;
PG is a protecting group and
all other variables are as defined in any of Schemes 1-5 above.

The protecting group is amino protecting group which when removed yields the amine. The cleavage of the amino protecting group can be accomplished through many different methods conventional in the art. See, Kocienski, P. J. Protecting Groups, Georg Thieme Verlag, Stuttgart, 1994; and Greene, T. W., Wuts, P. G. M. Protecting Groups in Organic Synthesis (2^{nd} Edition), J. Wiley and Sons, 1991.

Following the removal of the protecting group, the resulting amine is reacted using a coupling protocol to yield the compound of formula (I-MM). The reaction can be carried out by reacting the deprotected compound of formula (I-LL) with a carboxylic acid in the presence of a suitable coupling reagent in an inert solvent, and optionally a base. Suitable coupling reagents include but are not limited to 1,1-carbonyldiimidazole, oxalyl chloride, dicyclohexylcarbodiimide and 0-(7-azabenzotriazol-1-yl)-1,1,3,3-tetramethyluronium hexafluorophosphate. Suitable bases include but are not limited to triethylamine, diisoproylethylamine and the like. The reaction may be optionally heated to a temperature of from about 0° C. to about 80° C. Examples of suitable solvents include but are not limited to dimethylformamide, dichloromethane and tetrahydrofuran.

Based upon this disclosure and the examples contained herein one skilled in the art can readily convert a compound of formula (I) or a pharmaceutically acceptable salt, solvate or physiologically functional derivative thereof into another compound of formula (I), or a pharmaceutically acceptable salt, solvate or physiologically functional derivative thereof.

The present invention also provides radiolabeled compounds of formula (I) and biotinylated compounds of formula (I) and solid-support-bound versions thereof. Radiolabeled compounds of formula (I) and biotinylated compounds of formula (I) can be prepared using conventional techniques. For example, radiolabeled compounds of formula (I) can be prepared by reacting the compound of formula (I) with tritium gas in the presence of an appropriate catalyst to produce radiolabeled compounds of formula (I).

In one embodiment, the compounds of formula (I) are tritiated.

The radiolabeled compounds of formula (I) and biotinylated compounds of formula (I) are useful in assays for the identification of compounds which inhibit PLK, for the identification of compounds for the treatment of a condition mediated by PLK, for the treatment of susceptible neoplasms, for the treatment of conditions characterized by inappropriate proliferation, for the inhibition of proliferation of a cell and for the inhitibion of mitosis in a cell. Accordingly, the present invention provides an assay method for identifying such compounds, which method comprises the step of specifically binding the radiolabeled compound of formula (I) or the biotinylated compound of formula (I) to the target protein or cellular homogenates. More specifically, suitable assay methods will include competition binding assays. The radiolabeled compounds of formula (I) and biotinylated compounds of formula (I) and solid-support-bound verstions thereof, can be employed in assays according to the methods conventional in the art.

The following examples are intended for illustration only and are not intended to limit the scope of the invention in any way, the invention being defined by the claims which follow.

Reagents are commercially available or are prepared according to procedures in the literature. In the following structures, “Me” refers to the group —CH₃.

### EXAMPLE 1

### Methyl 2-chloro-3-oxo-2,3-dihydro-2-thiophenecarboxylate

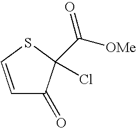

To a solution of methyl 3-hydroxy-2-thiophenecarboxylate (5.00 g, 31.6 mmol) in chloroform (10 mL) was added 1M sulfuryl chloride in dichloromethane (34.8 mL, 34.8 mmol) dropwise over 2 minutes under a N₂ atmosphere. The mixture was stirred for 4 hours at room temperature and the volatiles removed under reduced presssure. The solids were recrystallized from hexane to give methyl 2-chloro-3-oxo-2,3-dihydro-2-thiophenecarboxylate (4.60 g, 76%) as white needles. ¹H NMR (CDCl₃): δ 8.38 (d, 1H), 6.23 (d, 1H), 3.84 (s, 3H); MS m/z 193 (M+1).

### EXAMPLE 2A

### Methyl 5-(1H-benzimidazol-1-yl)-3-hydroxy-2-thiophenecarboxylate

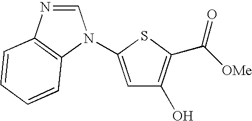

To a solution of methyl 2-chloro-3-oxo-2,3-dihydro-2-thiophenecarboxylate (0.050 g, 0.26 mmol) in chloroform (1.0 mL) (and in a separate reaction acetic acid (1.0 mL)) was added benzimidazole (0.061 g, 0.52 mmol) to each reaction. The chloroform reaction was stirred for 22 hours at room temperature and then diluted with chloroform (2.0 mL). The organic phase was washed with water (1.0 mL) and the phases were separated. The organic phase was analyzed by LC-MS and then concentrated under reduced pressure to give a solid residue. The residue was triturated with water (2 mL), filtered and dried. The acetic acid reaction was stirred at room temperature for 66 hours, and analyzed by LC-MS. The reaction was diluted with water (5 mL), then cooled on ice for 30 minutes and the solids collected by filtration and dried at 50° C. under vacuum. The solids from both the chloroform and acetic acid reactions were analyzed by ¹H-nmr. When both reactions were of sufficient purity they were combined to give methyl 5-(1H-benzimidazol-1-yl)-3-hydroxy-2-thiophenecarboxylate (0.058 g, 41%) as an orange-brown solid. ¹H NMR (DMSO-d₆): δ10.87 (br s, 1H), 8.69 (s, 1H), 7.80 (m, 2H), 7.39 (m, 2H), 7.14 (s, 1H), 3.79 (s, 3H). MS m/z 275 (M+1).

### EXAMPLE 2B

### Methyl 5-(1H-benzimidazol-1-yl)-3-[(2-methylbenzyl)oxy]-2-thiophenecarboxylate and 5-(1H-Benzimidazol-1-yl)-3-[(2-methylbenzyl)oxy]-2-thiophenecarboxamide

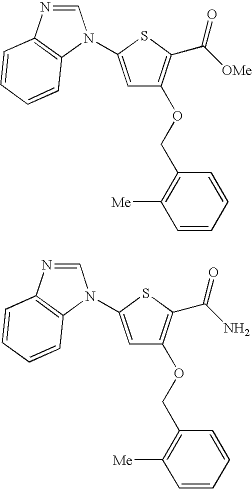

To a mixture of methyl 5-(1H-benzimidazol-1-yl)-3-hydroxy-2-thiophenecarboxylate (0.058 g, 0.21 mmol) and potassium carbonate (0.032 g, 0.23 mmol) in dimethylformamide (0.50 mL) was added α-bromo-o-xylene (31 μL, 0.23 mmol). The mixture was stirred for 6 hours at room temperature and then diluted with water (1.0 mL). The mixture was extracted with ether (2×3 mL) and the combined ether extract was concentrated to dryness under reduced pressure. The residue was treated with 2M ammonia in methanol (3 mL) in a Pyrex test tube sealed with a Teflon-lined screw cap, and the reaction heated to 80° C. with magnetic stirring for 3 days. The reaction was cooled and fresh 2M ammonia in methanol (2 mL) was added and the test tube re-sealed and heated at 80° C. for an additional 2 days. The reaction was cooled and silica gel (0.5 g) was added to the reaction mixture, followed by evaporation of the volatiles under reduced pressure. The pre-adsorbed solids were loaded into a solid loading cartridge and subjected to a gradient elution using ethyl acetate:hexane (25:75) to ethyl acetate (100%) using a RediSep silica gel cartridge (4.2 g; ISCO). The methyl ester (higher Rf) was readily separated from the carboxamide product and the appropriate fractions were combined and concentrated under reduced pressure to give methyl 5-(1H-benzimidazol-1-yl)-3-[(2-methylbenzyl)oxy]-2-thiophenecarboxylate (0.0092 g) as an off-white solid. ¹H NMR (DMSO-d₆): δ 8.72 (s, 1H), 7.86 (d, 1H), 7.81 (d, 1H), 7.76 (s, 1H), 7.55 (d, 1H), 7.42 (m, 1H), 7.38 (dd, 1H), 7.26 (m, 3H), 5.38 (s, 2H), 3.77 (s, 3H), 2.39 (s, 3H). MS m/z 379 (M+1); and 5-(1H-benzimidazol-1-yl)-3-[(2-methylbenzyl)oxy]-2-thiophenecarboxamide (0.0136 g) as a tan solid. ¹H NMR (DMSO-d₆): δ 8.65 (s, 1H), 7.80 (d, 1H), 7.68 (s+br s, 2H), 7.49 (d, 1H), 7.40 (m, 3H), 7.28 (m, 3H), 6.85 (br s, 1H), 5.43 (s, 2H), 2.39 (s, 3H). MS m/z 364 (M+1).

### EXAMPLE 3

### Methyl 5-(1H-benzimidazol-1-yl)-3-[(2-methylbenzyl)oxy]-2-thiophenecarboxylate

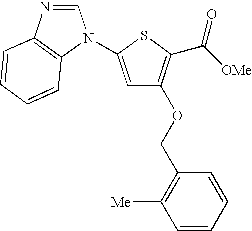

To a mixture of methyl 5-(1H-benzimidazol-1-yl)-3-hydroxy-2-thiophenecarboxylate (0.500 g, 1.82 mmol) and potassium carbonate (0.277 g, 2.01 mmol) in dimethylformamide (5.0 mL) was added α-bromo-o-xylene (0.27 mL, 2.01 mmol). The mixture was stirred for 18 hours at room temperature and then diluted with water (20 mL) and extracted with ether (2×50 mL). The organic layer was washed with water (10 mL), saturated brine (10 mL) and dried (MgSO₄). Concentration of the organic phase under reduced pressure gave 0.395 g of crude methyl 5-(1H-benzimidazol-1-yl)-3-[(2-methylbenzyl)oxy]-2-thiophenecarboxylate as a yellow solid. ¹H NMR (DMSO-d₆): δ 8.71 (s, 1H), 7.84 (d, 1H), 7.79 (d, 1H), 7.75 (s, 1H), 7.53 (d, 1H), 7.42 (dd, 1H), 7.38 (dd, 1H), 7.24 (m, 3H), 5.36 (s, 2H), 3.75 (s, 3H), 2.37 (s, 3H). MS m/z 379 (M+1).

### EXAMPLE 4

### 5-(1H-Benzimidazol-1-yl)-3-[(2-methylbenzyl)oxy]-2-thiophenecarboxamide

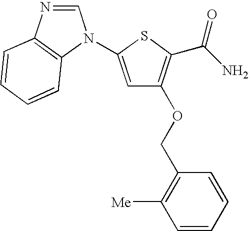

A mixture of methyl 5-(1H-benzimidazol-1-yl)-3-[(2-methylbenzyl)oxy]-2-thiophenecarboxylate (0.114 g, 0.302 mmol) and 2M methanolic ammonia (5 mL) was heated at 80° C. for 48 h in a Pyrex test tube fitted with a Teflon-lined screw cap. The reaction was cooled and charged with fresh 2M methanolic ammonia (2 mL) and heated at 80° C. for 72 h. The reaction was again cooled and recharged with fresh 2M methanolic ammonia (2 mL) and heated at 80° C. for 48 h. The reaction mixture was concentrated under reduced pressure and the solid residue was dissolved in methanol:ethyl acetate (1:1). Silica gel (0.5 g) was added to the solution and the volatiles were removed under reduced pressure. The pre-adsorbed material was packed into a solid loading cartridge and eluted onto a RediSep silica gel cartridge (4.2 g; ISCO) using ethyl acetate; collected 18 mL fractions. The appropriate fractions were combined and concentrated to dryness to give a solid residue. The solids were triturated with methanol:ether (1:2) and collected by filtration, rinsed with ether (2 mL) and dried to give 0.021 g of 5-(1H-benzimidazol-1-yl)-3-[(2-methylbenzyl)oxy]-2-thiophenecarboxamide as a light yellow solid. ¹H NMR (DMSO-d₆): 88.65 (s, 1H), 7.80 (d, 1H), 7.69 (s, 1H), 7.77 Et 6.85 (2×br s, 2H), 7.48 (d, 1H), 7.40 (m, 3H), 7.28 (m, 3H), 5.43 (s, 2H), 2.39 (s, 3H). MS m/z 364 (M+1).

### EXAMPLE 5

### 5-(1H-Benzimidazol-1-yl)-3-[(2-methylbenzyl)oxy]-2-thiophenecarboxylic acid

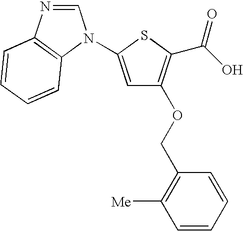

To a solution of methyl 5-(1H-benzimidazol-1-yl)-3-[(2-methylbenzyl)oxy]-2-thiophenecarboxylate (0.393 g, 1.04 mmol) in dioxane (4.0 mL) was added aqueous 1M lithium hydroxide (4.0 mL). The mixture was stirred for 18 hours at room temperature. The reaction mixture was acidified to pH 1-2 with 1N hydrochloric acid (4 mL) and the solids were collected by filtration and dried to give 0.334 g of 5-(1H-benzimidazol-1-yl)-3-[(2-methylbenzyl)oxy]-2-thiophenecarboxylic acid as a yellow solid. ¹H NMR (DMSO-d₆): δ 12.8 (br s, 1H), 8.69 (s, 1H), 7.80 (2×d, 2H), 7.70 (s, 1H), 7.52 (d, 1H), 7.40 (m, 2H), 7.24 (m, 3H), 5.32 (s, 2H), 2.37 (s, 3H). MS m/z 365 (M+1).

### EXAMPLE 6

### 5-(1H-Benzimidazol-1-yl)-N-methyl-3-[(2-methylbenzyl)oxy]-2-thiophenecarboxamide

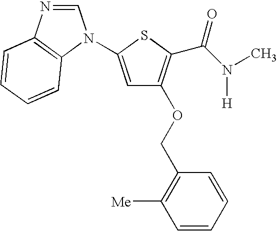

To a mixture of 5-(1H-benzimidazol-1-yl)-3-[(2-methylbenzyl)oxy]-2-thiophenecarboxylic acid (0.050 g, 0.14 mmol) in dichloromethane (2 mL) was added 1-chloro-2,N,N-trimethylpropenylamine (0.027 mL, 0.20 mmol) and the reaction mixture was stirred for 1 hour at room temperature. Methylamine (8M) in ethanol (52 mL, 0.42 mmol) was added to the reaction mixture, followed by addition of diisopropylethylamine (49 μL, 0.28 mmol). The reaction was complete after two hours. After stirring for 66 hours the reaction was partitioned between dichloromethane (3 mL) and water (1 mL). The biphasic mixture was separated and the organic phase dried over MgSO₄. The organic phase was concentrated under reduced pressure and the residue was triturated with ether. The solids were collected by filtration and dried to give 0.037 g of 5-(1H-benzimidazol-1-yl)-N-methyl-3-[(2-methylbenzyl)oxy]-2-thiophenecarboxamide as a yellow solid. ¹H NMR (DMSO-d₆): δ 8.63 (s, 1H), 7.80 (d, 1H), 7.74 (d, 1H), 7.63 (s, 1H), 7.42 (m, 4H), 7.27 (m, 3H), 5.44 (s, 2H), 2.81 (d, 3H), 2.39 (s, 3H). MS m/z 378 (M+1).

### EXAMPLE 7

### 5-(1H-Benzimidazol-1-yl)-N,N-dimethyl-3-[(2-methylbenzyl)oxy]-2-thiophenecarboxamide

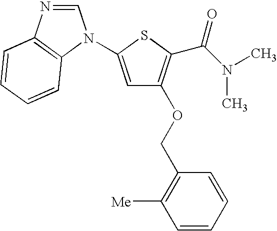

In a similar manner as described for Example 6, 5-(1H-benzimidazol-1-yl)-3-[(2-methylbenzyl)oxy]-2-thiophenecarboxylic acid (0.050 g, 0.14 mmol) in dichloromethane (2 mL), 1-chloro-2,N,N-trimethylpropenylamine (0.027 mL, 0.20 mmol), dimethylamine (2M) in tetrahydrofuran (210 μL, 0.42 mmol) and diisopropylethylamine (49 μL, 0.28 mmol) gave 5-(1H-benzimidazol-1-yl)-N,N-dimethyl-3-[(2-methylbenzyl)oxy]-2-thiophenecarboxamide (0.032 g, 60%) as a tan solid. ¹H NMR (DMSO-d₆): δ 8.63 (s, 1H), 7.79 (2×d, 2H), 7.64 (s, 1H), 7.40 (m, 3H), 7.26 (m, 3H), 5.30 (s, 2H), 2.98 (s, 6H), 2.34 (s, 3H). MS m/z 392 (M+1).

### EXAMPLE 8

### 5-(1H-Benzimidazol-1-yl)-N-isopropyl-3-[(2-methylbenzyl)oxy]-2-thiophenecarboxamide

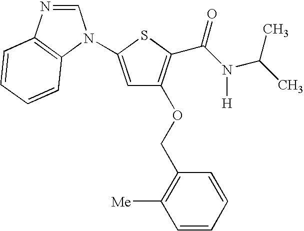

In a similar manner as described for Example 6, 5-(1H-benzimidazol-1-yl)-3-[(2-methylbenzyl)oxy]-2-thiophenecarboxylic acid (0.050 g, 0.14 mmol) in dichloromethane (2 mL), 1-chloro-2,N,N-trimethylpropenylamine (0.027 mL, 0.20 mmol), isopropylamine (36 μL, 0.42 mmol) and diisopropylethylamine (49 μL, 0.28 mmol) gave 5-(1H-benzimidazol-1-yl)-N-isopropyl-3-[(2-methylbenzyl)oxy]-2-thiophenecarboxamide (0.033 g, 59%) as a yellow solid. ¹H NMR (DMSO-d₆): δ 8.66 (s, 1H), 7.81 (2×d, 2H), 7.73 (s, 1H), 7.52 (d, 1H), 7.44 (m, 1H), 7.38 (m, 1H), 7.30 (m, 3H), 7.14 (d, 1H), 5.44 (s, 2H), 3.99 (m, 1H), 2.41 (s, 3H), 1.06 (d, 6H). MS m/z 406 (M+1).

### EXAMPLE 9

### 5-(1H-Benzimidazol-1-yl)-N-(2-hydroxyethyl)-3-[(2-methylbenzyl)oxy]-2-thiophenecarboxamide

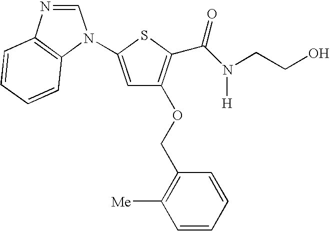

In a similar manner as described for Example 6, 5-(1H-benzimidazol-1-yl)-3-(2-methylbenzyloxy)-2-thiophenecarboxylic acid (0.050 g, 0.14 m mol) in dichloromethane (2 mL), 1-chloro-2,N,N-trimethylpropenylamine (0.027 mL, 0.20 mmol), ethanolamine (25 μL, 0.42 mmol) and diisopropylethylamine (49 μL, 0.28 m mol) gave 5-(1H-benzimidazol-1-yl)-N-(2-hydroxyethyl)-3-[(2-methylbenzyl)oxy]-2-thiophenecarboxamide (0.036 g, 64%) as a yellow solid. ¹H NMR (DMSO-d₆): δ 8.65 (s, 1H), 7.80 (2×d, 2H), 7.71 (s, 1H), 7.54 (m, 2H), 7.44 (m, 1H), 7.37 (m, 1H), 7.27 (m, 3H), 5.45 (s, 2H), 4.80 (t, 1H), 3.46 (m, 2H), 3.36 (m, 2H), 2.40 (s, 3H). MS m/z 408 (M+1).

### EXAMPLE 10

### 5-(1H-Benzimidazol-1-yl)-3-[(2-methylbenzyl)oxy]-N-phenyl-2-thiophenecarboxamide

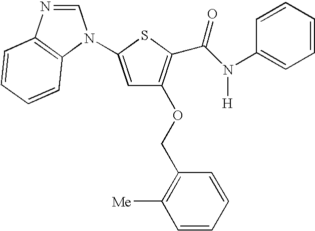

In a similar manner as described for Example 6, 5-(1H-benzimidazol-1-yl)-3-[(2-methylbenzyl)oxy]-2-thiophenecarboxylic acid (0.050 g, 0.14 mmol) in dichloromethane (2 mL), 1-chloro-2,N,N-trimethylpropenylamine (0.027 mL, 0.20 mmol), aniline (38 μL, 0.42 mmol) and diisopropylethylamine (49 μL, 0.28 mmol) gave 5-(1H-benzimidazol-1-yl)-3-[(2-methylbenzyl)oxy]-N-phenyl-2-thiophenecarboxamide (0.044 g, 73%) as a yellow solid. ¹H NMR (DMSO-d₆): δ 9.30 (s, 1H), 8.72 (s, 1H), 7.85 (m, 2H), 7.81 (s, 1H), 7.61 (d, 1H), 7.41 (m, 4H), 7.32 (m, 5H), 7.09 (m, 1H), 5.56 (s, 2H), 2.44 (s, 3H). MS m/z 440 (M+1).

### EXAMPLE 11

### 5-(1H-Benzimidazol-1-yl)-N-benzyl-3-[(2-methylbenzyl)oxy]-2-thiophenecarboxamide

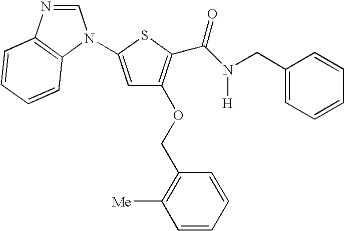

In a similar manner as described for Example 6, 5-(1H-benzimidazol-1-yl)-3-[(2-methylbenzyl)oxy]-2-thiophenecarboxylic acid (0.050 g, 0.14 mmol) in dichloromethane (2 mL), 1-chloro-2,N,N-trimethylpropenylamine (0.027 mL, 0.20 mmol), benzylamine (46 μL, 0.42 mmol) and diisopropylethylamine (49 μL, 0.28 mmol) gave 5-(1H-benzimidazol-1-yl)-N-benzyl-3-[(2-methylbenzyl)oxy]-2-thiophenecarboxamide (0.038 g, 61%) as a yellow solid. ¹H NMR (DMSO-d₆): δ 8.65 (s, 1H), 7.81 (m, 3H), 7.69 (s, 1H), 7.42 (m, 3H), 7.27 (m, 8H), 5.43 (s, 2H), 4.49 (d, 2H), 2.29 (s, 3H). MS m/z 454 (M+1).

### EXAMPLE 12

### 5-(1H-Benzimidazol-1-yl)-3-benzyloxy-2-thiophenecarboxamide

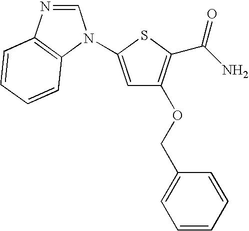

In a similar manner as described for Example 4, methyl 5-(1H-benzimidazol-1-yl)-3-[(2-methylbenzyl)oxy]-2-thiophenecarboxylate (0.109 g, 0.299 mmol) and 2M methanolic ammonia (5 mL) gave 5-(1H-benzimidazol-1-yl)-3-benzyloxy-2-thiophenecarboxamide (0.031 g, 30%) as a white solid. ¹H NMR (DMSO-d₆): δ 8.63 (s, 1H), 7.76 (dd, 2H), 7.70 Et 7.01 (2×br s, 2H), 7.64 (s, 1H), 7.55 (d, 2H), 7.44 (m, 5H), 5.42 (s, 2H), MS m/z 350 (M+1).

### EXAMPLE 13

### 5-(1H-Benzimidazol-1-yl)-3-[(3-methylbenzyl)oxy]-2-thiophenecarboxamide

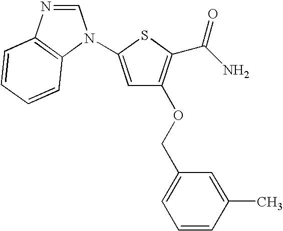

In a similar manner as described for Example 4, methyl 5-(1H-benzimidazol-1-yl)-3-[(3-methylbenzyl)oxy]-2-thiophenecarboxylate (0.114 g, 0.301 mmol) and 2M methanolic ammonia (5 mL) gave 5-(1H-benzimidazol-1-yl)-3-[(3-methylbenzyl)oxy]-2-thiophenecarboxamide (0.019 g, 17%) as a white solid. ¹H NMR (DMSO-d₆): δ 8.63 (s, 1H), 7.77 (dd, 2H), 7.70 εt 7.00 (2×br s, 2H), 7.63 (s, 1H), 7.36 (m, 5H), 7.19 (d, 1H), 5.37 (s, 2H), 2.33 (s, 3H). MS m/z 364 (M+1).

### EXAMPLE 14

### 5-(1H-Benzimidazol-1-yl)-3-[(3-methoxybenzyl)oxy]-2-thiophenecarboxamide

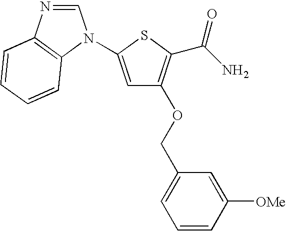

In a similar manner as described for Example 4, methyl 5-(1H-benzimidazol-1-yl)-3-[(3-methoxybenzyl)oxy]-2-thiophenecarboxylate (0.118 g, 0.299 mmol) and 2M methanolic ammonia (5 mL) gave 5-(1H-benzimidazol-1-yl)-3-[(3-methoxybenzyl)oxy]-2-thiophenecarboxamide (0.034 g, 30%) as an off-white solid. ¹H NMR (DMSO-d₆): δ 8.63 (s, 1H), 7.77 (dd, 2H), 7.66 Et 7.05 (2×br s, 2H), 7.63 (s, 1H), 7.38 (m, 3H), 7.12 (m, 2H), 6.94 (d, 1H), 5.38 (s, 2H), 3.76 (s, 3H). MS m/z 380 (M+1).

### EXAMPLE 15

### 5-(1H-Benzimidazol-1-yl)-3-[(3-chlorobenzyl)oxy]-2-thiophenecarboxamide

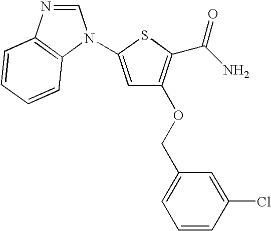

In a similar manner as described for Example 4, methyl 5-(1H-benzimidazol-1-yl)-3-[(3-chlorobenzyl)oxy]-2-thiophenecarboxylate (0.120 g, 0.301 mmol) and 2M methanolic ammonia (5 mL) gave 5-(1H-benzimidazol-1-yl)-3-[(3-chlorobenzyl)oxy]-2-thiophenecarboxamide (0.031 g, 27%) as a white solid. ¹H NMR (DMSO-d₆): δ 8.62 (s, 1H), 7.80 (d, 1H), 7.70 (m, 4H), 7.63 (s, 1H), 7.54 Et 7.09 (2×br s, 2H), 7.42 (m, 3H), 5.41 (s, 2H). MS m/z 384 (M+1).

### EXAMPLE 16

### 5-(1H-Benzimidazol-1-yl)-3-[(4-methylbenzyl)oxy]-2-thiophenecarboxamide

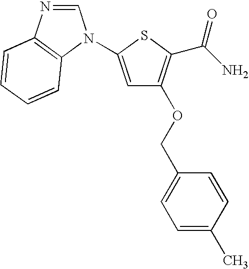

In a similar manner as described for Example 4, methyl 5-(1H-benzimidazol-1-yl)-3-[(4-methylbenzyl)oxy]-2-thiophenecarboxylate (0.114 g, 0.301 mmol) and 2M methanolic ammonia (5 mL) gave 5-(1H-benzimidazol-1-yl)-3-[(4-methylbenzyl)oxy]-2-thiophenecarboxamide (0.0069 g, 6%) as an off-white solid. ¹H NMR (DMSO-d₆): δ 8.63 (s, 1H), 7.78 (dd, 2H), 7.69 εt 6.98 (2×br s, 2H), 7.64 (s, 1H), 7.40 (m, 4H), 7.24 (d, 2H), 5.36 (s, 2H), 2.31 (s, 3H). MS m/z 364 (M+1).

### EXAMPLE 17

### 5-(1H-Benzimidazol-1-yl)-3-[(4-chlorobenzyl)oxy]-2-thiophenecarboxamide

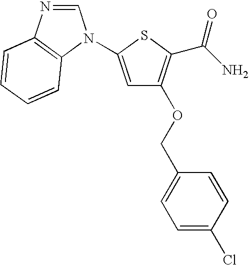

In a similar manner as described for Example 4 methyl 5-(1H-benzimidazol-1-yl)-3-[(4-chlorobenzyl)oxy]-2-thiophenecarboxylate (0.120 g, 0.301 mmol) and 2M methanolic ammonia (5 mL) gave 5-(1H-benzimidazol-1-yl)-3-[(4-chlorobenzyl)oxy]-2-thiophenecarboxamide (0.015 g, 13%) as an off-white solid. ¹H NMR (DMSO-d₆): δ 8.62 (s, 1H), 7.78 (dd, 2H), 7.70 Et 7.03 (2×br s, 2H), 7.62 (s, 1H), 7.54 (AB q, 4H), 7.40 (m, 2H), 5.41 (s, 2H). MS m/z 384 (M+1).

### EXAMPLE 18A

### Methyl 3-hydroxy-5-(5-methyl-1H-benzimidazol-1-yl)-2-thiophenecarboxylate and Methyl 3-hydroxy-5-(6-methyl-1H-benzimidazol-1-yl)-2-thiophenecarboxylate

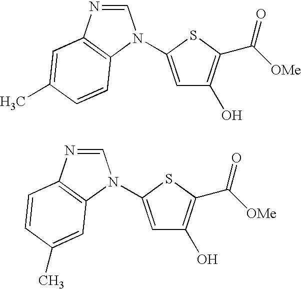

In a similar manner as described for Example 2A, methyl 2-chloro-3-oxo-2,3-dihydro-2-thiophenecarboxylate (0.050 g, 0.26 mmol) and 5-methyl-1H-benzimidazole (0.069 g, 0.52 mmol) in chloroform (1.0 mL), and in a separate reaction acetic acid (1.0 mL), gave a 1:1 isomer mixture of methyl 3-hydroxy-5-(5-methyl-1H-benzimidazol-1-yl)-2-thiophenecarboxylate and methyl 3-hydroxy-5-(6-methyl-1H-benzimidazol-1-yl)-2-thiophenecarboxylate (0.063 g, 42%) as a light yellow solid. ¹H NMR (DMSO-d₆): δ 10.84 (br s, 2H), 8.63, 8.59 (2×s, 2H), 7.65 (m, 4H), 7.22 (m, 2H), 7.12 (d, 2H), 3.79, 3.78 (2×s, 6H), 2.47, 2.44 (2×s, 6H). MS m/z 289 (M+1).

### EXAMPLE 18B

### Methyl 5-(5-methyl-1H-benzimidazol-1-yl)-3-[(2-methylbenzyl)oxy]-2-thiophenecarboxylate/Methyl 5-(6-methyl-1H-benzimidazol-1-yl)-3-[(2-methylbenzyl)oxy]-2-thiophenecarboxylate and 5-(5-Methyl-1H-benzimidazol-1-yl)-3-[(2-methylbenzyl)oxy]-2-thiophenecarboxamide/5-(6-Methyl-1H-benzimidazol-1-yl)-3-[(2-methylbenzyl)oxy]-2-thiophenecarboxamide

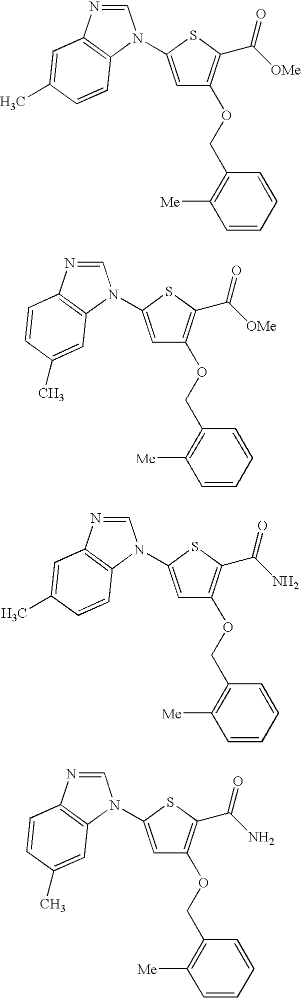

In a similar manner as described for Example 2B, a 1:1 isomer mixture of methyl 3-hydroxy-5-(5-methyl-1H-benzimidazol-1-yl)-2-thiophenecarboxylate and methyl 3-hydroxy-5-(6-methyl-1H-benzimidazol-1-yl)-2-thiophenecarboxylate (0.055 g, 0.19 mmol), potassium carbonate (0.029 g, 0.21 mmol), α-bromo-o-xylene (28 μL, 0.21 mmol) and dimethylformamide (0.50 mL), followed by 2M methanolic ammonia (3 mL), gave a 1:1 isomer mixture of methyl 5-(5-methyl-1H-benzimidazol-1-yl)-3-[(2-methyl-benzyl)oxy]-2-thiophenecarboxylate and methyl 5-(6-methyl-1H-benzimidazol-1-yl)-3-[(2-methylbenzyl)oxy]-2-thiophenecarboxylate (0.017 g, 23%) as an amber oil. ¹H NMR (DMSO-d₆): δ 8.67 (s, 1H), 8.62 (s, 1H), 7.74 (d, 1H), 7.73 (s, 2H), 7.67 (d, 1H), 7.60 (s, 2H), 7.54 (d, 2H), 7.26 (m, 8H), 5.37 (s, 4H), 4.09 (q, 2H), 3.77, 3.76 (2×s, 6H), 3.16 (d, 4H), 2.45, 2.39 (2×s, 6H). MS m/z 393 (M+1); and a 1:1 isomer mixture of 5-(5-methyl-1H-benzimidazol-1-yl)-3-[(2-methylbenzyl)oxy]-2-thiophene-carboxamide and 5-(6-methyl-1H-benzimidazol-1-yl)-3-[(2-methylbenzyl)oxy]-2-thiophenecarboxamide (0.057 g, 79%) as a tan solid. ¹H NMR (DMSO-d₆): δ 8.59, 8.55 (2×s, 2H), 7.67 (m, 4H), 8.64 (s, 2H), 8.59, 8.53 (2×s, 2H), 7.50 Et 6.87 (2 br s, 4H), 7.28 (m, 8H), 5.42 (s, 4H), 3.32, 3.31 (2×s, 6H), 2.45, 2.39 (2×s, 6H). MS m/z 365 (M+1).

### EXAMPLE 19A

### Methyl 3-hydroxy-5-(5,6-dimethyl-1H-benzimidazol-1-yl)-2-thiophenecarboxylate

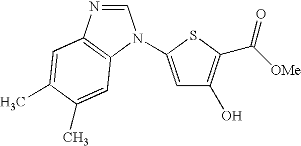

In a similar manner as described for Example 2A, methyl 2-chloro-3-oxo-2,3-dihydro-2-thiophenecarboxylate (0.050 g, 0.26 mmol) and 5,6-dimethyl-1H-benzimidazole (0.076 g, 0.52 mmol) in chloroform (1.0 mL), and in a separate reaction acetic acid (1.0 mL), gave methyl 3-hydroxy-5-(5,6-dimethyl-1H-benzimidazol-1-yl)-2-thiophenecarboxylate (0.079 g, 50%) as a light yellow solid. ¹H NMR (DMSO-d₆): δ 10.81 (br s, 1H), 8.54 (s, 1H), 7.59 (s, 1H), 7.56 (s, 1H), 7.11 (s, 1H), 3.79 (s, 3H), 2.37 (s, 3H), 2.33 (s, 3H). MS m/z 303 (M+1).

### EXAMPLE 19B

### Methyl 5-(5,6-dimethyl-1H-benzimidazol-1-yl)-3-[(2-methyl benzyl)oxy]-2-thiophenecarboxylate and 5-(5,6-Dimethyl-1H-benzimidazol-1-yl)-3-[(2-methylbenzyl)oxy]-2-thiophenecarboxamide

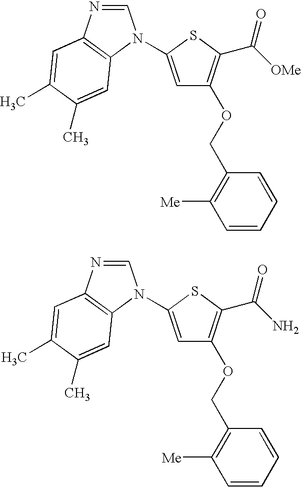

In a similar manner as described for Example 2B, methyl 3-hydroxy-5-(5,6-dimethyl-1H-benzimidazol-1-yl)-2-thiophenecarboxylate (0.074 g, 0.24 mmol), potassium carbonate (0.037 g, 0.27 mmol), α-bromo-o-xylene (36 mL, 0.27 mmol) and dimethylformamide (0.50 mL), followed by 2M methanolic ammonia (3 mL), gave methyl 5-(5,6-dimethyl-1H-benzimidazol-1-yl)-3-[(2-methyl benzyl)oxy]-2-thiophenecarboxylate (0.011 g, 11%) as a pale yellow solid. ¹H NMR (DMSO-d₆): δ 8.58 (s, 1H), 7.70 (s, 1H), 7.58 (m, 3H), 7.26 (m, 3H), 5.37 (s, 2H), 3.76 (s, 3H), 2.39 (s, 6H), 2.34 (s, 3H). MS m/z 407 (M+1); and 5-(5,6-dimethyl-1H-benzimidazol-1-yl)-3-[(2-methylbenzyl)oxy]-2-thiophenecarboxamide (0.0066 g, 7%) as an off-white solid. ¹H NMR (DMSO-d₆): δ 8.50 (s, 1H), 7.68, 6.85 (2×br s, 2H), 7.62 (s, 1H), 7.54 (d, 2H), 7.50 (d, 1H), 7.28 (m, 3H), 5.42 (s, 2H), 2.39 (s, 3H), 2.37 (s, 3H), 2.34 (s, 3H). MS m/z 392 (M+1).

### EXAMPLE 20A

### Methyl 5-(5-chloro-1H-benzimidazol-1-yl)-3-hydroxy-2-thiophenecarboxylate and Methyl 5-(6-chloro-1H-benzimidazol-1-yl)-3-hydroxy-2-thiophenecarboxylate

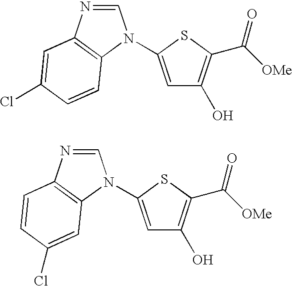

In a similar manner as described for Example 2A, methyl 2-chloro-3-oxo-2,3-dihydro-2-thiophenecarboxylate (0.050 g, 0.26 mmol) and 5-chloro-1H-benzimidazole (0.079 g, 0.52 mmol) in chloroform (1.0 mL), and in a separate reaction acetic acid (1.0 mL), gave a 1:1 isomer mixture of methyl 5-(5-chloro-1H-benzimidazol-1-yl)-3-hydroxy-2-thiophenecarboxylate and methyl 5-(6-chloro-1H-benzimidazol-1-yl)-3-hydroxy-2-thiophenecarboxylate (0.103 g, 64%) as a light yellow solid. ¹H NMR (DMSO-d₆): δ 10.91, 10.89 (2×br s, 2H), 8.76, 8.71 (2×s, 2H), 7.89 (s, 1H), 7.82 (d, 1H), 7.81 (s, 2H), 7.42 (m, 2H), 7.17, 7.15 (2×s, 2H), 3.79 (2×s, 6H). MS m/z 309 (M+1).

### EXAMPLE 20B

### Methyl 5-(5-chloro-1H-benzimidazol-1-yl)-3-[(2-methylbenzyl)oxy]-2-thiophenecarboxylate/Methyl 5-(6-chloro-1H-benzimidazol-1-yl)-3-[(2-methylbenzyl)oxy]-2-thiophenecarboxylate and 5-(5-Chloro-1H-benzimidazol-1-yl)-3-[(2-methylbenzyl)oxy]-2-thiophenecarboxamide/5-(6-Chloro-1H-benzimidazol-1-yl)-3-[(2-methylbenzyl)oxy]-2-thiophenecarboxamide

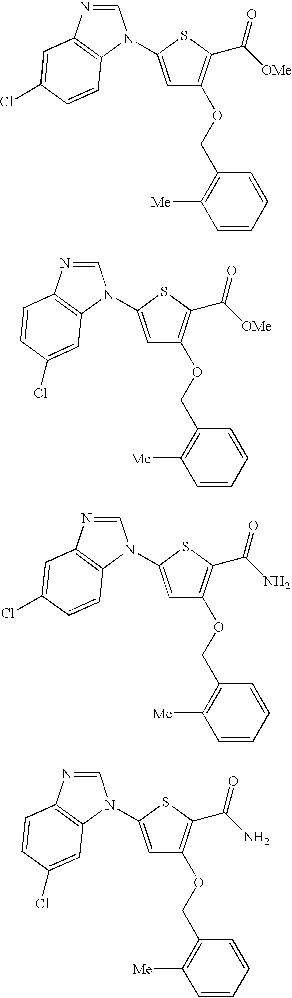

In a similar manner as described for Example 2B, a 1:1 isomer mixture of methyl 5-(5-chloro-1H-benzimidazol-1-yl)-3-hydroxy-2-thiophenecarboxylate and methyl 5-(6-chloro-1H-benzimidazol-1-yl)-3-hydroxy-2-thiophenecarboxylate (0.095 g, 0.31 mmol), potassium carbonate (0.047 g, 0.34 mmol), α-bromo-o-xylene (46 μL, 0.34 mmol) and dimethylformamide (0.50 mL), followed by workup, gave a solid mixture. Treatment of the residual solids with 2M methanolic ammonia (3 mL) at elevated temperature, followed by chromatography, gave a mixture of methyl 5-(5-chloro-1H-benzimidazol-1-yl)-3-[(2-methylbenzyl)oxy]-2-thiophenecarboxylate and methyl 5-(6-chloro-1H-benzimidazol-1-yl)-3-[(2-methylbenzyl)oxy]-2-thiophenecarboxylate (0.016 g, 6%) as a pale yellow solid. ¹H NMR (DMSO-d₆): δ 8:79 (s, 1H), 7.90 (d, 1H), 7.86 (d, 1H), 7.78 (s, 1H), 7.50 (m, 2H), 7.26 (m, 3H), 5.37 (s, 2H), 3.77 (s, 3H), 2.38 (s, 3H). MS m/z 413 (M+1); and a mixture of 5-(5-chloro-1H-benzimidazol-1-yl)-3-[(2-methylbenzyl)oxy]-2-thiophenecarboxamide and 5-(6-chloro-1H-benzimidazol-1-yl)-3-[(2-methylbenzyl)oxy]-2-thiophenecarboxamide (0.021 g, 8.5%) as a pale yellow solid. ¹H NMR (DMSO-d₆): δ 8.72, 8.67 (2×s, 2H), 7.80 (m, 4H), 7.72 Et 6.88 (2×br s, 4H), 7.70 (s, 2H), 7.44 (m, 4H), 7.28 (m, 6H), 5.43, 5.42 (2×s, 4H), 2.39 (2×s, 6H). MS m/z 398 (M+1).

### EXAMPLE 21

### Methyl 5-(1H-benzimidazol-1-yl)-3-isopropoxy-2-thiophenecarboxylate

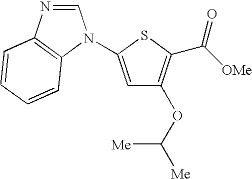

To a mixture of methyl 5-(1H-benzimidazol-1-yl)-3-hydroxy-2-thiophenecarboxylate (0.150 g, 0.55 mmol) and potassium carbonate (0.083 g, 0.60 mmol) in dimethyl-formamide (5.0 mL) was added 2-iodopropane (60 μL, 0.60 mmol). The mixture was heated at 65° C. for 3 hours and then additional 2-iodopropane (164 μL) was added to the reaction. The mixture was heated at 80° C. for 64 hours and then diluted with water (2.0 mL) and extracted with ether (2×5.0 mL). The organic layer was washed with saturated brine (2.0 mL) and dried (MgSO₄). The organic layer was filtered and concentrated under reduced pressure to give a residue which was dissolved in EtOAc and pre-adsorbed to silica gel (1.5 g). Elution of the silica-adsorbed material on a RediSep column (4.2 g; ISCO) using a gradient elution EtOAc:hexanes (25:75) to EtOAc (100) gave 0.082 g of methyl 5-(1H-benzimidazol-1-yl)-3-isopropoxy-2-thiophenecarboxylate as a yellow solid. MS m/z 317 (M+1).

### EXAMPLE 22

### 5-(1H-benzimidazol-1-yl)-3-isopropoxy-2-thiophenecarboxamide

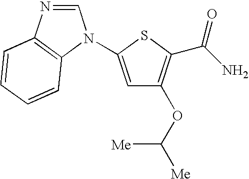

In a similar manner as described for Example 4, methyl 5-(1H-benzimidazol-1-yl)-3-isopropoxy-2-thiophenecarboxylate (0.080 g, 0.25 mmol) and 7M methanolic ammonia (3.0 mL) gave 5-(1H-benzimidazol-1-yl)-3-isopropoxy-2-thiophenecarboxamide (0.045 g, 60%) as an off-white solid. ¹H NMR (DMSO-d₆): δ 8.64 (s, 1H), 7.78 (2×d, 2H), 7.68 Et 6.93 (2×br s, 2H), 7.55 (s, 1H), 7.37 (2×t, 2H), 4.80 (m, 1H), 1.36 (d, 6H). MS m/z 302 (M+1).

### EXAMPLE 23

### 5-(1H-Benzimidazol-1-yl)-3-[(2-methylbenzyl)oxy]thiophene-2-carbonitrile

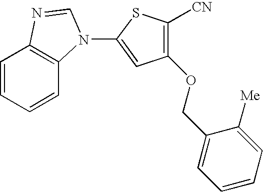

5-(1H-Benzimidazol-1-yl)-3-[(2-methylbenzyl)oxy]-2-thiophenecarboxamide (0.0285 g, 0.0784 mmol) was dissolved in 2 mL of pyridine and cooled to 0° C. Trifluoroacetic anhydride (0.017 mL, 0.120 mmol) was added dropwise via syringe. The mixture was stirred for 15 minutes and warmed to room temperature. After 1 hour, 2 mL of dichloromethane followed by five drops of trifluoroacetic anhydride were added to dissolve the insoluble components of the mixture. After 14 hours, the reaction was poured into dichloromethane and brine. The layers were separated and the aqueous layer was washed twice with dichloromethane. The combined organic layers were dried over MgSO₄, filtered, and concentrated in vacuo. Purification by flash chromatography provided 0.0075 g (28%) of 5-(1H-benzimidazol-1-yl)-3-[(2-methylbenzyl)oxy]thiophene-2-carbonitrile as a pale yellow solid. ¹H NMR (300 MHz, DMSO-d₆) δ 8.71 (s, 1H), 7.83 (s+m, 3H), 7.49-7.25 (m, 6H), 5.44 (s, 2H), 2.40 (s, 3H). MS (m/z) 346 (m+1).

### EXAMPLE 24

### {5-(1H-Benzimidazol-1-yl)-3-[(2-methylbenzyl)oxy]thien-2-yl}methanol

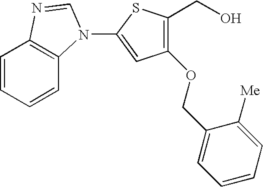

Methyl 5-(1H-benzimidazol-1-yl)-3-[(2-methylbenzyl)oxy]-2-thiophenecarboxylate (0.276 g, 0.729 mmol) was dissolved in 7 mL of dichloromethane and cooled to −78° C. Diisobutylaluminum hydride (1.5 M in toluene, 2.0 mL, 3.0 mmol) was added dropwise via syringe. After 1 hour, an additional quantity of diisobutylaluminum hydride (1.5 M in toluene, 1.0 mL, 1.5 mmol) was added dropwise via syringe. The reaction was allowed to stir for an additional 10 minutes. Methanol (1-2 mL) was added dropwise by pipet, and the mixture was warmed to room temperature. Dilute aqueous hydrochloric acid (5 percent HCl w/v) was added carefully by pipet. The mixture was poured into ethyl acetate and water, and the layers were separated. The organics were washed with brine, and the combined aqueous layers were extracted with ethyl acetate. The combined organic layers were dried over MgSO₄, filtered, and concentrated in vacuo. Purification by flash chromatography provided afforded 0.175 g (68%) of {5-(1H-benzimidazol-1-yl)-3-[(2-methylbenzyl)oxy]thien-2-yl}methanol as a tan solid. ¹H NMR (300 MHz, DMSO-d₆) δ 8.52 (s, 1H), 7.78 (d, J=7.4 Hz, 1H), 7.64 (d, J=7.4 Hz, 1H), 7.48 (s, 1H), 7.45-7.19 (m, 6H), 5.42 (br s, 1H), 5.16 (s, 2H), 2.37 (s, 3H). MS (m/z) 351 (m+1).

### EXAMPLE 25

### 5-(1H-Benzimidazol-1-yl)-3-[(2-methylbenzyl)oxy]thiophene-2-carbaldehyde

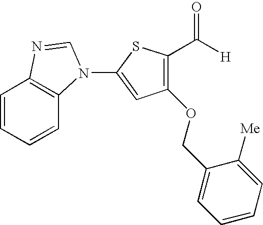

{5-(1H-benzimidazol-1-yl)-3-[(2-methylbenzyl)oxy]thien-2-yl}methanol (0.0535 g, 0.153 mmol) was dissolved in 5 mL of dichloromethane with stirring. Manganese dioxide (0.133 g, 1.53 mmol) was added in single portion. The mixture was allowed to stir for 1 hour and then filtered through a celite pad, washing well with dichloromethane. The solvent was removed in vacuo, and the solid dried under high vacuum conditions to yield 0.0508 g (95%) of 5-(1H-benzimidazol-1-yl)-3-[(2-methylbenzyl)oxy]thiophene-2-carbaldehyde as a tan solid. ¹H NMR (300 MHz, DMSO-d₆) δ 9.96 (s, 1H), 8.79 (s, 1H), 7.93 (d, J=7.9 Hz, 1H), 7.83 (s, 1H), 7.83 (d, J=7.6 Hz, 1H), 7.77-7.35 (m, 3H), 7.31-7.22 (m, 3H), 5.47 (s, 2H), 2.40 (s, 3H).

### EXAMPLE 26

### (+/−)-1-{5-(1H-Benzimidazol-1-yl)-3-[(2-methylbenzyl)oxy]thien-2-yl}ethanol

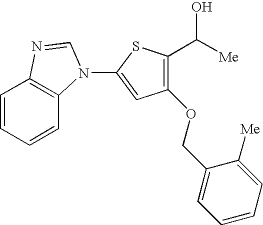

Methyl magnesium bromide (0.35 mL, 3.0 M in diethyl ether, 1.05 mmol) was added to 3 mL of diethyl ether with stirring. The solution was cooled to 0° C., and 5-(1H-benzimidazol-1-yl)-3-[(2-methylbenzyl)oxy]thiophene-2-carbaldehyde (0.0943 g, 0.271 mmol) in 3 mL of dichloromethane was added dropwise via syringe. The reaction was stirred for 30 minutes and quenched by the addition of 5 mL of water. The mixture was warmed to room temperature and enough 5% HCl solution was added to dissolve the magnesium salts. The mixture was poured into ethyl acetate, and the layers were separated. The organic layer was washed with brine, and the combined aqueous layers were extracted with ethyl acetate. The combined organic layers were dried over MgSO₄, filtered, and concentrated in vacuo to afford 0.0965 g (98%) of (+/−)-1-{5-(1H-benzimidazol-1-yl)-3-[(2-methylbenzyl)oxy]thien-2-yl}ethanol as a brown solid. ¹H NMR (300 MHz, DMSO-d₆) δ 8.51 (s, 1H), 7.77 (d, J=7.3 Hz, 1H), 7.64 (d, J=7.5 Hz, 1H), 7.48-7.22 (m, 7H), 5.61 (m, 1H), 5.15 (s, 2H), 5.08 (m, 1H), 2.38 (s, 3H), 1.39, 1.36 (2×s, 3H).

### EXAMPLE 27

### 1-{5-(1H-Benzimidazol-1-yl)-3-[(2-methylbenzyl)oxy]thien-2-yl}ethanone

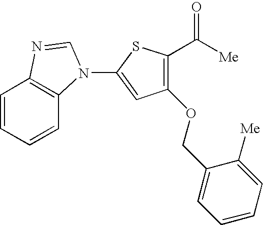

Using a procedure as described in Example 25 afforded 1-{5-(1H-benzimidazol-1-yl)-3-[(2-methylbenzyl)oxy]thien-2-yl}ethanone. ¹H NMR (300 MHz, DMSO-d₆) δ 8.76 (s, 1H), 7.90 (d, J=7.9 Hz, 1H), 7.82 (d, J=27.6 Hz, 1H), 7.78 (s, 1H), 7.55-7.24 (m, 6H), 5.44 (s, 2H), 2.46 (s, 3H), 2.41 (s, 3H).

### EXAMPLE 28

### 1-{4-[(2-Methylbenzyl)oxy]thien-2-yl}-1H-benzimidazole

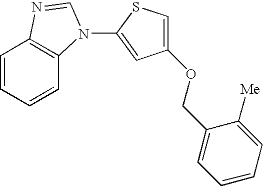

5-(1H-benzimidazol-1-yl)-3-[(2-methylbenzyl)oxy]-2-thiophenecarboxylic acid (0.105 g, 0.288 mmol) was dissolved in 4 mL of acetic acid in a flask fitted with a reflux condenser. The flask was placed in an oil bath set to 80° C. After 65 hours, the reaction was cooled to room temperature and poured into ethyl acetate. The solution was washed with saturated NaHCO₃ (3×) and brine. The organic layer was dried over MgSO₄, filtered, and concentrated in vacuo. The crude product was filtered through a short column of silica gel washing with 1:1 ethyl acetate/hexanes. The filtrate was concentrated in vacuo to afford 0.0850 g (92%) of 1-{4-[(2-methylbenzyl)oxy]thien-2-yl}-1H-benzimidazole as a dark orange oil, which later solidified upon standing. ¹H NMR (300 MHz, DMSO-d₆) δ 8.54 (s, 1H), 7.77 (d, J=7.3 Hz, 1H), 7.69 (d, J=7.5 Hz, 1H), 7.46-7.20 (m, 7H), 6.80 (d, J=1.9 Hz, 1H), 5.11 (s, 2H), 2.36 (s, 3H).

### EXAMPLE 29

### {5-(1H-Benzimidazol-1-yl)-3-[(2-methylbenzyl)oxy]thien-2-yl}methyl acetate

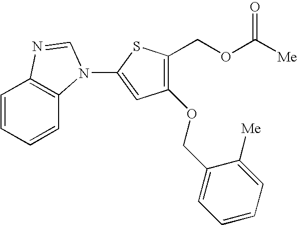

{5-(1H-benzimidazol-1-yl)-3-[(2-methyl benzyl)oxy]thien-2-yl}methanol (0.0278 g, 0.0793 mmol) was dissolved in 4 mL of dichloromethane with stirring. 4-Dimethylamino-pyridine (0.0194 g, 0.159 mmol) was added in a single portion. Acetic anhydride (0.075 mL, 0.795 mmol) was added via syringe. After two hours, the reaction was poured into ethyl acetate. The organic layer was washed with 5% HCl, saturated NaHCO₃, and Brine. The organic layer was dried over MgSO₄, filtered, and concentrated in vacuo. The residue was filtered through a short column of silica gel washing with 1:1 ethyl acetate/hexanes. The filtrate was concentrated in vacuo to provide 0.0276 g (89%) of {5-(1H-benzimidazol-1-yl)-3-[(2-methylbenzyl)oxy]thien-2-yl}methyl acetate as a dark oil, which later solidified upon standing. ¹H NMR (300 MHz, DMSO-d₆) δ 8.56 (s, 1H), 7.79 (d, J=7.4 Hz, 1H), 7.68 (d, J=7.5 Hz, 1H), 7.59 (s, 1H), 7.46-7.19 (m, 6H), 5.23 (s, 2H), 5.14 (s, 2H), 2.36 (s, 3H), 2.03 (s, 3H).

### EXAMPLE 30

### Methyl 5-(1H-benzimidazol-1-yl)-3-{[(trifluoromethyl)sulfonyl]oxy}-thiophene-2-carboxylate

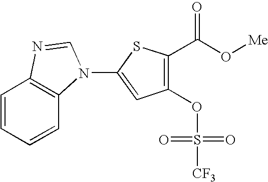

Methyl 5-(1H-benzimidazol-1-yl)-3-hydroxy-2-thiophenecarboxylate (0.275 g, 1.00 mmol) was dissolved in 7 mL of dichloromethane with stirring. N,N-Diisopropylethyl-amine (0.230 mL, 1.32 mmol) was added via syringe. N-Phenyltrifluoromethane-sulfonamide (0.429 g, 1.20 mmol) was added in a single portion. After 18 hours, the reaction was poured into dichloromethane and brine. The layers were separated, and the aqueous washed with dichloromethane. The combined organic layers were dried over MgSO₄, filtered, and concentrated in vacuo. Purification by flash chromatography afforded 0.406 g (100%) of methyl 5-(1H-benzimidazol-1-yl)-3-{[(trifluoromethyl)-sulfonyl]oxy}-thiophene-2-carboxylate as a white solid. ¹H NMR (300 MHz, DMSO-d₆) δ 8.77 (s, 1H), 7.88 (s, 1H), 7.85 (d, J=8.4 Hz, 1H), 7.83 (d, J=8.5 Hz, 1H), 7.49-7.38 (m, 2H), 3.91 (s, 3H).

### EXAMPLE 31

### Methyl 3-anilino-5-(1H-benzimidazol-1-yl)thiophene-2-carboxylate

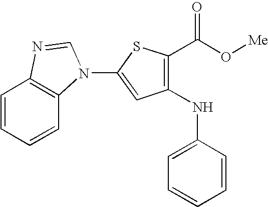

Methyl 5-(1H-benzimidazol-1-yl)-3-{[(trifluoromethyl)sulfonyl]oxy}-thiophene-2-carboxylate (0.200 g, 0.492 mmol), cesium carbonate (0.224 g, 0.687 mmol), rac-2,2′-bis(diphenylphosphino)-1,1′-binaphthyl (0.0306 g, 0.0490 mmol), and tris(dibenzylidene-acetone)dipalladium(0) (0.0225 g, 0.0250 mmol) were combined in flask equipped with a reflux condenser. 5 mL of toluene was added followed by aniline (0.0540 mL, 0.593 mmol). The mixture was heated to 110° C. and maintained at that temperature for 18 hours. The mixture was cooled to room temperature and adsorbed onto silica gel. Purification by flash chromatography afforded 0.138 g (80%) of methyl 3-anilino-5-(1H-benzimidazol-1-yl)thiophene-2-carboxylate as an off-white solid. ¹H NMR (300 MHz, DMSO-d₆) δ 9.01 (s, 1H), 8.77 (s, 1H), 7.84 (d, J=7.7 Hz, 1H), 7.79 (d, J=7.6 Hz, 1H), 7.51 (s, 1H), 7.45-7.33 (m, 6H), 7.08 (m, 1H), 3.84 (s, 3H).

### EXAMPLE 32

### Methyl 5-(1H-benzimidazol-1-yl)-3-(benzoylamino)thiophene-2-carboxylate

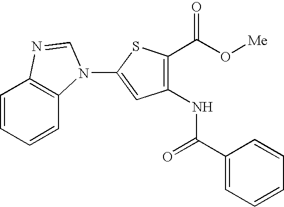

Methyl 5-(1H-benzimidazol-1-yl)-3-{[(trifluoromethyl)sulfonyl]oxy}-thiophene-2-carboxylate (0.350 g, 0.861 mmol), cesium carbonate (0.393 g, 1.21 mmol), rac-2,2′-bis(diphenylphosphino)-1,1′-binaphthyl (0.0536 g, 0.0860 mmol), and tris(dibenzylidene-acetone)dipalladium(0) (0.0394 g, 0.0430 mmol) were combined in flask equipped with a reflux condenser. 12 mL of toluene was added followed by benzamide (0.125 g, 1.03 mmol). The mixture was heated to 100° C. and maintained at that temperature for 40 hours. The mixture was cooled to room temperature and adsorbed onto silica gel. Purification by flash chromatography afforded 0.282 g (87%) of methyl 5-(1H-benzimidazol-1-yl)-3-(benzoylamino)thiophene-2-carboxylate as a white solid. ¹H NMR (300 MHz, DMSO-d₆) δ 11.11 (s, 1H), 8.81 (s, 1H), 8.40 (s, 1H), 8.00 (m, 2H), 7.83 (m, 2H), 7.72-7.60 (m, 3H), 7.50-7.38 (m, 2H), 3.93 (s, 3H). MS (m/z) 378 (m+1).

### EXAMPLE 33

### 5-(1H-Benzimidazol-1-yl)-3-(benzoylamino)thiophene-2-carboxylic acid

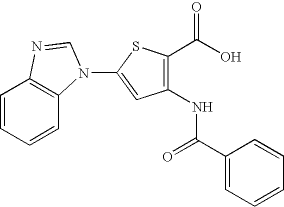

Methyl 5-(1H-benzimidazol-1-yl)-3-(benzoylamino)thiophene-2-carboxylate (0.275 g, 0.729 mmol) was dissolved in 15 mL of dioxane with stirring. 15 mL of 1M LiOH solution was added, and the mixture was stirred for 16 hours at room temperature. 15 mL of 2M HCl solution was added slowly via pipet, resulting in the formation of a solid. The mixture was filtered and the solid was washed with diethyl ether. The solid was collected and dried under high vacuum to yield 0.0963 g (36%) of 5-(1H-benzimidazol-1-yl)-3-(benzoylamino)thiophene-2-carboxylic acid as a tan solid. ¹H NMR (300 MHz, DMSO-d₆) δ 11.31 (s, 1H), 8.79 (s, 1H), 8.39 (s, 1H), 7.97 (m, 2H), 7.83 (m, 2H), 7.73-7.60 (m, 3H), 7.50-7.36 (m, 2H).

### EXAMPLE 34

### 5-(5-Chloro-1H-benzimidazol-1-yl)-3-[(2-methylbenzyl)oxy]thiophene-2-carboxylic acid

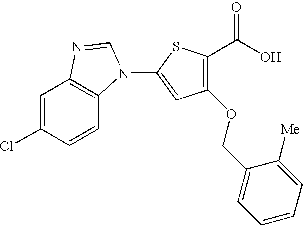

An analogous procedure to that described in Example 33 with methyl 5-(5-chloro-1H-benzimidazol-1-yl)-3-[(2-methylbenzyl)oxy]thiophene-2-carboxylate (0.323 g, 0.782 mmol) provided 0.253 g (81%) of 5-(5-chloro-1H-benzimidazol-1-yl)-3-[(2-methylbenzyl)oxy]thiophene-2-carboxylic acid as a pale yellow solid. ¹H NMR (300 MHz, DMSO-d₆) δ 12.81 (br s, 1H), 8.77 (s, 1H), 7.90 (d, J=1.9 Hz, 1H), 7.85 (d, J=8.7 Hz, 1H), 7.72 (s, 1H), 7.54-7.44 (m, 2), 7.28-7.20 (m, 3H), 5.33 (s, 2H), 2.38 (s, 3H).

### EXAMPLE 35

### 5-(6-Chloro-1H-benzimidazol-1-yl)-3-[(2-methylbenzyl)oxy]thiophene-2-carboxylic acid

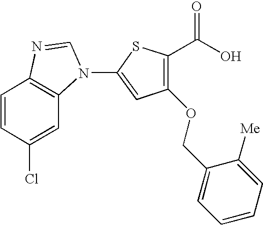

An analogous procedure to that described in Example 33 with methyl 5-(6-chloro-1H-benzimidazol-1-yl)-3-[(2-methylbenzyl)oxy]thiophene-2-carboxylate (0.176 g, 0.426 mmol) provided 0.150 g (88%) of 5-(6-chloro-1H-benzimidazol-1-yl)-3-[(2-methylbenzyl)oxy]thiophene-2-carboxylic acid as a pale yellow solid. ¹H NMR (300 MHz, DMSO-d₆) δ 12.81 (s, 1H), 8.71 (s, 1H), 7.82 (m, 2H), 7.72 (s, 1H), 7.54 (m, 1H), 7.40 (dd, J=8.7, 1.8 Hz, 1H), 7.29-7.21 (m, 3H), 5.35 (s, 2H), 2.39 (s, 3H).

### EXAMPLE 36

### 5-(5-Chloro-1H-benzimidazol-1-yl)-N-methoxy-N-methyl-3-[(2-methylbenzyl)oxy]thiophene-2-carboxamide

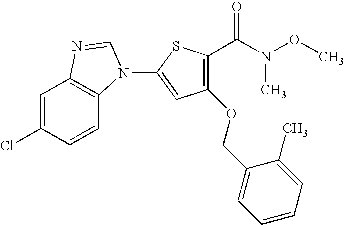

5-(5-Chloro-1H-benzimidazol-1-yl)-3-[(2-methylbenzyl)oxy]thiophene-2-carboxylic acid (0.100 g, 0.251 mmol), N,O-dimethylhydroxylamine hydrochloride (0.0490 g, 0.502 mmol), and 4-dimethylaminopyridine (0.0062 g, 0.051 mmol) were dissolved in 5 mL of dichloromethane. Triethylamine (0.077 mL, 0.550 mmol) was added via syringe, followed by the addition of 1-(3-dimethylaminopropyl)-3-ethylcarbodiimide hydrochloride (0.0870 g, 0.454 mmol) in a single portion. The reaction was stirred for 65 hours and poured into ethyl acetate and water. The layers were separated, and the organic layer was washed with brine. The combined aqueous layers were extracted with ethyl acetate, and the combined organic layers were dried over MgSO₄. Filtration, concentration in vacuo, and purification by flash chromatography afforded 0.0772 g (70%) of 5-(5-chloro-1H-benzimidazol-1-yl)-N-methoxy-N-methyl-3-[(2-methylbenzyl)oxy]thiophene-2-carboxamide as an oil which solidified upon standing. ¹H NMR (300 MHz, DMSO-d₆) δ 8.76 (s, 1H), 7.90 (d, J=2.0 Hz, 1H), 7.84 (d, J=8.8 Hz, 1H), 7.71 (s, 1H), 7.55 (d, J=7.4 Hz, 1H), 7.46 (dd, J=8.8 Hz, 2.0 Hz, 1H), 7.29-7.20 (m, 3H), 5.30 (s, 2H), 3.69 (s, 3H), 3.21 (s, 3H), 2.37 (s, 3H).

### EXAMPLE 37

### 5-(6-Chloro-1H-benzimidazol-1-yl)-N-methoxy-N-methyl-3-[(2-methylbenzyl)oxy]thiophene-2-carboxamide

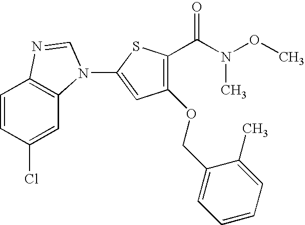

An analogous procedure to that described in Example 36 with 5-(6-chloro-1H-benzimidazol-1-yl)-3-[(2-methylbenzyl)oxy]thiophene-2-carboxylic acid (0.0430 g, 0.108 mmol) afforded 0.0423 g (89%) of 5-(6-chloro-1H-benzimidazol-1-yl)-N-methoxy-N-methyl-3-[(2-methylbenzyl)oxy]thiophene-2-carboxamide as an oil which solidified upon standing. ¹H NMR (300 MHz, DMSO-d₆) δ 8.70 (s, 1H), 7.82 (d, J=8.8 Hz, 1H), 7.80 (s, 1H), 7.71 (s, 1H), 7.56 (d, J=7.3 Hz, 1H), 7.41 (dd, J=8.5, 2.1 Hz, 1H), 7.29-7.20 (m, 3H), 5.32 (s, 2H), 3.68 (s, 3H), 3.32 (s, 3H), 2.38 (s, 3H).

### EXAMPLE 38

### 1-[5-(5-Chloro-1H-benzimidazol-1-yl)-3-[(2-methylbenzyl)oxy]thien-2-yl]ethanone

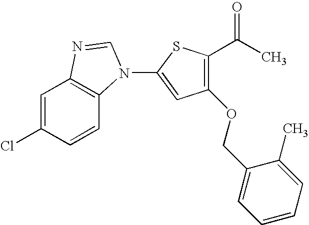

5-(5-Chloro-1H-benzimidazol-1-yl)-N-methoxy-N-methyl-3-[(2-methylbenzyl)-oxy]thiophene-2-carboxamide (0.0750 g, 0.170 mmol) was dissolved in 5 mL of tetrahydrofuran and cooled to −78° C. Methyl magnesium bromide (0.170 mL, 3.0 M in diethyl ether, 0.510 mmol) was added dropwise via syringe. After 5 minutes, the reaction was warmed to 0° C., where it was maintained for an additional 30 minutes. The reaction was quenched by the dropwise addition of 2 mL of 5% HCl. The mixture was poured into ethyl acetate and brine, and the layers were separated. The aqueous layer was extracted with ethyl acetate, and the combined organic layers were dried over MgSO₄. Filtration, concentration in vacuo, and purification by flash chromatography provided 0.0658 g (98%) of 1-{5-(5-chloro-1H-benzimidazol-1-yl)-3-[(2-methylbenzyl)oxy]thien-2-yl}ethanone as a bright yellow solid. ¹H NMR (300 MHz, DMSO-d₆) δ 8.83 (s, 1H), 7.91 (m, 2H), 7.79 (s, 1H), 7.53 (m, 1H), 7.49 (dd, J=8.8, 2.1 Hz, 1H), 7.29 (s, 1H), 7.28 (m, 2H), 5.43 (s, 2H), 2.46 (s, 3H), 2.41 (s, 3H).

### EXAMPLE 39

### 1-{5-(6-Chloro-1H-benzimidazol-1-yl)-3-[(2-methylbenzyl)oxy]thien-2-yl}ethanone

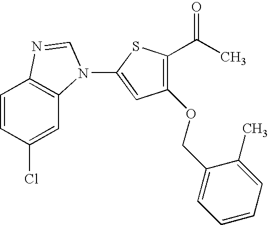

An analogous procedure to that described in Example 38 with 5-(6-Chloro-1H-benzimidazol-1-yl)-N-methoxy-N-methyl-3-[(2-methylbenzyl)-oxy]thiophene-2-carboxamide (0.0400 g, 0.0905 mmol) provided 0.0320 g (89%) of 1-{5-(6-chloro-1H-benzimidazol-1-yl)-3-[(2-methylbenzyl)oxy]thien-2-yl}ethanone as a yellow solid. ¹H NMR (300 MHz, DMSO-d₆) δ 8.77 (s, 1H), 7.89 (d, J=1.7 Hz, 1H), 7.83 (d, J=8.6 Hz, 1H), 7.78 (s, 1H), 7.55 (d, J=6.6 Hz, 1H), 7.43 (dd, J=8.6 Hz, 1.9 Hz, 1H), 7.33-7.25 (m, 3H), 5.45 (s, 2H), 2.46 (s, 3H), 2.41 (s, 3H).

### EXAMPLE 40

### Methyl 5-(5-fluoro-1H-benzimidazol-1-yl)-3-hydroxythiophene-2-carboxylate and Methyl 5-(6-fluoro-1H-benzimidazol-1-yl)-3-hydroxythiophene-2-carboxylate

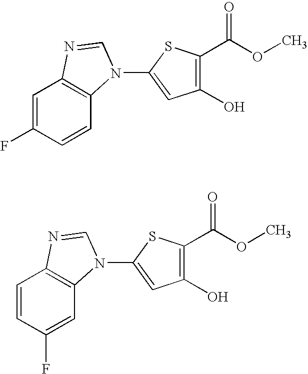

Methyl 2-chloro-3-oxo-2,3-dihydro-2-thiophenecarboxylate (0.250 g, 1.30 mmol) was dissolved in 15 mL of chloroform with stirring. 5-fluorobenzimidazole (0.389 g, 2.86 mmol) was added, and the mixture was allowed to stir for 65 hours. The reaction was poured into half-saturated NaCl and dichloromethane. The layers were separated, and the aqueous layer was extracted twice with dichloromethane. The combined organic layers were dried over MgSO₄, filtered, and concentrated in vacuo. Purification by flash chromatography afforded 0.267 g (70%) of a 1:1 regioisomeric mixture of methyl 5-(5-fluoro-1H-benzimidazol-1-yl)-3-hydroxythiophene-2-carboxylate and methyl 5-(6-fluoro-1H-benzimidazol-1-yl)-3-hydroxythiophene-2-carboxylate as a tan solid. ¹H NMR (300 MHz, DMSO-d₆) δ 10.90, 10.87 (2×s, 1H), 8.75, 8.68 (2×s, 1H), 7.84-7.79 (m, 1H), 7.66-7.59 (m, 1H), 7.32-7.20 (m, 1H), 7.15 (s, 1H), 3.79 (s, 3H).

### EXAMPLE 41

### Methyl 3-hydroxy-5-(5-methoxy-1H-benzimidazol-1-yl)thiophene-2-carboxylate and Methyl 3-hydroxy-5-(6-methoxy-1H-benzimidazol-1-yl)thiophene-2-carboxylate

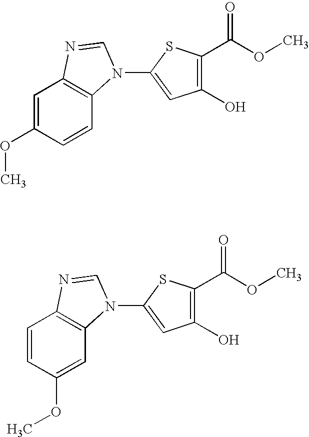

An analogous procedure to that described in Example 40 with 5-methoxybenzimidazole (0.424 g, 2.86 mmol) provided 0.260 g (66%) of a 1:1 regioisomeric mixture of methyl 5-(5-methoxy-1H-benzimidazol-1-yl)-3-hydroxythiophene-2-carboxylate and methyl 5-(6-methoxy-1H-benzimidazol-1-yl)-3-hydroxythiophene-2-carboxylate as a tan solid. ¹H NMR (300 MHz, DMSO-d₆) δ 10.85 (s, 1H), 8.63, 8.52 (2×s, 1H), 7.70, 7.67 (2×d, J=8.0 Hz, 1H), 7.33, 7.23 (2×d, J=2.4 Hz, 1H), 7.14, 7.11 (2×s, 1H), 7.03, 6.97 (2×dd, J=9.0, 2.4 Hz, 1H), 3.84, 3.82, 3.79, 3.78 (4×s, 12H).

### EXAMPLE 42

### Methyl 5-(5-bromo-1H-benzimidazol-1-yl)-3-hydroxythiophene-2-carboxylate and Methyl 5-(6-bromo-1H-benzimidazol-1-yl)-3-hydroxythiophene-2-carboxylate

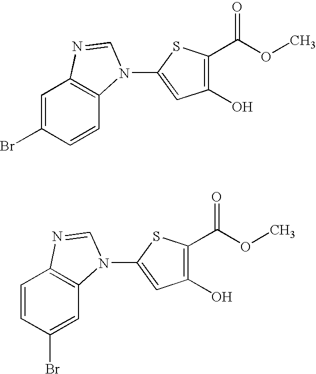

An analogous procedure to that described in Example 40 with 5-bromobenzimidazole (2.20 g, 11.2 mmol) provided 1.03 g (53%) of a 1:1 regioisomeric mixture of methyl 5-(5-bromo-1H-benzimidazol-1-yl)-3-hydroxythiophene-2-carboxylate and methyl 5-(6-bromo-1H-benzimidazol-1-yl)-3-hydroxythiophene-2-carboxylate as a tan solid. ¹H NMR (300 MHz, DMSO-d₆) δ 10.90 (s, 1H), 8.74, 8.70 (2×s, 1H), 8.02, 7.93 (2×d, J=1.8 Hz, 1H), 7.77 (m, 1H), 7.54 (m, 1H), 7.17, 7.15 (2×s, 1H), 3.79 (s, 3H).

### EXAMPLE 43

### Methyl 5-(5,6-dichloro-1H-benzimidazol-1-yl)-3-hydroxythiophene-2-carboxylate

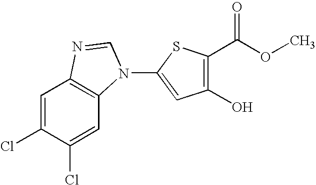

An analogous procedure to that described in Example 40 with 5,6-dichlorobenzimidazole (2.15 g, 11.5 mmol) provided 0.359 g (18%) of methyl 5-(5,6-dichloro-1H-benzimidazol-1-yl)-3-hydroxythiophene-2-carboxylate as a tan solid. ¹H NMR (300 MHz, DMSO-d₆) δ 10.90 (s, 1H), 8.78 (s, 1H), 8.12 (s, 1H), 8.02 (s, 1H), 7.18 (s, 1H), 3.79 (s, 3H).

### EXAMPLE 44

### Methyl 5-(5,6-dimethoxy-1H-benzimidazol-1-yl)-3-hydroxythiophene-2-carboxylate

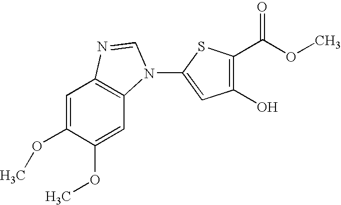

An analogous procedure to that described in Example 40 with 5,6-dimethoxy-benzimidazole (2.00 g, 11.22 mmol) provided 0.632 g (34%) of methyl 5-(5,6-dimethoxy-1H-benzimidazol-1-yl)-3-hydroxythiophene-2-carboxylate as a tan solid. ¹H NMR (300 MHz, DMSO-d₆) δ 10.81 (s, 1H), 8.46 (s, 1H), 7.34 (s, 1H), 7.24 (s, 1H), 7.13 (s, 1H), 3.85 (s, 3H), 3.82 (s, 3H), 3.79 (s, 3H).

### EXAMPLE 45

### Methyl 5-(5,6-dichloro-1H-benzimidazol-1-yl)-3-[(2-methylbenzyl)oxy]thiophene-2-carboxylate

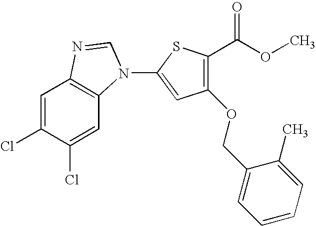

Methyl 5-(5,6-dichloro-1H-benzimidazol-1-yl)-3-hydroxythiophene-2-carboxylate (0.0900 g, 0.262 mmol) was dissolved in 5 mL of N,N-dimethylformamide with stirring. Solid potassium carbonate (0.0430 g, 0.311 mmol) was added in a single portion. 2-Methylbenzyl bromide (0.042 mL, 0.31 mmol) was added via syringe. The reaction was stirred for 65 hours and poured into ethyl acetate and water. The layers were separated, and the organic layer was washed with brine. The combined aqueous layers were extracted with ethyl acetate, and the combined organic layers were dried over MgSO₄. The solution was filtered, concentrated in vacuo, and purified by flash chromatography to afford 0.107 g (91%) of methyl 5-(5,6-dichloro-1H-benzimidazol-1-yl)-3-[(2-methylbenzyl)oxy]thiophene-2-carboxylate as an off-white solid. ¹H NMR (300 MHz, DMSO-d₆) δ 8.80 (s, 1H), 8.14 (s, 1H), 8.05 (s, 1H), 7.79 (s, 1H), 7.55 (d, J=7.5 Hz, 1H), 7.28-7.24 (m, 3H), 5.38 (s, 2H), 3.77 (s, 3H), 2.39 (s, 3H).

### EXAMPLE 46

### Methyl 5-(5-fluoro-1H-benzimidazol-1-yl)-3-[(2-methylbenzyl)-oxy]thiophene-2-carboxylate and Methyl 5-(6-fluoro-1H-benzimidazol-1-yl)-3-[(2-methylbenzyl)oxy]thiophene-2-carboxylate

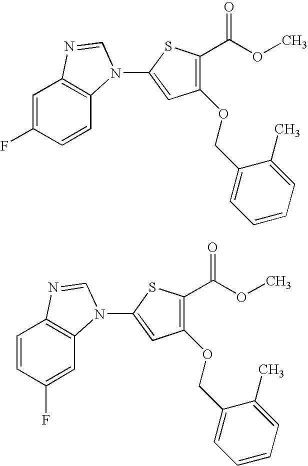

An analogous procedure to that described in Example 45 with a 1:1 regioisomeric mixture of methyl 5-(5-fluoro-1H-benzimidazol-1-yl)-3-hydroxythiophene-2-carboxylate and methyl 5-(6-fluoro-1H-benzimidazol-1-yl)-3-hydroxythiophene-2-carboxylate (0.262 g, 0.896 mmol) provided 0.291 g (82%) of a 1:1 regioisomeric mixture of methyl 5-(5-fluoro-1H-benzimidazol-1-yl)-3-[(2-methylbenzyl)-oxy]thiophene-2-carboxylate and methyl 5-(6-fluoro-1H-benzimidazol-1-yl)-3-[(2-methylbenzyl)oxy]thiophene-2-carboxylate as an off-white solid. ¹H NMR (300 MHz, DMSO-d₆) δ 8.78, 8.71 (2×s, 1H), 7.95-7.50 (m, 5H), 7.35-7.22 (m, 3H), 5.39, 5.37 (2×s, 2H), 3.77 (s, 3H), 2.39 (s, 3H).

### EXAMPLE 47

### Methyl 5-(5-methoxy-1H-benzimidazol-1-yl)-3-[(2-methylbenzyl)oxy]-thiophene-2-carboxylate and Methyl 5-(6-methoxy-1H-benzimidazol-1-yl)-3-[(2-methylbenzyl)oxy]thiophene-2-carboxylate

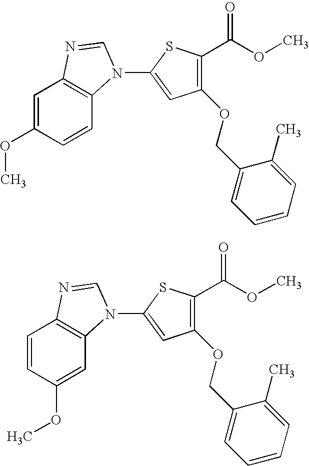

An analogous procedure to that described in Example 45 with a 1:1 regioisomeric mixture of methyl 5-(5-methoxy-1H-benzimidazol-1-yl)-3-hydroxythiophene-2-carboxylate and methyl 5-(6-methoxy-1H-benzimidazol-1-yl)-3-hydroxythiophene-2-carboxylate (0.255 g, 0.838 mmol) gave 0.249 g (73%) of a 1:1 regioisomeric mixture of methyl 5-(5-methoxy-1H-benzimidazol-1-yl)-3-[(2-methylbenzyl)oxy]-thiophene-2-carboxylate and methyl 5-(6-methoxy-1H-benzimidazol-1-yl)-3-[(2-methylbenzyl)oxy]thiophene-2-carboxylate as an off-white solid. ¹H NMR (300 MHz, DMSO-d₆) δ 8.67, 8.55 (2×s, 1H), 7.95, 7.76-7.67, 7.56-7.53 (m, 3H), 7.34, 7.30-7.21, 7.07-6.97 (m, 5H), 5.38, 5.37 (2×s, 2H), 3.84, 3.83, 3.77, 3.76 (4×s, 12H), 2.39 (s, 3H).

### EXAMPLE 48

### Methyl 5-(5-bromo-1H-benzimidazol-1-yl)-3-[(2-methylbenzyl)oxy]-25 thiophene-2-carboxylate and Methyl 5-(6-bromo-1H-benzimidazol-1-yl)-3-[(2-methylbenzyl)oxy]-thiophene-2-carboxylate

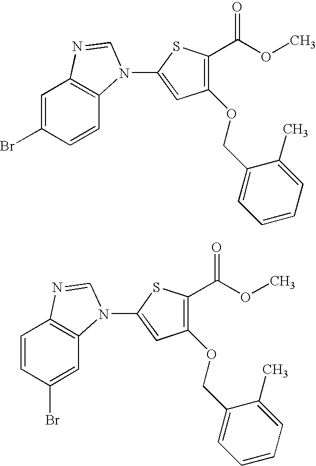

An analogous procedure to that described in Example 45 with a 1:1 regioisomeric mixture of methyl 5-(5-bromo-1H-benzimidazol-1-yl)-3-hydroxythiophene-2-carboxylate and methyl 5-(6-bromo-1H-benzimidazol-1-yl)-3-hydroxythiophene-2-carboxylate (0.750 g, 2.12 mmol) provided 0.681 g (70%) of a 1:1 regioisomeric mixture of methyl 5-(5-bromo-1H-benzimidazol-1-yl)-3-[(2-methylbenzyl)-oxy]thiophene-2-carboxylate and methyl 5-(6-bromo-1H-benzimidazol-1-yl)-3-[(2-methylbenzyl)oxy]thiophene-2-carboxylate as an off-white solid. ¹H NMR (300 MHz, DMSO-d₆) δ 8.77, 8.71 (2×s, 1H), 8.04, 7.95 (2×d, J=1.8 Hz, 1H), 7.83-7.75, 7.60-7.52, 7.27-7.11(m, 7H), 5.38, 5.37 (2×s, 2H), 3.77 (s, 3H), 2.40, 2.39 (s, 3H).

### EXAMPLE 49

### Methyl 5-(6-chloro-1H-benzimidazol-1-yl)-3-[(2-chloro-4-fluorobenzyl)oxy]thiophene-2-carboxylate

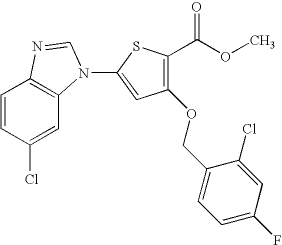

An analogous procedure to that described in Example 45 with methyl 5-(6-chloro-1H-benzimidazol-1-yl)-3-hydroxythiophene-2-carboxylate (0.100 g, 0.324 mmol) and 2-chloro-4-fluorobenzyl bromide (0.0869 g, 0.389 mmol) provided 0.131 g (90%) of methyl 5-(6-chloro-1H-benzimidazol-1-yl)-3-[(2-chloro-4-fluorobenzyl)oxy]-thiophene-2-carboxylate as an off-white solid. ¹H NMR (300 MHz, DMSO-d₆) δ 8.75 (s, 1H), 7.89 (d, J=1.9 Hz, 1H), 7.84-7.78 (m, 2H), 7.78 (s, 1H), 7.56 (dd, J=8.8, 2.7 Hz, 1H), 7.42 (dd, J=8.6, 1.9 Hz, 1H), 7.35 (ddd, J=8.7, 8.7, 2.7 Hz, 1H), 5.42 (s, 2H), 3.78 (s, 3H).

### EXAMPLE 50

### Methyl 5-(6-chloro-1H-benzimidazol-1-yl)-3-[(2,4-difluorobenzyl)oxy]-thiophene-2-carboxylate

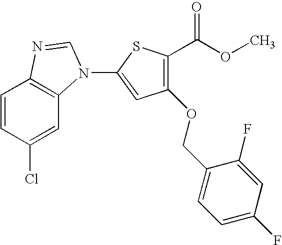

An analogous procedure to that described in Example 45 with methyl 5-(6-chloro-1H-benzimidazol-1-yl)-3-hydroxythiophene-2-carboxylate (0.100 g, 0.324 mmol) and 2,4-difluorobenzyl bromide (0.054 mL, 0.39 mmol) provided 0.122 g (87%) of methyl 5-(6-chloro-1H-benzimidazol-1-yl)-3-[(2,4-difluorobenzyl)oxy]thiophene-2-carboxylate as an off-white solid. ¹H NMR (300 MHz, DMSO-d₆) δ 8.74 (s, 1H), 7.89 (d, J=1.9 Hz, 1H), 7.83 (d, J=8.6 Hz, 1H), 7.77-7.69 (m, 1H), 7.76 (s, 1H), 7.42 (dd, J=8.6, 1.9 Hz, 1H), 7.35 (m, 1H), 7.19 (m, 1H), 5.41 (s, 2H), 3.77 (s, 3H).

### EXAMPLE 51

### Methyl 5-(6-chloro-1H-benzimidazol-1-yl)-3-(pyridin-3-ylmethoxy)thiophene-2-carboxylate

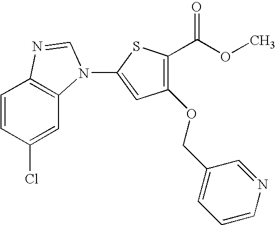

An analogous procedure to that described in Example 45 with methyl 5-(6-chloro-1H-benzimidazol-1-yl)-3-hydroxythiophene-2-carboxylate (0.100 g, 0.324), 3-(bromomethyl)pyridine hydrobromide (0.0980 g, 0.387 mmol), and potassium carbonate (0.107 g, 0.774 mmol) yielded 0.0393 g (30%) of methyl 5-(6-chloro-1H-benzimidazol-1-yl)-3-(pyridin-3-ylmethoxy)thiophene-2-carboxylate as a tan solid. ¹H NMR (300 MHz, DMSO-d₆) δ 8.72 (s, 1H), 8.72 (m, 1H), 8.58 (dd, J=4.8, 1.5 Hz, 1H), 7.93 (m, 1H), 7.86 (d, J=1.9 Hz, 1H), 7.83 (d, J=8.7 Hz, 1H), 7.73 (s, 1H), 7.48 (m, 1H), 7.42 (dd, J=8.7, 1.9 Hz, 1H), 5.45 (s, 2H), 3.79 (s, 3H).

### EXAMPLE 52

### Methyl 5-(1H-benzimidazol-1-yl)-3-(prop-2-ynyloxy)thiophene-2-carboxylate

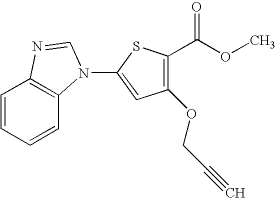

An analogous procedure to that described in Example 45 with methyl 5-(1H-benzimidazol-1-yl)-3-hydroxy-2-thiophenecarboxylate (0.250 g, 0.911 mmol) and propargyl bromide (0.12 mL 80% in toluene, 1.08 mmol) afforded 0.211 g (74%) of methyl 5-(1H-benzimidazol-1-yl)-3-(prop-2-ynyloxy)thiophene-2-carboxylate as a tan solid. ¹H NMR (300 MHz, DMSO-d₆) δ 8.70 (s, 1H), 7.88 (d, J=7.7 Hz, 1H), 7.81 (d, J=7.5 Hz, 1H), 7.61 (s, 1H), 7.49-7.36 (m, 2H), 5.07 (d, J=2.3 Hz, 2H), 3.78 (s, 3H), 3.73 (t, J=2.3 Hz, 1H). MS (m/z) 313 (m+1).

### EXAMPLE 53

### Methyl 5-(5-bromo-1H-benzimidazol-1-yl)-3-{[2-(trifluoromethyl)-benzyl]oxy}thiophene-2-carboxylate and Methyl 5-(6-bromo-1H-benzimidazol-1-yl)-3-{[2-(trifluoromethyl)benzyl]oxy}thiophene-2-carboxylate

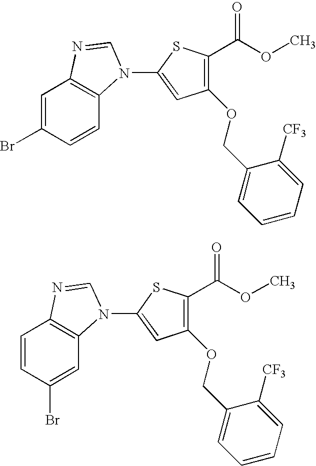

An analogous procedure to that described in Example 45 with a 1:1 regioisomeric mixture of methyl 5-(5-bromo-1H-benzimidazol-1-yl)-3-hydroxythiophene-2-carboxylate and methyl 5-(6-bromo-1H-benzimidazol-1-yl)-3-hydroxythiophene-2-carboxylate (0.200 g, 0.566 mmol) and 2-trifluoromethylbenzyl bromide (0.163 g, 0.682 mmol) provided a 1:1 regioisomeric mixture of products. The mixture was separated by flash chromatography to afford 0.0952 g (33%) of methyl 5-(5-bromo-1H-benzimidazol-1-yl)-3-{[2-(trifluoromethyl)-benzyl]oxy}thiophene-2-carboxylate as an off-white solid: ¹H NMR (300 MHz, DMSO-d₆) δ 8.79 (s, 1H), 8.04 (d, J=1.8 Hz, 1H), 7.97 (d, J=7.6 Hz, 1H), 7.85-7.77 (m, 2H), 7.75 (s, 1H), 7.62 (m, 1H), 7.60 (d, J=1.9 Hz, 1H), 7.58 (d, J=1.8 Hz, 1H), 5.50 (s, 2H), 3.78 (s, 3H), and 0.0970 g (34%) of methyl 5-(6-bromo-1H-benzimidazol-1-yl)-3-{[2-(trifluoromethyl)-benzyl]oxy}thiophene-2-carboxylate as an off-white solid: ¹H NMR (300 MHz, DMSO-d₆) δ 8.73 (s, 1H), 7.99-7.94 (m, 2H), 7.85-7.71 (m, 4H), 7.62 (m, 1H), 7.53 (dd, J=8.6, 1.5 Hz, 1H), 5.52 (s, 2H), 3.78 (s, 3H).

### EXAMPLE 54

### Methyl 5-(5,6-dimethoxy-1H-benzimidazol-1-yl)-3-[(2-trifluoromethyl-benzyl)oxy]thiophene-2-carboxylate

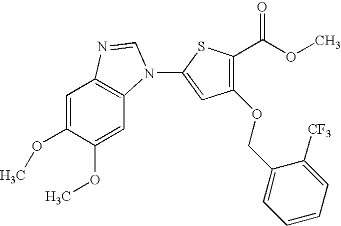

An analogous procedure to that described in Example 45 with methyl 5-(5,6-dimethoxy-1H-benzimidazol-1-yl)-3-hydroxythiophene-2-carboxylate (0.108 g, 0.323 mmol) and 2-trifluoromethylbenzyl bromide (0.232 g, 0.971 mmol) afforded 0.109 g (69%) of methyl 5-(5,6-dimethoxy-1H-benzimidazol-1-yl)-3-[(2-trifluoromethylbenzyl)-oxy]thiophene-2-carboxylate as an off-white solid. ¹H NMR (300 MHz, DMSO-d₆) δ 8.50 (s, 1H), 7.96 (d, J=7.5 Hz, 1H), 7.84-7.76 (m, 2H), 7.66 (s, 1H), 7.61 (dd, J=7.7, 7.7 Hz, 1H), 7.35 (s, 1H), 7.26 (s, 1H), 5.53 (s, 2H), 3.84 (s, 3H), 3.83 (s, 3H), 3.78 (s, 3H).

### EXAMPLE 55

### Methyl 3-[(2,6-dichlorobenzyl)oxy]-5-(5,6-dimethoxy-1H-benzimidazol-1-yl)thiophene-2-carboxylate

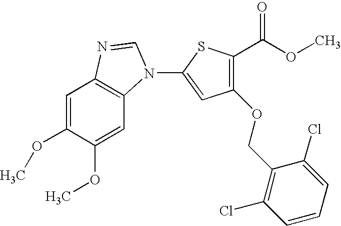

An analogous procedure to that described in Example 45 with methyl 5-(5,6-dimethoxy-1H-benzimidazol-1-yl)-3-hydroxythiophene-2-carboxylate (0.100 g, 0.299 mmol) and 2,6-dichlorobenzyl bromide (0.0869 g, 0.362 mmol) provided 0.117 g (79%) of methyl 3-[(2,6-dichlorobenzyl)oxy]-5-(5,6-dimethoxy-1H-benzimidazol-1-yl)thiophene-2-carboxylate as an off-white solid. ¹H NMR (300 MHz, DMSO-d₆) a 8.52 (s, 1H), 7.78 (s, 1H), 7.62 (d, J=1.5 Hz, 1H), 7.59 (s, 1H), 7.51 (dd, J=9.3, 6.8 Hz, 1H), 7.35 (s, 1H), 7.31 (s, 1H), 5.52 (s, 2H), 3.87 (s, 3H), 3.83 (s, 3H), 3.71 (s, 3H).

### EXAMPLE 56

### Methyl 3-[(2-bromobenzyl)oxy]-5-(5,6-dimethoxy-1H-benzimidazol-1-yl)thiophene-2-carboxylate

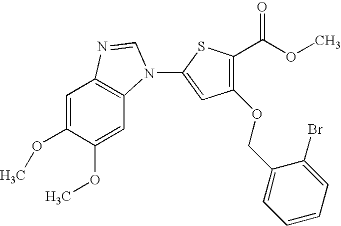

An analogous procedure to that described in Example 45 with methyl 5-(5,6-dimethoxy-1H-benzimidazol-1-yl)-3-hydroxythiophene-2-carboxylate (0.100 g, 0.299 mmol) and 2-bromobenzyl bromide (0.0905 g, 0.362 mmol) provided 0.114 g (76%) of methyl 3-[(2-bromobenzyl)oxy]-5-(5,6-dimethoxy-1H-benzimidazol-1-yl)thiophene-2-carboxylate as an off-white solid. ¹H NMR (300 MHz, DMSO-d₆) δ 8.51 (s, 1H), 7.73 (ddd, J=7.6, 7.6, 1.0 Hz, 1H), 7.68 (m, 1H), 7.68 (s, 1H), 7.49 (ddd, J=7.6, 7.6, 1.2 Hz, 1H), 7.35 (s, 1H), 7.34 (ddd, J=7.6, 7.6, 1.6 Hz, 1H), 7.26 (s, 1H), 5.40 (s, 2H), 3.84 (s, 3H), 3.83 (s, 3H), 3.79 (s, 3H).

### EXAMPLE 57

### Methyl 5-(5,6-dichloro-1H-benzimidazol-1-yl)-3-(3-furylmethoxy)-thiophene-2-carboxylate

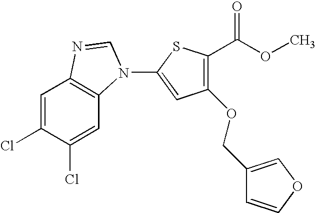

Methyl 5-(5,6-dichloro-1H-benzimidazol-1-yl)-3-hydroxythiophene-2-carboxylate (0.0900 g, 0.262 mmol) and triphenylphosphine (0.0890 g, 0.339 mmol) were dissolved in 4 mL of tetrahydrofuran with stirring. The reaction was cooled to 0° C., and 3-furanmethanol (0.030 mL, 0.35 mmol) was added via syringe. Diethyl azodicarboxylate (0.053 mL, 0.34 mmol) was added dropwise via syringe. The reaction was warmed to room temperature and stirred for 3 hours. The mixture was adsorbed onto silica gel and purification by flash chromatography afforded 0.0725 g (65%) of methyl 5-(5,6-dichloro-1H-benzimidazol-1-yl)-3-(3-furylmethoxy)-thiophene-2-carboxylate as an inseparable mixture with diethyl hydrazine-1,2-dicarboxylate, which could be easily removed in the workup of the following reaction. ¹H NMR (300 MHz, DMSO-d₆) δ 8.79 (s, 1H), 8.14 (s, 1H), 8.07 (s, 1H), 7.85 (dd, J=1.6, 0.9 Hz, 1H), 7.72 (s, 1H), 7.70 (dd, J=1.6, 1.6 Hz, 1H), 6.61 (dd, J=1.9, 0.8 Hz, 1H), 5.25 (s, 2H), 3.77 (s, 3H).

### EXAMPLE 58

### Methyl 5-(5,6-dichloro-1H-benzimidazol-1-yl)-3-(2-furylmethoxy)-thiophene-2-carboxylate

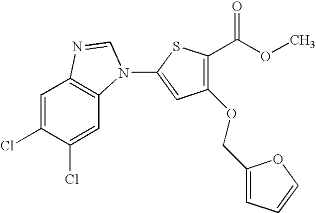

An analogous procedure to that described in Example 57 with methyl 5-(5,6-dichloro-1H-benzimidazol-1-yl)-3-hydroxythiophene-2-carboxylate (0.0900 g, 0.262 mmol) and furfuryl alcohol (0.029 mL, 0.34 mmol) provided 0.0525 g (47%) of methyl 5-(5,6-dichloro-1H-benzimidazol-1-yl)-3-(2-furylmethoxy)-thiophene-2-carboxylate as an inseparable mixture with diethyl hydrazine-1,2-dicarboxylate, which could be easily removed in the workup of the following reaction. ¹H NMR (300 MHz, DMSO-d₆) δ 8.79 (s, 1H), 8.14 (s, 1H), 8.09 (s, 1H), 7.76 (s, 1H), 7.75 (dd, J=1.9, 0.8 Hz, 1H), 6.71 (dd, J=3.2, 0.8 Hz, 1H), 6.51 (dd, J=3.2, 1.9 Hz, 1H), 5.36 (s, 2H), 3.75 (s, 3H).

### EXAMPLE 59

### Methyl 5-(5,6-dichloro-1H-benzimidazol-1-yl)-3-(thien-3-ylmethoxy)-thiophene-2-carboxylate

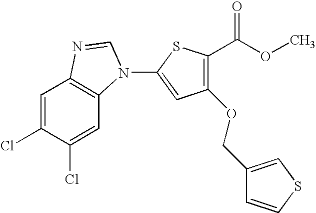

An analogous procedure to that described in Example 57 with methyl 5-(5,6-dichloro-1H-benzimidazol-1-yl)-3-hydroxythiophene-2-carboxylate (0.0900 g, 0.262 mmol) and 3-thiophenemethanol (0.032 mL, 0.34 mmol) provided 0.0745 g (65%) of methyl 5-(5,6-dichloro-1H-benzimidazol-1-yl)-3-(thien-3-ylmethoxy)-thiophene-2-carboxylate as an inseparable mixture with diethyl hydrazine-1,2-dicarboxylate, which could be easily removed in the workup of the following reaction. ¹H NMR (300 MHz, DMSO-d₆) δ 8.78 (s, 1H), 8.14 (s, 1H), 8.04 (s, 1H), 7.71 (s, 1H), 7.66 (m, 1H), 7.60 (dd, J=5.0, 2.9 Hz, 1H), 7.22 (dd, J=5.0, 1.2 Hz, 1H), 5.38 (s, 2H), 3.78 (s, 3H).

### EXAMPLE 60

### Methyl 5-(5,6-dichloro-1H-benzimidazol-1-yl)-3-(thien-2-yl methoxy)-thiophene-2-carboxylate

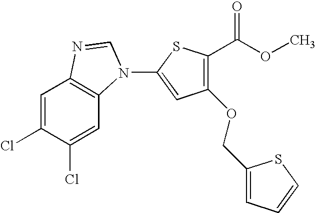

An analogous procedure to that described in Example 57 with methyl 5-(5,6-dichloro-1H-benzimidazol-1-yl)-3-hydroxythiophene-2-carboxylate (0.0775 g, 0.226 mmol) and 2-thiophenemethanol (0.028 mL, 0.30 mmol) provided 0.0599 g (60%) of methyl 5-(5,6-dichloro-1H-benzimidazol-1-yl)-3-(thien-2-ylmethoxy)-thiophene-2-carboxylate as an inseparable mixture with diethyl hydrazine-1,2-dicarboxylate, which could be easily removed in the workup of the following reaction. ¹H NMR (300 MHz, DMSO-d₆) δ 8.78 (s, 1H), 8.14 (s, 1H), 8.05 (s, 1H), 7.75 (s, 1H), 7.61 (dd, J=5.0, 1.2 Hz, 1H), 7.30 (dd, J=3.5, 1.2 Hz, 1H), 7.07 (dd, J=5.0, 3.5 Hz, 1H), 5.57 (s, 2H), 3.77 (s, 3H).

### EXAMPLE 61

### 5-(6-Chloro-1H-benzimidazol-1-yl)-3-[(2-methylbenzyl)oxy]thiophene-2-carboxamide

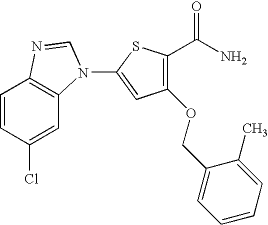

Methyl 5-(6-chloro-1H-benzimidazol-1-yl)-3-[(2-methylbenzyl)oxy]thiophene-2-carboxylate (0.172 g, 0.417 mmol) was placed in sealed tube. Ammonia in methanol (15.0 mL, 2.0 M in MeOH, 30 mmol) was added, and the vessel was sealed. The tube was placed in an oil bath preheated to 80° C., and stirred at that temperature for 24 hours. The reaction was cooled to room temperature, and an additional 15.0 mL of the ammonia in methanol solution was added. The vessel was resealed and heating continued for an additional 44 hours. The reaction was cooled to room temperature and adsorbed onto silica gel. Purification by flash chromatography provided 0.0417 g (24%) of recovered starting material and 0.0820 g (49%) of 5-(6-chloro-1H-benzimidazol-1-yl)-3-[(2-methylbenzyl)oxy]thiophene-2-carboxamide as a yellow solid. ¹H NMR (300 MHz, DMSO-d₆) δ 8.68 (s, 1H), 7.82 (d, J=8.8 Hz, 1H), 7.78 (d, J=2.1 Hz, 1H), 7.72 (br s, 1H), 7.70 (s, 1H), 7.51 (d, J=7.0 Hz, 1H), 7.40 (dd, J=8.6, 2.1 Hz, 1H), 7.34-7.21 (m, 3H), 6.88 (br s, 1H), 5.44 (s, 2H), 2.40 (s, 3H).

### EXAMPLE 62

### 5-(6-Bromo-1H-benzimidazol-1-yl)-3-{[2-(trifluoromethyl)benzyl]-oxy}thiophene-2-carboxamide

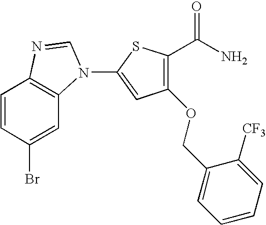

An analogous procedure to that described in Example 61 with methyl 5-(6-bromo-1H-benzimidazol-1-yl)-3-{[2-(trifluoromethyl)benzyl]oxy}thiophene-2-carboxylate (0.0950 g, 0.186 mmol) afforded 0.0557 g (60%) of 5-(6-bromo-1H-benzimidazol-1-yl)-3-{[2-(trifluoromethyl)benzyl]-oxy}thiophene-2-carboxamide as an off-white solid. ¹H NMR (300 MHz, DMSO-dB) δ 8.67 (s, 1H), 7.91 (d, J=1.6 Hz, 1H), 7.89-7.71 (m, 5H), 7.68 (s, 1H), 7.67 (m, 1H), 7.52 (dd, J=8.6, 1.8 Hz, 1H), 6.81 (br s, 1H), 5.56 (s, 2H).

### EXAMPLE 63

### 5-(5,6-Dimethoxy-1H-benzimidazol-1-yl)-3-{[2-(trifluoromethyl)-benzyl]oxy}thiophene-2-carboxamide

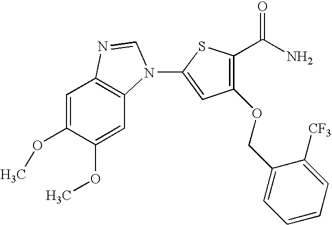

An analogous procedure to that described in Example 61 with methyl 5-(5,6-dimethoxy-1H-benzimidazol-1-yl)-3-[(2-trifluoromethylbenzyl)oxy]thiophene-2-carboxylate provided 0.0351 g (34%) of 5-(5,6-dimethoxy-1H-benzimidazol-1-yl)-3-{[2-(trifluoromethyl)-benzyl]oxy}thiophene-2-carboxamide as a light tan solid. ¹H NMR (300 MHz, DMSO-d₆) δ 8.43 (s, 1H), 7.90-7.58 (m, 5H), 7.60 (s, 1H), 7.34 (s, 1H), 7.21 (s, 1H), 6.82 (br s, 1H), 5.56 (s, 2H).

### EXAMPLE 64

### 3-[(2,6-Dichlorobenzyl)oxy]-5-(5,6-dimethoxy-1H-benzimidazol-1-yl)thiophene-2-carboxamide

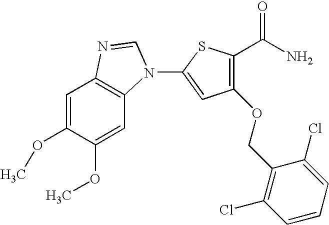

An analogous procedure to that described in Example 61 with methyl 3-[(2,6-dichlorobenzyl)oxy]-5-(5,6-dimethoxy-1H-benzimidazol-1-yl)thiophene-2-carboxylate (0.115 g, 0.233 mmol) afforded 0.0392 g (35%) of 3-[(2,6-dichlorobenzyl)oxy]-5-(5,6-dimethoxy-1H-benzimidazol-1-yl)thiophene-2-carboxamide as an off-white solid. ¹H NMR (300 MHz, DMSO-d₆) δ 8.46 (s, 1H), 7.79 (s, 1H), 7.68 (br s, 1H), 7.63 (d, J=1.5 Hz, 1H), 7.60 (s, 1H), 7.52 (dd, J=9.1, 6.9 Hz, 1H), 7.35 (s, 1H), 7.30 (s, 1H), 6.63 (br s, 1H), 5.58 (s, 2H), 3.87 (s, 3H), 3.83 (s, 3H).

### EXAMPLE 65

### 3-[(2-Bromobenzyl)oxy]-5-(5,6-dimethoxy-1H-benzimidazol-1-yl)thiophene-2-carboxamide

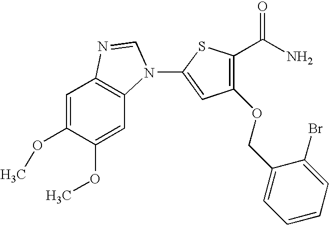

An analogous procedure to that described in Example 61 with methyl 3-[(2-bromobenzyl)oxy]-5-(5,6-dimethoxy-1H-benzimidazol-1-yl)thiophene-2-carboxylate (0.112 g, 0.222 mmol) afforded 0.0296 g (27%) of 3-[(2-bromobenzyl)oxy]-5-(5,6-dimethoxy-1H-benzimidazol-1-yl)thiophene-2-carboxamide as a yellow solid. ¹H NMR (300 MHz, DMSO-d₆) δ 8.43 (s, 1H), 7.78-7.64 (m, 3H), 7.66 (s, 1H), 7.47 (m, 1H), 7.86 (m, 1H), 7.34 (s, 1H), 7.21 (s, 1H), 6.91 (br s, 1H), 5.46 (s, 2H).

### EXAMPLE 66

### 5-(5,6-Dichloro-1H-benzimidazol-1-yl)-3-[(2-methylbenzyl)oxy]-thiophene-2-carboxylic acid

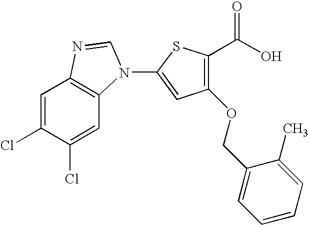

An analogous procedure to that described in Example 33 with methyl 5-(5,6-dichloro-1H-benzimidazol-1-yl)-3-[(2-methylbenzyl)oxy]thiophene-2-carboxylate (0.105 g, 0.235 mmol) yielded 0.0695 g (68%) of 5-(5,6-dichloro-1H-benzimidazol-1-yl)-3-[(2-methylbenzyl)oxy]thiophene-2-carboxylic acid as a light tan solid. ¹H NMR (300 MHz, DMSO-d₆) δ 12.84 (s, 1H), 8.78 (s, 1H), 8.14 (s, 1H), 8.04 (s, 1H), 7.73 (s, 1H), 7.53 (m, 1H), 7.29-7.22 (m, 3H), 5.35 (s, 2H), 2.39 (s, 3H).

### EXAMPLE 67

### 5-(5-Fluoro-1H-benzimidazol-1-yl)-3-[(2-methylbenzyl)oxy]thiophene-2-carboxylic acid and 5-(6-Fluoro-1H-benzimidazol-1-yl)-3-[(2-methylbenzyl)oxy]-thiophene-2-carboxylic acid

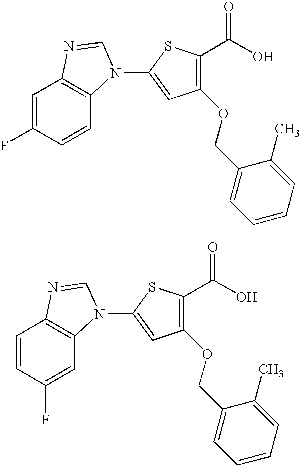

An analogous procedure to that described in Example 33 with a 1:1 regioisomeric mixture of methyl 5-(5-fluoro-1H-benzimidazol-1-yl)-3-[(2-methylbenzyl)-oxy]thiophene-2-carboxylate and methyl 5-(6-fluoro-1H-benzimidazol-1-yl)-3-[(2-methylbenzyl)-oxy]thiophene-2-carboxylate (0.285 g, 0.719 mmol) provided 0.215 g (78%) of a 1:1 regioisomeric mixture of 5-(5-fluoro-1H-benzimidazol-1-yl)-3-[(2-methyl benzyl)oxy]-thiophene-2-carboxylic acid and 5-(6-fluoro-1H-benzimidazol-1-yl)-3-[(2-methylbenzyl)oxy]-thiophene-2-carboxylic acid as a yellow solid. ¹H NMR (300 MHz, DMSO-d₆) δ 12.81 (br s, 1H), 8.76, 8.69 (2×s, 1H), 7.84 (m, 1H), 7.72, 7.70 (2×s, 1H), 7.66 (m, 1H), 7.53 (d, J=6.3 Hz, 1H), 7.36-7.19 (m, 4H), 5.35, 5.34 (2×s, 2H), 2.38 (s, 3H).

### EXAMPLE 68

### 5-(5-Methoxy-1H-benzimidazol-1-yl)-3-[(2-methylbenzyl)oxy]thiophene-2-carboxylic acid and 5-(6-Methoxy-1H-benzimidazol-1-yl)-3-[(2-methylbenzyl)-oxy]thiophene-2-carboxylic acid

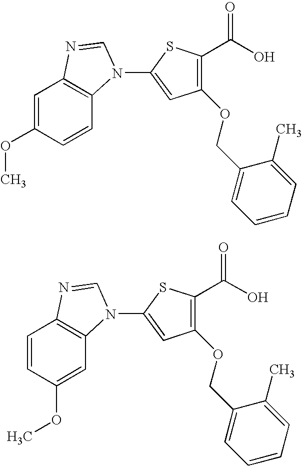

An analogous procedure to that described in Example 33 with a 1:1 regioisomeric mixture of methyl 5-(5-methoxy-1H-benzimidazol-1-yl)-3-[(2-methylbenzyl)oxy]-thiophene-2-carboxylate and methyl 5-(6-methoxy-1H-benzimidazol-1-yl)-3-[(2-methylbenzyl)oxy]thiophene-2-carboxylate (0.243 g, 0.595 mmol) provided 0.217 g (92%) of a 1:1 regioisomeric mixture of 5-(5-methoxy-1H-benzimidazol-1-yl)-3-[(2-methylbenzyl)oxy]thiophene-2-carboxylic acid and 5-(6-methoxy-1H-benzimidazol-1-yl)-3-[(2-methylbenzyl)oxy]thiophene-2-carboxylic acid as a pale yellow solid. ¹H NMR (300 MHz, DMSO-d₆) δ 8.93, 8.79 (2×s, 1H), 7.80-7.68 (m, 2H), 7.53 (d, J=6.6 Hz, 1H), 7.35, 7.31-7.17 (m, 4H), 7.10, 7.04 (2×dd, J=9.0, 2.4 Hz, J=8.9, 2.3 Hz, 1H), 5.34 (s, 2H), 2.38 (s, 3H).

### EXAMPLE 69

### 5-(5-Bromo-1H-benzimidazol-1-yl)-3-[(2-methylbenzyl)oxy]thiophene-2-carboxylic acid and 5-(6-Bromo-1H-benzimidazol-1-yl)-3-[(2-methylbenzyl)oxy]thiophene-2-carboxylic acid

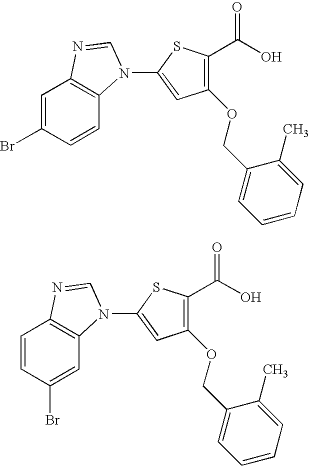

An analogous procedure to that described in Example 33 with a 1:1 regioisomeric mixture of methyl 5-(5-bromo-1H-benzimidazol-1-yl)-3-[(2-methylbenzyl)-oxy]thiophene-2-carboxylate and methyl 5-(6-bromo-1H-benzimidazol-1-yl)-3-[(2-methylbenzyl)-oxy]thiophene-2-carboxylate (0.100 g, 0.219 mmol) provided 0.0599 g (62%) of a 1:1 regioisomeric mixture of 5-(5-bromo-1H-benzimidazol-1-yl)-3-[(2-methylbenzyl)oxy]-thiophene-2-carboxylic acid and 5-(6-bromo-1H-benzimidazol-1-yl)-3-[(2-methylbenzyl)oxy]thiophene-2-carboxylic acid as a yellow solid. ¹H NMR (300 MHz, DMSO-d₆) δ 12.81 (br s, 1H), 8.75, 8.70 (s, 1H), 8.04, 7.93 (2×d, J=1.8 Hz, J=1.8 Hz, 1H), 7.81, 7.77 (2×d, J=8.8 Hz, J=8.7 Hz, 1H), 7.73, 7.72 (2×s, 1H), 7.61-7.50 (m, 2H), 7.31-7.20 (m, 3H), 5.35, 5.33 (2×s, 2H), 2.39, 2.38 (s, 3H).

### EXAMPLE 70

### 5-(6-Chloro-1H-benzimidazol-1-yl)-3-[(2-Chloro-4-fluorobenzyl)oxy]-thiophene-2-carboxylic acid

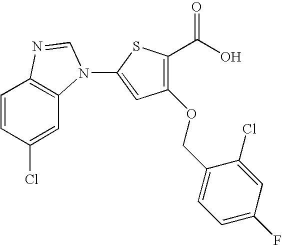

An analogous procedure to that described in Example 33 with methyl 5-(6-chloro-1H-benzimidazol-1-yl)-3-[(2-chloro-4-fluorobenzyl)oxy]thiophene-2-carboxylate (0.128 g, 0.284 mmol) yielded 0.0805 g (650%) of 5-(6-chloro-1H-benzimidazol-1-yl)-3-[(2-chloro-4-fluorobenzyl)oxy]-thiophene-2-carboxylic acid as a white solid. ¹H NMR (300 MHz, DMSO-d₆) δ 12.88 (br s, 1H), 8.73 (s, 1H), 7.93-7.74 (m, 3H), 7.71 (s, 1H), 7.55 (dd, J=8.8, 2.5 Hz, 1H), 7.41 (dd, J=8.6, 1.9 Hz, 1H), 7.34 (ddd, J=9.7, 8.5, 2.5 Hz, 1H), 5.39 (s, 2H).

### EXAMPLE 71

### 5-(6-Chloro-1H-benzimidazol-1-yl)-3-[(2,4-difluorobenzyl)oxy]-thiophene-2-carboxylic acid

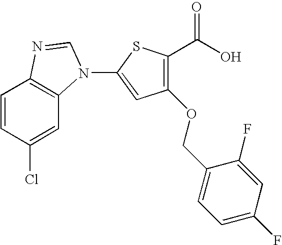

An analogous procedure to that described in Example 33 with methyl 5-(6-chloro-1H-benzimidazol-1-yl)-3-[(2,4-difluorobenzyl)oxy]thiophene-2-carboxylate (0.119 g, 0.274 mmol) yielded 0.0860 g (750%) of 5-(6-chloro-1H-benzimidazol-1-yl)-3-[(2,4-difluorobenzyl)oxy]-thiophene-2-carboxylic acid as an off-white solid. ¹H NMR (300 MHz, DMSO-d₆) δ 8.72 (s, 1H), 7.87 (d, J=1.8 Hz, 1H), 7.82 (d, J=8.6 Hz, 1H), 7.72 (m, 1H), 7.71 (s, 1H), 7.41 (dd, J=8.6, 2.0 Hz, 1H), 7.34 (m, 1H), 7.18 (m, 1H), 5.38 (s, 2H).

### EXAMPLE 72

### 5-(6-Chloro-1H-benzimidazol-1-yl)-3-(pyridin-3-ylmethoxy)thiophene-2-carboxylic acid

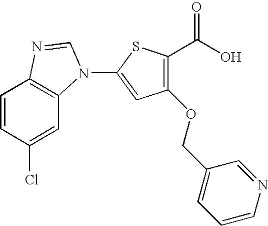

An analogous procedure to that described in Example 33 with methyl 5-(6-chloro-1H-benzimidazol-1-yl)-3-(pyridin-3-ylmethoxy)thiophene-2-carboxylate (0.0380 g, 0.0950 mmol) afforded 0.010 g (27%) of 5-(6-chloro-1H-benzimidazol-1-yl)-3-(pyridin-3-ylmethoxy)thiophene-2-carboxylic acid as a tan solid. ¹H NMR (300 MHz, DMSO-d₆) δ 8.88 (s, 1H), 8.77 (m, 1H), 8.72 (s, 1H), 8.32 (d, J=7.9 Hz, 1H), 7.87-7.79 (m, 3H), 7.69 (s, 1H), 7.42 (dd, J=8.6, 2.0 Hz, 1H), 5.52 (s, 2H).

### EXAMPLE 73

### 5-(1H-Benzimidazol-1-yl)-3-(prop-2-ynyloxy)thiophene-2-carboxylic acid

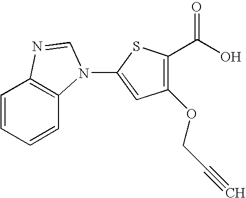

An analogous procedure to that described in Example 33 with methyl 5-(1H-benzimidazol-1-yl)-3-(prop-2-ynyloxy)thiophene-2-carboxylate (0.183 g, 0.586 mmol) gave 0.175 g (100%) of 5-(1H-benzimidazol-1-yl)-3-(prop-2-ynyloxy)thiophene-2-carboxylic acid as a tan solid. ¹H NMR (300 MHz, CD₃OD) δ 9.83 (s, 1H), 7.98 (m, 2H), 7.77 (m, 2H), 7.71 (s, 1H), 5.02 (d, J=2.3 Hz, 2H), 3.17 (t, J=2.3 Hz, 1H). MS (m/z) 299 (m+1).

### EXAMPLE 74

### 5-(6-Bromo-1H-benzimidazol-1-yl)-3-{[2-(trifluoromethyl)-benzyl]oxy}thiophene-2-carboxylic acid

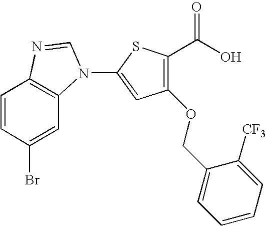

An analogous procedure to that described in Example 33 with methyl 5-(6-bromo-1H-benzimidazol-1-yl)-3-{[2-(trifluoromethyl)-benzyl]oxy}thiophene-2-carboxylate (0.0155 g, 0.0303 mmol) gave 0.0080 g (53%) of 5-(6-bromo-1H-benzimidazol-1-yl)-3-{[2-(trifluoromethyl)-benzyl]oxy}thiophene-2-carboxylic acid as a yellow solid. ¹H NMR (300 MHz, DMSO-d₆) δ 8.71 (s, 1H), 7.98-7.93 (m, 2H), 7.84-7.74 (m, 3H), 7.68 (s, 1H), 7.62 (m, 1H), 7.53 (dd, J=8.6, 1.9 Hz, 1H), 5.50 (s, 2H).

### EXAMPLE 75

### 5-(5,6-Dimethoxy-1H-benzimidazol-1-yl)-3-{[2-(trifluoromethyl)-benzyl]-oxy}thiophene-2-carboxylic acid

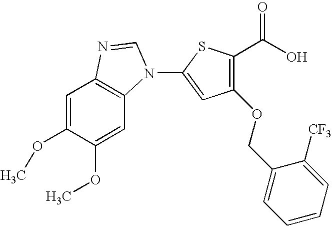

An analogous procedure to that described in Example 33 with methyl 5-(5,6-dimethoxy-1H-benzimidazol-1-yl)-3-[(2-trifluoromethylbenzyl)-oxy]thiophene-2-carboxylate (0.0691 g, 0.140 mmol) yielded 0.0558 g (83%) of 5-(5,6-dimethoxy-1H-benzimidazol-1-yl)-3-{[2-(trifluoromethyl)benzyl]-oxy}thiophene-2-carboxylic acid as a yellow solid. ¹H NMR (300 MHz, DMSO-d₆) δ 8.49 (s, 1H), 7.95 (d, J=7.6 Hz, 1H), 7.84-7.74 (m, 2H), 7.65-7.56 (m, 2H), 7.34 (s, 1H), 7.25 (s, 1H), 5.50 (s, 2H), 3.84 (s, 3H), 3.83 (s, 3H).

### EXAMPLE 76

### 3-[(2-Bromobenzyl)oxy]-5-(5,6-dimethoxy-1H-benzimidazol-1-yl)thiophene-2-carboxylic acid

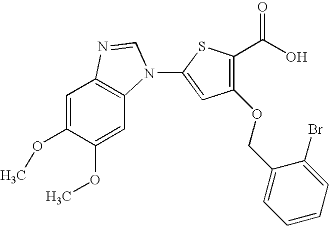

An analogous procedure to that described in Example 33 with methyl 3-[(2-bromobenzyl)oxy]-5-(5,6-dimethoxy-1H-benzimidazol-1-yl)thiophene-2-carboxylate (0.0719 g, 0.143 mmol) afforded 0.0597 g (85%) of 3-[(2-bromobenzyl)oxy]-5-(5,6-dimethoxy-1H-benzimidazol-1-yl)thiophene-2-carboxylic acid as a yellow solid. ¹H NMR (300 MHz, DMSO-d₆) δ 8.54 (s, 1H), 7.77-7.66 (m, 2H), 7.63 (s, 1H), 7.47 (m, 1H), 7.38-7.29 (m, 2H), 7.26 (s, 1H), 5.37 (s, 2H), 3.84 (s, 3H), 3.83 (s, 3H).

### EXAMPLE 77

### 5-(5,6-Dichloro-1H-benzimidazol-1-yl)-3-(3-furylmethoxy)thiophene-2-carboxylic acid

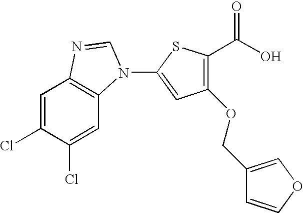

An analogous procedure to that described in Example 33 with methyl 5-(5,6-dichloro-1H-benzimidazol-1-yl)-3-(3-furylmethoxy)-thiophene-2-carboxylate (0.0715 g, 0.169 mmol) provided 0.0476 g (69%) of 5-(5,6-dichloro-1H-benzimidazol-1-yl)-3-(3-furylmethoxy)thiophene-2-carboxylic acid as a tan-orange solid. ¹H NMR (300 MHz, DMSO-d₆) δ 12.82 (br s, 1H), 8.78 (s, 1H), 8.13 (s, 1H), 8.06 (s, 1H), 7.85 (s, 1H), 7.69 (m, 1H), 7.68 (s, 1H), 6.61 (m, 1H), 5.21 (s, 2H).

### EXAMPLE 78

### 5-(5,6-Dichloro-1H-benzimidazol-1-yl)-3-(2-furyl methoxy)thiophene-2-carboxylic acid

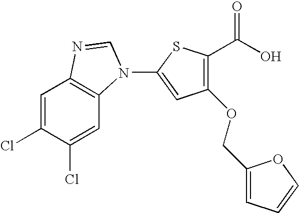

An analogous procedure to that described in Example 33 with methyl 5-(5,6-dichloro-1H-benzimidazol-1-yl)-3-(2-furylmethoxy)-thiophene-2-carboxylate (0.0525 g, 0.124 mmol) gave 0.0289 g (570%) of 5-(5,6-dichloro-1H-benzimidazol-1-yl)-3-(2-furylmethoxy)thiophene-2-carboxylic acid as a yellow solid. ¹H NMR (300 MHz, DMSO-d₆) δ 12.85 (br s, 1H), 8.78 (s, 1H), 8.14 (s, 1H), 8.08 (s, 1H), 7.74 (dd, J=1.9, 0.7 Hz, 1H), 7.71 (s, 1H), 6.70 (d, J=3.2 Hz, 1H), 6.51 (dd, J=3.2, 1.9 Hz, 1H), 5.32 (s, 2H).

### EXAMPLE 79

### 5-(5,6-Dichloro-1H-benzimidazol-1-yl)-3-(thien-3-ylmethoxy)-thiophene-2-carboxylic acid

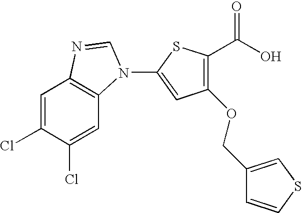

An analogous procedure to that described in Example 33 with methyl 5-(5,6-dichloro-1H-benzimidazol-1-yl)-3-(thien-3-ylmethoxy)-thiophene-2-carboxylate (0.0730 g, 0.166 mmol) afforded 0.0476 g (67%) of 5-(5,6-dichloro-1H-benzimidazol-1-yl)-3-(thien-3-ylmethoxy)thiophene-2-carboxylic acid as a yellow solid. ¹H NMR (300 MHz, DMSO-d₆) δ 12.84 (br s, 1H), 8.77 (s, 1H), 8.13 (s, 1H), 8.02 (s, 1H), 7.67 (s, 1H), 7.66 (m, 1H), 7.59 (dd, J=5.0, 3.0 Hz, 1H), 7.22 (dd, J=5.0, 1.2 Hz, 1H), 5.35 (s, 2H).

### EXAMPLE 80

### 5-(5,6-Dichloro-1H-benzimidazol-1-yl)-3-(thien-2-ylmethoxy)-thiophene-2-carboxylic acid

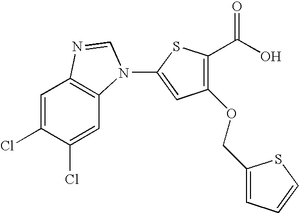

An analogous procedure to that described in Example 33 with methyl 5-(5,6-dichloro-1H-benzimidazol-1-yl)-3-(thien-2-ylmethoxy)-thiophene-2-carboxylate (0.0580 g, 0.132 mmol) afforded 0.0341 g (61%) of 5-(5,6-dichloro-1H-benzimidazol-1-yl)-3-(thien-2-ylmethoxy)thiophene-2-carboxylic acid as a pale yellow solid. ¹H NMR (300 MHz, DMSO-d₆) δ 8.77 (s, 1H), 8.13 (s, 1H), 8.03 (s, 1H), 7.70 (s, 1H), 7.61 (dd, J=5.0, 1.1 Hz, 1H), 7.30 (dd, J=3.5, 1.1 Hz, 1H), 7.07 (dd, J=5.0, 3.5 Hz, 1H), 5.54 (s, 2H).

### EXAMPLE 81

### Methyl 3-[(2-chloro-4-fluorobenzyl)oxy]-5-(5,6-dimethoxy-1H-benzimidazol-1-yl)thiophene-2-carboxylate

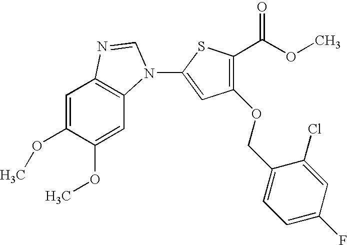

An analogous procedure to that described in Example 45 with methyl 5-(5,6-dimethoxy-1H-benzimidazol-1-yl)-3-hydroxythiophene-2-carboxylate (0.100 g, 0.299 mmol) and 2-chloro-4-flurobenzyl bromide (0.0809 g, 0.362 mmol) provided 0.0963 g (68%) of methyl 3-[(2-chloro-4-fluorobenzyl)oxy]-5-(5,6-dimethoxy-1H-benzimidazol-1-yl)thiophene-2-carboxylate as a yellow solid. ¹H NMR (300 MHz, DMSO-d₆) δ 8.50 (s, 1H), 7.80 (dd, J=8.6, 6.2 Hz, 1H), 7.70 (s, 1H), 7.55 (dd, J=8.8, 2.6 Hz, 1H), 7.39-7.31 (m, 1H), 7.35 (s, 1H), 7.27 (s, 1H), 5.41 (s, 2H), 3.85 (s, 3H), 3.83 (s, 3H), 3.78 (s, 3H).

### EXAMPLE 82

### N-({5-(5-methoxy-1H-benzimidazol-1-yl)-3-[(2-methylbenzyl)-oxy]thien-2-yl}carbonyl)methanesulfonamide and N-({5-(6-methoxy-1H-benzimidazol-1-yl)-3-[(2-methylbenzyl)oxy]thien-2-yl}carbonyl)methanesulfonamide

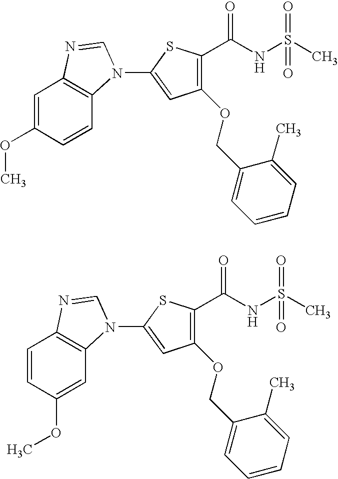

A 1:1 regioisomeric mixture of 5-(5-methoxy-1H-benzimidazol-1-yl)-3-[(2-methylbenzyl)oxy]thiophene-2-carboxylic acid and 5-(6-methoxy-1H-benzimidazol-1-yl)-3-[(2-methylbenzyl)oxy]thiophene-2-carboxylic acid (0.100 g, 0.254 mmol), 4-dimethylaminopyridine (0.0403 g, 0.330 mmol), and methanesulfonamide (0.0313 g, 0.329 mmol) were dissolved in 4 mL of dichloromethane with stirring. Triethylamine (0.046 mL, 0.33 mmol) was added via syringe followed by the addition 1-(3-dimethylaminopropyl)-3-ethylcarbodiimide hydrochloride (0.0633 g, 0.330 mmol) in a single portion. The mixture was stirred for 12 hours and subsequently poured into 5010 aqueous HCl solution and ethyl acetate. The layers were separated, and the organic layer was washed with brine. The combined aqueous layers were extracted with ethyl acetate, and the combined organic layers were dried over MgSO₄. Filtration, concentration in vacuo, and purification by flash chromatography afforded 0.0826 g (69%) of a 1:1 regioisomeric mixture of N-({5-(5-methoxy-1H-benzimidazol-1-yl)-3-[(2-methylbenzyl)oxy]thien-2-yl}carbonyl)-methanesulfonamide and N-({5-(6-methoxy-1H-benzimidazol-1-yl)-3-[(2-methylbenzyl)oxy]thien-2-yl}carbonyl)methanesulfonamide as a pale green solid. ¹H NMR (300 MHz, DMSO-d₆) δ 9.97 (br s, 1H), 8.70, 8.58 (2×s, 1H), 7.83-7.68 (m, 2H), 7.55 (m, 1H), 7.37-7.21 (m, 4H), 7.07, 7.01 (2×dd, J=8.8, 2.3 Hz, 1H), 5.51 (s, 2H), 3.85, 3.83 (2×s, 3H), 3.37, 3.36 (2×s, 3H), 2.41 (s, 3H). MS (m/z) 472 (m+1).

### EXAMPLES 83-158

Unless otherwise noted, the following compounds were prepared similarly according to general procedures outlined for Examples 2A, 33, 40, 45, 57 (or Intermediate Example 21), and 61 (where 7M NH₃ in MeOH was used instead of 2M NH₃ in MeOH).

### EXAMPLE 83

### 5-(5-Chloro-2-methyl-1H-benzimidazol-1-yl)-3-[(2-methylbenzyl)oxy]thiophene-2-carboxylic acid and 5-(6-Chloro-2-methyl-1H-benzimidazol-1-yl)-3-[(2-methylbenzyl)oxy]thiophene-2-carboxylic acid

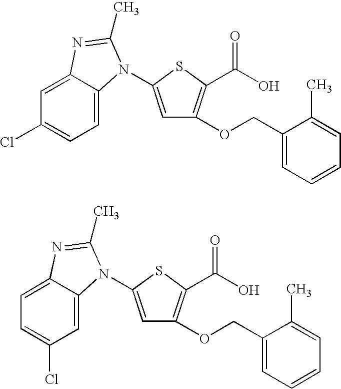

¹H NMR (400 MHz, CD₃OD) δ 7.61-7.56 (m, 1H); 7.46 (d, J=7.2 Hz, 1H); 7.28-7.21 (m, 6H); 5.34 (s, 2H); 2.52 (s, 3H); 2.43 (s, 3H). MS (ES−, m/z) 411 (m⁻¹).

### EXAMPLE 84

### 3-(Benzyloxy)-5-(5-chloro-1H-benzimidazol-1-yl)thiophene-2-carboxamide

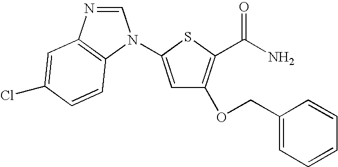

¹H NMR (400 MHz, DMSO-d₆) δ 8.70 (s, 1H), 7.88 (d, J=2.01 Hz, 1H), 7.78-7.70 (m, 2H), 7.65 (s, 1H), 7.56-7.52 (m, 2H), 7.46-7.35 (m, 4H), 7.01 (s, 1H), 5.40 (s, 2H). MS (ES+, m/z) 383 (m+1).

### EXAMPLE 85

### 5-(5-Chloro-1H-benzimidazol-1-yl)-3-({2-[(phenylsulfonyl)methyl]benzyl}oxy)thiophene-2-carboxylic acid and 5-(6-Chloro-1H-benzimidazol-1-yl)-3-({2-[(phenylsulfonyl)methyl]benzyl}oxy)thiophene-2-carboxylic acid

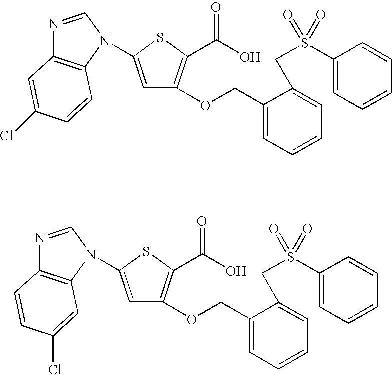

¹H NMR (400 MHz, DMSO-d₆) δ 8.75 (s, 1H), 8.70 (s, 1H), 7.91 (d, J=1.96 Hz, 1H), 7.84-7.58 (m, 17H), 7.47 (dd, J=1.96 Hz, 8.74 Hz, 1H), 7.43-7.26 (m, 5H), 7.12 (t, J=7.76 Hz, 2H), 5.38 (s, 4H), 4.93 (s, 4H). MS (ES+, m/z) 540 (m+1).

### EXAMPLE 86

### 5-(5-Chloro-1H-benzimidazol-1-yl)-3-{1-[3-(trifluoromethyl)phenyl]ethoxy}thiophene-2-carboxylic acid and 5-(6-Chloro-1H-benzimidazol-1-yl)-3-{1-[3-(trifluoromethyl)phenyl]ethoxy}thiophene-2-carboxylic acid

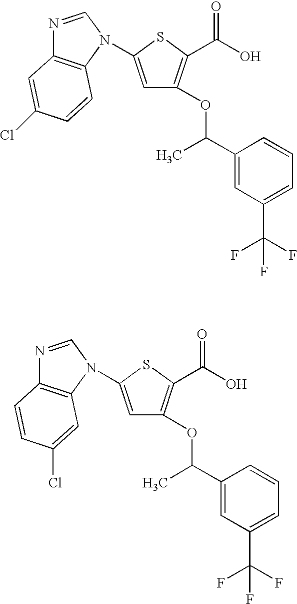

¹H NMR (400 MHz, DMSO-d₆) δ 12.94 (br s, 2H), 8.70 (s, 1H), 8.65 (s, 1H), 7.94-7.63 (m, 13H), 7.58 (s, 2H), 7.41 (t, J=8.03, 2H), 5.88 (dd, J=6.06 Hz, 11.06 Hz, 2H), 1.64 (d, J=6.24 Hz, 6H). MS (ES+, m/z) 467 (m+1).

### EXAMPLE 87

### 5-[6-(2,2,2-Trifluoroethoxy)-1H-benzimidazol-1-yl]-3-{[2-(trifluoromethyl)benzyl]oxy}thiophene-2-carboxamide

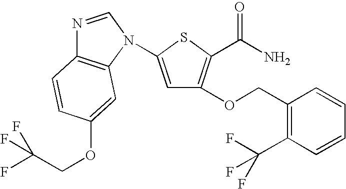

¹H NMR (400 MHz, DMSO-d₆) δ 8.55 (s, 1H), 7.86-7.63 (m, 7H), 7.38 (d, J=2.38 Hz, 1H), 7.10 (dd, J=2.29 Hz, 8.88 Hz, 1H), 6.82 (br s, 1H), 5.56 (s, 2H), 4.86 (q, J=8.85 Hz, 2H). MS (ES+, m/z) 516 (m+1).

### EXAMPLE 88

### 5-(2,2-Difluoro-5H-[1,3]dioxolo[4,5-[4-benzimidazol-5-yl)-3-{[2-(trifluoromethyl)benzyl]oxy}thiophene-2-carboxamide

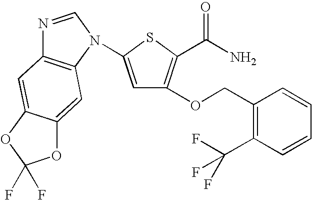

¹H NMR (400 MHz, DMSO-d₆) δ 8.66 (s, 1H), 7.92 (s, 1H), 7.88 (s, 1H), 7.87-7.64 (m, 6H), 6.79 (br s, 1H), 5.56 (s, 2H). MS (ES+, m/z) 498 (m+1).

### EXAMPLE 89

### 5-(7,8-Dihydro-1H,6H-[1,4]dioxepino[2,3-]benzimidazol-1-yl)-3-{[2-(trifluoromethyl)benzyl]oxy}thiophene-2-carboxamide

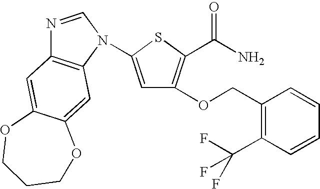

¹H NMR (400 MHz, DMSO-d₆) δ 8.56 (s, 1H), 7.85 (s, 1H), 7.83 (s, 1H), 7.77 (t, J=7.60 Hz, 1H), 7.69 (br s, 1H), 7.64 (t, J=7.60 Hz, 1H), 7.60 (s, 1H), 7.36 (s, 2H), 6.76 (br s, 1H), 5.54 (s, 2H), 4.15-4.06 (m, 4H), 2.11 (t, J=4.94 Hz, 2H). MS (ES+, m/z) 490 (m+1).

### EXAMPLE 90

### 5-(5,6-Dimethoxy-1H-benzimidazol-1-yl)-3-{[3-(dimethylamino)benzyl]oxy}thiophene-2-carboxamide

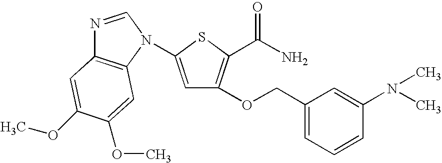

¹H NMR (400 MHz, DMSO-d₆) δ 8.40 (s, 1H), 7.73 (br s, 1H), 7.60 (s, 1H), 7.33 (s, 1H), 7.20 (t, J=7.87 Hz, 1H), 7.15 (s, 1H), 7.07 (br s, 1H), 6.88 (s, H), 6.79 (d, J=7.51 Hz, 1H), 6.70 (dd, J=2.29 Hz, 8.33 Hz, 1H), 5.34 (s, 2H), 3.82 (s, 6H), 2.89 (s, 6H).

### EXAMPLE 91

### 3-[(6-Chloro-1,3-benzodioxol-5-yl)methoxy]-5-(5,6-dimethoxy-1H-benzimidazol-1-yl)thiophene-2-carboxamide

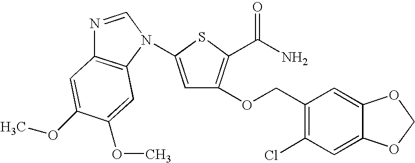

¹H NMR (400 MHz, DMSO-d₆) δ 8.43 (s, 1H), 7.73 (br s, 1H), 7.67 (s, 1H), 7.35 (s, 1H), 7.33 (s, 1H), 6.90 (br s, 1H), 6.11 (s, 2H), 5.36 (s, 2H), 3.86 (s, 3H), 3.83 (s, 3H). MS (ES+, m/z) 488 (m+1).

### EXAMPLE 92

### 5-(5,6-Dimethoxy-1H-benzimidazol-1-yl)-3-[(2-nitrobenzyl)oxy]thiophene-2-carboxamide

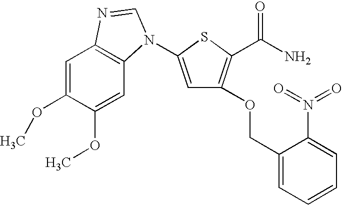

¹H NMR (400 MHz, DMSO-d₆) δ 8.38 (s, 1H), 8.19 (d, J=8.1 Hz, 1H), 7.84 (t, J=7.6 Hz, 1H), 7.78-7.76 (m, 2H), 7.65 (m, 1H), 7.57 (s, 1H), 7.32 (s, 1H), 7.09 (br s, 1H), 7.07 (s, 1H) 5.79 (s, 2H), 3.81 (s, 3H), 3.76 (s, 3H). MS (ES+, m/z) 455 (m+1).

### EXAMPLE 93

### 3-(1,1′-Biphenyl-2-ylmethoxy)-5-(5,6-dimethoxy-1H-benzimidazol-1-yl)thiophene-2-carboxamide

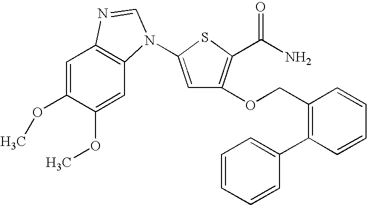

¹H NMR (400 MHz, DMSO-d₆) δ 8.36 (s, 1H), 7.72 (m, 1H), 7.61 (br s, 1H), 7.52-7.48 (m, 2H), 7.46-7.33 (m, 8H), 7.15 (s, 1H), 6.62 (br s, 1H) 5.34 (s, 2H), 3.83 (s, 3H), 3.82 (s, 3H). MS (ES+, m/z) 486 (m+1).

### EXAMPLE 94

### 5-(5,6-Dimethoxy-1H-benzimidazol-1-yl)-3-[(3-iodobenzyl)oxy]-thiophene-2-carboxamide

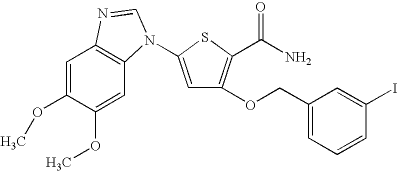

¹H NMR (400 MHz, DMSO-d₆) 68.40 (s, 1H), 7.96 (m, 1H), 7.73 (d, J=7.3 Hz, 1H), 7.59-7.57 (m, 3H), 7.34 (s, 1H), 7.15 (s, 1H), 7.10 (br s, 1H), 5.38 (s, 2H), 3.84 (s, 3H), 3.83 (s, 3H). MS (ES+, m/z) 536 (m+1).

### EXAMPLE 95

### 3-[(2-Cyanobenzyl)oxy]-5-(5,6-dimethoxy-1H-benzimidazol-1-yl)thiophene-2-carboxamide

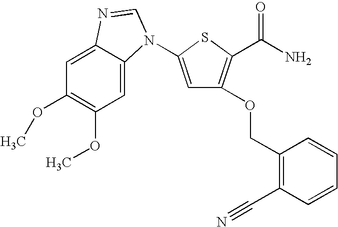

¹H NMR (400 MHz, DMSO-d₆) δ 8.47 (s, 1H), 7.98 (d, J=7.3 Hz, 1H), 7.85-7.77 (m, 3H), 7.70 (s, 1H), 7.62 (m, 1H), 7.35 (s, 1H), 7.22 (s, 1H), 6.92 (br s, 1H), 5.60 (s, 2H), 3.85 (s, 3H), 3.83 (s, 3H). MS (ES+, m/z) 435 (m+1).

### EXAMPLE 96

### 3-[(3-Aminobenzyl)oxy]-5-(5,6-dimethoxy-1H-benzimidazol-1-yl)thiophene-2-carboxamide

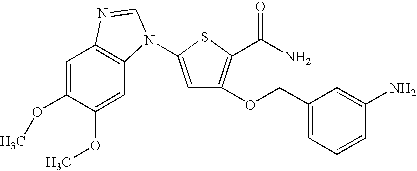

¹H NMR (400 MHz, DMSO-d₆) δ 8.41 (s, 1H), 7.73 (br s, 1H), 7.53 (s, 1H), 7.34 (s, 1H), 7.16 (s, 1H), 7.04 (t, J=7.7 Hz, 1H), 7.00 (br s, 1H), 6.67-6.63 (m, 2H), 6.64 (d, J=7.8 Hz, 1H), 5.27 (s, 2H), 5.18 (d, J=7.8 Hz, 2H), 3.83 (m, 6H). MS (ES+, m/z) 425 (m+1).

### EXAMPLE 97

### 5-(5,6-Dimethoxy-1H-benzimidazol-1-yl)-3-{[2-(methylthio)benzyl]-oxy}thiophene-2-carboxamide

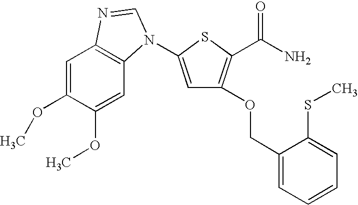

¹H NMR (400 MHz, DMSO-d₆) δ 8.42 (s, 1H), 7.70 (br s, 1H), 7.66 (s, 1H), 7.55 (d, J=7.5 Hz, 1H), 7.41 (m, 2H), 7.33 (s, 1H), 7.21 (s, 2H), 6.87 (br s, 1H), 5.40 (s, 2H), 3.84 (s, 3H), 3.81 (s, 3H), 2.50 (s, 3H). MS (ES+, m/z) 456 (m+1).

### EXAMPLE 98

### 5-(5,6-Dimethoxy-1H-benzimidazol-1-yl)-3-{[2-(methylsulfinyl)benzyl]oxy}thiophene-2-carboxamide

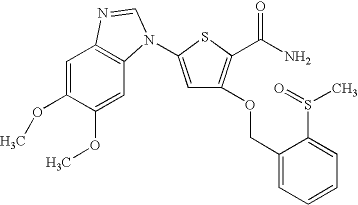

¹H NMR (400 MHz, DMSO-d₆) 88.42 (s, 1H), 7.99 (d, J=7.7 Hz, 1H), 7.73-7.68 (m, 3H), 7.65-7.62 (m, 2H), 7.34 (s, 1H), 7.21 (s, 2H), 6.92 (br s, 1H), 5.50 (m, 2H), 3.84 (s, 3H), 3.83 (s, 3H), 2.77 (s, 3H). MS (ES+, m/z) 472 (m+1).

### EXAMPLE 99

### 5-(5,6-Dimethoxy-1H-benzimidazol-1-yl)-3-{[2-(methylsulfonyl)benzyl]oxy}thiophene-2-carboxamide

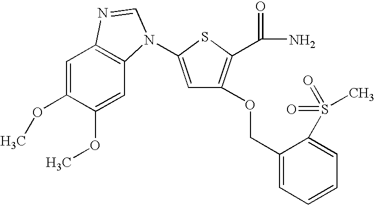

¹H NMR (400 MHz, DMSO-d₆) δ 8.42 (s, 1H), 8.03 (d, J=7.8 Hz, 1H), 7.86-7.79 (m, 2H), 7.70-7.67 (m, 2H), 7.59 (s, 1H), 7.33 (s, 1H), 7.19 (s, 1H), 7.11 (br s, 1H), 5.79 (s, 2H), 3.82 (m, 6H), 3.34 (s, 3H). MS (ES+, m/z) 488 (m+1).

### EXAMPLE 100

### 3-[(2-Aminopyridin-4-yl)methoxy]-5-(5,6-dimethoxy-1H-benzimidazol-1-yl)thiophene-2-carboxamide

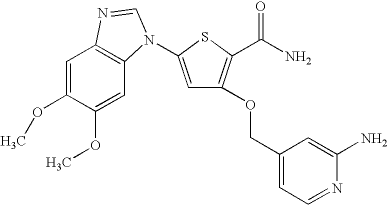

¹H NMR (400 MHz, DMSO-d₆) 58.39 (s, 1H), 7.91 (d, J=5.1 Hz, 1H), 7.76 (br s, 1H), 7.46 (s, 1H), 7.33 (s, 1H), 7.12 (s, 1H), 7.07 (br s, 1H), 6.56 (d, J=5.2 Hz, 1H), 6.49 (s, 1H) 6.03 (s, 2H), 5.31 (s, 2H), 3.82 (s, 3H), 3.81 (s, 3H). MS (ES+, m/z) 426 (m+1).

### EXAMPLE 101

### 3-[(2-Chloropyridin-3-yl)methoxy]-5-(5,6-dimethoxy-1H-benzimidazol-1-yl)thiophene-2-carboxamide

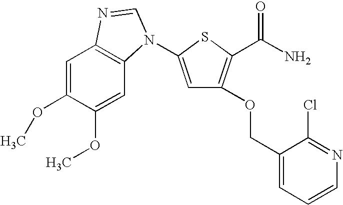

¹H NMR (400 MHz, DMSO-d₆) δ 8.45 (dd, J=4.8, 1.9 Hz, 1H), 8.43 (s, 1H), 8.11 (dd, J=7.7, 1.8 Hz, 1H), 7.75 (s, 1H), 7.66 (s, 1H), 7.53 (dd, J=7.4, 4.8 Hz, 1H), 7.34 (s, 1H), 7.21 (s, 1H), 7.00 (br s, 1H), 5.49 (s, 2H), 3.84 (s, 3H), 3.83 (s, 3H). MS (ES+, m/z) 445 (m+1).

### EXAMPLE 102

### 5-(5,6-Dimethoxy-1H-benzimidazol-1-yl)-3-[(2-fluoropyridin-3-yl)methoxy]thiophene-2-carboxamide

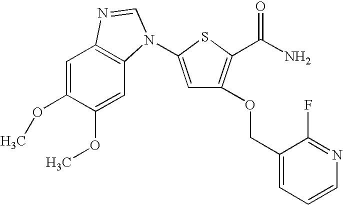

¹H NMR (400 MHz, DMSO-d₆) δ 8.43 (s, 1H), 8.28 (d, J=4.5 Hz, 1H), 8.19 (m, 1H), 7.87 (m, 1H), 7.67 (s, 1H), 7.45 (m, 1H), 7.34 (s, 1H), 7.21 (s, 1H), 6.97 (br s, 1H), 5.49 (s, 2H), 3.85 (s, 3H), 3.83 (s, 3H). MS (ES+, m/z) 429 (m+1).

### EXAMPLE 103

### 5-(5,6-Dimethoxy-1H-benzimidazol-1-yl)-3-[(2-vinylbenzyl)oxy]thiophene-2-carboxamide

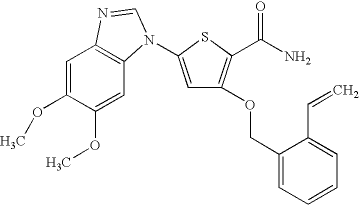

¹H NMR (400 MHz, DMSO-d₆) δ 8.46 (s, 1H), 7.73-7.70 (m, 2H), 7.61 (m, 1H), 7.48-7.36 (m, 2H), 7.26 (s, 1H), 7.24-7.14 (m, 3H), 6.82 (br s, 1H), 5.87 (d, J=16.6 Hz, 1H), 5.54 (d, J=11.8 Hz, 1H), 5.54 (s, 2H), 3.88 (s, 3H), 3.86 (s, 3H). MS (ES+, m/z) 436 (m+1).

### EXAMPLE 104

### 3-{[4-(Aminocarbonyl)benzyl]oxy}-5-(5,6-dimethoxy-1H-benzimidazol-1-yl)thiophene-2-carboxamide

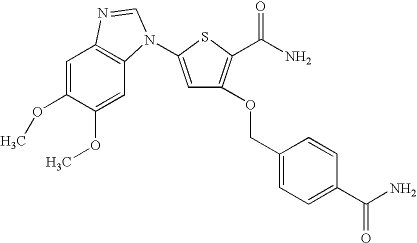

¹H NMR (400 MHz, DMSO-d₆) δ 8.43 (s, 1H), 8.01 (br s, 1H), 7.93 (d, J=8.2 Hz, 2H), 7.76 (brs, 1H), 7.65 (d, J=8.2 Hz, 2H), 7.61 (s, 1H), 7.43 (br s, 1H), 7.36 (s, 1H), 7.16 (s, 1H), 7.12 (br s, 1H), 5.51 (s, 2H), 3.85 (m, 6H). MS (ES+, m/z) 453 (m+1).

### EXAMPLE 105

### 3-[(2-Acetylbenzyl)oxy]-5-(5,6-dimethoxy-1H-benzimidazol-1-yl)thiophene-2-carboxamide

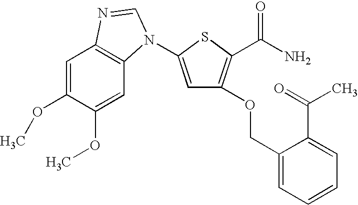

¹H NMR (400 MHz, DMSO-d₆) δ 8.43 (s, 1H), 8.05 (d, J=7.8 Hz, 1H), 7.72-7.64 (m, 2H), 7.59-7.55 (m, 2H), 7.35 (s, 1H), 7.17 (s, 1H), 7.17 (m, 2H), 5.66 (s, 2H), 3.85 (s, 3H), 3.84 (s, 3H), 2.65 (s, 3H). MS (ES+, m/z) 452 (m+1).

### EXAMPLE 106

### 5-(5,6-Dimethoxy-1H-benzimidazol-1-yl)-3-[(2-ethynylbenzyl)oxy]thiophene-2-carboxamide

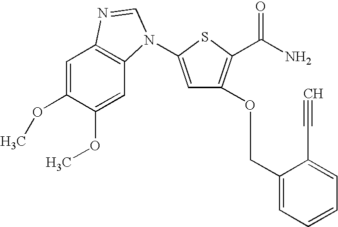

¹H NMR (400 MHz, DMSO-d₆) δ 8.43 (s, 1H), 7.72 (br s, 1H), 7.66-7.51 (m, 3H), 7.49-7.41 (m, 2H), 7.34 (s, 1H), 7.20 (s, 1H), 6.94 (br s, 1H), 5.50 (s, 2H), 4.54 (s, 1H), 3.85 (s, 3H), 3.83 (s, 3H). MS (ES+, m/z) 434 (m+1).

### EXAMPLE 107

### 5-(5,6-Dimethoxy-1H-benzimidazol-1-yl)-3-{[2-(trifluoromethoxy)benzyl]oxy}thiophene-2-carboxamide

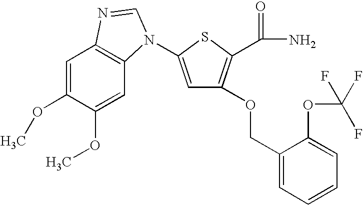

¹H NMR (400 MHz, DMSO-d₆) δ 8.43 (s, 1H), 7.76 (m, 2H), 7.65 (s, 1H), 7.56 (m, 1H), 7.50-7.46 (m, 2H), 7.34 (s, 1H), 7.22 (s, 1H), 6.86 (br s, 1H), 5.48 (s, 2H), 3.84 (s, 3H), 3.83 (s, 3H). MS (ES+, m/z) 494 (m+1).

### EXAMPLE 108

### 3-{[2-(Difluoromethoxy)benzyl]oxy}-5-(5,6-dimethoxy-1H-benzimidazol-1-yl)thiophene-2-carboxamide

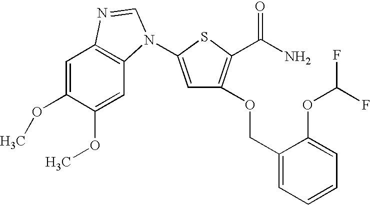

¹H NMR (400 MHz, DMSO-d₆) δ 8.42 (s, 1H), 7.72 (br s, 1H), 7.67 (d, J=7.7 Hz, 1H), 7.64 (s, 1H), 7.51-7.47 (m, 2H), 7.34 (s, 1H), 7.32-7.28 (m, 2H), 7.21 (s, 1H), 6.91 (br s, 1H), 5.43 (s, 2H), 3.84 (s, 3H), 3.83 (s, 3H). MS (ES+, m/z) 476 (m+1).

### EXAMPLE 109

### 3-{[2-(1,2-Dihydroxyethyl)benzyl]oxy}-5-(5,6-dimethoxy-1H-benzimidazol-1-yl)thiophene-2-carboxamide

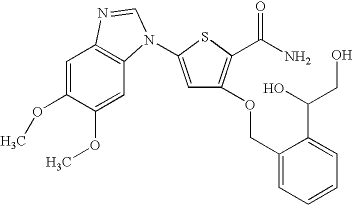

¹H NMR (400 MHz, DMSO-d₆) 68.40 (s, 1H), 7.64 (brs, 1H), 7.59 (s, 1H), 7.52 (d, J=7.5 Hz, 1H), 7.46 (d, J=7.3 Hz, 1H), 7.37 (m, 1H), 7.32-7.28 (m, 2H), 7.17 (s, 1H), 6.92 (br s, 1H), 5.49 (m, 2H), 5.35 (d, J=4.0 Hz, 1H), 4.87 (m, 1H), 4.81 (t, J=5.8 Hz, 1H), 3.98 (m, 1H), 3.80 (s, 6H), 3.53 (m, 1H). MS (ES+, m/z) 470 (m+1).

### EXAMPLE 110

### 5-(5,6-Dimethoxy-1H-benzimidazol-1-yl)-3-[(2-formylbenzyl)oxy]thiophene-2-carboxamide

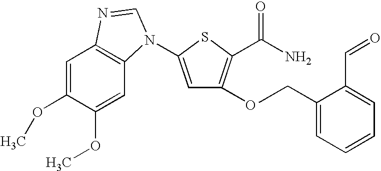

¹H NMR (400 MHz, DMSO-d₆) δ 10.28 (s, 1H), 8.41 (s, 1H), 8.02 (m, 1H), 7.73-7.64 (m, 4H), 7.58 (s, 1H), 7.34 (s, 1H), 7.15 (s, 1H), 7.02 (m, 1H), 5.81 (s, 2H), 3.82 (s, 3H), 3.81 (s, 3H), 2.65 (s, 3H). MS (ES+, m/z) 438 (m+1).

### EXAMPLE 111

### 3-(Cyclohexylmethoxy)-5-(5,6-dimethoxy-1H-benzimidazol-1-yl)thiophene-2-carboxamide

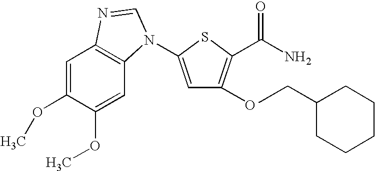

¹H NMR (400 MHz, DMSO-d₆) δ 8.43 (s, 1H), 7.73 (br s, 1H), 7.55 (s, 1H), 7.34 (s, 1H), 7.25 (s, 1H), 6.89 (s, 1H), 4.13 (d, J=6.3 Hz, 2H), 3.85 (s, 3H), 3.83 (s, 3H), 1.88-1.64 (m, 6H), 1.31-1.01 (m, 5H). MS (ES+, m/z) 416 (m+1).

### EXAMPLE 112

### 5-(5,6-Dimethoxy-1H-benzimidazol-1-yl)-3-(tetrahydro-2H-pyran-2-ylmethoxy) thiophene-2-carboxamide

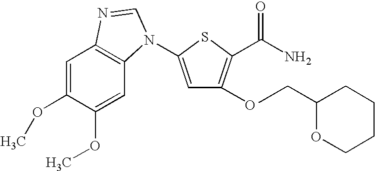

¹H NMR (400 MHz, DMSO-d₆) δ 8.42 (s, 1H), 7.72 (br s, 1H), 7.53 (s, 1H), 7.33 (s, 1H), 7.25 (s, 1H), 7.09 (br s, 1H), 4.30 (dd, J=10.8, 3.2 Hz, 1H), 4.20 (dd, J=10.6, 6.9 Hz, 1H), 3.92 (d, J=11.1 Hz, 1H), 3.86 (s, 3H), 3.83 (s, 3H), 3.72 (m, 1H), 1.83 (m, 1H), 1.64 (d, J=13.1 Hz, 1H), 1.54-1.47 (m, 4H), 1.38 (m, 1H). MS (ES+, m/z) 418 (m+1).

### EXAMPLE 113

### 5-(5,6-Dimethoxy-1H-benzimidazol-1-yl)-3-(2-morpholin-4-ylethoxy)thiophene-2-carboxamide

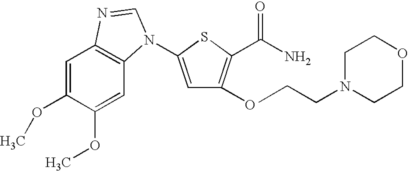

¹H NMR (400 MHz, DMSO-d₆) δ 8.41 (s, 1H), 7.75 (br s, 1H), 7.56 (s, 1H), 7.52 (br s, 1H), 7.34 (s, 1H), 7.25 (s, 1H), 4.38 (t, J=4.6 Hz, 2H), 3.85 (s, 3H), 3.83 (s, 3H), 3.59 (m, 4H), 2.72 (t, J=6.7 Hz, 2H), 2.47 (m, 4H). MS (ES+, m/z) 433 (m+1).

### EXAMPLE 114

### 5-(5,6-Dimethoxy-1H-benzimidazol-1-yl)-3-(2-phenylethoxy)-thiophene-2-carboxamide

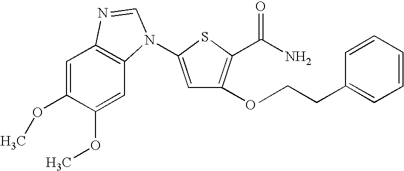

¹H NMR (400 MHz, DMSO-d₆) δ 8.42 (s, 1H), 7.64 (br s, 1H), 7.56 (s, 1H), 7.37-7.31 (m, 5H), 7.26-7.23 (m, 2H), 6.75 (br s, 1H), 4.53 (t, J=6.8 Hz, 2H), 3.85 (s, 3H), 3.82 (s, 3H), 3.15 (t, J=6.7 Hz, 2H). MS (ES+, m/z) 424 (m+1).

### EXAMPLE 115

### 5-(5,6-Dimethoxy-1H-benzimidazol-1-yl)-3-(3-phenylpropoxy)thiophene-2-carboxamide

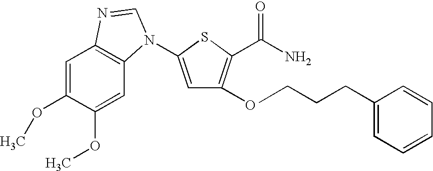

¹H NMR (400 MHz, DMSO-d₆) δ 8.42 (s, 1H), 7.70 (br s, 1H), 7.52 (s, 1H), 7.34 (s, 1H), 7.31-7.24 (m, 5H), 7.19 (m, 1H), 6.97 (br s, 1H), 4.31 (t, J=6.8 Hz, 2H), 3.83 (s, 3H), 3.83 (s, 3H), 2.76 (t, J=6.7 Hz, 2H), 2.13 (m, 2H). MS (ES+, m/z) 438 (m+1).

### EXAMPLE 116

### 5-(1H-Benzimidazol-1-yl)-3-{[2-(trifluoromethyl)benzyl]oxy}thiophene-2-carboxamide

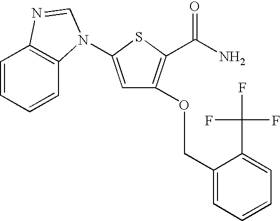

¹H NMR (400 MHz, DMSO-d₆) δ 8.67 (s, 1H), 7.87-7.85 (m, 2H), 7.82-7.77 (m, 3H), 7.72-7.64 (m, 3H), 7.45-7.36 (m, 2H), 6.79 (br s, 1H), 5.56 (s, 2H). MS (ES+, m/z) 418 (m+1).

### EXAMPLE 117

### 5-(1H-Benzimidazol-1-yl)-3-[(2-nitrobenzyl)oxy]thiophene-2-carboxamide

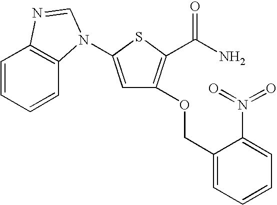

¹H NMR (400 MHz, DMSO-d₆) δ 8.61 (s, 1H), 8.19 (d, J=7.6 Hz, 1H), 7.84-7.62 (m, 7H), 7.41-7.35 (m, 2H), 7.05 (br s, 1H), 5.78 (s, 2H). MS (ES+, m/z) 395 (m+1).

### EXAMPLE 118

### 5-(6-Methoxy-1H-benzimidazol-1-yl)-3-{[2-(trifluoromethyl)benzyl]oxy}thiophene-2-carboxamide

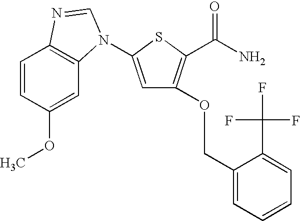

¹H NMR (300 MHz, DMSO-d₆) δ 8.49 (s, 1H), 7.87-7.61 (m, 7H), 7.21 (d, J=2.4 Hz, 1H), 6.98 (dd, J=8.9, 2.4 Hz, 1H), 6.81 (br s, 1H), 5.56 (s, 2H), 3.83 (s, 3H).

### EXAMPLE 119

### 3-[(2-Bromobenzyl)oxy]-5-[6-(trifluoromethyl)-1H-benzimidazol-1-yl]thiophene-2-carboxamide

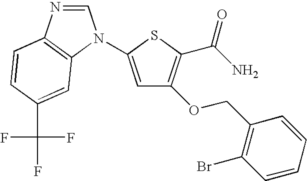

¹H NMR (300 MHz, DMSO-d₆) δ 8.86 (s, 1H), 8.05-7.99 (m, 2H), 7.83-7.67 (m, 5H), 7.47 (ddd, J=8.8, 7.5, 1.3 Hz, 1H), 7.37 (ddd, J=9.4, 7.6, 1.8 Hz, 1H), 6.95 (br s, 1H), 5.46 (s, 2H).

### EXAMPLE 120

### 3-[(3-Bromopyridin-4-yl)methoxy]-5-(5,6-dimethoxy-1H-benzimidazol-1-yl)thiophene-2-carboxamide

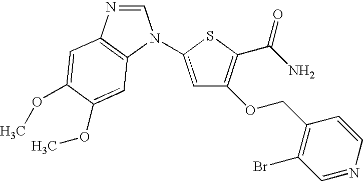

¹H NMR (400 MHz, DMSO-d₆) δ 8.81 (s, 1H), 8.63 (d, J=5.0 Hz, 1H), 8.42 (s, 1H), 7.77 (br s, 1H), 7.62 (d, J=5.0 Hz, 1H), 7.59 (s, 1H), 7.33 (s, 1H), 7.19 (s, 1H), 7.08 (br s, 1H), 5.49 (s, 2H), 3.824 (s, 3H), 3.818 (s, 3H). MS (ES+, m/z) 489, 491 (m+1).

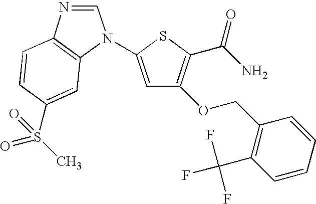

### EXAMPLE 121

### 5-[6-(Methylsulfonyl)-1H-benzimidazol-1-yl]-3-{[2-(trifluoromethyl)benzyl]oxy}-thiophene-2-carboxamide

¹H NMR (400 MHz, DMSO-d₆) δ 8.93 (s, 1H), 8.24 (d, J=1.7 Hz, 1H), 8.05 (d, J=8.60 Hz, 1H), 7.92 (dd, J=8.4, 1.7 Hz, 1H), 7.89-7.77 (m, 5H), 7.65 (m, 1H), 6.84 (br s, 1H), 5.54 (s, 2H), 3.29 (s, 3H). MS (ES+, m/z) 496 (m+1).

### EXAMPLE 122

### 5-[6-[(Methylsulfonyl)amino]-1H-benzimidazol-1-yl]-3-{[2-(trifluoromethyl)benzyl]-oxy}thiophene-2-carboxamide

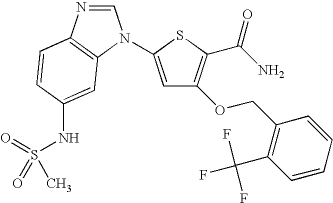

¹H NMR (400 MHz, DMSO-d₆) δ 9.85 (s, 1H), 8.64 (s, 1H), 7.86 (d, J=7.9 Hz, 1H), 7.82-7.71 (m, 6H), 7.66 (m, 1H), 7.25 (dd, J=8.8, 2.0 Hz, 1H), 6.79 (br s, 1H), 5.52 (s, 2H), 2.98 (s, 3H).

### EXAMPLE 123

### 5-(6,7-Dihydro-1H-[1,4]dioxino[2,3-f]benzimidazol-1-yl)-3-{[2-(trifluoromethyl)benzyl]-oxy}thiophene-2-carboxamide

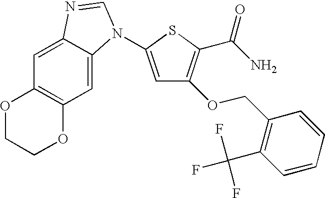

¹H NMR (300 MHz, DMSO-d₆) δ 8.48 (s, 1H), 7.87-7.74 (m, 3H), 7.70-7.61 (m, 2H), 7.60 (s, 1H), 7.25 (s, 1H), 7.24 (s, 1H), 6.76 (br s, 1H), 5.55 (s, 2H), 4.29 (m, 4H). MS (ES+, m/z) 476 (m+1).

### EXAMPLE 124

### 5-(6,7-Dihydro-1H-[1,4]dioxino[2,3-f]benzimidazol-1-yl)-3-{[1-(methylsulfonyl)-piperidin-4-yl)methoxy]thiophene-2-carboxamide

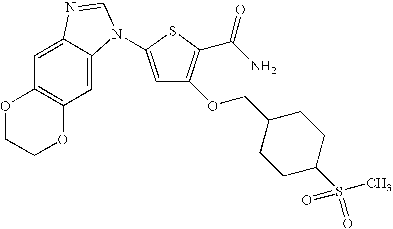

H NMR (300 MHz, DMSO-d₆) δ 8.48 (s, 1H), 7.71 (br s, 1H), 7.51 (s, 1H), 7.27 (s, 1H), 7.23 (s, 1H), 6.87 (br s, 1H), 4.29 (br s, 4H), 4.21 (m, 2H), 3.60 (m, 2H), 2.85 (s, 3H), 2.74 (m, 2H), 2.08-1.81 (m, 3H), 1.36 (m, 2H). MS (ES+, m/z) 493 (m+1).

### EXAMPLE 125

### 1-[5-(Aminocarbonyl)-4-({[2-(trifluoromethyl)phenyl]methyl}oxy)-2-thienyl]-1H-benzimidazole-5-carboxamide

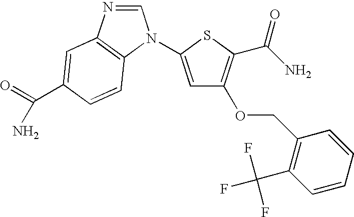

¹H NMR (400 MHz, DMSO-d₆) δ 8.75 (s, 1H), 8.36 (d, J=0.9 Hz, 1H), 8.10 (br s, 1H), 7.99 (dd, J=8.6, 1.4 Hz, 1H), 7.88-7.62 (m, 7H), 7.41 (br s, 1H), 6.80 (br s, 1H), 5.56 (s, 2H). MS (ES+, m/z) 461 (m+1).

### EXAMPLE 126

### 3-[1-(2-Chlorophenyl)ethoxy]-5-(5,6-dimethoxy-1H-benzimidazol-1-yl)thiophene-2-carboxamide

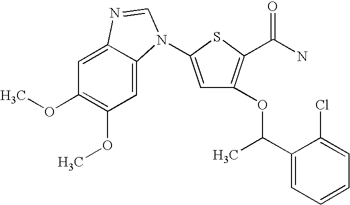

¹H NMR (400 MHz, DMSO-d₆) δ 8.35 (s, 1H), 7.83 (br s, 1H), 7.68 (dd, J=7.7, 2.0 Hz, 1H), 7.48 (dd, J=7.8, 1.2 Hz, 1H), 7.43 (ddd, J=7.5, 7.4, 1.2 Hz, 1H), 7.35 (ddd, J=7.8, 7.6, 1.8 Hz, 1H), 7.32 (s, 1H), 7.14 (br s, 1H), 7.13 (s, 1H), 7.02 (s, 1H), 6.01 (q, J=6.4 Hz, 1H), 3.81 (s, 3H), 3.79 (s, 3H), 1.72 (d, J=6.4 Hz, 3H). MS (ES+, m/z) 458 (m+1).

### EXAMPLE 127

### 5-(5,6-Dimethoxy-1H-benzimidazol-1-yl)-3-[1-(2-methylphenyl)ethoxy] thiophene-2-carboxamide

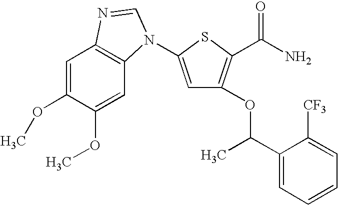

¹H NMR (400 MHz, DMSO-d₆) δ 8.31 (s, 1H), 7.96-7.92 (m, 1H), 7.84 (br s, 1H), 7.81-7.73 (m, 2H), 7.58-7.52 (m, 1H), 7.31 (s, 1H), 7.15 (br s, 1H), 7.05 (s, 1H), 7.01 (s, 1H), 6.01-5.96 (m, 1H), 3.81 (s, 3H), 3.78 (s, 3H), 1.75 (d, J=6.0 Hz, 3H). MS (ES+, m/z) 492 (m+1).

### EXAMPLE 128

### 5-(5,6-Dimethoxy-1H-benzimidazol-1-yl)-3-[(4-methoxybenzyl)oxy]thiophene-2-carboxamide

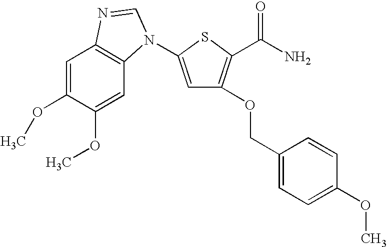

¹H NMR (400 MHz, DMSO-d₆) δ 8.39 (s, 1H), 7.68 (br s, 1H), 7.60 (s, 1H), 7.48 (d, J=8.8 Hz, 2H), 7.32 (s, 1H), 7.15, (s, 1H), 6.99 (br s, 1H), 6.95 (d, J=8.8 Hz, 2H), 5.32 (s, 2H), 3.83 (s, 3H), 3.81 (s, 3H), 3.74 (s, 3H). MS (ES+, m/z) 440 (m+1).

### INTERMEDIATE EXAMPLE 1

### Methyl 5-(1H-benzimidazol-1-yl)-3-(phenylethynyl)-2-thiophenecarboxylate

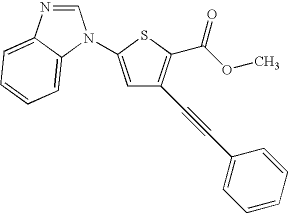

Methyl 5-(1H-benzimidazol-1-yl)-3-{[(trifluoromethyl)sulfonyl]oxy}-2-thiophenecarboxylate (0.300 g, 0.738 mmol) was dissolved in 7 mL of N,N-dimethylformamide with stirring. Triethylamine (0.21 mL, 1.5 mmol) was added via syringe. Copper (I) iodide (0.0141 g, 0.0740 mmol) was added followed by trans-dichlorobis(triphenylphosphine) palladium (II) (0.0258 g, 0.0368 mmol). Phenylacetylene (0.12 mL, 1.1 mmol) was added via syringe, and the mixture was heated to 80° C. for 16 hours. The mixture was cooled to room temperature and poured into ethyl acetate and water. The layers were separated, and the organic layer was washed with brine. The combined aqueous layers were extracted with ethyl acetate. The combined organic layers were dried over MgSO₄, filtered, and concentrated in vacuo. Purification by flash chromatography afforded 0.212 g (80%) of methyl 5-(1H-benzimidazol-1-yl)-3-(phenylethynyl)-2-thiophenecarboxylate. ¹H NMR (300 MHz, DMSO-d₆) δ 8.76 (s, 1H), 7.85 (m, 2H), 7.80 (s, 1H), 7.64-7.58 (m, 2H), 7.52-7.35 (m, 5H), 3.92 (s, 3H). MS (ES+, m/z) 359 (m+1).

### EXAMPLE 129

### 5-(1H-Benzimidazol-1-yl)-3-(phenylethynyl)thiophene-2-carboxamide

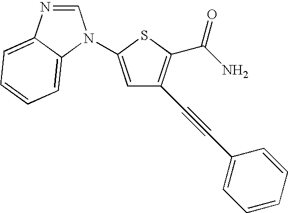

5-(1H-benzimidazol-1-yl)-3-(phenylethynyl)thiophene-2-carboxamide was prepared from methyl 5-(1H-benzimidazol-1-yl)-3-(phenylethynyl)-2-thiophenecarboxylate using procedure similarly described in Example 61 except 7M NH₃ in MeOH was used instead of 2M NH₃ in MeOH. ¹H NMR (400 MHz, DMSO-d₆) δ 8.71 (s, 1H), 8.09 (br s, 1H), 7.85-7.80 (m, 2H), 7.72 (s, 1H), 7.67-7.63 (m, 2H), 7.53-7.36 (m, 6H). MS (ES+, m/z) 344 (m+1).

### INTERMEDIATE EXAMPLE 2

### Methyl 5-(1H-benzimidazol-1-yl)-3-(2-phenylethyl)-2-thiophenecarboxylate

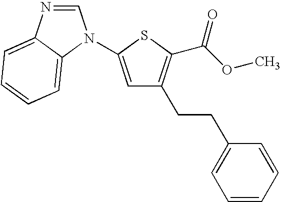

Methyl 5-(1H-benzimidazol-1-yl)-3-(phenylethynyl)-2-thiophenecarboxylate (0.110 g, 0.307 mmol) was dissolved in 10 mL of ethyl acetate with stirring. 10% Palladium on carbon (0.0327 g, 0.0307 mmol) was added, and the reaction placed under 1 atmosphere of hydrogen for 16 hours. The mixture was filtered through celite, washing with ethyl acetate. The filtrate was concentrated to afford 0.109 g (98%) of methyl 5-(1H-benzimidazol-1-yl)-3-(2-phenylethyl)-2-thiophenecarboxylate. ¹H NM R (300 MHz, DMSO-d₆) δ 8.67 (s, 1H), 7.80 (d, J=7.5 Hz, 1H), 7.72 (d, J=7.2 Hz, 1H), 7.49 (s, 1H), 7.46-7.17 (m, 7H), 3.84 (s, 3H), 3.32 (m, 2H), 2.95 (m, 2H). MS (ES+, m/z) 363 (m+1).

### EXAMPLE 130

### 5-(1H-Benzimidazol-1-yl)-3-(2-phenylethyl)thiophene-2-carboxamide

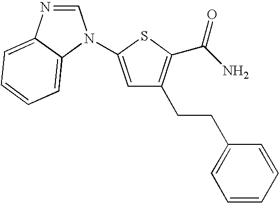

5-(1H-benzimidazol-1-yl)-3-(2-phenylethyl)thiophene-2-carboxamide was prepared from methyl 5-(1H-benzimidazol-1-yl)-3-(2-phenylethyl)-2-thiophenecarboxylate using procedure similarly described in Example 61 except 7M NH₃ in MeOH was used instead of 2M NH₃ in MeOH. ¹H NMR (300 MHz, DMSO-d₆) δ 8.58 (s, 1H), 7.79 (d, J=7.3 Hz, 1H), 7.65 (d, J=8.1 Hz, 1H), 7.56 (br s, 2H), 7.44-7.17 (m, 8H), 3.22 (m, 2H), 2.95 (m, 2H). MS (ES+, m/z) 348 (m+1).

### EXAMPLE 131

### 5-(1H-Benzimidazol-1-yl)-3-[methyl(phenyl)amino]thiophene-2-carboxamide

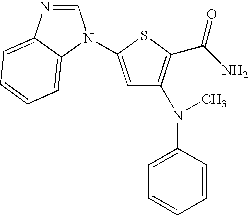

Compound was prepared using procedures similarly described for Example 31 and 61. ¹H NMR (300 MHz, DMSO-d₆) δ 8.65 (s, 1H), 7.83-7.67 (m, 3H), 7.46-7.23 (m, 6H), 6.91-6.84 (m, 3H), 3.28 (s, 3H). MS (ES+, m/z) 349 (m+1).

### EXAMPLE 132

### 5-(1H-Benzimidazol-1-yl)-3-[(phenylsulfonyl)amino]thiophene-2-carboxamide

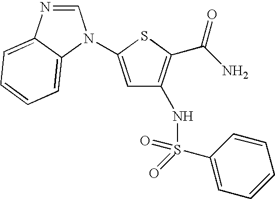

Compound was prepared using procedures similarly described for Example 32 except sulfonamide was used instead of benzamide and Example 61. ¹H NMR (300 MHz, DMSO-d₆) δ 11.40 (s, 1H), 8.75 (s, 1H), 7.95-7.90 (m, 3H), 7.88 (br s, 1H), 7.82 (m, 1H), 7.71 (m, 1H), 7.65-7.58 (m, 3H), 7.51 (s, 1H), 7.45 (m, 1H), 7.40 (m, 1H). MS (ES+, m/z) 399 (m+1).

### INTERMEDIATE EXAMPLE 3

### Methyl 5-(1H-benzimidazol-1-yl)-3-({[(phenylmethyl)oxy]carbonyl]amino)-2-thiophenecarboxylate

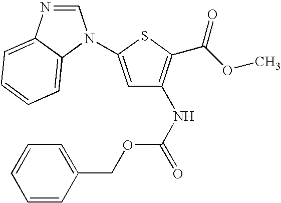

Methyl 5-(1H-benzimidazol-1-yl)-3-{[(trifluoromethyl)sulfonyl]oxy}-2-thiophenecarboxylate (1.11 g, 2.73 mmol), cesium carbonate (1.25 g, 3.84 mmol), 2,2′-bis(diphenylphosphino)-1,1′-binaphthyl (0.0850 g, 0.137 mmol), and tris(dibenzylideneacetone dipalladium (0) (0.0625 g, 0.0683 mmol) were combined in a reaction flask with 30 mL of toluene with stirring. Benzyl carbamate (0.495 g, 3.27 mmol) was added, and the reaction was heated to 100° C. for 40 hours. The reaction was cooled to room temperature, adsorbed directly onto silica gel, and purified by flash chromatography to afford 0.604 g (54%) of methyl 5-(1H-benzimidazol-1-yl)-3-({[(phenylmethyl)oxy]carbonyl}amino)-2-thiophenecarboxylate. ¹H NMR (400 MHz, DMSO-d₆) δ 9.67 (s, 1H), 8.76 (s, 1H), 8.03 (s, 1H), 7.84-7.77 (m, 2H), 7.49-7.28 (m, 7H), 5.26 (s, 2H), 3.86 (s, 3H). MS (ES+, m/z) 408 (m+1).

### INTERMEDIATE EXAMPLE 4

### Methyl 5-(1H-benzimidazol-1-yl)-3-({[(phenylmethyl)oxy]carbonyl}{[2-(trifluoromethyl)phenyl]methyl}amino)-2-thiophenecarboxylate

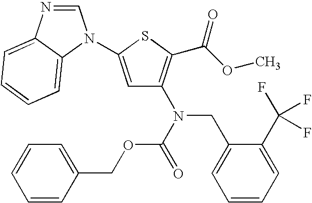

Methyl 5-(1H-benzimidazol-1-yl)-3-({[(phenylmethyl)oxy]carbonyl}amino)-2-thiophenecarboxylate (0.400 g, 0.982 mmol) and cesium carbonate (1.02 g, 3.13 mmol) were placed in a flask with 12 mL of N,N-dimethylformamide with stirring. 2-(Trifluoromethyl)benzyl bromide (0.704 g, 2.95 mmol) was added, and the reaction was stirred for 16 hours. The mixture was poured into water and ethyl acetate, and the layers were separated. The organic layer was washed with brine, and the combined aqueous layers were extracted with ethyl acetate. The combined organic layers were dried over MgSO₄, filtered, and concentrated in vacuo. Flash chromatography provided somewhat impure methyl 5-(1H-benzimidazol-1-yl)-3-({[(phenylmethyl)oxy]-carbonyl}{[2-(trifluoromethyl)phenyl]methyl}amino)-2-thiophenecarboxylate that was carried directly into the next step. MS (ES+, m/z) 567 (m+1).

### INTERMEDIATE EXAMPLE 5

### 5-(1H-benzimidazol-1-yl)-3-({[(phenylmethyl)oxy]carbonyl]-{[2-(trifluoromethyl)phenyl]methyl lamino)-2-thiophenecarboxylic acid

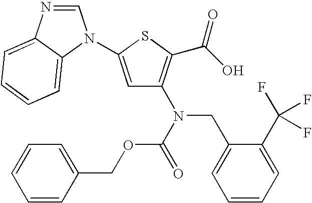

Methyl 5-(1H-benzimidazol-1-yl)-3-({[(phenyl methyl)oxy]carbonyl}{[2-(trifluoromethyl)phenyl]methyl}amino)-2-thiophenecarboxylate (0.555 g, 0.982 mmol) was dissolved in 10 ml of tetrahydrofuran with stirring. 10 ml of 1N LiOH solution was added, and the mixture was allowed to stir for 16 hours. The mixture was poured into diethyl ether and water, and the layers were separated. The organic layer was washed with water, and the diethyl ether layer was subsequently discarded. The combined aqueous layers were acidified to pH 2 with concentrated HCl and extracted with ethyl acetate three times. The combined organic layers were dried over MgSO₄, filtered, and concentrated in vacuo to afford 0.528 g (97%) of 5-(1H-benzimidazol-1-yl)-3-({[(phenylmethyl)oxy]carbonyl}{[2-(trifluoromethyl)phenyl]methyl}amino)-2-thiophenecarboxylic acid as an off-white solid. ¹H NMR (400 MHz, DMSO-d₆) δ 13.60 (br s, 1H), 8.58 (s, 1H), 7.85 (d, J=7.7 Hz, 1H), 7.76 (d, J=7.0 Hz, 1H), 7.70 (d, J=7.7 Hz, 1H), 7.65 (dd, J=7.7, 7.7 Hz, 1H), 7.54-7.43 (m, 4H), 7.41-7.24 (m, 6H), 5.14 (s, 2H), 5.14 (s, 2H). MS (ES+, m/z) 552 (m+1).

### INTERMEDIATE EXAMPLE 6

### Benzyl 2-(aminocarbonyl)-5-(1H-benzimidazol-1-yl)thien-3-yl[2-(trifluoromethyl)-benzyl]carbamate

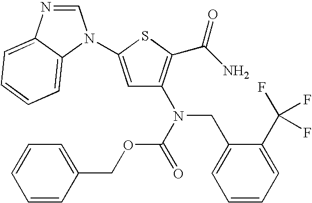

5-(1H-Benzimidazol-1-yl)-3-({[(phenylmethyl)oxy]carbonyl}{[2-(trifluoromethyl)phenyl]methyl}amino)-2-thiophenecarboxylic acid (0.200 g, 0.363 mmol) and ammonium chloride (0.0388 g, 0.725 mmol) were added to a flask with 5 mL of N,N-dimethylformamide with stirring. N-Methylmorpholine (0.080 mL, 0.73 mmol) was added via syringe. 1-Hydroxybenzotriazole (0.0981 g, 0.726 mmol) was added followed by 1-(3-dimethylaminopropyl)-3-ethylcarbodiimide hydrochloride (0.0974 g, 0.508 mmol). The mixture was stirred for 16 hours and poured into ethyl acetate and 1N HCl. The layers were separated, and the organic layer was washed with brine. The combined aqueous layers were extracted with ethyl acetate. The combined organic layers were dried over MgSO₄, filtered, and concentrated in vacuo. Purification by flash chromatography afforded 0.171 g (86%) of benzyl 2-(aminocarbonyl)-5-(1H-benzimidazol-1-yl)thien-3-yl[2-(trifluoromethyl)-benzyl]carbamate as an off-white solid. ¹H NMR (400 MHz, DMSO-d₆) δ 8.53 (s, 1H), 7.85-7.75 (m, 2H), 7.75-7.69 (m, 3H), 7.66 (dd, J=7.4, 7.4 Hz, 1H), 7.50 (dd, J=7.5, 7.5 Hz, 1H), 7.47-7.27 (m, 9H), 5.16 (s, 2H), 5.11 (br s, 2H). MS (ES+, m/z) 551 (m+1).

### EXAMPLE 133

### 5-(1H-Benzimidazol-1-yl)-3-[[2-(trifluoromethyl)benzyl]amino]-thiophene-2-carboxamide

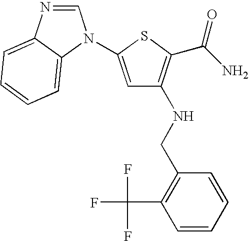

Phenylmethyl [2-(aminocarbonyl)-5-(1H-benzimidazol-1-yl)-3-thienyl]{[2-(trifluoromethyl)phenyl]methyl}carbamate (0.157 g, 0.285 mmol) was dissolved in 10 mL of ethyl acetate with stirring. 100% Palladium on carbon (0.0606 g, 0.0570 mmol) was added, and the solution was placed under 1 atmosphere of hydrogen. The reaction was stirred for 48 hours and was judged incomplete. The reaction mixture was filtered through a celite pad and washed with ethyl acetate. The filtrate was concentrated in vacuo and purified by flash chromatography to afford 0.0257 g (22%) of pure product and 0.112 mg of a mixture of starting material and product. ¹H NMR (400 MHz, DMSO-d₆) δ 8.61 (s, 1H), 8.16 (dd, J=6.6, 6.6 Hz, 1H), 7.81-7.74 (m, 2H), 7.73-7.67 (m, 2H), 7.64 (d, J=7.7 Hz, 1H), 7.50 (dd, J=7.5, 7.5 Hz, 1H), 7.43-7.33 (m, 2H), 7.19 (s, 1H), 7.14 (br s, 2H), 4.71 (d, J=6.2 Hz, 2H). MS (ES+, m/z) 417 (m+1).

### INTERMEDIATE EXAMPLE 7

### 2-(Methyloxy)-5-nitrophenyl 2,2-dimethylpropanoate

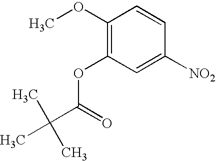

Commercially available 2-Methoxy-5-nitrophenol (10.0 g, 59.1 mmol) was dissolved in 150 mL of dichloromethane with 4-dimethylaminopyridine (0.722 g, 5.91 mmol). Triethylamine (9.88 mL, 70.9 mmol) was added via syringe. Pivaloyl chloride (8.01 mL, 65.0 mmol) was added slowly via syringe. The reaction was stirred for ten minutes and poured into 1N HCl. The layers were separated, and the aqueous layer was washed with dichloromethane. The combined organic layers were washed with saturated NaHCO₃ and brine. The organic layers were dried over MgSO₄, filtered, and concentrated in vacuo. The isolated solid was triturated with hexanes, filtered, and washed with hexanes and 2-methylbutane. The solid was air dried and collected to afford 13.0 g (87%) of 2-(methyloxy)-5-nitrophenyl 2,2-dimethylpropanoate. ¹H NMR (400 MHz, CDCl₃) δ 8.16 (dd, J=9.2, 2.8 Hz, 1H), 7.95 (d, J=2.8 Hz, 1H), 7.01 (d, J=9.2 Hz, 1H), 3.92 (s, 3H), 1.38 (s, 9H).

### INTERMEDIATE EXAMPLE 8

### 5-Amino-2-(methyloxy)phenyl 2,2-dimethylpropanoate

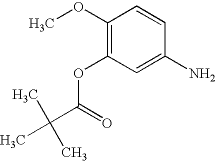

2-(Methyloxy)-5-nitrophenyl 2,2-dimethylpropanoate (13.0 g, 51.4 mmol) was dissolved in 150 mL of ethyl acetate with stirring. 10% Palladium on carbon (1.64 g, 1.54 mmol) was added and the solution was stirred under 1 atmosphere of hydrogen for 16 hours. The reaction was filtered through celite and washed well with ethyl acetate. The filtrate was concentrated in vacuo to afford 11.3 g (98%) of 5-amino-2-(methyloxy)phenyl 2,2-dimethylpropanoate as a pink solid. ¹H NMR (400 MHz, CDCl₃) δ 6.80 (d, J=8.6 Hz, 1H), 6.55 (dd, J=8.6, 2.8 Hz, 1H), 6.45 (d, J=2.8 Hz, 1H), 3.73 (s, 3H), 1.35 (s, 9H).

### INTERMEDIATE EXAMPLE 9

### 2-(Methyloxy)-4-nitro-5-[(trifluoroacetyl)amino]phenyl 2,2-dimethylpropanoate

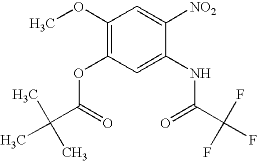

5-Amino-2-(methyloxy)phenyl 2,2-dimethylpropanoate (10.53 g, 47.1 mmol) was dissolved in 200 mL of chloroform with stirring. Ammonium nitrate (6.79 g, 84.8 mmol) was added in a single portion. The mixture was cooled to 0° C., and trifluoroacetic anhydride (36 mL, 260 mmol) was added dropwise via addition funnel over 1 hour. The reaction was warmed to room temperature and stirred for an additional six hours. The reaction was quenched by the careful addition of 100 mL of saturated NaHCO₃ and stirred for 15 minutes. The mixture was poured into a separatory funnel, and the layers were separated. The aqueous layer was washed with dichloromethane. The combined organic layers were dried over MgSO₄, filtered, and concentrated in vacuo to provide 16.5 g (96%) of 2-(methyloxy)-4-nitro-5-[(trifluoroacetyl)amino]phenyl 2,2-dimethylpropanoate as a yellow solid. ¹H NMR (400 MHz, CDCl₃) δ 11.36 (br s, 1H), 8.49 (s, 1H), 7.84 (s, 1H), 3.91 (s, 3H), 1.38 (s, 9H).

### INTERMEDIATE EXAMPLE 10

### 5-Amino-2-(methyloxy)-4-nitrophenol

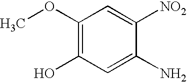

2-(Methyloxy)-4-nitro-5-[(trifluoroacetyl)amino]phenyl 2,2-dimethylpropanoate (16.5 g, 45.2 mmol) was dissolved in 200 mL of methanol and 200 mL of water with stirring. Potassium carbonate (31.2 g, 226 mmol) was added and the solution was stirred for sixteen hours at room temperature. At this point the reaction was judged incomplete and heated to reflux for two hours. The mixture was cooled to room temperature, and the majority of the methanol was removed in vacuo. Ethyl acetate was added, and the pH of the solution was adjusted to approximately 7 using concentrated HCl. The layers were separated, and the organic layer was washed with brine. The combined aqueous layers were saturated with NaCl and further extracted with ethyl acetate (2×) and 20% isopropanol in ethyl acetate (4×). The combined organic extracts were dried over MgSO₄, filtered, and concentrated in vacuo. The isolated solid was triturated with diethyl ether, filtered, and washed with diethyl ether and 2-methylbutane. The orange solid was air dried and collected to yield 7.15 g (86%) of 5-amino-2-(methyloxy)-4-nitrophenol. ¹H NMR (400 MHz, CDCl₃) δ 10.66 (br s, 1H), 7.36 (br s, 2H), 7.32 (s, 1H), 6.36 (s, 1H), 3.73 (s, 3H). MS (ES+, m/z) 185 (m+1).

### INTERMEDIATE EXAMPLE 11

### 5-{[(1,1-Dimethylethyl)(diphenyl)silyl]oxy}-4-(methyloxy)-2-nitroaniline

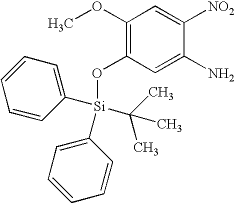

5-Amino-2-(methyloxy)-4-nitrophenol (6.90 g, 37.5 mmol) was dissolved in 100 mL of acetonitrile with stirring. Triethylamine (6.30 mL, 45.2 mmol) was added via syringe. t-Butylchlorodiphenylsilane (9.75 mL, 37.5 mmol) was added slowly via syringe. The reaction was stirred for 2 hours and judged incomplete. Additional triethylamine (1.57 mL, 11.3 mmol) and t-butylchlorodiphenylsilane (2.94 mL, 11.3 mmol) were added via syringe. The reaction was stirred an additional 15 minutes and poured into ethyl acetate and 1N NaOH. The layers were separated, and the organic layer was washed with brine. The combined aqueous layers were extracted with ethyl acetate. The combined organic layers were dried over MgSO₄, filtered, and concentrated in vacuo. The isolated material was passed through a plug of silica gel, and the fractions containing product concentrated. The isolated viscous oil was a mixture of unidentified silyl byproducts and 5-{[(1,1-dimethylethyl)(diphenyl)silyl]oxy}-4-(methyloxy)-2-nitroaniline. The yield was not determined, and the impure material carried forward to the next step. MS (ES+, m/z) 423 (m+1).

### INTERMEDIATE EXAMPLE 12

### [S-{[(1,1-Dimethylethyl)(diphenyl)silyl]oxy}-4-(methyloxy)-2-nitrophenyl]formamide

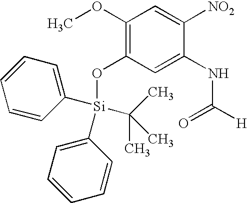

Acetic anhydride (17.7 mL, 188 mL) was slowly added to formic acid (14.1 mL, 374 mmol) with stirring. The mixture was placed in a 50° C. oil bath for one hour. After cooling to room temperature, the impure material containing 5-{[(1,1-dimethylethyl)(diphenyl)-silyl]oxy}-4-(methyloxy)-2-nitroaniline was dissolved in 100 mL of dichloromethane and added to the reaction. The reaction was allowed to stir for 14 hours and quenched by the careful addition of 100 mL of water. The reaction was slowly poured into saturated NaHCO₃ and dichloromethane. The layers were separated, and the aqueous layer washed with dichloromethane. The combined organic layers were dried over MgSO₄, filtered, and concentrated to provide impure material containing [5-{[(1,1-dimethylethyl)(diphenyl)-silyl]oxy}-4-(methyloxy)-2-nitrophenyl]formamide. The yield was not determined, and the impure material carried forward to the next step. MS (ES+, m/z) 451 (m+1).

### INTERMEDIATE EXAMPLE 13

### [2-Amino-5-{[(1,1-dimethylethyl)(diphenyl)silyl]oxy}-4-(methyloxy)phenyl]-formamide

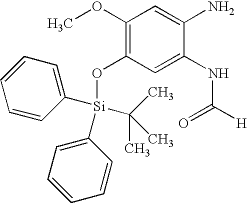

The impure material containing [5-{[(1,1-dimethylethyl)(diphenyl)-silyl]oxy}-4-(methyloxy)-2-nitrophenyl]formamide was dissolved in 200 mL of ethyl acetate with stirring. 10% Palladium on carbon (1.20 g, 1.13 mmol) was added, and the mixture was placed under 1 atmosphere of hydrogen for 24 hours. The reaction was filtered through celite washing with ethyl acetate and chloroform. The filtrate was concentrated in vacuo to afford impure material containing [2-amino-5-{[(1,1-dimethylethyl)(diphenyl)-silyl]oxy}-4-(methyloxy)phenyl]-formamide. The yield was not determined, and the impure material carried forward to the next step. MS (ES+, m/z) 421 (m+1).

### INTERMEDIATE EXAMPLE 14

### 5-{[(1,1-Dimethylethyl)(diphenyl)silyl]oxy}-6-(methyloxy)-1H-benzimidazole

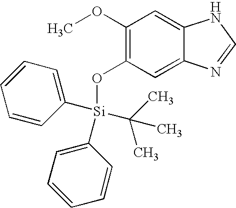

The impure material containing [2-amino-5-{[(1,1-dimethylethyl)(diphenyl)-silyl]oxy}-4-(methyloxy)phenyl]-formamide was dissolved in 200 mL of chloroform with stirring. Magnesium sulfate (13.54 g, 112 mmol) was added in a single portion. Pyridinium p-toluenesulfonate (11.3 g, 45.0 mmol) was added, and the reaction was allowed to stir for 16 hours. The reaction was judged incomplete, therefore the mixture was heated to between 40 and 50° C. for 8 additional hours. The reaction was cooled to room temperature and solid NaHCO₃ (10 g) was added. The mixture was stirred for 30 minutes and filtered to remove all solid particles. The filtrate was concentrated to approximately 100-200 mL total volume at which point significant solid formation had occurred. 200 mL of diethyl ether and hexanes (1:1) was added, and the mixture was filtered. The solid was washed with hexanes and 2-methylbutane. The solid was air dried, collected, and determined to be the tosyl salt of the desired product. The solid was placed in a separatory funnel with 1N NaOH and extracted twice with isopropanol in dichloromethane (1:4). The combined organic layers were dried over MgSO₄, filtered, and concentrated in vacuo to afford 7.80 g (52% over 4 steps) of 5-{[(1,1-dimethylethyl)(diphenyl)silyl]oxy}-6-(methyloxy)-1H-benzimidazole. ¹H NMR (400 MHz, DMSO-d₆) δ 12.00 (br s, 1H), 7.93 (s, 1H), 7.72-7.67 (m, 4H), 7.49-7.39 (m, 6H), 7.07 (s, 1H), 6.78 (s, 1H), 3.65 (s, 3H), 1.07 (s, 9H). MS (ES+, m/z) 403 (m+1).

### INTERMEDIATE EXAMPLE 15

### Methyl 5-[6-{[(1,1-dimethylethyl)(diphenyl)silyl]oxy}-5-(methyloxy)-1H-benzimidazol-1-yl]-3-hydroxy-2-thiophenecarboxylate and Methyl 5-[5-f [(1,1-dimethylethyl)(diphenyl)silyl]oxy}-6-(methyloxy)-1H-benzimidazol-1-yl]-3-hydroxy-2-thiophenecarboxylate

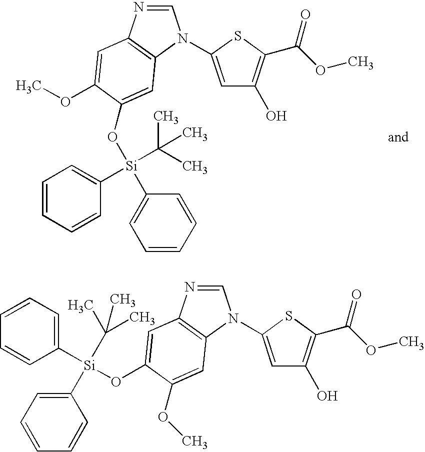

5-{[(1,1-Dimethylethyl)(diphenyl)silyl]oxy}-6-(methyloxy)-1H-benzimidazole (4.12 g, 10.2 mmol) was dissolved in 50 mL of chloroform with stirring. Methyl 2-chloro-3-oxo-2,3-dihydro-2-thiophenecarboxylate (0.982 g, 5.10 mmol) was added in a single portion. The reaction was allowed to stir for 5 days. 50 mL of water was added, and the pH was adjusted to approximately 6-7 using saturated NaHCO₃. The layers were separated, and the aqueous layer was extracted with dichloromethane (1×) and ethyl acetate (1×). The combined organic layers were dried over MgSO₄, filtered, and concentrated in vacuo. The residue was purified by flash chromatography to afford 2.40 g (84%) of a 1.2-1.4:1 regioisomeric mixture of methyl 5-[6-{[(1,1-dimethylethyl)(diphenyl)silyl]oxy}-5-(methyloxy)-1H-benzimidazol-1-yl]-3-hydroxy-2-thiophenecarboxylate and methyl 5-[5-{[(1,1-dimethylethyl)(diphenyl)silyl]oxy}-6-(methyloxy)-1H-benzimidazol-1-yl]-3-hydroxy-2-thiophenecarboxylate. ¹H NMR (300 MHz, DMSO-d₆) δ 10.84, 10.74 (br s, 1H), 8.50, 8.42 (s, 1H), 7.76-7.69 (m, 4H), 7.54-0.41 (m, 6H), 7.36, 7.23 (s, 1H), 7.13, 7.00 (s, 1H), 6.93, 6.91 (s, 1H), 3.86, 3.82 (s, 3H), 0.744, 3.737 (s, 3H), 1.12, 1.11 (s, 9H). MS (ES+, m/z) 403 (m+1).

### INTERMEDIATE EXAMPLE 16

### Methyl 5-[6-{[(1,1-dimethylethyl)(diphenyl)silyl]oxy}-5-(methyloxy)-1H-benzimidazol-1-yl]-3-(I [2-(trifluoromethyl)phenyl]methyl}oxy)-2-thiophenecarboxylate and Methyl 5-[5-{[(1,1-dimethylethyl)(diphenyl)silyl]oxy}-6-(methyloxy)-1H-benzimidazol-1-yl]-3-({[2-(trifluoromethyl)phenyl]methyl}oxy)-2-thiophenecarboxylate

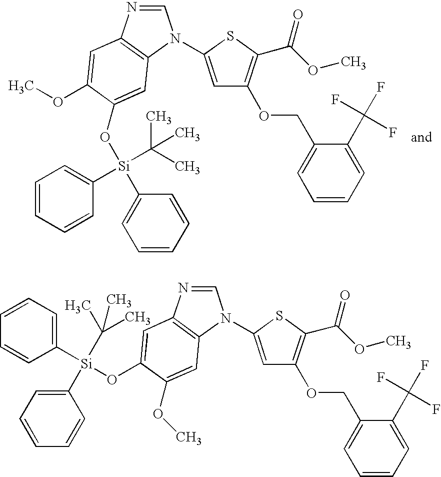

A regioisomeric mixture of methyl 5-[6-{[(1,1-dimethylethyl)(diphenyl)silyl]oxy}-5-(methyloxy)-1H-benzimidazol-1-yl]-3-hydroxy-2-thiophenecarboxylate and methyl 5-[5-{[(1,1-dimethylethyl)(diphenyl)silyl]oxy}-6-(methyloxy)-1H-benzimidazol-1-yl]-3-hydroxy-2-thiophenecarboxylate (3.97 g, 7.11 mmol) was dissolved in 40 mL of N,N-dimethylformamide with stirring. Potassium carbonate (1.18 g, 8.54 mmol) was added in a single portion. 2-Trifluoromethyl)benzyl bromide (2.04 g, 8.53 mmol) was added in a single portion. The reaction was allowed to stir for 16 hours and poured into water and ethyl acetate. The layers were separated, and the organic layer was washed with brine. The combined aqueous layers were extracted with ethyl acetate. The combined organic layers were dried over MgSO₄, filtered, and concentrated in vacuo. Purification by flash chromatography afforded 2.65 g (52%) of methyl 5-[5-{[(1,1-dimethylethyl)(diphenyl)-silyl]oxy}-6-(methyloxy)-1H-benzimidazol-1-yl]-3-({[2-(trifluoromethyl)phenyl]-methyl}oxy)-2-thiophenecarboxylate and 2.13 g (42%) of methyl 5-[6-{[(1,1-dimethylethyl)(diphenyl)silyl]oxy}-5-(methyloxy)-1H-benzimidazol-1-yl]-3-({[2-(trifluoromethyl)phenyl]methyl}oxy)-2-thiophenecarboxylate. Data for (5-OTBDPS, 6-OMe): ¹H NMR (300 MHz, DMSO-d₆) δ 8.47 (s, 1H), 7.98 (d, J=7.4 Hz, 1H), 7.88-7.60 (m, 8H), 7.55-7.42 (m, 6H), 7.25 (s, 1H), 6.94 (s, 1H), 5.54 (s, 2H), 3.81 (s, 3H), 3.73 (s, 3H), 1.11 (s, 9H). MS (ES+, m/z) 717 (m+1). Data for (5-OMe, 6-OTBDPS): ¹H NMR (300 MHz, DMSO-d₆) δ 8.57 (s, 1H), 7.98 (d, J=7.6 Hz, 1H), 7.88-7.79 (m, 2H), 7.76-7.61 (m, 5H), 7.56 (s, 1H), 7.51-7.41 (m, 6H), 7.37 (s, 1H), 7.05 (s, 1H), 5.43 (s, 2H), 3.84 (s, 3H), 3.74 (s, 3H), 1.12 (s, 9H). MS (ES+, m/z) 717 (m+1).

### INTERMEDIATE EXAMPLE 17

### Methyl 5-[5-hydroxy-6-(methyloxy)-1H-benzimidazol-1-yl]-3-({[2-(trifluoromethyl)phenyl]methyl}oxy)-2-thiophenecarboxylate

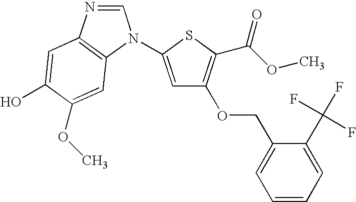

Methyl 5-[5-{[(1,1-dimethylethyl)(diphenyl)-silyl]oxy}-6-(methyloxy)-1H-benzimidazol-1-yl]-3-({[2-(trifluoromethyl)phenyl]-methyl}oxy)-2-thiophenecarboxylate (1.54 g, 2.15 mmol) was dissolved in 20 mL of tetrahydrofuran with stirring. The solution was cooled to 0° C., and tetrabutylammonium fluoride (3.20 mL, 1.0M in THF, 3.20 mmol) was added slowly via syringe. The reaction was stirred for ten minutes and quenched by the addition of 50 mL of 0.5N HCl. The mixture was poured into ethyl acetate, and the layers were separated. The organic layer was washed with brine, and the combined aqueous layers were extracted with ethyl acetate. The combined organic layers were dried over MgSO₄, filtered, and concentrated in vacuo. Purification by flash chromatography provided 0.761 g (74%) of methyl 5-[5-hydroxy-6-(methyloxy)-1H-benzimidazol-1-yl]-3-({[2-(trifluoromethyl)phenyl]methyl}oxy)-2-thiophenecarboxylate as an off-white solid. ¹H NMR (400 MHz, DMSO-d₆) δ 9.07 (s, 1H), 8.47 (s, 1H), 7.96 (d, J=7.9 Hz, 1H), 7.84-7.76 (m, 2H), 7.65 (s, 1H), 7.62 (dd, J=7.9, 7.7 Hz, 1H), 7.24 (s, 1H), 7.13 (s, 1H), 5.53 (s, 2H), 3.86 (s, 3H), 3.78 (s, 3H).

### INTERMEDIATE EXAMPLE 18

### Methyl 5-[6-hydroxy-5-(methyloxy)-1H-benzimidazol-1-yl]-3-({[2-(trifluoromethyl)phenyl]methyl}oxy)-2-thiophenecarboxylate

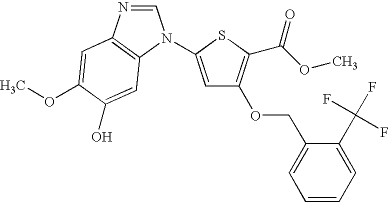

This compound was prepared in a similar manner to that previously described for the synthesis of methyl 5-[5-hydroxy-6-(methyloxy)-1H-benzimidazol-1-yl]-3-({[2-(trifluoromethyl)phenyl]-methyl}oxy)-2-thiophenecarboxylate. Reaction of methyl 5-[6-{[(1,1-dimethylethyl)(diphenyl)silyl]oxy}-5-(methyloxy)-1H-benzimidazol-1-yl]-3-({[2-(trifluoromethyl)phenyl]methyl}oxy)-2-thiophenecarboxylate (3.22 g, 4.49 m mol) with tetrabutylammonium fluoride (6.74 mL, 1.0M in THF, 6.74 mmol) afforded 1.76 g (82%) of methyl 5-[6-hydroxy-5-(methyloxy)-1H-benzimidazol-1-yl]-3-({[2-(trifluoromethyl)-phenyl]methyl}oxy)-2-thiophenecarboxylate as a pale yellow solid. ¹H NMR (400 MHz, DMSO-d₆) δ 8.72 (s, 1H), 7.98 (d, J=7.7 Hz, 1H), 7.85-7.77 (m, 2H), 7.72 (s, 1H), 7.62 (dd, J=7.9, 7.7 Hz, 1H), 7.32 (s, 1H), 7.30 (s, 1H), 5.50 (s, 2H), 3.86 (s, 3H), 3.78 (s, 3H).

### INTERMEDIATE EXAMPLE 19

### 5-{[(1,1-Dimethylethyl)(diphenyl)silyl]oxy}-1H-benzimidazole

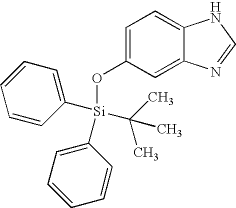

This compound was prepared in four steps from commercially available 4-amino-3-nitrophenol using a procedure similar to that outlined for the synthesis of 5-{[(1,1-dimethylethyl)(diphenyl)silyl]oxy}-6-(methyloxy)-1H-benzimidazole. ¹H NM R (400 MHz, DMSO-d₆) δ 12.15 (br s, 1H), 8.03 (s, 1H), 7.74-7.67 (m, 4H), 7.51-7.39 (m, 6H), 7.37 (d, J=8.6 Hz, 1H), 6.81 (d, J=2.2 Hz, 1H), 6.75 (dd, J=8.6, 2.2 Hz, 1H), 1.05 (s, 9H). MS (ES+, m/z) 373 (m+1).

### INTERMEDIATE EXAMPLE 20

### Methyl 5-(6-{[(1,1-dimethylethyl)(diphenyl)silyl]oxy}-1H-benzimidazol-1-yl)-3-hydroxy-2-thiophenecarboxylate and Methyl 5-(5-{[(1,1-dimethylethyl)-(diphenyl)silyl]oxy}-1H-benzimidazol-1-yl)-3-hydroxy-2-thiophenecarboxylate

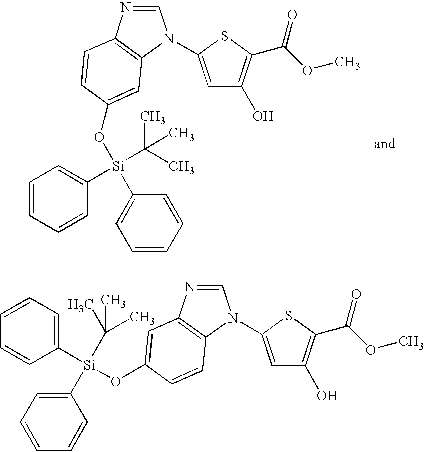

5-{[(1,1-dimethylethyl)(diphenyl)silyl]oxy}-6-(methyloxy)-1H-benzimidazole (9.43 g, 25.3 mmol) was dissolved in 125 mL of chloroform with stirring. Methyl 2-chloro-3-oxo-2,3-dihydro-2-thiophenecarboxylate (2.44 g, 12.7 mmol) was added in a single portion. The reaction was allowed to stir for 10 days. 100 mL of water was added, and the pH was adjusted to approximately 6-7 using saturated NaHCO₃. The layers were separated, and the aqueous layer was extracted with dichloromethane (1×) and ethyl acetate (1×). The combined organic layers were dried over MgSO₄, filtered, and concentrated in vacuo. The residue was purified by flash chromatography to afford 5.48 g (82%) of a 1.0-1.1:1 regioisomeric mixture of methyl 5-(6-{[(1,1-dimethylethyl)(diphenyl)silyl]oxy}-1H-benzimidazol-1-yl)-3-hydroxy-2-thiophenecarboxylate and methyl 5-(5-{[(1,1-dimethylethyl)-(diphenyl)silyl]oxy}-1H-benzimidazol-1-yl)-3-hydroxy-2-thiophenecarboxylate. ¹H NMR (300 MHz, DMSO-d₆) δ 10.85, 10.78 (br s, 1H), 8.59, 8.54 (s, 1H), 7.78-7.70 (m, 4H), 7.64, 7.60 (dd, J=8.8, 0.6 Hz and d, J=8.8 Hz, 1H), 7.56-7.43 (m, 6H), 7.10, 6.96 (s, 1H), 7.05-6.88 (m, 2H), 3.85, 3.81 (s, 3H), 1.11, 1.09 (s, 9H). MS (ES+, m/z) 529 (m+1).

### INTERMEDIATE EXAMPLE 21

### Methyl 5-(6-{[(1,1-dimethylethyl)(diphenyl)silyl]oxy}-1H-benzimidazol-1-yl)-3-({[2-(trifluoromethyl)phenyl]methyl}oxy)-2-thiophenecarboxylate and Methyl 5-(5-{[(1,1-dimethylethyl)(diphenyl)silyl]oxy}-1H-benzimidazol-1-yl)-3-({[2-(trifluoromethyl)phenyl]methyl}oxy)-2-thiophenecarboxylate

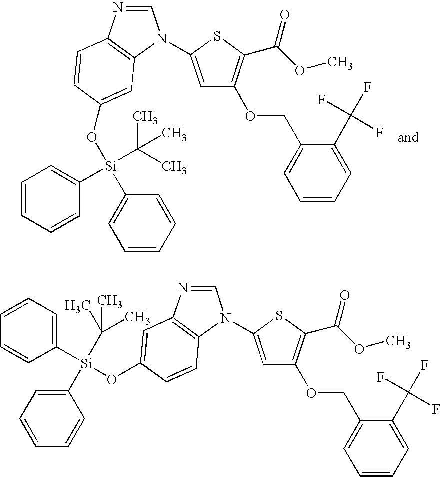

Polystyrene triphenylphoshine (9.84 g, 1.58 mmol/gram, 15.5 mmol) was stirred in 100 mL of dichloromethane for ten minutes. The regioisomeric mixture of methyl 5-(6-{[(1,1-dimethylethyl)(diphenyl)silyl]oxy}-1H-benzimidazol-1-yl)-3-hydroxy-2-thiophenecarboxylate and methyl 5-(5-{[(1,1-dimethylethyl)-(diphenyl)silyl]oxy}-1H-benzimidazol-1-yl)-3-hydroxy-2-thiophenecarboxylate (5.48 g, 10.4 mmol) was added in a single portion. 2-(Trifluromethyl)benzyl alcohol (1.68 mL, 12.6 mmol) was added via syringe, and the solution was cooled to 0° C. Di-Tert-butyl azodicarboxylate (3.58 g, 15.5 mmol) was dissolved in 20 mL of dichloromethane and added dropwise via addition funnel. The reaction was warmed to room temperature and stirred for 1.5 hours. The mixture was filtered through filter paper, and the solid was washed with dichloromethane and methanol. The filtrate was concentrated and purified by flash chromatography to afford 2.89 g (41%) of methyl 5-(5-{[(1,1-dimethylethyl)(diphenyl)silyl]oxy}-1H-benzimidazol-1-yl)-3-({[2-(trifluoromethyl)phenyl]methyl}oxy)-2-thiophenecarboxylate and 2.69 g (38%) of methyl 5-(6-{[(1,1-dimethylethyl)(diphenyl)silyl]oxy}-1H-benzimidazol-1-yl)-3-({[2-(trifluoromethyl)phenyl]methyl}oxy)-2-thiophenecarboxylate. Data for 5-OTBDPS regioisomer: ¹H NMR (300 MHz, DMSO-d₆) δ 8.66 (s, 1H), 7.98 (d, J=7.6 Hz, 1H), 7.86-7.60 (m, 9H), 7.56-7.44 (m, 6H), 7.01 (s, 1H), 6.99 (dd, J=6.7, 2.4 Hz, 1H), 5.51 (s, 2H), 3.79 (s, 3H), 1.10 (s, 9H). MS (ES+, m/z) 687 (m+1). Data for 6-OTBDPS regioisomer: ¹H NMR (300 MHz, DMSO-d₆) δ 8.60 (s, 1H), 7.99 (d, J=7.6 Hz, 1H), 7.87-7.57 (m, 9H), 7.54-7.42 (m, 6H), 7.07 (d, J=2.0 Hz, 1H), 6.92 (dd, J=8.8, 2.3 Hz, 1H), 5.46 (s, 2H), 3.84 (s, 3H), 1.11 (s, 9H). MS (ES+, m/z) 687 (m+1).

### INTERMEDIATE EXAMPLE 22

### Methyl 5-(6-hydroxy-1H-benzimidazol-1-yl)-3-({[2-(trifluoromethyl)-phenyl]methyl}oxy)-2-thiophenecarboxylate

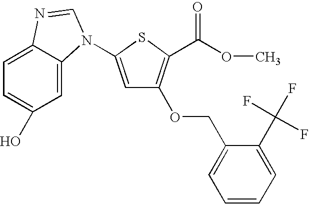

This compound was prepared in a similar manner to that previously described for the synthesis of methyl 5-[5-hydroxy-6-(methyloxy)-1H-benzimidazol-1-yl]-3-({[2-(trifluoromethyl)phenyl]-methyl}oxy)-2-thiophenecarboxylate. Reaction of methyl 5-(6-{[(1,1-dimethylethyl)(diphenyl)silyl]oxy}-1H-benzimidazol-1-yl)-3-({[2-(trifluoromethyl)-phenyl]methyl}oxy)-2-thiophenecarboxylate (2.69 g, 3.92 mmol) with tetrabutylammonium fluoride (5.9 mL, 1.0M in THF, 5.9 mmol) afforded 1.42 g (81%) of methyl 5-(6-hydroxy-1H-benzimidazol-1-yl)-3-({[2-(trifluoromethyl)-phenyl]methyl}-oxy)-2-thiophenecarboxylate as an off-white solid. ¹H NMR (300 MHz, DMSO-d₆) δ 9.72 (s, 1H), 8.60 (s, 1H), 8.01 (d, J=7.7 Hz, 1H), 7.89-7.79 (m, 2H), 7.75 (s, 1H), 7.67 (d, J=7.7 Hz, 1H), 7.62 (d, J=8.7 Hz, 1H), 7.27 (d, J=1.8 Hz, 1H), 6.87 (dd, J=8.7, 2.2 Hz, 1H), 5.53 (s, 2H), 3.81 (s, 3H).

### INTERMEDIATE EXAMPLE 23

### Methyl 5-(5-hydroxy-1H-benzimidazol-1-yl)-3-({[2-(trifluoromethyl)-phenyl]methyl}oxy)-2-thiophenecarboxylate

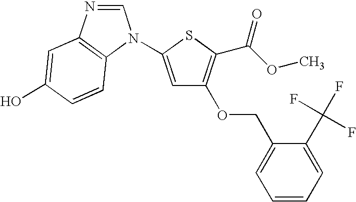

This compound was prepared in a similar manner to that previously described for the synthesis of methyl 5-[5-hydroxy-6-(methyloxy)-1H-benzimidazol-1-yl]-3-({[2-(trifluoromethyl)phenyl]-methyl}oxy)-2-thiophenecarboxylate. Reaction of methyl 5-(5-{[(1,1-dimethylethyl)(diphenyl)silyl]oxy}-1H-benzimidazol-1-yl)-3-({[2-(trifluoromethyl)-phenyl]methyl}oxy)-2-thiophenecarboxylate (2.89 g, 4.21 mmol) with tetrabutylammonium fluoride (6.3 mL, 1.0M in THF, 6.3 mmol) afforded 1.56 g (83%) of methyl 5-(5-hydroxy-1H-benzimidazol-1-yl)-3-({[2-(trifluoromethyl)-phenyl]methyl}-oxy)-2-thiophenecarboxylate as an off-white solid. ¹H NMR (400 MHz, DMSO-d₆) δ 9.46 (s, 1H), 8.64 (s, 1H), 7.97 (d, J=7.0 Hz, 1H), 7.86-7.76 (m, 2H), 7.72-7.59 (m, 3H), 7.09 (s, 1H), 6.92 (d, J=8.1 Hz, 1H), 5.51 (s, 2H), 3.77 (s, 3H).

### INTERMEDIATE EXAMPLE 24

### Methyl 5-(6-(methyloxy)-5-{[3-(2-oxo-1-pyrrolidinyl)propyl]oxy}-1H-benzimidazol-1-yl)-3-({[2-(trifluoromethyl)-phenyl]methyl}oxy)-2-thiophenecarboxylate

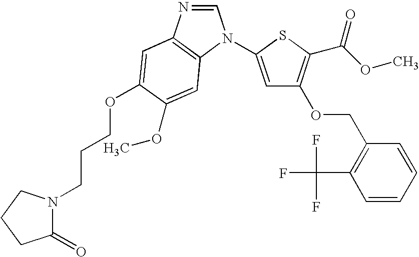

Polystyrene-triphenylphosphine (0.397 g, 1.58 mmol/gram, 0.627 mmol) was placed in a flask with 6 mL of dichloromethane and stirred for 5 minutes. 5-[5-Hydroxy-6-(methyloxy)-1H-benzimidazol-1-yl]-3-({[2-(trifluoromethyl)phenyl]methyl}oxy)-2-thiophenecarboxylate (0.150 g, 0.314 mmol) was added in a single portion. 1-(3-Hydroxypropyl)pyrrolidinone (0.059 mL, 0.412 mmol) was added via syringe, and the mixture was cooled to 0° C. Di-tert-butyl azodicarboxylate (0.144 g, 0.625 mmol) was dissolved in 1 mL dichloromethane and added dropwise via syringe. The reaction was warmed to room temperature and stirred for 1.5 hours. The reaction was filtered through filter paper and washed with dichloromethane and methanol. The filtrate was concentrated in vacuo and purified by flash chromatography to afford 0.152 g (80%) of methyl 5-(6-(methyloxy)-5-{[3-(2-oxo-1-pyrrolidinyl)propyl]oxy}-1H-benzimidazol-1-yl)-3-({[2-(trifluoromethyl)phenyl]methyl}oxy)-2-thiophenecarboxylate. ¹H NMR (400 MHz, DMSO-d₆) δ 8.52 (s, 1H), 7.97 (d, J=7.9 Hz, 1H), 7.84-7.77 (m, 2H), 7.68 (s, 1H), 7.62 (dd, J=7.3, 7.3 Hz, 1H), 7.34 (s, 1H), 7.28 (s, 1H), 5.54 (s, 2H), 4.02 (t, J=6.3 Hz, 2H), 3.86 (s, 3H), 3.79 (s, 3H), 3.41-3.29 (m, 4H), 2.21 (t, J=8.1 Hz, 2H), 1.99-1.88 (m, 4H). MS (ES+, m/z) 604 (m+1).

### EXAMPLE 134

### 5-(6-(Methyloxy)-5-{[3-(2-oxo-1-pyrrolidinyl)propyl]oxy}-1H-benzimidazol-1-yl)-3-({[2-(trifluoromethyl)phenyl]methyl}oxy)-2-thiophenecarboxamide

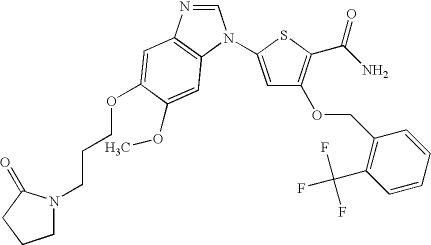

5-(6-(methyloxy)-5-{[3-(2-oxo-1-pyrrolidinyl)propyl]oxy}-1H-benzimidazol-1-yl)-3-({[2-(trifluoromethyl)phenyl]methyl}oxy)-2-thiophenecarboxamide was prepared from methyl 5-(6-(methyloxy)-5-{[3-(2-oxo-1-pyrrolidinyl)propyl]oxy}-1H-benzimidazol-1-yl)-3-({[2-(trifluoromethyl)phenyl]methyl}oxy)-2-thiophenecarboxylate using procedure similarly described in Example 61 except 7M NH₃ in MeOH was used instead of 2M NH₃ in MeOH. ¹H NMR (400 MHz, DMSO-d₆) δ 8.42 (s, 1H), 7.86-7.60 (m, 5H), 7.59 (s, 1H), 7.30 (s, 1H), 7.20 (s, 1H), 6.80 (br s, 1H), 5.54 (s, 2H), 3.99 (t, J=6.2 Hz, 2H), 3.82 (s, 3H), 3.39-3.28 (m, 4H), 2.18 (t, J=8.1 Hz, 2H), 1.97-1.85 (m, 4H). MS (ES+, m/z) 549 (m+1).

### INTERMEDIATE EXAMPLE 25

### Methyl 5-[6-{[3-(dimethylamino)propyl]oxy}-5-(methyloxy)-1H-benzimidazol-1-yl]-3-({[2-(trifluoromethyl) phenyl]methyl}oxy)-2-thiophenecarboxylate

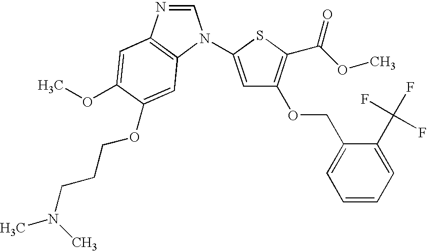

Methyl 5-[6-hydroxy-5-(methyloxy)-1H-benzimidazol-1-yl]-3-({[2-(trifluoromethyl)-phenyl]methyl}oxy)-2-thiophenecarboxylate (0.150 g, 0.313 mmol) and triphenylphosphine (0.361 g, 1.38 mmol) were stirred in 6 mL of dichloromethane. 3-Dimethylamino-1-propanol (0.13 mL, 1.1 mmol) was added via syringe, and the solution was cooled to 0° C. Diethyl azodicarboxylate (0.12 mL, 0.76 mmol) was added dropwise via syringe, and the solution was warmed to room temperature. After 3 hours the reaction was quenched by the addition of 2-3 mL of methanol. The reaction mixture was absorbed directly onto silica gel, and purification by flash chromatography afforded 0.112 g (63%) of methyl 5-[6-{[3-(dimethylamino)propyl]oxy}-5-(methyloxy)-1H-benzimidazol-1-yl]-3-({[2-(trifluoromethyl)phenyl]methyl}oxy)-2-thiophenecarboxylate. ¹H NM R (400 M Hz, DMSO-d₆) δ 8.51 (s, 1H), 7.97 (d, J=7.7 Hz, 1H), 7.84-7.77 (m, 2H), 7.67 (s, 1H), 7.62 (dd, J=7.5, 7.5 Hz, 1H), 7.35 (s, 1H), 7.29 (s, 1H), 5.53 (s, 2H), 4.06 (t, J=6.4 Hz, 2H), 3.84 (s, 3H), 3.79 (s, 3H), 2.47 (t, J=7.0 Hz, 2H), 2.21 (s, 6H), 1.91 (m, 2H). MS (ES+, m/z) 564 (m+1).

### EXAMPLE 135

### 5-[6-f [3-(Dimethylamino)propyl]oxy}-5-(methyloxy)-1H-benzimidazol-1-yl]-3-({[2-(trifluoromethyl)phenyl]methyl}oxy)-2-thiophenecarboxamide

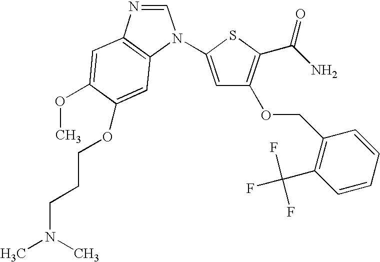

5-[6-{[3-(dimethylamino)propyl]oxy}-5-(methyloxy)-1H-benzimidazol-1-yl]-3-({[2-(trifluoromethyl)phenyl]methyl}oxy)-2-thiophenecarboxamide was prepared from methyl 5-[6-{[3-(dimethylamino)propyl]oxy}-5-(methyloxy)-1H-benzimidazol-1-yl]-3-({[2-(trifluoromethyl)phenyl]methyl}oxy)-2-thiophenecarboxylate using procedure similarly described in Example 61 except 7M NH₃ in MeOH was used instead of 2M NH₃ in MeOH.

¹H NMR (400 MHz, DMSO-d₆) δ 8.42 (s, 1H), 7.88-7.60 (m, 5H), 7.59 (s, 1H), 7.32 (s, 1H), 7.20 (s, 1H), 6.8 (br s, 1H), 5.53 (s, 2H), 4.02 (t, J=6.3 Hz, 2H), 3.81 (s, 3H), 2.35 (t, J=7.0 Hz, 2H), 2.11 (s, 6H), 1.86 (m, 2H). MS (ES+, m/z) 549 (m+1).

### INTERMEDIATE EXAMPLE 26

### Methyl 5-[6-[(2-chloroethyl)oxy]-5-(methyloxy)-1H-benzimidazol-1-yl]-3-({[2-(trifluoromethyl)phenyl]methyl}oxy)-2-thiophenecarboxylate

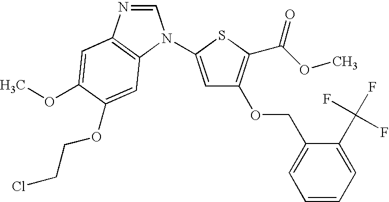

This compound was prepared in a similar manner to that previously described for the synthesis of methyl 5-[6-{[3-(dimethylamino)propyl]oxy}-5-(methyloxy)-1H-benzimidazol-1-yl]-3-({[2-(trifluoromethyl)phenyl]methyl}oxy)-2-thiophenecarboxylate. Reaction of methyl 5-[6-hydroxy-5-(methyloxy)-1H-benzimidazol-1-yl]-3-({[2-(trifluoromethyl)-phenyl]methyl}oxy)-2-thiophenecarboxylate (0.150 g, 0.313 mmol), triphenylphosphine (0.740 g, 2.82 mmol), 2-chloroethanol (0.13 mL, 1.9 mmol), and diethyl azodicarboxylate (0.25 mL, 1.6 mmol) provided 0.117 g (69010) of methyl 5-[6-[(2-chloroethyl)oxy]-5-(methyloxy)-1H-benzimidazol-1-yl]-3-({[2-(trifluoromethyl)phenyl]-methyl}oxy)-2-thiophenecarboxylate. ¹H NMR (400 MHz, DMSO-d₆) δ 8.52 (s, 1H), 7.95 (d, J=7.7 Hz, 1H), 7.83-7.75 (m, 2H), 7.67 (s, 1H), 7.60 (dd, J=7.9, 7.7 Hz, 1H), 7.37 (s, 1H), 7.31 (s, 1H), 5.51 (s, 2H), 4.30 (t, J=5.1 Hz, 2H), 3.97 (t, J=5.1 Hz, 2H), 3.84 (s, 3H), 3.77 (s, 3H). MS (ES+, m/z) 541 (m+1).

### INTERMEDIATE EXAMPLE 27

### 5-[6-[(2-Chloroethyl)oxy]-5-(methyloxy)-1H-benzimidazol-1-yl]-3-({[2-(trifluoromethyl)phenyl]methyl}oxy)-2-thiophenecarboxylic acid

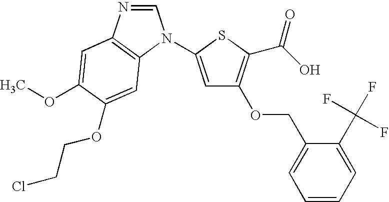

Methyl 5-[6-[(2-chloroethyl)oxy]-5-(methyloxy)-1H-benzimidazol-1-yl]-3-({[2-(trifluoromethyl)phenyl]-methyl}oxy)-2-thiophenecarboxylate (0.115 g, 0.213 mmol) was dissolved in 10 mL of methanol with stirring. A 1.0M lithium hydroxide solution (10 mL, 10 mmol) was added and the mixture was stirred for 24 hours. The reaction was judged incomplete so it was heated to 40° C. for an additional 24 hours. The reaction was cooled to room temperature and poured into 0.5N NaOH and diethyl ether. The layers were separated, and the aqueous layer was washed with diethyl ether. The diethyl ether layers were discarded, and the aqueous layer acidified with concentrated HCl. The aqueous layer was extracted with ethylacetate (2×) and dichloromethane. The combined organic layers were dried over MgSO₄, filtered, and concentrated to yield 0.0800 g (71%) of 5-[6-[(2-chloroethyl)oxy]-5-(methyloxy)-1H-benzimidazol-1-yl]-3-({[2-(trifluoromethyl)-phenyl]methyl}oxy)-2-thiophenecarboxylic acid as a white solid. ¹H NMR (400 MHz, DMSO-d₆) δ 12.85 (br s, 1H), 8.52 (s, 1H), 7.96 (d, J=7.5 Hz, 1H), 7.84-7.75 (m, 2H), 7.64-7.58 (m, 2H), 7.38 (s, 1H), 7.31 (s, 1H), 5.50 (s, 2H), 4.31 (t, J=5.1 Hz, 2H), 3.98 (t, J=5.1 Hz, 2H), 3.85 (s, 3H). MS (ES+, m/z) 527 (m+1).

### INTERMEDIATE EXAMPLE 28

### 5-[6-[(2-Chloroethyl)oxy]-5-(methyloxy)-1H-benzimidazol-1-yl]-3-({[2-(trifluoromethyl)phenyl]methyl}oxy)-2-thiophenecarboxamide

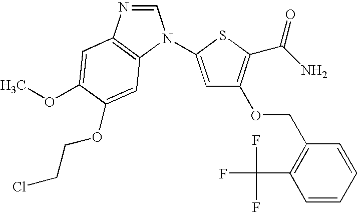

5-[6-[(2-Chloroethyl)oxy]-5-(methyloxy)-1H-benzimidazol-1-yl]-3-({[2-(trifluoro-methyl)phenyl]methyl}oxy)-2-thiophenecarboxylic acid (0.0790 g, 0.150 mmol) and ammonium chloride (0.0160 g, 0.299 mmol) were placed in a flask. 5 mL of N,N-dimethylformamide was added, and the mixture was stirred. N-Methylmorpholine (0.032 mL, 0.29 mmol) was added via syringe. 1-Hydroxybenzotriazole (0.0405 g, 0.300 mmol) was added in a single portion. 1-(3-Dimethylaminopropyl)-3-ethylcarbodiimide hydrochloride (0.0403 g, 0.210 mmol) was added, and the mixture was stirred for 64 hours. The reaction was poured into ethyl acetate and 1N HCl, and the layers were separated. The organic layer was washed with brine, and the combined aqueous layers were extracted with ethyl acetate. The combined organic layers were dried over MgSO₄, filtered, and concentrated. Purification by flash chromatography provided 0.0760 g (96%) of 5-[6-[(2-chloroethyl)oxy]-5-(methyloxy)-1H-benzimidazol-1-yl]-3-({[2-(trifluoromethyl)phenyl]methyl}oxy)-2-thiophenecarboxamide as an off-white solid. ¹H NMR (300 MHz, DMSO-d₆) δ 8.49 (s, 1H), 7.91-7.64 (m, 5H), 7.65 (s, 1H), 7.41 (s, 1H), 7.31 (s, 1H), 6.84 (br s, 1H), 5.59 (s, 2H), 4.34 (t, J=5.0 Hz, 2H), 4.02 (t, J=5.0 Hz, 2H), 3.88 (s, 3H).

### EXAMPLE 136

### 5-(5-(Methyloxy)-6-{[2-(4-methyl-1-piperazinyl)ethyl]oxy}-1H-benzimidazol-1-yl)-3-({[2-(trifluoromethyl)phenyl]methyl}oxy)-2-thiophenecarboxamide

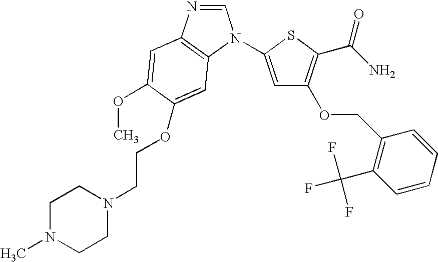

5-[6-[(2-Chloroethyl)oxy]-5-(methyloxy)-1H-benzimidazol-1-yl]-3-({[2-(trifluoromethyl)-phenyl]methyl}oxy)-2-thiophenecarboxamide (0.0750 g, 0.143 mmol) was dissolved in 3 mL of 1-methylpiperazine and heated to 90° C. with an oil bath. After 3 hours cool to room temperature and adsorb onto a mixture of NaHCO₃ and silica gel (1:5). The sample was purified by flash chromatography and concentrated in vacuo. The residue was dissolved in approximately 5 mL of methanol and 1 mL of 1N HCl in diethyl ether was added with swirling. Excess diethyl ether was added to induce precipitation of a white solid. The mixture was filtered, and the solid washed with diethyl ether. The solid was air dried and collected to provide 0.0496 g (52%) of 5-(5-(methyl oxy)-6-{[2-(4-methyl-1-piperazinyl)ethyl]oxy}-1H-benzimidazol-1-yl)-3-({[2-(trifluoromethyl)phenyl]methyl}-oxy)-2-thiophenecarboxamide as its di-HCl salt. For NMR analysis solid Na₂CO₃ was added to the NMR tube to free base the sample in situ. ¹H NMR (400 MHz, DMSO-d₆) δ 8.42 (s, 1H), 7.88-7.58 (m, 5H), 7.59 (s, 1H), 7.32 (s, 1H), 7.24 (s, 1H), 6.80 (br s, 1H), 5.54 (s, 2H), 4.10 (t, J=5.7 Hz, 2H), 3.81 (s, 3H), 2.69 (t, J=5.8 Hz, 2H), 2.48-2.15 (m, 8H), 2.10 (s, 3H). MS (ES+, m/z) 590 (m+1).

Unless otherwise noted, the following compounds were prepared according to general procedures outlined for Examples 134, 135 and 136 with appropriate intermediates.

### EXAMPLE 137

### 5-(5-(Methyloxy)-6-{[2-(4-morpholinyl)ethyl]oxy}-1H-benzimidazol-1-yl)-3-({[2-(trifluoromethyl)phenyl]methyl}oxy)-2-thiophenecarboxamide

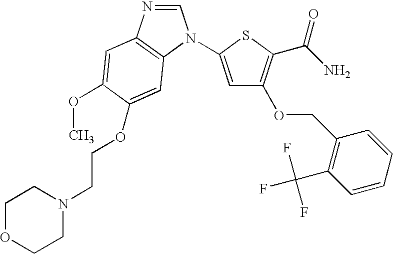

¹H NMR (400 MHz, DMSO-d₆) δ 8.44 (s, 1H), 7.87-7.63 (m, 5H), 7.62 (s, 1H), 7.34 (s, 1H), 7.27 (s, 1H), 6.82 (br s, 1H), 5.56 (s, 2H), 4.13 (t, J=5.9 Hz, 2H), 3.83 (s, 3H), 3.59-3.54 (m, 4H), 2.73 (t, J=5.9 Hz, 2H). MS (ES+, m/z) 577 (m+1).

### EXAMPLE 138

### 5-[6-(2-Morpholin-4-ylethoxy)-1H-benzimidazol-1-yl]-3-{[2-(trifluoromethyl)benzyl]oxy}thiophene-2-carboxamide

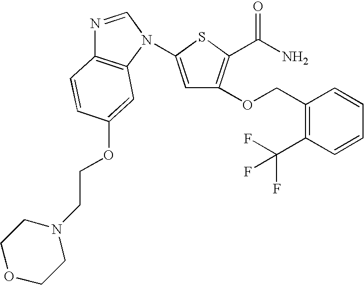

¹H NMR (400 MHz, DMSO-d₆) δ 8.50 (s, 1H), 7.86 (d, J=8.06 Hz, 2H), 7.79 (t, J=7.6 Hz, 1H), 7.73 (br s, 1H), 7.68 (s, 1H), 7.65 (d, J=6.41 Hz, 2H), 7.23 (d, J=1.65 Hz, 1H), 6.99 (dd, J=2.01 Hz, J=8.79 Hz, 1H), 6.82 (br s, 1H), 5.56 (s, 1H), 4.15 (t, J=5.58 Hz, 2H), 3.58 (t, J=4.39 Hz, 4H), 2.73 (t, J=5.58 Hz, 2H). MS (ES+, m/z) 547 (m+1).

### EXAMPLE 139

### 5-[6-(2-Pyrrolidin-1-ylethoxy)-1H-benzimidazol-1-yl]-3-{[2-(trifluoromethyl)benzyl]oxy}thiophene-2-carboxamide

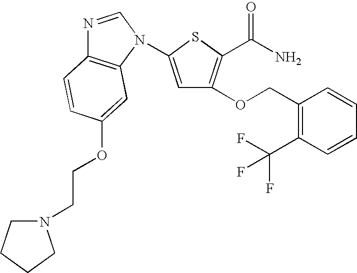

¹H NMR (400 MHz, DMSO-d₆) δ 8.50 (s, 1H), 7.86 (s, 1H), 7.84 (s, 1H), 7.79 (t, J=7.60 Hz, 1H), 7.73 (br s, 1H), 7.68-7.64 (m, 3H), 7.23 (d, J=1.83 Hz, 1H), 6.99 (dd, J=2.11 Hz, 8.70 Hz, 1H), 6.82 (br s, 1H), 5.56 (s, 2H), 4.14 (t, J=5.58 Hz, 2H), 2.85 (br s, 2H), 2.57 (br s, 4H), 1.70 (br s, 4H). MS (ES+, m/z) 531 (m+1).

### EXAMPLE 140

### 5-[5-Fluoro-6-(2-morpholin-4-ylethoxy)-1H-benzimidazol-1-yl]-3-{[2-(trifluoromethyl)benzyl]oxy}thiophene-2-carboxamide

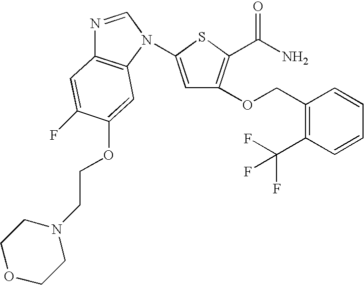

¹H NMR (400 MHz, DMSO-d₆) δ 8.54 (s, 1H), 7.85-7.63 (m, 7H), 7.43 (d, J=7.48 Hz, 1H), 6.83 (br s, 1H), 5.55 (s, 2H), 4.23 (t, J=5.64 Hz, 2H), 3.57 (t, J=4.43 Hz, 4H), 2.75 (t, J=5.64 Hz, 2H). MS (ES+, m/z) 565 (M+1).

### EXAMPLE 141

### 5-(5-Hydroxy-1H-benzimidazol-1-yl)-3-[(2-methylbenzyl)oxy]-thiophene-2-carboxylic acid

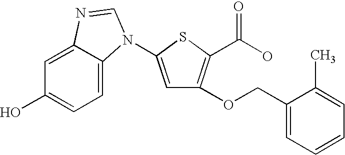

¹H NMR (400 MHz, CD₃OD) δ 9.64 (s, 1H); 7.69-7.63 (m, 2H); 7.49 (d, J=7.4 Hz, 1H); 7.24-7.18 (m, 5H); 5.37 (s, 2H); 2.43 (s, 3H). MS (ES+, m/z) 380 (M+).

### EXAMPLE 142

### 5-[5-(2-Methoxyethoxy)-1H-benzimidazol-1-yl]-3-[(2-methylbenzyl)oxy]thiophene-2-carboxamide

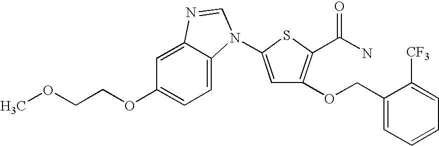

¹H NMR (400 MHz, CD₃OD) δ 8.45 (s, 1H); 7.83-7.78 (m, 2H); 7.73 (t, J=7.1 Hz, 1H); 7.65-7.59 (m, 2H); 7.43 (s, 1H); 7.29 (d, J=2.2 Hz, 1H); 7.10 (dd, J=2.2, 4.7 Hz, 1H); 5.58 (s, 2H); 4.20-4.18 (m, 2H); 3.80-3.78 (m, 2H); 3.44 (s, 3H). MS (ES+, m/z) 491 (M+).

### INTERMEDIATE EXAMPLE 29

### 1,1-Dimethylethyl 4-[({1-[5-[(methyloxy)carbonyl]-4-({[2-(trifluoromethyl)phenyl]methyl}oxy)-2-thienyl]-1H-benzimidazol-6-yl}oxy)methyl]-1-piperidinecarboxylate

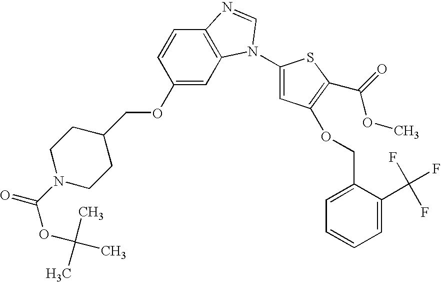

Methyl 5-(6-hydroxy-1H-benzimidazol-1-yl)-3-({[2-(trifluoromethyl)-phenyl]methyl}-oxy)-2-thiophenecarboxylate (0.150 g, 0.335 mmol) and 1,1-dimethylethyl 4-({[(4-methyl phenyl)sulfonyl]oxy}methyl)-1-piperidinecarboxylate (0.161 g, 0.436 mmol) were dissolved in 5 mL of N,N-dimethylformamide with stirring. Cesium carbonate (0.164 g, 0.503 mmol) was added in a single portion, and the reaction was heated to 60° C. with an oil bath. The reaction was stirred at this temperature for seven hours and cooled to room temperature. The mixture was poured into water and ethyl acetate, and the layers were separated. The organic layer was washed with brine, and the combined aqueous layers were extracted with ethyl acetate. The combined organic layers were dried over MgSO₄, filtered, and concentrated in vacuo. The residue was purified by flash chromatography to provide 0.186 g (86%) of 1,1-dimethylethyl 4-[({1-[5-[(methyloxy)carbonyl]-4-({[2-(trifluoromethyl)phenyl]methyl}oxy)-2-thienyl]-1H-benzimidazol-6-yl}oxy)methyl]-1-piperidinecarboxylate. ¹H NMR (400 MHz, DMSO-d₆) 58.59 (s, 1H), 8.00 (d, J=8.0 Hz, 1H), 7.87-7.79 (m, 2H), 7.74-7.60 (m, 3H), 7.28 (d, J=2.1 Hz, 1H), 7.03 (dd, J=8.8, 2.2 Hz, 1H), 5.56 (s, 2H), 4.02 (m, 2H), 3.95 (d, J=6.5 Hz, 1H), 3.82 (s, 3H), 2.78 (br s, 1H), 2.00 (br s, 1H), 1.87-1.76 (m, 2H), 1.43 (s, 9H), 1.84-1.12 (m, 2H). MS (ES+, m/z) 646 (m+1).

### EXAMPLE 143

### 5-{6-[(4-Piperidinylmethyl)oxy]-1H-benzimidazol-1-yl}-3-({[2-(trifluoromethyl)phenyl]-methyl}oxy)-2-thiophenecarboxamide

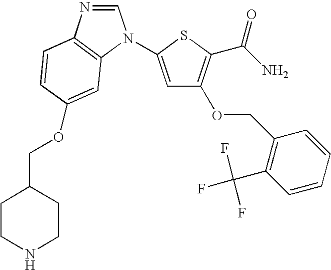

1,1-Dimethylethyl 4-[({1-[5-(aminocarbonyl)-4-({[2-(trifluoromethyl)phenyl]-methyl}-oxy)-2-thienyl]-1H-benzimidazol-6-yl}oxy)methyl]-1-piperidinecarboxylate was dissolved in 7 mL of methanol with stirring. 4 mL of concentrated HCl was added and the solution was heated to 45° C. for 1 hour. The solution was cooled to room temperature and concentrated in vacuo to afford 0.0866 g (87%) of 5-{6-[(4-piperidinylmethyl)oxy]-1H-benzimidazol-1-yl}-3-({[2-(trifluoromethyl)phenyl]-methyl}oxy)-2-thiophenecarboxamide as its HCl salt. For ¹H NMR analysis solid Na₂CO₃ was added to the NMR tube to free base the sample in situ. ¹H NMR (400 MHz, DMSO-d₆) δ 8.47 (s, 1H), 7.85-7.81 (m, 2H), 7.80-7.71 (m, 2H), 7.67-7.60 (m, 3H), 7.16 (d, J=2.2 Hz, 1H), 6.96 (dd, J=8.8, 2.2 Hz, 1H), 6.81 (brs, 1H), 5.54 (s, 2H), 4.09 (m, 2H), 3.89-3.81 (m, 2H), 2.93 (d, J=10.6 Hz, 1H), 1.83 (br s, 1H), 1.73-1.62 (m, 2H), 1.27-1.05 (m, 2H). MS (ES+, m/z) 531 (m+1).

### EXAMPLE 144

### 5-(1H-Benzimidazol-1-yl)-3-(benzyloxy)-N-hydroxythiophene-2-carboxamide

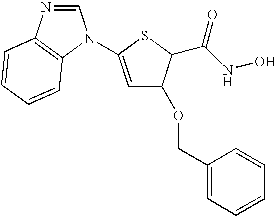

To a cooled (0° C.) solution of 5-(1H-benzimidazol-1-yl)-3-(benzyloxy)thiophene-2-carboxylic acid (100 mg, 0.28 mmol) in dichloromethane (2.0 mL) was added dimethylformamide (22 microL, 0.28 mmol) followed by a 2.0M solution of oxalyl chloride in dichloromethane (310 microL, 0.62 mmol). The reaction was stirred at 0° C. for 40 minutes then added to a solution of hydroxylamine hydrochloride (78 mg, 1.12 mmol) and triethylamine (233 microL, 1.67 mmol) in 85:15 tetrahydrofuran/H₂O (1 mL). The reaction was stirred at room temperature for 45 minutes then poured into 1M aqueous HCl and extracted with dichloromethane. The organic extracts were washed with brine and dried over Na₂SO₄. Filtration and concentration followed by reverse-phase PREP HPLC (30-to-70% acetonitrile/H₂O with 0.1% formic acid) gave 5-(1H-benzimidazol-1-yl)-3-(benzyloxy)-N-hydroxythiophene-2-carboxamide (10 mg, 10%) as an off-white solid. ¹H NMR (400 MHz, CDCl₃) δ 9.51 (s, 1H), 8.08 (s, 1H), 8.89-8.84 (m, 1H), 7.60-7.56 (m, 1H), 7.47-7.37 (m, 8H), 6.95 (s, 1H), 5.30 (s, 2H). MS (ES+, m/z) 365 (m+1).

### EXAMPLE 145

### 5-(5,6-Dimethoxy-1H-benzimidazol-1-yl)-3-{[2-(trifluoromethyl)-benzyl]oxy}thiophene-2-carbothioamide

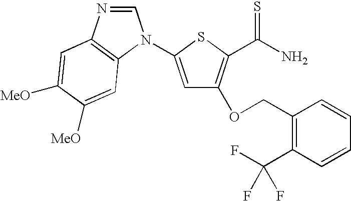

To a solution of 5-(5,6-dimethoxy-1H-benzimidazol-1-yl)-3-{[2-(trifluoromethyl)benzyl]oxy}thiophene-2-carboxamide (50 mg, 0.10 mmol) in 1,4-dioxane (1.5 mL) was added Lawesson's Reagent (32 mg, 0.08 mmol). The reaction was heated to 80° C. for 3 hrs, cooled to room temperature and additional Lawesson's Reagent was added (32 mg, 0.08 mmol). The reaction was heated to 80° C. for 2 hrs then cooled to room temperature. The reaction was poured into aqueous 1M HCl and extracted with dicholormethane. The organic extracts were dried over Na₂SO₄. Filtration and concentration followed by reverse-phase PREP HPLC (30-to-70% acetonitrile/H₂O with 0.1% formic acid) gave 5-(5,6-dimethoxy-1H-benzimidazol-1-yl)-3-{[2-(trifluoromethyl)benzyl]oxy}thiophene-2-carbothioamide (25 mg, 48%) as a light yellow solid. ¹H NMR (400 MHz, DMSO-d₆) δ 9.63 (s, 1H), 8.35 (m, 2H), 7.76-7.65 (m, 3H), 7.56-7.51 (m, 1H), 7.47 (s, 1H), 7.23 (s, 1H), 7.11 (s, 1H), 5.49 (s, 2H), 3.72 (s, 3H), 3.71 (s, 3H). MS (ES+, m/z) 493 (m+1).

### EXAMPLE 146

### 5-(5,6-Dimethoxy-1H-benzimidazol-1-yl)-3-{[2-(trifluoromethyl)benzyl]oxy}thiophene-2-carbonitrile

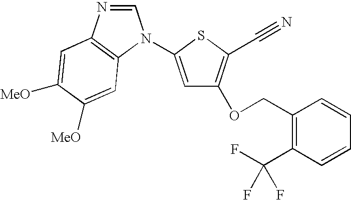

To a solution of 5-(5,6-dimethoxy-1H-benzimidazol-1-yl)-3-{[2-(trifluoromethyl)benzyl]oxy}thiophene-2-carboxamide (150 mg, 0.31 mmol) in dichloromethane (2 mL) was added 2-chloro-1,3-dimethylimidazolinium chloride (120 mg, 0.71 mmol) and trifluoroacetic acid (50 microL, 0.65 mmol). To this solution was added triethylamine (200 microL, 1.44 mmol). The mixture was stirred 18 hrs, then additional 2-chloro-1,3-dimethylimidazolinium chloride (120 mg, 0.71 mmol) and trifluoroacetic acid (50 microL, 0.65 mmol) were added, followed by triethylamine (200 microL, 1.44 mmol). The mixture was stirred for 4 hrs then poured into H₂O and extracted with dichloromethane. The organic extracts were washed with aqueous 5% HCl, aqueous (saturated) NaHCO₃, brine and dried over Na₂SO₄. Filtration and concentration followed by silica gel chromatography (eluting with a 40-to-95% EtOAc/hexane gradient) gave 5-(5,6-dimethoxy-1H-benzimidazol-1-yl)-3-{[2-(trifluoromethyl)benzyl]oxy}thiophene-2-carbonitrile (66 mg, 46%) as a yellow solid. ¹H NMR (400 MHz, CDCl₃) δ 7.90 (s, 1H), 7.80-7.72 (m, 2H), 7.66 (t, J=7.60 Hz, 1H), 7.51 (t, J=7.51 Hz, 1H), 7.31 (s, 1H), 6.99 (s, 1H), 6.82 (s, 1H), 5.55 (s, 2H), 3.96 (s, 3H), 3.92 (s, 3H). MS (ES+, m/z) 459 (m+1).

### EXAMPLE 147

### 5,6-Dimethoxy-1-(5-(1H-tetraazol-5-yl)-4-{[2-(trifluoromethyl)-benzyl]oxy}thien-2-yl)-1H-benzimidazole

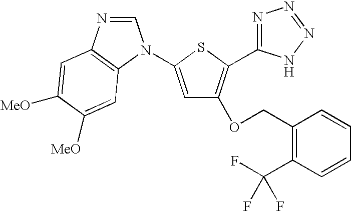

To a Smithcreator Microwave reaction vessal was added 5-(5,6-dimethoxy-1H-benzimidazol-1-yl)-3-{[2-(trifluoromethyl)benzyl]oxy}thiophene-2-carbonitrile (53 mg, 0.11 mmol), sodium azide (20 mg, 0.31 mmol), ammonium chloride (16 mg, 0.31 mmol) and dimethylformamide (2.0 mL). The reaction vessal was sealed and heated at 120° C. for 20 minutes in the Smithereator Microwave. The reaction vessal was cooled to room temperature, opened, and additional sodium azide (20 mg, 0.31 mmol) and ammonium chloride (16 mg, 0.31 mmol) was added. The vessal was sealed and heated at 120° C. for 10 minutes on the microwave, then cooled to room temperature and opened. The mixture was poured into aqueous (saturated) NaHCO₃ and washed with diethyl ether. The aqueous layer was then acidified to pH 1.0 by addition of concentrated HCl, then extracted with ethyl acetate. The organic extract was washed with brine and dried over Na₂SO₄. Filtration and concentration followed by reverse-phase PREP HPLC (30-to-70% acetonitrile/H₂O with 0.1% formic acid) gave 5,6-dimethoxy-1-(5-(1H-tetraazol-5-yl)-4-{[2-(trifluoromethyl)benzyl]oxy}thien-2-yl)-1H-benzimidazole (25 mg, 43%) as a white solid. ¹H NMR (400 MHz, CDCl₃) δ 7.97 (s, 1H), 7.81 (d, J=7.87 Hz, 1H), 7.69-7.56 (m, 3H), 7.32 (s, 1H), 7.08 (s, 1H), 6.99 (s, 1H), 5.57 (s, 2H), 3.95 (s, 3H), 3.93 (s, 3H). MS (ES+, m/z) 502 (m+1).

### INTERMEDIATE EXAMPLE 30

### Methyl 3-hydroxy-5-[2-(methylthio)-1H-benzimidazol-1-yl]thiophene-2-carboxylate

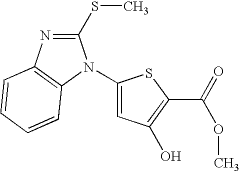

A mixture of 2-(methylthio)-1H-benzimidazole (5.0 g, 25.9 mmol) and methyl 2-chloro-3-oxo-2,3-dihydrothiophene-2-carboxylate (8.53 g, 51.9 mmol) were dissolved in chloroform (100 mL) and glacial acetic acid (12 mL). Stirred at room temperature for 72 hrs. Poured reaction mixture into separatory funnel containing dichloromethane (150 mL), washed with distilled water (2×100 mL). Extracted combined aqueous layers with dichloromethane (2×50 mL). Washed combined organic layers with distilled water (3×100 mL). Dried organic layer (MgSO₄), filtered and concentrated under reduced pressure. Dissolved residue in dichloromethane and methanol, and added silica gel (35 g). Following evaporation of the volatiles under reduced pressure, the pre-adsorbed solids were loaded into a solid loading cartridge and subjected to an isocratic elution with dichloromethane (100%) using a RediSep silica gel cartridge (330 g; ISCO). The appropriate fractions were combined and concentrated under reduced pressure to give methyl 3-hydroxy-5-[2-(methylthio)-1H-benzimidazol-1-yl]thiophene-2-carboxylate (4.75 g) as an off-white solid. ¹H NMR (400 MHz, CDCl₃): δ 9.77 (s, 1H), 7.71-7.68 (m, 1H), 7.36-7.34 (m, 1H), 7.30-7.26 (m, 1H), 7.24-7.20 (m, 1H), 6.87 (s, 1H), 3.93 (s, 3H), 2.78 (s, 3H). MS (ES+, m/z) 321 (M+1).

### INTERMEDIATE EXAMPLE 31

### Methyl 5-[2-(methylthio)-1H-benzimidazol-1-yl]-3-{[2-(trifluoromethyl)benzyl]oxy}thiophene-2-carboxylate

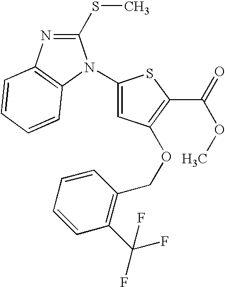

In a similar manner as described for Example 54, 3-hydroxy-5-[2-(methylthio)-1H-benzimidazol-1-yl]thiophene-2-carboxylate (4.5 g, 14.0 mmol) and 1-(bromomethyl)-2-(trifluoromethyl)benzene (3.36 g, 14.0 mmol) gave methyl 5-[2-(methylthio)-1H-benzimidazol-1-yl]-3-{[2-(trifluoromethyl)benzyl]oxy}thiophene-2-carboxylate (5.99 g) as a tan solid. ¹H NMR (400 MHz, CDCl₃): δ 7.93 (d, J=7.7 Hz, 1H), 7.8-7.76 (m, 2H), 7.65-7.58 (m, 3H), 7.37-7.34 (m, 1H), 7.29-7.21 (m, 2H), 5.46 (s, 2H), 3.77 (s, 3H), 2.71 (s, 3H). MS (ES+, m/z) 479 (M+1).

### INTERMEDIATE EXAMPLE 32

### Methyl 5-[2-(methylsulfonyl)-1H-benzimidazol-1-yl]-3-{[2-(trifluoromethyl)benzyl]oxy}thiophene-2-carboxylate

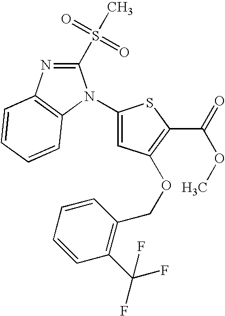

To a solution of methyl 5-[2-(methylthio)-1H-benzimidazol-1-yl]-3-{[2-(trifluoromethyl)benzyl]oxy}thiophene-2-carboxylate (150 mg, 0.31 mmol) in dichloromethane (5 mL) under nitrogen atmosphere was added 3-chloroperoxybenzoic acid (77%) (178 mg, 0.79 mmol) and stirred at room temperature for 24 hours. Concentrated under reduced pressure to give an off-white solid. Dissolved in chloroform (100 mL) and poured reaction mixture into separatory funnel. Washed with saturated NaHCO₃ aqueous solution (2×50 mL), and brine (2×50 mL). Dried organic layer (MgSO₄), filtered and concentrated under reduced pressure to give a gold oil. Dissolved in dichloromethane (25 mL) and added silica gel (500 mg), followed by evaporation of the volatiles under reduced pressure. The pre-adsorbed solids were loaded into a solid loading cartridge and subjected to a gradient elution using ethyl acetate:hexanes (20:80) to ethyl acetate:hexanes (50:50) using a RediSep silica gel cartridge (12 g; ISCO). The appropriate fractions were combined and concentrated under reduced pressure to give methyl 5-[2-(methylsulfonyl)-1H-benzimidazol-1-yl]-3-{[2-(trifluoromethyl)benzyl]oxy}thiophene-2-carboxylate (130 mg) as a white solid. ¹H NMR (400 MHz, CDCl₃): δ 7.97 (d, J=7.8 Hz, 1H), 7.89-7.86 (m, 1H), 7.69-7.62 (m, 2H), 7.49-7.39 (m, 4H), 7.16 (s, 1H), 5.46 (s, 2H), 3.91 (3, 3H), 3.50 (s, 3H). MS (ES+, m/z) 511 (M+1).

### INTERMEDIATE EXAMPLE 33

### 5-[2-(Methylthio)-1H-benzimidazol-1-yl]-3-{[2-(trifluoromethyl) benzyl]oxy}thiophene-2-carboxamide

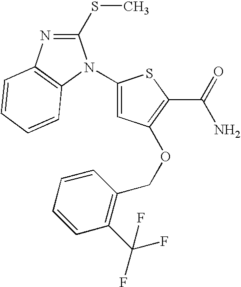

In a similar manner as described for Example 61, methyl 5-[2-(methylthio)-1H-benzimidazol-1-yl]-3-{[2-(trifluoromethyl)benzyl]oxy}thiophene-2-carboxylate (160 mg, 0.343 mmol) and 7N NH₃ in methanol (10 mL, 70.0 mmol) gave 5-[2-(methylthio)-1H-benzimidazol-1-yl]-3-{[2-(trifluoromethyl)benzyl]oxy}thiophene-2-carboxamide (136 mg) as a white solid. ¹H NMR (400 MHz, DMSO-d₆): δ 7.84-7.75 (m, 4H), 7.65-7.62 (m, 2H), 7.56 (s, 1H), 7.32-7.30 (m, 1H), 7.28-7.20 (m, 2H), 6.87 (bs, 1H), 5.50 (s, 2H), 2.70 (s, 3H). MS (ES+, m/z) 464 (M+1).

### Intermediate Exmaple 34

### 5-[2-(Methylsulfonyl)-1H-benzimidazol-1-yl]-3-{[2-(trifluoromethyl)benzyl]oxy}thiophene-2-carboxamide

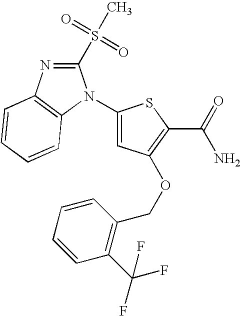

To a solution of 5-[2-(methylthio)-1H-benzimidazol-1-yl]-3-{[2-(trifluoromethyl)benzyl]oxy}thiophene-2-carboxamide (1.25 g, 2.69 mmol) in dichloromethane (50 mL) under nitrogen atmosphere was added 3-chloroperoxybenzoic acid (77%) (1.86 g, 8.29 mmol) and stirred at room temperature for 24 hours. Concentrated under reduced pressure to give an off-white solid. Dissolved in dichloromethane and methanol, added silica gel (10.0 g), followed by evaporation of the volatiles under reduced pressure. The pre-adsorbed solids were loaded into a solid loading cartridge and subjected to a gradient elution using ethyl acetate:hexanes (15:85) to ethyl acetate:hexanes (60:40) using a RediSep silica gel cartridge (40 g; ISCO). The appropriate fractions were combined and concentrated under reduced pressure to give 5-[2-(methylsulfonyl)-1H-benzimidazol-1-yl]-3-{[2-(trifluoromethyl)benzyl]oxy}thiophene-2-carboxamide (869 mg) as an off-white solid. ¹H NMR (400 MHz, CDCl₃): δ 7.89-7.87 (m, 1H), 7.76-7.74 (m, 1H), 7.66-7.61 (m, 2H), 7.54-7.44 (m, 4H), 7.25 (s, 1H), 7.02 (bs, 1H), 5.69 (bs, 1H), 5.44 (s, 2H), 3.51 (s, 3H). MS (ES+, m/z) 496 (M+1).

### EXAMPLE 148

### 5-(2-Amino-1H-benzimidazol-1-yl)-3-{[2-(trifluoromethyl)benzyl]oxy}thiophene-2-carboxamide

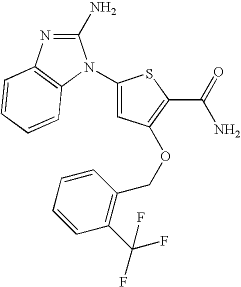

Method A: In a sealed tube, a mixture of 5-[2-(methylsulfonyl)-1H-benzimidazol-1-yl]-3-{[2-(trifluoromethyl)benzyl]oxy}thiophene-2-carboxamide (410 mg, 0.827-mmol) in 7N NH₃ in methanol (20 mL, 140 mmol) was heated to 80° C. for 24 hours. Cooled reaction mixture to room temperature, and filtered precipitate over glass-fritted funnel. The filtrate was concentrated under reduced pressure to give a solid residue (180 mg), which was dissolved in methanol and dichloromethane. Added silica gel (250 mg), followed by evaporation of the volatiles under reduced pressure. The pre-adsorbed solids were loaded into a solid loading cartridge and subjected to a gradient elution using dichloromethane:methanol (100:0) to dichloromethane:methanol (85:15) using a RediSep silica gel cartridge (4 g; ISCO). The appropriate fractions were combined and concentrated under reduced pressure to give 5-(2-amino-1H-benzimidazol-1-yl)-3-{[2-(trifluoromethyl)benzyl]oxy}thiophene-2-carboxamide (25 mg) as a tan solid. ¹H NMR (400 MHz, DMSO-d₆): δ 8.83 (s, 2H), 7.90-7.75 (m, 4H), 7.65-7.62 (m, 2H), 7.42 (d, J=7.9 Hz, 1H), 7.34-7.23 (m, 2H), 7.17 (d, J=8.6 Hz, 1H), 6.93 (bs, 1H), 5.47 (s, 2H). MS (ES+, m/z) 433 (M+1).

Method B: In a sealed tube, a mixture of methyl 5-[2-(methylsulfonyl)-1H-benzimidazol-1-yl]-3-{[2-(trifluoromethyl)benzyl]oxy}thiophene-2-carboxylate in 7N NH₃ in methanol were reacted together to give the 5-(2-amino-1H-benzimidazol-1-yl)-3-{[2-(trifluoromethyl)benzyl]oxy}thiophene-2-carboxamide.

### EXAMPLE 149

### Methyl 5-(5,6-dimethoxy-1H-benzimidazol-1-yl)-3-{[(2-nitrophenyl)sulfonyl]oxy}thiophene-2-carboxylate

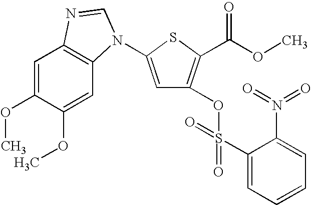

To a solution of 5-(5,6-dimethoxy-1H-benzimidazol-1-yl)-3-hydroxythiophene-2-carboxamide (170 mg, 0.50 mmol) and N,N-diisopropylethylamine (0.12 mL, 0.70 mmol) in dichloromethane (5 mL) was added 2-nitrobenzenesulfonyl chloride (130 mg, 0.60 mmol). The solution was stirred 1 h, at which time silica gel (5 g) was added. The volatiles were evaporated under reduced pressure, and the pre-adsorbed solids were loaded into a solid loading cartridge and subjected to a gradient elution using hexanes:ethyl acetate (80:20) to hexanes:ethyl acetate (0:100) using a RediSep silica gel cartridge (4 g; ISCO). The appropriate fractions were combined and concentrated under reduced pressure to give methyl 5-(5,6-dimethoxy-1H-benzimidazol-1-yl)-3-{[(2-nitrophenyl)sulfonyl]oxy}thiophene-2-carboxylate (240 mg) as a white solid. ¹H NMR (400 MHz, CDCl₃) δ 8.31 (dd, J=8.0, 1.5 Hz, 1H), 7.96 (s, 1H), 7.91-7.81 (m, 3H), 7.32 (s, 1H), 7.19 (s, 1H), 7.15 (s, 1H), 3.98 (s, 3H), 3.97 (s, 3H), 3.76 (s, 3H). MS (ES+, m/z) 520 (m+1).

### EXAMPLE 150

### Methyl 5-(5,6-dimethoxy-1H-benzimidazol-1-yl)-3-{[(trifluoromethyl)sulfonyl]oxy}thiophene-2-carboxylate

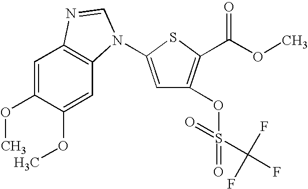

Compound was prepared according to general procedure outlined for Example 30. ¹H NMR (400 MHz, DMSO-d₆) δ 8.52 (s, 1H), 7.84 (s, 1H), 7.35 (s, 1H), 7.26 (m, 3H), 3.89 (s, 3H), 3.84 (s, 3H), 3.81 (s, 3H). MS (ES+, m/z) 467 (m+1).

### EXAMPLE 151

### 5-(5,6-Dimethoxy-1H-benzimidazol-1-yl)-3-{[(2-methylphenyl)sulfonyl]oxy}thiophene-2-carboxylic acid

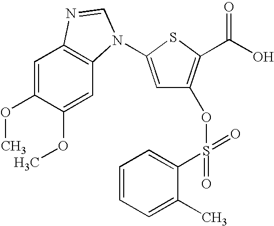

To a solution of methyl 5-(5,6-dimethoxy-1H-benzimidazol-1-yl)-3-{[(2-methylphenyl)sulfonyl]oxy}thiophene-2-carboxylate (100 mg, 0.20 mmol) in tetrahydrofuran (2 mL) was added 0.1N NaOH (2 mL, 0.20 mmol). The solution was stirred 1 h, at which time the solution was neutralized by the addition of 0.1N HCl (2 mL, 0.20 mmol), and a white solid precipitated. Vacuum filtration provided 5-(5,6-dimethoxy-1H-benzimidazol-1-yl)-3-{[(2-methyl phenyl)sulfonyl]oxy}thiophene-2-carboxylic acid (7 mg) as a white solid. ¹H NMR (400 MHz, CDCl₃) δ 9.50 (s, 1H), 7.88 (d, J=8.0 Hz, 1H), 7.54 (dd, J=8.3, 7.1 Hz, 1H), 7.49 (s, 1H), 7.42 (d, J=9.3 Hz, 1H), 7.32-7.27 (m 1H), 7.19 (s, 1H), 7.15 (s, 1H), 4.03 (s, 3H), 4.02 (s, 3H), 2.81 (s, 3H). MS (ES+, m/z) 475 (m+1).

### EXAMPLE 152

### 5-(5,6-Dimethoxy-1H-benzimidazol-1-yl)-3-hydroxythiophene-2-carboxamide trifluoroacetate

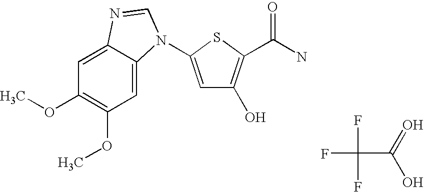

To solid 5-(5,6-dimethoxy-1H-benzimidazol-1-yl)-3-[(4-methoxybenzyl)oxy]thiophene-2-carboxamide (400 mg, 0.91 mmol) was added trifluoroacetic acid (2 mL). The bright red solution was stirred 10 minutes, at which time ether (20 mL) was added, and a pink solid precipitated. Vacuum filtration provided 5-(5,6-dimethoxy-1H-benzimidazol-1-yl)-3-hydroxythiophene-2-carboxamide trifluoroacetate (300 mg) as a pink solid. ¹H NMR (400 MHz, DMSO-d₆) δ 8.70 (s, 1H), 7.33 (s, 1H), 7.23 (s, 1H), 7.10 (s, 1H), 7.05 (br s, 1H), 3.83 (s, 3H), 3.82 (s, 3H). MS (ES+, m/z) 320 (m+1).

### EXAMPLE 153

### 2-(Aminocarbonyl)-5-(5,6-dimethoxy-1H-benzimidazol-1-yl)thien-3-yl 2-nitrobenzenesulfonate

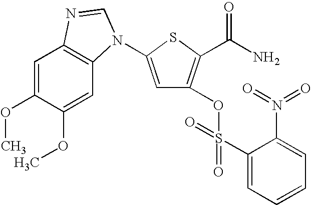

To a solution 5-(5,6-dimethoxy-1H-benzimidazol-1-yl)-3-hydroxythiophene-2-carboxamide trifluoroacetate (44 mg, 0.10 mmol) and N,N-diisopropylethylamine (0.058 mL, 0.33 mmol) in dichloromethane (2 mL) was added 2-nitrobenzenesulfonyl chloride (24 mg, 0.11 mmol). The solution was stirred 3 h, at which time silica gel (2 g) was added. The volatiles were evaporated under reduced pressure, and the pre-adsorbed solids were loaded into a solid loading cartridge and subjected to a gradient elution using ethyl acetate (100%) to ethyl acetate:methanol (80:20) using a RediSep silica gel cartridge (4 g; ISCO). The appropriate fractions were combined and concentrated under reduced pressure to give methyl 2-(aminocarbonyl)-5-(5,6-dimethoxy-1H-benzimidazol-1-yl)thien-3-yl 2-nitrobenzenesulfonate (37 mg) as a white solid. ¹H NMR (400 MHz, DMSO-d₆) δ 8.38 (s, 1H), 8.22-7.93 (m, 4H), 7.80 (br s, 1H), 7.40 (s, 1H), 7.34 (br s, 1H), 7.33 (s, 1H), 7.15 (s, 1H), 3.82 (s, 3H), 3.81 (s, 3H). MS (ES+, m/z) 505 (m+1).

### EXAMPLE 154

### 2-(Aminocarbonyl)-5-(5,6-dimethoxy-1H-benzimidazol-1-yl)thien-3-yl 2-methylbenzenesulfonate

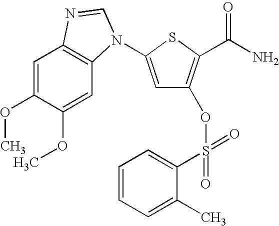

2-(Aminocarbonyl)-5-(5,6-dimethoxy-1H-benzimidazol-1-yl)thien-3-yl 2-methylbenzenesulfonate was prepared using similar procedure described above for the preparation of 2-(aminocarbonyl)-5-(5,6-dimethoxy-1H-benzimidazol-1-yl)thien-3-yl 2-nitrobenzenesulfonat except 2-methylsulfonyl chloride was used instead of 2-nitrobenzenesulfonyl chloride. ¹H NMR (400 MHz, DMSO-d₆) δ 8.35 (s, 1H), 7.91 (dd, J=8.0, 1.2 Hz, 1H), 7.79, (br s, 1H), 7.72 (ddd, J=7.7, 7.4, 1.3 Hz, 1H), 7.56 (d, J=7.4 Hz, 1H), 7.45 (dd, J=7.7, 7.7 Hz, 1H), 7.34 (br s, 1H), 7.32 (s, 1H), 7.15 (s, 1H), 7.05 (s, 1H), 3.80 (s, 3H), 3.80 (s, 3H), 2.68 (s, 3H). MS (ES+, m/z) 474 (m+1).

### INTERMEDIATE EXAMPLE 35

### 1-(5-(Methoxycarbonyl)-4-{[2-(trifluoromethyl)benzyl]oxy}thien-2-yl)-1H-benzimidazole-5-carboxylic acid

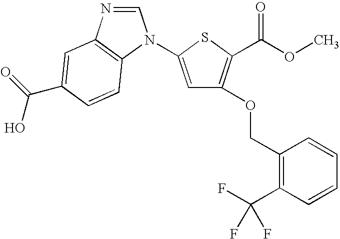

To a solution of vinyl 1-(5-(methoxycarbonyl)-4-{[2-(trifluoromethyl)benzyl]oxy}thien-2-yl)-1H-benzimidazole-5-carboxylate (500 mg, 0.97 mmol) in tetrahydrofuran (3.0 mL) was added morpholine (178 microL, 2.04 mmol) followed by tetrakis(triphenylphosphine)-palladium (0) (56 mg, 0.05 mmol). The reaction was stirred at room temperature for 1 hour then poured into 0.5M aqueous HCl and ethyl acetate. The organic layer was washed with water, brine, and dried over Na₂SO₄. Filtration and concentration gave 1-(5-(methoxycarbonyl)-4-{[2-(trifluoromethyl)benzyl]oxy}thien-2-yl)-1H-benzimidazole-5-carboxylic acid (455 mg, 98%) as a tan solid. ¹H NMR (400 MHz, DMSO-d₆) δ 13.02 (b, 1H), 8.87 (s, 1H), 8.33 (s, 1H), 8.03 (dd, J=8.60 and 1.46 Hz, 1H), 7.92-7.98 (m, 2H), 7.77-7.83 (m, 3H), 7.59-7.64 (m, 1H), 5.51 (s, 2H), 3.78 (s, 3H). MS (ES+, m/z) 476 (m+1).

### EXAMPLE 155

### 1-(5-(Aminocarbonyl)-4-{[2-(trifluoromethyl)benzyl]oxy}thien-2-yl)-N-[2-(methylsulfonyl)ethyl]-1H-benzimidazole-5-carboxamide

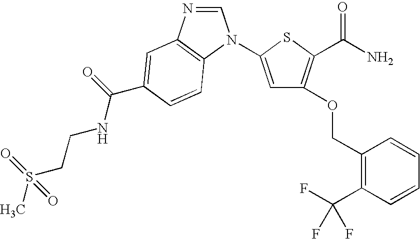

To a solution of 1-(5-(methoxycarbonyl)-4-{[2-(trifluoromethyl)benzyl]oxy}thien-2-yl)-1H-benzimidazole-5-carboxylic acid (35 mg, 0.073 mmol), 2-(methylsulfonyl)ethanamine (14 mg, 0.11 mmol) and diisopropylethylamine (35 microL, 0.20 mmol) in dimethylformamide (1.0 mL) was added [0-(7-azabenzotriazol-1-yl)-1,1,3,3-tetramethyluronium hexafluorophosphate] (35 mg, 0.092 mmol). The reaction was stirred for 12 hours then poured into aqueous saturated NaHCO₃ and extracted with ethyl acetate. The combined organics were washed with water, brine, and dried over Na₂SO₄. Filtration and concentration gave crude methyl 5-[5-({[2-(methylsulfonyl)ethyl]amino}carbonyl)-1H-benzimidazol-1-yl]-3-{[2-(trifluoromethyl)benzyl]oxy}thiophene-2-carboxylate (40 mg, 95%) as a light brown oil. The oil was stirred as a solution in 7 M ammonia in methanol (10 mL, 70 mmol), at 80° C. in a sealed, thick-walled glass pressure tube for 16 hours. The reaction was cooled to room temperature, concentrated and purified by reverse-phase PREP HPLC (10-90% gradient of acetonitrile/H₂O with 0.1% formic acid) to give 1-(5-(aminocarbonyl)-4-[2-(trifluoromethyl)benzyl]oxy}thien-2-yl)-N-[2-(methylsulfonyl)ethyl]-1H-benzimidazole-5-carboxamide (23 mg, 55%) as a white solid. ¹H NMR (400 MHz, DMSO-d₆) δ 8.84 (t, J=5.58 Hz, 1H), 8.76 (s, 1H), 8.30 (s, 1H), 7.94 (dd, J=8.60 and 1.28 Hz, 1H), 7.70-7.89 (m, 6H), 7.65 (t, J=7.60 Hz, 1H), 6.79 (b, 1H), 5.55 (s, 2H), 3.71 (q, J=6.41 Hz 2H), 3.41 (t, J=6.87 Hz, 2H), 3.04 (s, 3H). MS (ES+, m/z) 566 (m+1).

### EXAMPLE 156

### 1-(5-(Aminocarbonyl)-4-{[2-(trifluoromethyl)benzyl]oxy}thien-2-yl)-N-[2-(2-oxoimidazolidin-1-yl)ethyl]-1H-benzimidazole-5-carboxamide

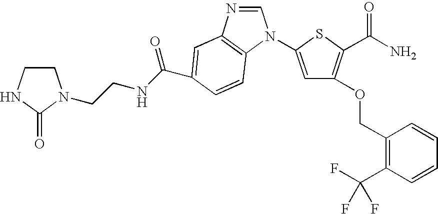

To a solution of 1-(5-(methoxycarbonyl)-4-{[2-(trifluoromethyl)benzyl]oxy}thien-2-yl)-1H-benzimidazole-5-carboxylic acid (112 mg, 0.23 mmol), 1-(2-aminoethyl)imidazolidin-2-one (85 mg, 0.35 mmol) and diisopropylethylamine (110 microL, 0.62 mmol) in dimethylformamide (2.0 mL) was added [0-(7-azabenzotriazol-1-yl)-1,1,3,3-tetramethyluronium hexafluorophosphate] (115 mg, 0.30 mmol). The reaction was stirred for 2 hours then poured into ethyl acetate and washed with aqueous 5% HCl, aqueous saturated NaHCO₃, water, brine, and dried over Na₂SO₄. Filtration and concentration gave crude methyl 5-[5-({[2-(2-oxoimidazolidin-1-yl)ethyl]amino}carbonyl)-1H-benzimidazol-1-yl]-3-{[2-(trifluoromethyl)-benzyl]oxy}thiophene-2-carboxylate (128 mg, 95%) as tan solid. The solid was stirred as a solution in 7 M ammonia in methanol (10 mL, 70 mmol), at 80° C. in a sealed, thick-walled glass pressure tube for 16 hours. The reaction was cooled to −10° C. and cold diethyl ether was added. The resulting slurry was filtered, washing the solids with cold diethyl ether. The solids were then dried under vacuum to give 1-(5-(aminocarbonyl)-4-{[2-(trifluoromethyl)benzyl]oxy}thien-2-yl)-N-[2-(2-oxoimidazolidin-1-yl)ethyl]-1H-benzimidazole-5-carboxamide (53 mg, 44%) as a white solid. ¹H NMR (400 MHz, DMSO-d₆) δ 8.75 (s, 1H), 8.64 (t, J=5.49 Hz, 1H), 8.28 (s, 1H), 7.70-7.94 (m, 7H), 7.65 (t, J=7.60 Hz, 1H), 6.79 (b, 1H), 6.28 (s, 1H), 5.55 (s, 2H), 3.36-3.44 (m, 4H), 3.18-3.27 (m, 4H). MS (ES+, m/z) 572 (m+1).

### INTERMEDIATE EXAMPLE 36

### Methyl 5-{6-[(tert-butoxycarbonyl)amino]-1H-benzimidazol-1-yl}-3-hydroxythiophene-2-carboxylate and methyl 5-{5-[(tert-butoxycarbonyl)amino]-1H-benzimidazol-1-yl}-3-hydroxythiophene-2-carboxylate

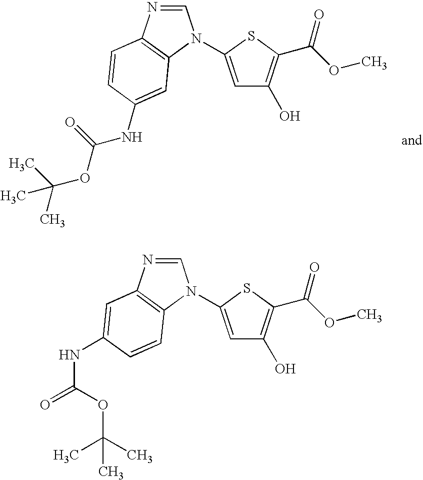

Compounds were prepared using procedure similarly described in Example 2A. MS (ES−, m/z) 388 (m⁻¹).

### INTERMEDIATE EXAMPLE 37

### Methyl 5-{6-[(tert-butoxycarbonyl)amino]-1H-benzimidazol-1-yl}-3-[1-(2-chlorophenyl)ethoxy]thiophene-2-carboxylate and Methyl 5-{5-[(tert-butoxycarbonyl)amino]-1H-benzimidazol-1-yl}-3-[1-(2-chlorophenyl)ethoxy]thiophene-2-carboxylate

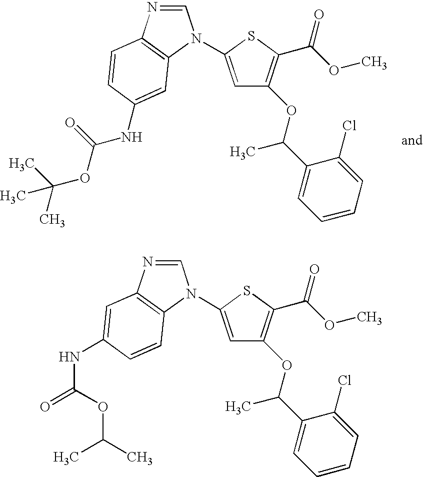

Compounds were prepared using procedure similarly described in Example 57 or Intermediate Example 21. MS (ES+, m/z) 428 (m+1).

### INTERMEDIATE EXAMPLE 38

### Methyl 5-(6-amino-1H-benzimidazol-1-yl)-3-[1-(2-chlorophenyl)ethoxy]thiophene-2-carboxylate and Methyl 5-(5-amino-1H-benzimidazol-1-yl)-3-[1-(2-chlorophenyl)ethoxy]thiophene-2-carboxylate

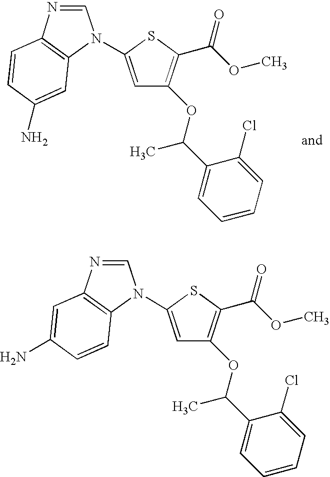

A regioisomeric mixture of methyl 5-{6-[(tert-butoxycarbonyl)amino]-1H-benzimidazol-1-yl}-3-[1-(2-chlorophenyl)ethoxy]thiophene-2-carboxylate and methyl 5-{5-[(tert-butoxycarbonyl)amino]-1H-benzimidazol-1-yl}-3-[1-(2-chlorophenyl)ethoxy]thiophene-2-carboxylate (0.610 g, 1.57 mmol) was dissolved in 20 mL of dichloromethane with stirring. Trifluoroacetic acid (6 mL) was added via syringe. The reaction was allowed to stir for 2 hours at room temperature and the reaction was then diluted with ethyl acetate and neutralised with bicarbonate. The layers were separated, and the organic layer was washed with brine. The combined aqueous layers were extracted with ethyl acetate. The combined organic layers were dried over MgSO₄, filtered, and concentrated in vacuo. Purification by flash chromatography afforded 0.1915 g (39%) of methyl 5-(6-amino-1H-benzimidazol-1-yl)-3-[1-(2-chlorophenyl)ethoxy]thiophene-2-carboxylate and 0.1182 g (24%) of methyl 5-(5-amino-1H-benzimidazol-1-yl)-3-[1-(2-chlorophenyl)ethoxy]thiophene-2-carboxylate. Data for (6-NH₂): ¹H NMR (400 MHz, DMSO-d₆) δ 8.32 (s, 1H), 7.75 (dd, J=7.8, 1.6 Hz, 1H), 7.50-7.30 (m, 6H), 6.92 (d, J=1.8 Hz, 1H), 6.62 (dd, J=8.6, 2.0 Hz, 1H), 5.93 (q, J=6.2 Hz, 1H), 5.30 (bs, 2H), 3.80 (s, 3H), 1.61 (d, J=6.2 Hz, 3H). MS (ES+, m/z) 428 (m+1). Data for (5-NH₂): ¹H NMR (400 MHz, DMSO-d₆) δ 8.44 (s, 1H), 7.72 (dd, J=7.7, 1.7 Hz, 1H), 7.49-7.39 (m, 2H), 7.38-7.31 (m, 2H), 7.30 (s, 1H), 6.84 (d, J=2.2 Hz, 1H), 6.69 (dd, J=8.7, 2.1 Hz, 1H), 5.96 (q, J=6.4 Hz, 1H), 5.05 (bs, 2H), 3.80 (s, 3H), 1.61 (d, J=6.4 Hz, 3H). MS (ES+, m/z) 428 (m+1).

### EXAMPLE 157

### 5-(5-Amino-1H-benzimidazol-1-yl)-3-[1-(2-chlorophenyl)ethoxy]thiophene-2-carboxamide

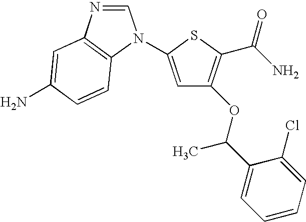

5-(5-Amino-1H-benzimidazol-1-yl)-3-[1-(2-chlorophenyl)ethoxy]thiophene-2-carboxamide was prepared from methyl 5-(5-amino-1H-benzimidazol-1-yl)-3-[1-(2-chlorophenyl)ethoxy]thiophene-2-carboxylate using procedure similarly described in Example 61 except 7M NH₃ in MeOH was used instead of 2M NH₃ in MeOH. ¹H NMR (400 MHz, DMSO-d₆) δ 8.33 (s, 1H), 7.77 (bs, 1H), 7.67 (dd, J=7.7, 1.7 Hz, 1H), 7.50 (dd, J=8.0, 1.4 Hz, 1H), 7.48-7.33 (m, 2H), 7.23 (d, J=8.8 Hz, 1H), 7.09 (bs, 1H), 7.07 (s, 1H), 6.85 (d, J=1.8 Hz, 1H), 6.68 (dd, J=8.6, 2.0 Hz, 1H), 5.98 (q, J=6.4 Hz, 1H), 5.06 (bs, 2H), 1.72 (d, J=6.4 Hz, 3H). MS (ES+, m/z) 413 (m+1).

### INTERMEDIATE EXAMPLE 39

### Methyl 3-[1-(2-chlorophenyl)ethoxy]-5-(6-{[(1-methylpiperidin-3-yl)carbonyl]amino}-1H-benzimidazol-1-yl)thiophene-2-carboxylate

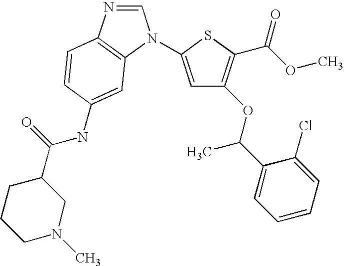

A soultion of 1-methylpiperidine-3-carboxylic acid hydrochloride (63 mg, 0.35 mmol), HATU (133 mg, 0.35 mmol) and diisopropylethylamine (0.12 mL, 0.70 mmol) in DMF (3 mL) was added to a stirring solution of methyl 5-(6-amino-1H-benzimidazol-1-yl)-3-[1-(2-chlorophenyl)ethoxy]thiophene-2-carboxylate (149 mg, 0.35 mmol) in DMF (3 mL). The resultant solution was allowed to stir at room temperature for 2 h. The reaction mixture was then diluted with EtOAc and washed several times with water. The organic layer was dried over sodium sulfate, filtered and concentrated in vacuo. The residue was purified by flash column chromatography to yield methyl 3-[1-(2-chlorophenyl)ethoxy]-5-(6-{[(1-methylpiperidin-3-yl)carbonyl]amino}-1H-benzimidazol-1-yl)thiophene-2-carboxylate (123 mg, 64%). Data: ¹H NMR (400 MHz, CDCl₃) δ 8.39 (bs, 1H), 7.91 (s, 1H), 7.73-7.66 (m, 2H), 7.35-7.27 (m, 2H), 7.25-7.19 (m, 1H), 7.15 (bs, 1H), 6.72 (s, 1H), 5.83 (q, J=6.4 Hz, 1H), 3.90 (s, 3H), 3.03 (bs, 2H), 2.86 (bs, 2H), 2.52 (bs, 3H), 1.90 (bs, 4H), 1.73 (d, J=6.4 Hz, 3H). MS (ES+, m/z) 553 (m+1).

### INTERMEDIATE EXAMPLE 40

### Methyl 3-[1-(2-chlorophenyl)ethoxy]-5-(5-{[(1-methylpiperidin-3-yl)carbonyl]amino}-1H-benzimidazol-1-yl)thiophene-2-carboxylate

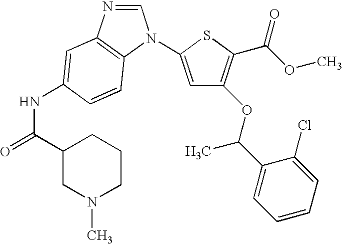

Compound was prepared using procedure similarly described in Intermediate Example 39. ¹H NMR (400 MHz, CDCl₃) δ 7.97 (bs, 2H), 7.69-7.62 (m, 2H), 7.41-7.29 (m, 3H), 7.27-7.22 (m, 1H), 6.69 (s, 1H), 5.82 (q, J=6.3 Hz, 1H), 3.91 (s, 3H), 3.04 (bs, 2H), 2.85 (bs, 2H), 2.48 (bs, 3H), 1.99 (bs, 2H), 1.86 (bs, 2H), 1.74 (d, J=6.3 Hz, 3H). MS (ES−, m/z) 551 (m⁻¹).

### EXAMPLE 158

### 3-[1-(2-Chlorophenyl)ethoxy]-5-(6-{[(1-methylpiperidin-3-yl)carbonyl]amino}-1H-benzimidazol-1-yl)thiophene-2-carboxamide

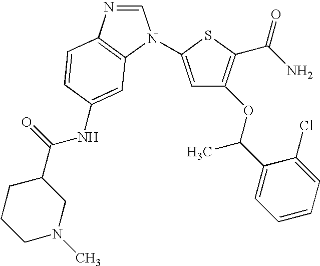

Compound was prepared using procedure similarly described in Intermediate Example 61 except 7M NH₃ in MeOH was used instead of 2M NH₃ in MeOH. ¹H NMR (400 MHz, DMSO-d₆) δ 10.19 (s, 1H), 8.50 (s, 1H), 8.38 (s, 1H), 7.84 (bs, 1H), 7.73-7.66 (m, 2H), 7.51-7.32 (m, 4H), 7.30 (s, 1H), 7.11 (bs, 1H), 5.94 (q, J=6.4 Hz, 1H), 2.90-2.86 (m, 1H), 2.75-2.71 (m, 1H), 2.63-2.57 (m, 1H), 2.20 (s, 3H), 2.10-2.01 (m, 1H), 1.93-1.79 (m, 2H), 1.74 (d, J=6.4 Hz, 3H), 1.72-1.67 (m, 1H), 1.53-1.38 (m, 2H). MS (ES+, m/z) 538 (m+1).

### EXAMPLE 159

### Biological Examples

I. Assay for Inhibition of PLK1

A. Preparation of 6× N-Terminal His-Tagged PLK Kinase Domain

6× N-terminal His-tagged PLK kinase domain (amino acids 21-346 preceded by MKKGHHHHHHD) SEQ ID: No. 1. was prepared from baculovirus infected T. ni cells under polyhedrin promoter control. All procedures were performed at 4° C. Cells were lysed in 50 mM HEPES, 200 mM NaCl, 50 mM imidazole, 5% glycerol; pH 7.5. The homogenate was centrifuged at 14 K rpm in a SLA-1500 rotor for 1 hr and the supernatant filtered through a 1.2 micron filter. The supernatant was loaded onto a Nickel chelating Sepharose (Amersham Pharmacia) column and washed with lysis buffer. Protein was eluted using 20%, 30% and 100% buffer B steps where buffer B is 50 mM HEPES, 200 mM NaCl, 300 mM imidazole, 5% glycerol; pH 7.5. Fractions containing PLK were determined by SDS-PAGE. Fractions containing PLK were diluted five-fold with 50 mM HEPES, 1 mM DTT, 5% glycerol; pH 7.5, then loaded on an SP Sepharose (Amersham Pharmacia) column. After washing the column with 50 mM HEPES, 1 mM DTT, 5% glycerol; pH 7.5, PLK was step eluted with 50 mM HEPES, 1 mM DTT, 500 mM NaCl; 5% glycerol; pH 7.5. PLK was concentrated using a 10 kDa molecular weight cutoff membrane and then loaded onto a Superdex 200 gel filtration (Amersham Pharmacia) column equilibrated in 25 mM HEPES, 1 mM DTT, 500 mM NaCl, 5% glycerol; pH 7.5. Fractions containing PLK were determined by SDS-PAGE. PLK was pooled, aliquoted and stored at −80° C. Samples were quality controlled using mass spectrometry, N-terminal sequencing and amino acid analysis.

B. Enzyme Activity+/−Inhibitors was Determined as Follows:

Compounds were added to the plate (1 μl in 100% DMSO). DMSO (2% final) and EDTA (55.5 mM final) were used as controls. Reaction Mix A is prepared as follows at 4° C.:
Reaction Mix A (substrate Mix):
   25 mM HEPES, pH 7.2
   15 mM MgCl2
   2 μM ATP
   0.1 μCi/well ³³P-γ ATP (10 Ci/mMol)
   2 μM substrate peptide (Biotin-Ahx-SFNDTLDFD) SEQ ID:No. 2.
Reaction Mix B is prepared as follows at 4° C.:
   Reaction Mix B (Enzyme Mix)
      25 mM HEPES, pH 7.2
      15 mM MgCl₂
      0.15 mg/ml BSA
      2 mM DTT
      2-10 nM PLK1 kinase domain

Reaction Mix A (20 μl) is added per well. Reaction Mix B (20 μl) is added per well. Incubate 1.5 hrs. at RT. The enzymatic reaction is stopped with 175 μl of SPA/EDTA bead mix (29 mM EDTA, 2.5 mg/ml Streptavidin-coated SPA in Standard Dulbecco's PBS (without Mg²⁺ and Ca²⁺), 60 μM ATP). Plates are sealed spun (after a 1 hr incubation at RT) at 1,000×g for 7 min or settled overnight, then plates counted in Packard TopCount for 30 seconds/well.

C. Results

The data obtained is reported in Table 1 below. In Table 1, +=pIC50<5; ++=pIC50 5-7; +++=pIC50>7.

II. Methylene Blue Growth Inhibition Assay

Normal Human foreskin fibroblasts (HFF) and human colon (HCT116, RKO), lung (H460), prostate (PC3), and breast tumor (MCF7) cell lines were cultured in high glucose DMEM (Life Technologies) containing 10% fetal bovine serum (FBS) at 37° C. in a humidified 10% CO₂, 90% air incubator. Cells were harvested using trypsin/EDTA, counted using a haemocytometer, and plated in 100 μl of the appropriate media, at the following densities, in a 96-well tissue culture plate (Falcon 3075): HFF 5,000 cells/well, HCT116 3,000 cells/well, RKO 2,500 cells/well, H460 2,000 cells/well, PC₃ 8,000 cells/well, MCF7 4,000 cells/well. The next day, compounds were diluted in DMEM containing 100 μg/ml gentamicin, at twice the final required concentration, from 10 mM stock solutions in DMSO. 100 μl/well of these dilutions were added to the 100 μl of media currently on the cell plates. Medium containing 0.6% DMSO was added to control wells. Compounds diluted in DMEM were added to all cell lines. The final concentration of DMSO in all wells was 0.3%. Cells were incubated at 37° C., 10% CO₂ for 3 days. Medium was removed by aspiration. Cell biomass was estimated by staining cells with 90 μl per well methylene blue (Sigma M9140, 0.5% in 50:50 ethanol:water), and incubation at room temperature for at least 30 minutes. Stain was removed, and the plates rinsed under a gentle stream of water, and air-dried. To release stain from the cells 100 μl of solubilization solution was added (1% N-lauroyl sarcosine, Sodium salt, Sigma L5125, in PBS), and plates were shaken gently for about 30 minutes. Optical density at 620 nM was measured on a microplate reader. Percent inhibition of cell growth was calculated relative to vehicle treated control wells. Concentration of compound that inhibits 50% of cell growth (IC₅₀) was interpolated using nonlinear regression (Levenberg-Marquardt) and the equation, y=Vₘₐₓ*(1−(x/(K+x)))+Y², where “K” was equal to the IC₅₀. The data obtained reported in Table 1 below. In Table 1, +=10->30 uM; ++=1-10 uM: +++=<1 uM.

TABLE 1

| | Ave pIC50 PLK | MeB | IC50 |
|---|---|---|---|
| Example | Enzyme Inhibition | Cell Line | (μM) |
| 4 | +++ | | |
| 13 | +++ | | |
| 14 | +++ | | |
| 15 | +++ | | |
| 34 | +++ | H460 | + |
| | | HCT116 | + |
| | | HFF | + |
| | | MCF7 | + |
| | | PC3 | + |
| | | RKO | + |
| 35 | +++ | H460 | + |
| | | HCT116 | + |
| | | HFF | + |
| | | MCF7 | + |
| | | PC3 | + |
| | | RKO | + |
| 39 | ++ | H460 | + |
| | | HCT116 | + |
| | | HFF | + |
| | | MCF7 | + |
| | | PC3 | + |
| | | RKO | + |
| 61 | +++ | H460 | ++ |
| | | HCT116 | ++ |
| | | HFF | + |
| | | MCF7 | ++ |
| | | PC3 | + |
| | | RKO | ++ |
| 62 | +++ | H460 | +++ |
| | | HCT116 | +++ |
| | | HFF | ++ |
| | | MCF7 | +++ |
| | | PC3 | + |
| | | RKO | +++ |
| 63 | +++ | H460 | +++ |
| | | HCT116 | +++ |
| | | HFF | ++ |
| | | MCF7 | +++ |
| | | PC3 | ++ |
| | | RKO | +++ |
| 64 | +++ | H460 | +++ |
| | | HCT116 | ++ |
| | | HFF | + |
| | | MCF7 | ++ |
| | | PC3 | + |
| | | RKO | ++ |
| 65 | +++ | H460 | +++ |
| | | HCT116 | +++ |
| | | HFF | + |
| | | MCF7 | +++ |
| | | PC3 | + |
| | | RKO | +++ |
| 66 | +++ | H460 | + |
| | | HCT116 | + |
| | | HFF | + |
| | | MCF7 | + |
| | | PC3 | + |
| | | RKO | + |
| 67 | +++ | H460 | + |
| | | HCT116 | + |
| | | HFF | + |
| | | MCF7 | + |
| | | PC3 | + |
| | | RKO | + |
| 68 | +++ | H460 | + |
| | | HCT116 | + |
| | | HFF | + |
| | | MCF7 | + |
| | | PC3 | + |
| | | RKO | + |
| 69 | +++ | H460 | + |
| | | HCT116 | + |
| | | HFF | + |
| | | MCF7 | + |
| | | PC3 | + |
| | | RKO | + |
| 70 | +++ | H460 | + |
| | | HCT116 | + |
| | | HFF | + |
| | | MCF7 | + |
| | | PC3 | + |
| | | RKO | + |
| 71 | +++ | H460 | + |
| | | HCT116 | + |
| | | HFF | + |
| | | MCF7 | + |
| | | PC3 | + |
| | | RKO | + |
| 72 | +++ | H460 | + |
| | | HCT116 | + |
| | | HFF | + |
| | | MCF7 | + |
| | | PC3 | + |
| | | RKO | + |
| 74 | +++ | H460 | ++ |
| | | HCT116 | + |
| | | HFF | + |
| | | MCF7 | + |
| | | PC3 | + |
| | | RKO | + |
| 75 | +++ | H460 | + |
| | | HCT116 | ++ |
| | | HFF | + |
| | | MCF7 | + |
| | | PC3 | + |
| | | RKO | + |
| 76 | +++ | H460 | + |
| | | HCT116 | + |
| | | HFF | + |
| | | MCF7 | + |
| | | PC3 | + |
| | | RKO | + |
| 77 | +++ | H460 | + |
| | | HCT116 | + |
| | | HFF | + |
| | | MCF7 | + |
| | | PC3 | + |
| | | RKO | + |
| 78 | +++ | H460 | + |
| | | HCT116 | + |
| | | HFF | + |
| | | MCF7 | + |
| | | PC3 | + |
| | | RKO | + |
| 79 | +++ | H460 | + |
| | | HCT116 | + |
| | | HFF | + |
| | | MCF7 | + |
| | | PC3 | + |
| | | RKO | + |
| 80 | +++ | H460 | + |
| | | HCT116 | + |
| | | HFF | + |
| | | MCF7 | + |
| | | PC3 | + |
| | | RKO | + |
| 83 | ++ | | |
| 84 | +++ | | |
| 85 | ++ | H460 | + |
| | | HCT116 | + |
| | | HFF | + |
| | | MCF7 | + |
| | | PC3 | + |
| | | RKO | + |
| 86 | +++ | | |
| 87 | +++ | H460 | ++ |
| | | HCT116 | ++ |
| | | HFF | + |
| | | MCF7 | ++ |
| | | PC3 | + |
| | | RKO | ++ |
| 88 | ++ | H460 | + |
| | | HCT116 | + |
| | | HFF | + |
| | | MCF7 | ++ |
| | | PC3 | + |
| | | RKO | + |
| 89 | +++ | H460 | ++ |
| | | HCT116 | ++ |
| | | HFF | + |
| | | MCF7 | ++ |
| | | PC3 | + |
| | | RKO | ++ |
| 90 | +++ | H460 | ++ |
| | | HCT116 | ++ |
| | | HFF | + |
| | | MCF7 | ++ |
| | | PC3 | + |
| | | RKO | ++ |
| 91 | +++ | A549 | +++ |
| | | H460 | +++ |
| | | HCT116 | +++ |
| | | HFF | + |
| | | MCF7 | +++ |
| | | PC3 | ++ |
| | | RKO | +++ |
| 92 | +++ | H460 | +++ |
| | | HCT116 | +++ |
| | | HFF | ++ |
| | | MCF7 | +++ |
| | | PC3 | ++ |
| | | RKO | +++ |
| 93 | ++ | H460 | + |
| | | HCT116 | + |
| | | HFF | + |
| | | MCF7 | + |
| | | PC3 | + |
| | | RKO | + |
| 94 | +++ | H460 | ++ |
| | | HCT116 | ++ |
| | | HFF | ++ |
| | | MCF7 | ++ |
| | | PC3 | ++ |
| | | RKO | ++ |
| 95 | +++ | H460 | ++ |
| | | HCT116 | ++ |
| | | HFF | + |
| | | MCF7 | ++ |
| | | PC3 | + |
| | | RKO | ++ |
| 96 | +++ | H460 | ++ |
| | | HCT116 | ++ |
| | | HFF | + |
| | | MCF7 | ++ |
| | | PC3 | + |
| | | RKO | ++ |
| 97 | +++ | H460 | ++ |
| | | HCT116 | ++ |
| | | HFF | ++ |
| | | MCF7 | ++ |
| | | PC3 | ++ |
| | | RKO | ++ |
| 98 | ++ | H460 | ++ |
| | | HCT116 | ++ |
| | | HFF | + |
| | | MCF7 | ++ |
| | | PC3 | ++ |
| | | RKO | ++ |
| 99 | +++ | H460 | ++ |
| | | HCT116 | ++ |
| | | HFF | + |
| | | MCF7 | ++ |
| | | PC3 | + |
| | | RKO | ++ |
| 100 | +++ | H460 | + |
| | | HCT116 | + |
| | | HFF | + |
| | | MCF7 | + |
| | | PC3 | + |
| | | RKO | + |
| 101 | +++ | A549 | ++ |
| | | H460 | ++ |
| | | HCT116 | ++ |
| | | HFF | + |
| | | MCF7 | ++ |
| | | PC3 | ++ |
| | | RKO | +++ |
| 102 | +++ | A549 | ++ |
| | | H460 | ++ |
| | | HCT116 | ++ |
| | | HFF | + |
| | | MCF7 | ++ |
| | | PC3 | + |
| | | RKO | +++ |
| 103 | +++ | H460 | ++ |
| | | HCT116 | ++ |
| | | HFF | + |
| | | MCF7 | ++ |
| | | PC3 | + |
| | | RKO | ++ |
| 104 | +++ | H460 | ++ |
| | | HCT116 | ++ |
| | | HFF | + |
| | | MCF7 | ++ |
| | | PC3 | + |
| | | RKO | ++ |
| 105 | +++ | H460 | ++ |
| | | HCT116 | ++ |
| | | HFF | + |
| | | MCF7 | ++ |
| | | PC3 | + |
| | | RKO | ++ |
| 106 | +++ | H460 | ++ |
| | | HCT116 | ++ |
| | | HFF | + |
| | | MCF7 | ++ |
| | | PC3 | + |
| | | RKO | ++ |
| 107 | +++ | A549 | +++ |
| | | H460 | +++ |
| | | HCT116 | ++ |
| | | HFF | ++ |
| | | MCF7 | ++ |
| | | PC3 | ++ |
| | | RKO | +++ |
| 108 | +++ | A549 | +++ |
| | | H460 | +++ |
| | | HCT116 | +++ |
| | | HFF | + |
| | | MCF7 | +++ |
| | | PC3 | + |
| | | RKO | +++ |
| 109 | ++ | H460 | + |
| | | HCT116 | + |
| | | HFF | + |
| | | MCF7 | + |
| | | PC3 | + |
| | | RKO | + |
| 110 | +++ | H460 | ++ |
| | | HCT116 | +++ |
| | | HFF | + |
| | | MCF7 | ++ |
| | | PC3 | ++ |
| | | RKO | +++ |
| 111 | +++ | H460 | ++ |
| | | HCT116 | ++ |
| | | HFF | + |
| | | MCF7 | ++ |
| | | PC3 | ++ |
| | | RKO | ++ |
| 112 | ++ | H460 | ++ |
| | | HCT116 | + |
| | | HFF | + |
| | | MCF7 | + |
| | | PC3 | + |
| | | RKO | ++ |
| 113 | ++ | H460 | + |
| | | HCT116 | + |
| | | HFF | + |
| | | MCF7 | + |
| | | PC3 | + |
| | | RKO | + |
| 114 | ++ | H460 | + |
| | | HCT116 | + |
| | | HFF | + |
| | | MCF7 | + |
| | | PC3 | + |
| | | RKO | + |
| 115 | +++ | H460 | ++ |
| | | HCT116 | ++ |
| | | HFF | + |
| | | MCF7 | ++ |
| | | PC3 | + |
| | | RKO | ++ |
| 116 | +++ | H460 | +++ |
| | | HCT116 | ++ |
| | | HFF | ++ |
| | | MCF7 | +++ |
| | | PC3 | + |
| | | RKO | +++ |
| 117 | +++ | H460 | +++ |
| | | HCT116 | +++ |
| | | HFF | + |
| | | MCF7 | +++ |
| | | PC3 | ++ |
| | | RKO | +++ |
| 118 | +++ | H460 | +++ |
| | | HCT116 | ++ |
| | | HFF | ++ |
| | | MCF7 | +++ |
| | | PC3 | + |
| | | RKO | +++ |
| 119 | +++ | H460 | ++ |
| | | HCT116 | ++ |
| | | HFF | + |
| | | MCF7 | ++ |
| | | PC3 | + |
| | | RKO | ++ |
| 120 | +++ | A549 | ++ |
| | | H460 | ++ |
| | | HCT116 | ++ |
| | | HFF | + |
| | | MCF7 | ++ |
| | | PC3 | + |
| | | RKO | +++ |
| 121 | +++ | A549 | ++ |
| | | H460 | ++ |
| | | HCT116 | ++ |
| | | HFF | + |
| | | MCF7 | ++ |
| | | PC3 | + |
| | | RKO | ++ |
| 122 | +++ | H460 | ++ |
| | | HCT116 | ++ |
| | | HFF | + |
| | | MCF7 | ++ |
| | | PC3 | + |
| | | RKO | ++ |
| 123 | +++ | H460 | ++ |
| | | HCT116 | ++ |
| | | HFF | ++ |
| | | MCF7 | ++ |
| | | PC3 | + |
| | | RKO | ++ |
| 124 | ++ | H460 | + |
| | | HCT116 | + |
| | | HFF | + |
| | | MCF7 | + |
| | | PC3 | + |
| | | RKO | + |
| 125 | +++ | H460 | ++ |
| | | HCT116 | ++ |
| | | HFF | + |
| | | MCF7 | ++ |
| | | PC3 | + |
| | | RKO | ++ |
| 126 | +++ | A549 | +++ |
| | | H460 | +++ |
| | | HCT116 | +++ |
| | | HFF | +++ |
| | | MCF7 | ++ |
| | | PC3 | ++ |
| | | RKO | +++ |
| 127 | +++ | A549 | +++ |
| | | H460 | +++ |
| | | HCT116 | +++ |
| | | HFF | ++ |
| | | MCF7 | +++ |
| | | PC3 | ++ |
| | | RKO | +++ |
| 128 | ++ | H460 | ++ |
| | | HCT116 | + |
| | | HFF | + |
| | | MCF7 | ++ |
| | | PC3 | + |
| | | RKO | ++ |
| 129 | ++ | H460 | + |
| | | HCT116 | + |
| | | HFF | + |
| | | MCF7 | + |
| | | PC3 | + |
| | | RKO | + |
| 130 | ++ | H460 | + |
| | | HCT116 | ++ |
| | | HFF | + |
| | | MCF7 | ++ |
| | | PC3 | + |
| | | RKO | ++ |
| 131 | ++ | H460 | ++ |
| | | HCT116 | ++ |
| | | HFF | + |
| | | MCF7 | ++ |
| | | PC3 | + |
| | | RKO | ++ |
| 132 | +++ | H460 | + |
| | | HCT116 | + |
| | | HFF | + |
| | | MCF7 | + |
| | | PC3 | + |
| | | RKO | + |
| 133 | ++ | H460 | + |
| | | HCT116 | ++ |
| | | HFF | + |
| | | MCF7 | + |
| | | PC3 | + |
| | | RKO | ++ |
| 134 | +++ | A549 | ++ |
| | | H460 | ++ |
| | | HCT116 | ++ |
| | | HFF | + |
| | | MCF7 | ++ |
| | | PC3 | + |
| | | RKO | ++ |
| 135 | +++ | H460 | +++ |
| | | HCT116 | +++ |
| | | HFF | ++ |
| | | MCF7 | +++ |
| | | PC3 | ++ |
| | | RKO | +++ |
| 136 | +++ | H460 | +++ |
| | | HCT116 | +++ |
| | | HFF | ++ |
| | | MCF7 | +++ |
| | | PC3 | ++ |
| | | RKO | +++ |
| 137 | +++ | H460 | ++ |
| | | HCT116 | +++ |
| | | HFF | + |
| | | MCF7 | ++ |
| | | PC3 | ++ |
| | | RKO | ++ |
| 138 | +++ | H460 | +++ |
| | | HCT116 | ++ |
| | | HFF | ++ |
| | | MCF7 | ++ |
| | | PC3 | + |
| | | RKO | +++ |
| 139 | +++ | H460 | ++ |
| | | HCT116 | ++ |
| | | HFF | + |
| | | MCF7 | ++ |
| | | PC3 | ++ |
| | | RKO | +++ |
| 140 | +++ | H460 | ++ |
| | | HCT116 | ++ |
| | | HFF | + |
| | | MCF7 | ++ |
| | | PC3 | ++ |
| | | RKO | +++ |
| 141 | +++ | | |
| 142 | +++ | H460 | ++ |
| | | HCT116 | ++ |
| | | HFF | + |
| | | MCF7 | ++ |
| | | PC3 | + |
| | | RKO | ++ |
| 143 | +++ | A549 | ++ |
| | | H460 | ++ |
| | | HCT116 | +++ |
| | | HFF | ++ |
| | | MCF7 | +++ |
| | | PC3 | ++ |
| | | RKO | +++ |
| 144 | ++ | | |
| 145 | +++ | H460 | + |
| | | HCT116 | + |
| | | HFF | + |
| | | MCF7 | + |
| | | PC3 | + |
| | | RKO | + |
| 146 | ++ | | |
| 147 | +++ | H460 | + |
| | | HCT116 | + |
| | | HFF | + |
| | | MCF7 | + |
| | | PC3 | + |
| | | RKO | + |
| 148 | ++ | H460 | ++ |
| | | HCT116 | ++ |
| | | HFF | + |
| | | MCF7 | ++ |
| | | PC3 | + |
| | | RKO | ++ |
| 149 | +++ | H460 | ++ |
| | | HCT116 | ++ |
| | | HFF | + |
| | | MCF7 | + |
| | | PC3 | + |
| | | RKO | + |
| 150 | ++ | H460 | + |
| | | HCT116 | + |
| | | HFF | + |
| | | MCF7 | + |
| | | PC3 | + |
| | | RKO | + |
| 151 | +++ | H460 | + |
| | | HCT116 | + |
| | | HFF | + |
| | | MCF7 | + |
| | | PC3 | + |
| | | RKO | + |
| 152 | ++ | H460 | + |
| | | HCT116 | + |
| | | HFF | + |
| | | MCF7 | + |
| | | PC3 | + |
| | | RKO | + |
| 153 | +++ | H460 | + |
| | | HCT116 | + |
| | | HFF | + |
| | | MCF7 | + |
| | | PC3 | + |
| | | RKO | + |
| 154 | +++ | A549 | +++ |
| | | H460 | +++ |
| | | HCT116 | ++ |
| | | HFF | + |
| | | MCF7 | +++ |
| | | PC3 | + |
| | | RKO | +++ |
| 155 | +++ | H460 | + |
| | | HCT116 | + |
| | | HFF | + |
| | | MCF7 | + |
| | | PC3 | + |
| | | RKO | + |
| 156 | +++ | H460 | + |
| | | HCT116 | + |
| | | HFF | + |
| | | MCF7 | + |
| | | PC3 | + |
| | | RKO | + |
| 157 | +++ | H460 | +++ |
| | | HCT116 | +++ |
| | | HFF | + |
| | | MCF7 | +++ |
| | | PC3 | +++ |
| | | RKO | +++ |

1. A compound of formula (I): 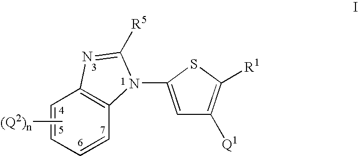
wherein:
R¹ is selected from the group consisting of H, alkyl, alkenyl, alkynyl, —C(O)R⁷, —CO₂R⁷, —C(O)NR⁷R⁸, —C(O)N(R⁷)OR⁸, —C(O)N(R⁷)—R²—OR⁸, —C(O)N(R⁷)-Ph, —C(O)N(R⁷)—R²-Ph, —C(O)N(R⁷)C(O)R⁸, —C(O)N(R⁷)CO₂R⁸, —C(O)N(R⁷)C(O)NR⁷R⁸, —C(O)N(R⁷)S(O)₂R⁸, —R²—OR⁷, —R²—O—C(O)R⁷, —C(S)R⁷, —C(S)NR⁷R⁸, —C(S)N(R⁷)-Ph, —C(S)N(R⁷)—R²-Ph, —R²—SR⁷, —C(═NR⁷)NR⁷R⁸, —C(═NR⁷)N(R⁸)-Ph, —C(═NR⁷)N(R⁸)—R²-Ph, —R²—NR⁷R⁸, —CN, —OR⁷, —S(O)_{f}R⁷, —S(O)₂NR⁷R⁸, —S(O)₂N(R⁷)-Ph, —S(O)₂N(R⁷)—R²-Ph, —NR⁷R⁸, —N(R⁷)-Ph, —N(R⁷)—R²-Ph, —N(R⁷)—SO₂R⁸ and Het;
Ph is phenyl optionally substituted from 1 to 3 times with a substituent selected from the group consisting of halo, alkyl, —OH, —R²—OH, —O-alkyl, —R²—O-alkyl, —NH₂, —N(H)alkyl, —N(alkyl)₂, —CN and —N₃;
Het is a 5-7 membered heterocycle having 1, 2, 3 or 4 heteroatoms selected from N, O and S, or a 5-6 membered heteroaryl having 1, 2, 3 or 4 heteroatoms selected from N, O and S, each optionally substituted from 1 to 2 times with a substituent selected from the group consisting of halo, alkyl, oxo, —OH, —R²—OH, —O-alkyl, —R²—O-alkyl, —NH₂, —N(H)alkyl, —N(alkyl)₂, —CN and —N₃;
Q¹ is a group of formula:
—(R²)ₐ—(Y¹)_{b}—(R²)_{c}—R³
a, b and c are the same or different and are each independently 0 or 1 and at least one of a or b is 1;
n is 0, 1, 2, 3 or 4;
Q² is a group of formula:
—(R²)ₐₐ—(Y²)_{bb}—(R²)_{cc}—R⁴
or two adjacent Q² groups are selected from the group consisting of alkyl, alkenyl, —OR⁷, —S(O)_{f}R⁷ and —NR⁷R⁸ and together with the carbon atoms to which they are bound, they form a C₅₋₆cycloalkyl, C₅₋₆cycloalkenyl, phenyl, 5-7 membered heterocycle having 1 or 2 heteroatoms selected from N, O and S, or 5-6 membered heteroaryl having 1 or 2 heteroatoms selected from N, O and S;
aa, bb and cc are the same or different and are each independently 0 or 1;
each Y¹ and Y² is the same or different and is independently selected from the group consisting of —O—, —S(O)_{f}—, —N(R⁷)—, —C(O)—, —OC(O)—, —CO₂—, —C(O)N(R⁷)—, —C(O)N(R⁷)S(O)₂—, —OC(O)N(R⁷)—, —OS(O)₂—, —S(O)₂N(R⁷)—, —S(O)₂N(R⁷)C(O)—, —N(R⁷)S(O)₂—, —N(R⁷)C(O)—, —N(R⁷)CO₂— and —N(R⁷)C(O)N(R⁷)—;
each R² is the same or different and is independently selected from the group consisting of alkylene, alkenylene and alkynylene;
each R³ and R⁴ is the same or different and is each independently selected from the group consisting of H, halo, alkyl, alkenyl, alkynyl, —C(O)R⁷, —C(O)NR⁷R³, —CO₂R⁷, —C(S)R⁷, —C(S)NR⁷R⁸, —C(═NR⁷)R³, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN, —N₃ and a group of formula (ii): 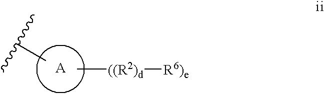
wherein:
Ring A is selected from the group consisting of C₅₋₁₀cycloalkyl, C₅₋₁₀cycloalkenyl, aryl, 5-10 membered heterocycle having 1, 2 or 3 heteroatoms selected from N, O and S and 5-10 membered heteroaryl having 1, 2 or 3 heteroatoms selected from N, O and S
each d is 0 or 1;
e is 0, 1, 2, 3 or 4;
each R⁶ is the same or different and is independently selected from the group consisting of H, halo, alkyl, alkenyl, alkynyl, cycloalkyl, cycloalkenyl, Ph, Het, —CH(OH)—R²—OH, —C(O)R⁷, —CO₂R⁷, —CO₂—R₂-Ph, —CO₂—R²-Het, —C(O)NR⁷R⁸, —C(O)N(R⁷)C(O)R⁷, —C(O)N(R⁷)CO₂R⁷, —C(O)N(R⁷)C(O)NR⁷R⁸, —C(O)N(R⁷)S(O)₂R⁷, —C(S)R⁷, —C(S)NR⁷R⁸, —C(═NR⁷)R⁸, —C(═NR⁷)NR⁷R⁸, —CR⁷═N—OR⁸, ═O, —OR⁷, —OC(O)R⁷, —OC(O)Ph, —OC(O)Het, —OC(O)NR⁷R⁸, —O—R²—S(O)₂R⁷, —S(O)_{f}R⁷, —S(O)₂NR⁷R⁸, —S(O)₂Ph, —S(O)₂Het, —NR⁷R⁸, —N(R⁷)C(O)R⁸, —N(R⁷)CO₂R⁸, —N(R⁷)—R²—CO₂R⁸, —N(R⁷)C(O)NR⁷R⁸, —N(R⁷)—R²—C(O)NR⁷R⁸, —N(R⁷)C(O)Ph, —N(R⁷)C(O)Het, —N(R⁷)Ph, —N(R⁷)Het, —N(R⁷)C(O)NR⁷—R²—NR⁷R⁸, —N(R⁷)C(O)N(R⁷)Ph, —N(R⁷)C(O)N(R⁷)Het, —N(R⁷)C(O)N(R⁷)—R²-Het, —N(R⁷)S(O)₂R⁸, —N(R⁷)—R²—S(O)₂R⁸, —NO₂, —CN and —N₃;
wherein when Q¹ is defined where b is 1 and c is 0, R³ is not halo, —C(O)R⁷, —C(O)NR⁷R⁸, —CO₂R⁷, —C(S)R⁷, —C(S)NR⁷R⁸, —C(═NR⁷)R⁸, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN or —N₃;
wherein when Q² is defined where bb is 1 and cc is 0, R⁴ is not halo, —C(O)R⁷, —C(O)NR⁷R⁸, —CO₂R⁷, —C(S)R⁷, —C(S)NR⁷R⁸, —C(═NR⁷)R⁸, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN or —N₃;
R⁵ is selected from the group consisting of H, halo, alkyl, cycloalkyl, OR⁷, —S(O)_{f}R⁷, —NR⁷R⁸, —NHC(O)R⁷, —NHC(O)NR⁷R⁸ and —NHS(O)₂R⁷;
f is 0, 1 or 2; and
each R⁷ and each R⁸ are the same or different and are each independently selected from the group consisting of H, alkyl, alkenyl, alkynyl, cycloalkyl and cycloalkenyl;
wherein when R¹ is —CO₂CH₃ and n is 0, Q¹ is not —OH;
or a pharmaceutically acceptable salt, solvate or physiologically functional derivative thereof.

2. The compound according to claim 1, wherein R¹ is selected from the group consisting of —C(O)R⁷, —CO₂R⁷ and —C(O)NR⁷R⁸.

3. The compound according to claim 1, wherein R¹ is selected from the group consisting of —CO₂R⁷ and —C(O)NR⁷R⁸.

4. The compound according to claim 1, wherein b is 1.

5. The compound according to claim 1, wherein Q¹ is defined wherein b is 1 and Y¹ is selected from —O—, —N(R⁷)—, —C(O)—, —OC(O)—, —C(O)N(R⁷)—, —OS(O)₂—, —S(O)₂N(R⁷)—, —N(R⁷)SO₂— and —N(R⁷)C(O)—.

6. The compound according to claim 5, wherein Q¹ is defined wherein b is 1 and Y¹ is selected from —O—, —N(R⁷)—, —C(O)—, —OS(O)₂—, —N(R⁷)SO₂— and —N(R⁷)C(O)—.

7. The compound according to claim 1, wherein c is 1.

8. The compound according to claim 1, wherein R³ is selected from the group consisting of H, alkyl, alkenyl, alkynyl, and a group of formula (ii):

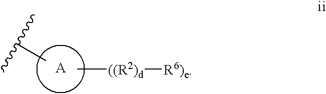

9. The compound according to claim 1, wherein R³ is a group of formula (ii) and Ring A is selected from aryl, 5-10 membered heterocycle having 1, 2 or 3 heteroatoms selected from N, O and S and 5-10 membered heteroaryl having 1, 2 or 3 heteroatoms selected from N, O and S.

10. The compound according to claim 1, wherein R³ is a group of formula (ii) and Ring A is selected from the group consisting of cycloalkyl, tetrahydropyran, tetrahydrofuran, morpholine, piperidine, phenyl, naphthyl, thiophene, furan, pyrrole, pyrrolidine, pyrrolidinone, imidazole, benzofuran, benzimidazole, pyridyl,

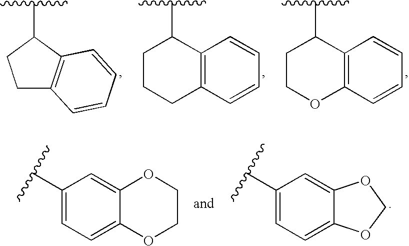

11. The compound according to claim 1, wherein Q¹ is selected from the group consisting of

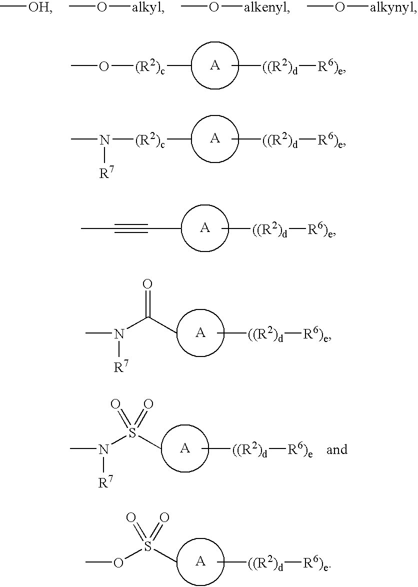

12. The compound according to claim 1, wherein R³ is a group of formula (ii) and e is 0, 1, 2 or 3.

13. The compound according to claim 1, wherein R³ is a group of formula (ii) and d is 0.

14. The compound according to claim 1, wherein wherein R³ is a group of formula (ii) and each R⁶ is the same or different and is independently selected from the group consisting of H, halo, alkyl, alkenyl, alkynyl, cycloalkyl, —OR⁷, —S(O)_{f}R⁷, —SO₂NR⁷R⁸, —NR⁷R⁸, —N(R⁷)S(O)₂R⁸, —NO₂ and —CN.

15. The compound according to claim 1, wherein n is 0, 1 or 2.

16. The compound according to claim 1, wherein Q² is defined wherein bb is 1 and Y² is —O—, —S(O)_{f}—, —N(R⁷)—, —C(O)—, —OC(O)—, —CO₂—, —C(O)N(R⁷)—, —OS(O)₂—, —N(R⁷)S(O)₂—, —N(R⁷)C(O)—, —N(R⁷)CO₂— and —N(R⁷)C(O)N(R⁷)—.

17. The compound according to claim 1, wherein cc is 1.

18. The compound according to claim 1, wherein each R⁴ is the same or different and is independently selected from the group consisting of H, halo, alkyl, alkenyl, alkynyl, —C(O)NR⁷R⁸, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN, —N₃ and a group of formula (ii):

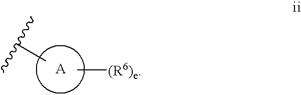

19. The compound according to claim 1, wherein R⁵ is H, halo, alkyl or —NR⁷R⁸.

20. A compound selected from the group consisting of:
5-(5,6-Dimethoxy-1H-benzimidazol-1-yl)-3-{[2-(trifluoromethyl)-benzyl]oxy}thiophene-2-carboxamide;
5-(5-(Methyloxy)-6-{[2-(4-methyl-1-piperazinyl)ethyl]oxy}-1H-benzimidazol-1-yl)-3-({[2-(trifluoromethyl)phenyl]methyl}oxy)-2-thiophenecarboxamide;
3-[1-(2-Chlorophenyl)ethoxy]-5-(5,6-dimethoxy-1H-benzimidazol-1-yl)thiophene-2-carboxamide;
5-(5,6-Dimethoxy-1-benzimidazol-1-yl)-3-[1-(2-methylphenyl)ethoxy]thiophene-2-carboxamide;
5-(5-Amino-1-H-benzimidazol-1-yl)-3-[1-(2-chlorophenyl)ethoxy]thiophene-2-carboxamide;
5-{6-[(4-Piperidinylmethyl)oxy]-1H-benzimidazol-1-yl}-3-({[2-(trifluoromethyl)phenyl]-methyl}oxy)-2-thiophenecarboxamide;
5-(6-(Methyloxy)-5-{[3-(2-oxo-1-pyrrolidinyl)propyl]oxy}-1H-benzimidazol-1-yl)-3-({[2-(trifluoromethyl)phenyl]methyl}oxy)-2-thiophenecarboxamide;
5-[6-{[3-(Dimethylamino)propyl]oxy}-5-(methyloxy)-1H-benzimidazol-1-yl]-3-({[2-(trifluoromethyl)phenyl]methyl}oxy)-2-thiophenecarboxamide;
5-(5-(Methyloxy)-6-{[2-(4-morpholinyl)ethyl]oxy}-1H-benzimidazol-1-yl)-3-({[2-(trifluoromethyl)phenyl]methyl}oxy)-2-thiophenecarboxamide;
5-[6-(2-Morpholin-4-ylethoxy)-1H-benzimidazol-1-yl]-3-{[2-(trifluoromethyl)benzyl]oxy}thiophene-2-carboxamide;
5-[6-(2-Pyrrolidin-1-ylethoxy)-1H-benzimidazol-1-yl]-3-{[2-(trifluoromethyl)benzyl]oxy}thiophene-2-carboxamide;
5-[5-Fluoro-6-(2-morpholin-4-ylethoxy)-1H-benzimidazol-1-yl]-3-{[2-(trifluoromethyl)benzyl]oxy}thiophene-2-carboxamide;
5-[6-(Methylsulfonyl)-1H-benzimidazol-1-yl]-3-{[2-(trifluoromethyl)benzyl]oxy}-thiophene-2-carboxamide;
3-[(3-Bromopyridin-4-yl)methoxy]-5-(5,6-dimethoxy-1 h-benzimidazol-1-yl)thiophene-2-carboxamide;
5-(5,6-Dimethoxy-1H-benzimidazol-1-yl)-3-{[2-(trifluoromethoxy)benzyl]oxy}thiophene-2-carboxamide;
3-{[2-(Difluoromethoxy)benzyl]oxy}-5-(5,6-dimethoxy-1H-benzimidazol-1-yl)thiophene-2-carboxamide;
3-[(2-Chloropyridin-3-yl)methoxy]-5-(5,6-dimethoxy-1H-benzimidazol-1-yl)thiophene-2-carboxamide;
5-(5,6-Dimethoxy-1H-benzimidazol-1-yl)-3-[(2-fluoropyridin-3-yl)methoxy]thiophene-2-carboxamide;
3-[(2-Aminopyridin-4-yl)methoxy]-5-(5,6-dimethoxy-1H-benzimidazol-1-yl)thiophene-2-carboxamide;
3-[(6-Chloro-1,3-benzodioxol-5-yl)methoxy]-5-(5,6-dimethoxy-1H-benzimidazol-1-yl)thiophene-2-carboxamide;
5-(5,6-Dimethoxy-1H-benzimidazol-1-yl)-3-[(2-nitrobenzyl)oxy]thiophene-2-carboxamide;
3-[(3-Aminobenzyl)oxy]-5-(5,6-dimethoxy-1H-benzimidazol-1-yl)thiophene-2-carboxamide;
5-(6-Bromo-1H-benzimidazol-1-yl)-3-{[2-(trifluoromethyl)benzyl]-oxy}thiophene-2-carboxamide;
3-[(2,6-Dichlorobenzyl)oxy]-5-(5,6-dimethoxy-1H-benzimidazol-1-yl)thiophene-2-carboxamide;
3-[(2-Bromobenzyl)oxy]-5-(5,6-dimethoxy-1H-benzimidazol-1-yl)thiophene-2-carboxamide;
5-(5,6-Dimethoxy-1H-benzimidazol-1-yl)-3-[(2-formylbenzyl)oxy]thiophene-2-carboxamide;
5-(1H-Benzimidazol-1-yl)-3-{[2-(trifluoromethyl)benzyl]oxy}thiophene-2-carboxamide;
5-(1H-Benzimidazol-1-yl)-3-[(2-nitrobenzyl)oxy]thiophene-2-carboxamide;
5-(6-Methoxy-1H-benzimidazol-1-yl)-3-{[2-(trifluoromethyl)benzyl]oxy}thiophene-2-carboxamide;
2-(Aminocarbonyl)-5-(5,6-dimethoxy-1H-benzimidazol-1-yl)thien-3-yl 2-methylbenzenesulfonate and pharmaceutically acceptable salts, solvates and physiologically functional derivatives thereof.

21. A pharmaceutical composition comprising a compound according to claim 1

22. The pharmaceutical composition according to claim 21 further comprising a pharmaceutically acceptable carrier, diluent or excipient.

23. The pharmaceutical composition according to claim 21 further comprising a chemotherapeutic agent.

24. A method for treating a condition mediated by PLK in an animal, said method comprising administering to the animal a therapeutically effective amount of a compound according to claim 1.

25. A method for treating a susceptible neoplasm in an animal, said method comprising administering to the animal a therapeutically effective amount of a compound according to claim 1.

26. The method according to claim 25, wherein said susceptible neoplasm is selected from the group consisting of breast cancer, colon cancer, lung cancer, prostate cancer, lymphoma, leukemia, endometrial cancer, melanoma, ovarian cancer, pancreatic cancer, squamous carcinoma, carcinoma of the head and neck, and esophageal carcinoma.

27. A method for treating a condition characterized by inappropriate cellular proliferation in an animal, said method comprising administering to the animal a therapeutically effective amount of a compound according to claim 1.

28. A method for inhibiting proliferation of a cell, said method comprising contacting the cell with an amount of a compound according to claim 1 sufficient to inhibit proliferation of the cell.

29. A method for inhibiting mitosis in a cell, said method comprising administering to the cell an amount of a compound according to claim 1 sufficient to inhibit mitosis in the cell.

30. A process for preparing a compound according to claim 1, said process comprising reacting a compound of formula (III): 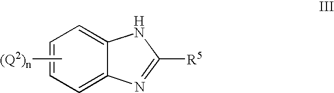
with a compound of formula (IV): 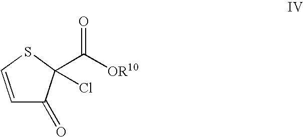
wherein R¹⁰ is selected from the group consisting of alkyl, alkenyl, alkynyl, cycloalkyl, cycloalkenyl and suitable carboxylic acid protecting groups.

31. The process according to claim 30, said process further comprising the step of converting a compound of formula (I) to a pharmaceutically acceptable salt, solvate or physiologically functional derivative thereof.

32. The process according to claim 30 further comprising the step of converting a compound of formula (I) or a pharmaceutically acceptable salt, solvate or physiologically functional derivative thereof to another compound of formula (I) or a pharmaceutically acceptable salt, solvate or physiologically functional derivative thereof.

33-42. (canceled)

43. A compound of formula (Ib): 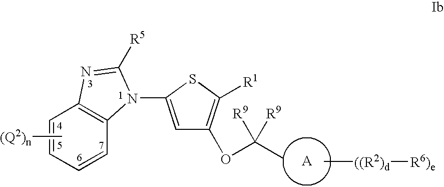
wherein:
R¹ is selected from the group consisting of H, alkyl, alkenyl, alkynyl, —C(O)R⁷, —CO₂R⁷, —C(O)NR⁷R⁸, —C(O)N(R⁷)OR⁸, —C(O)N(R⁷)—R²—OR⁸, —C(O)N(R⁷)-Ph, —C(O)N(R⁷)—R²-Ph, —C(O)N(R⁷)C(O)R⁸, —C(O)N(R⁷)CO₂R⁸, —C(O)N(R⁷)C(O)NR⁷R⁸, —C(O)N(R⁷)S(O)₂R⁸, —R²—OR⁷, —R²—O—C(O)R⁷, —C(S)R⁷, —C(S)NR⁷R⁸, —C(S)N(R⁷)-Ph, —C(S)N(R⁷)—R²-Ph, —R²—SR⁷, —C(═NR⁷) NR⁷R⁸, —C(═NR⁷)N(R⁸)-Ph, —C(═NR⁷)N(R⁸)—R²-Ph, —R²—NR⁷R⁸, —CN, —OR⁷, —S(O)_{f}R⁷, —S(O)₂NR⁷R⁸, —S(O)₂N(R⁷)-Ph, —S(O)₂N(R⁷)—R²-Ph, —NR⁷R⁸, —N(R⁷)-Ph, —N(R⁷)—R²-Ph, —N(R⁷)—SO₂R⁸ and Het;
Ph is phenyl optionally substituted from 1 to 3 times with a substituent selected from the group consisting of halo, alkyl, —OH, —R²—OH, —O-alkyl, —R²—O-alkyl, —NH₂, —N(H)alkyl, —N(alkyl)₂, —CN and —N₃;
Het is a 5-7 membered heterocycle having 1, 2, 3 or 4 heteroatoms selected from N, O and S, or a 5-6 membered heteroaryl having 1, 2, 3 or 4 heteroatoms selected from N, O and S, each optionally substituted from 1 to 2 times with a substituent selected from the group consisting of halo, alkyl, oxo, —OH, —R²—OH, —O-alkyl, —R²—O-alkyl, —NH₂, —N(H)alkyl, —N(alkyl)₂, —CN and —N₃;
n is 0, 1, 2, 3 or 4;
Q² is a group of formula:
—(R²)ₐₐ—(Y²)_{bb}—(R²)_{cc}—R⁴
or two adjacent Q² groups are selected from the group consisting of alkyl, alkenyl, —OR⁷, —S(O)_{f}R⁷ and —NR⁷R⁸ and together with the carbon atoms to which they are bound, they form a C₅₋₆cycloalkyl, C₅₋₆cycloalkenyl, phenyl, 5-7 membered heterocycle having 1 or 2 heteroatoms selected from N, O and S, or 5-6 membered heteroaryl having 1 or 2 heteroatoms selected from N, O and S;
aa, bb and cc are the same or different and are each independently 0 or 1;
each Y² is the same or different and is independently selected from the group consisting of —O—, —S(O)_{f}—, —N(R⁷)—, —C(O)—, —OC(O)—, —CO₂—, —C(O)N(R⁷)—, —C(O)N(R⁷)S(O)₂—, —OC(O)N(R⁷)—, —OS(O)₂—, —S(O)₂N(R⁷)—, —S(O)₂N(R⁷)C(O)—, —N(R⁷)S(O)₂—, —N(R⁷)C(O)—, —N(R⁷)CO₂— and —N(R⁷)C(O)N(R⁷)—;
each R² is the same or different and is independently selected from the group consisting of alkylene, alkenylene and alkynylene;
each R⁴ is the same or different and is each independently selected from the group consisting of H, halo, alkyl, alkenyl, alkynyl, —C(O)R⁷, —C(O)NR⁷R⁸, —CO₂R⁷, —C(S)R⁷, —C(S)NR⁷R⁸, —C(═NR⁷)R⁸, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN, —N₃ and a group of formula (ii): 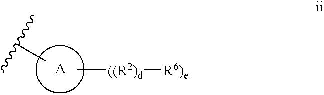
wherein:
Ring A is selected from the group consisting of C₅₋₁₀cycloalkyl, C₅₋₁₀cycloalkenyl, aryl, 5-10 membered heterocycle having 1, 2 or 3 heteroatoms selected from N, O and S and 5-10 membered heteroaryl having 1, 2 or 3 heteroatoms selected from N, O and S
each d is 0 or 1;
e is 0, 1, 2, 3 or 4;
each R⁶ is the same or different and is independently selected from the group consisting of H, halo, alkyl, alkenyl, alkynyl, cycloalkyl, cycloalkenyl, Ph, Het, —CH(OH)—R²—OH, —C(O)R⁷, —CO₂R⁷, —CO₂—R₂-Ph, —CO₂—R²-Het, —C(O)NR⁷R⁸, —C(O)N(R⁷)C(O)R⁷, —C(O)N(R⁷)CO₂R⁷, —C(O)N(R⁷)C(O)NR⁷R⁸, —C(O)N(R⁷)S(O)₂R⁷, —C(S)R⁷, —C(S)NR⁷R⁸, —C(═NR⁷)R⁸, —C(═NR⁷)NR⁷R⁸, —CR⁷═N—OR⁸, ═O, —OR⁷, —OC(O)R⁷, —OC(O)Ph, —OC(O)Het, —OC(O)NR⁷R⁸, —O—R²—S(O)₂R⁷, —S(O)_{f}R⁷, —S(O)₂NR⁷R⁸, —S(O)₂Ph, —S(O)₂Het, —NR⁷R⁸, —N(R⁷)C(O)R⁸, —N(R⁷)CO₂R⁸, —N(R⁷)—R²—CO₂R⁸, —N(R⁷)C(O)NR⁷R⁸, —N(R⁷)—R²—C(O)NR⁷R⁸, —N(R⁷)C(O)Ph, —N(R⁷)C(O)Het, —N(R⁷)Ph, —N(R⁷)Het, —N(R⁷)C(O)NR⁷—R²—NR⁷R⁸, —N(R⁷)C(O)N(R⁷)Ph, —N(R⁷)C(O)N(R⁷)Het, —N(R⁷)C(O)N(R⁷)—R²-Het, —N(R⁷)S(O)₂R⁸, —N(R⁷)—R²—S(O)₂R⁸, —NO₂, —CN and —N₃;
wherein when Q² is defined where bb is 1 and cc is 0, R⁴ is not halo, —C(O)R⁷, —C(O)NR⁷R⁸, —CO₂R⁷, —C(S)R⁷, —C(S)NR⁷R⁸, —C(═NR⁷)R⁸, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN or —N₃;
R⁵ is selected from the group consisting of H, halo, alkyl, cycloalkyl, OR⁷, —S(O)_{f}R⁷, —NR⁷R⁸, —NHC(O)R⁷, —NHC(O)NR⁷R⁸ and —NHS(O)₂R⁷;
f is 0, 1 or 2; and
each R⁷ and each R⁸ are the same or different and are each independently selected from the group consisting of H, alkyl, alkenyl, alkynyl, cycloalkyl and cycloalkenyl; and
each R⁹ is the same or different and is selected from H, halo and alkyl;
or a pharmaceutically acceptable salt, solvate or physiologically functional derivative thereof.

44. An R-isomer of a compound according to claim 43.

45. An R-isomer of a compound selected from
5-(5-Amino-1H-benzimidazol-1-yl)-3-[1-(2-chlorophenyl)ethoxy]thiophene-2-carboxamide;
3-[1-(2-Chlorophenyl)ethoxy]-5-(5,6-dimethoxy-1H-benzimidazol-1-yl)thiophene-2-carboxamide;
5-(5,6-Dimethoxy-1H-benzimidazol-1-yl)-3-[1-(2-methylphenyl)ethoxy]thiophene-2-carboxamide;
and pharmaceutically acceptable salts, solvates and physiologically functional derivatives thereof.
